(19) United States
(12) Patent Application Publication
(10) Pub. No.: US 2009/0017460 A1
(43) Pub. Date: Jan. 15, 2009

(54) DIFFERENTIAL EXPRESSION PROFILING ANALYSIS OF CELL CULTURE PHENOTYPES AND USES THEREOF

(75) Inventors: Karin Anderson, Georgetown, MA (US); Niall Barron, Shankill (IE); Martin Clynes, Clontarf (IE); Dana L. Di Nino, North Andover, MA (US); Padraig Doolan, Swords (IE); Patrick Gammell, Naas (IE); Kathleen Kopycinski, Somerville, MA (US); Kevin M. McCarthy, North Andover, MA (US); Paula Meleady, Ratoath (IE); Mark Melville, Melrose, MA (US); Chee-keng Ng, Arlington, MA (US); Ryan Nolan, Stoneham, MA (US)

Correspondence Address: CHOATE, HALL & STEWART LLP, TWO INTERNATIONAL PLACE, BOSTON, MA, 02110, US

(73) Assignees: Wyeth, Madison, NJ (US); Dublin City University, Dublin (IE)

(21) Appl. No.: 12/139,294

(22) Filed: Jun. 13, 2008

Related U.S. Application Data
(60) Provisional application No. 60/934,980, filed on Jun. 15, 2007.
(60) Provisional application No. 61/016,390, filed on Dec. 21, 2007.

(51) Int. Cl. C12Q 1/68; C12N 5/10

(52) U.S. Cl. 435/6; 435/375; 435/325

(57) ABSTRACT

The present invention provides, among other things, systems and methods for identifying genes and proteins that regulate and/or are indicative of cell phenotypes based on expression profiling analysis. The present invention further provides methods of manipulating identified genes and proteins to engineer improved cell lines.

### RELATED APPLICATIONS

This application claims priority to and the benefit of U.S. Application No. 60/934,980, filed on Jun. 15, 2007, and U.S. Application No. 61/016,390, filed on Dec. 21, 2007, the contents of both of which are hereby incorporated by reference in their entireties. This application also relates to U.S. application Ser. No. 11/788,872 and PCT/US2007/10002, both filed on Apr. 21, 2007, the contents of both of which are incorporated by reference herein.

### REFERENCE TO SEQUENCE LISTING

This application includes as part of the originally filed subject matter a Sequence Listing filed electronically on even date herewith. The electronically-filed Sequence Listing is a single text file, which is named “WYE-061.5T25.txt” (456 KB). The contents of the electronically-filed Sequence Listing are hereby incorporated by reference in their entireties.

### BACKGROUND OF THE INVENTION

Fundamental to the present-day study of biology is the ability to optimally culture and maintain cell lines. Cell lines not only provide an in vitro model for the study of biological systems and diseases, but are also used to produce organic reagents. Of particular importance is the use of genetically engineered prokaryotic or eukaryotic cell lines to generate mass quantities of recombinant proteins. A recombinant protein may be used in a biological study, or as a therapeutic compound for treating a particular ailment or disease.

The production of recombinant proteins for biopharmaceutical application typically requires vast numbers of cells and/or particular cell culture conditions that influence cell growth and/or expression. In some cases, production of recombinant proteins benefits from the introduction of chemical inducing agents (such as sodium butyrate or valeric acid) to the cell culture medium. Identifying the genes and related genetic pathways that respond to the culture conditions (or particular agents) that increase transgene expression may elucidate potential targets that can be manipulated to increase recombinant protein production and/or influence cell growth.

Research into optimizing recombinant protein production has been primarily devoted to examining gene regulation, cellular responses, cellular metabolism, and pathways activated in response to unfolded proteins. Currently, there is no available method that allows for the simultaneous monitoring of transgene expression and identification of the genetic pathways involved in transgene expression. For example, currently available methods for detecting transgene expression include those that measure only the presence and amount of known proteins (e.g., Western blot analysis, enzyme-linked immunosorbent assay, and fluorescence-activated cell sorting), or the presence and amount of known messenger RNA (mRNA) transcripts (e.g., Northern blot analysis and reverse transcription-polymerase chain reaction). These and similar methods are not only limited in the number of known proteins and/or mRNA transcripts that can be detected at one time, but they also require that the investigator know or “guess” what genes are involved in transgene expression prior to experimentation (so that the appropriate antibodies or oligonucleotide probes are used). Another limitation inherent in blot analyses and similar protocols is that proteins or mRNA that are the same size cannot be distinguished. Considering the vast number of genes contained within a single genome, identification of even a minority of genes involved in a genetic pathway using the methods described above is costly and time-consuming. Additionally, the requirement that the investigator have some idea regarding which genes are involved does not allow for the identification of genes and related pathways that were either previously undiscovered or unknown to be involved in the regulation of transgene expression.

### SUMMARY OF THE INVENTION

The present invention provides, among other things, systems and methods of identifying genes, proteins and/or other factors that regulate or are indicative of cell phenotypes (e.g., industrially relevant cell phenotypes) based on genomic or proteomic analysis methods. The present invention further provides methods for manipulating identified genes and proteins to engineer improved cell lines. Therefore, the present invention represents a significant advance in cell engineering for improved cell lines and cell culture conditions.

In some embodiments, the present invention provides methods for identifying proteins or genes regulating or indicative of a cell phenotype of interest, typically, under a cell culture condition. Inventive methods include steps of (a) obtaining a first control sample from a control cell culture at a first time point and generating a first control expression profile of the first control sample; (b) obtaining a second control sample from the control cell culture at a second time point and generating a second control expression profile of the second control sample; (c) comparing the first control expression profile to the second control expression profile to identify one or more differentially expressed proteins or genes in the control cell culture; (d) obtaining a first test sample from a test cell culture at a first time point and generating a first test expression profile of the first test sample; (e) obtaining a second test sample from the test cell culture at a second time point and generating a second test expression profile of the second test sample; (f) comparing the first test expression profile to the second test expression profile to identify one or more differentially expressed proteins or genes in the test cell culture; and (g) comparing the one or more differentially expressed proteins or genes in the control cell culture to the one or more differentially expressed proteins or genes in the test cell culture to classify the one or more differentially expressed proteins or genes into control cell-only, test cell-only, and common differentially expressed proteins or genes, wherein the cell phenotype of interest or a change of the cell phenotype of interest over time in the test cell culture is distinct from that in the control cell culture. In some embodiments, common differently expressed proteins or genes are referred to as process-related genes or proteins. In some embodiments, the test and control cell cultures contain Chinese hamster ovary (CHO) cells. In some embodiments, the test and control cell cultures are grown under a fed batch condition. In some embodiments, the first time point is taken during an exponential growth phase and the second time point is taken during a lag phase.

In some embodiments, cell culture phenotypes that can be analyzed using methods of the invention are selected from cell growth rate, cellular productivity (such as maximum cellular productivity or sustained high cellular productivity), peak cell density, sustained cell viability, rate of ammonia production or consumption, rate of lactate production or consumption and combinations thereof.

In some embodiments, expression profiles in accordance with the present invention are protein expression profiles. In some embodiments, protein expression profiles in accordance with the invention are generated by fluorescent two-dimensional differential in-gel electrophoresis. In other embodiments, expression profiles in accordance with the present invention are gene expression profiles. In some embodiments, gene expression profiles in accordance with the invention are generated using gene microarrays.

The present invention provides, among other things, methods for improving cell lines by modulating, i.e., up-regulating or down-regulating, one or more differentially expressed proteins or genes identified according to methods described herein. In some embodiments, the present invention provides methods for improving cell lines by modulating, i.e., up-regulating or down-regulating, one or more control cell-only or test cell-only differentially expressed proteins or genes identified according to methods described herein. As used herein, “up-regulating” includes providing exogenous nucleic acids (e.g., over-expression constructs) encoding proteins or genes of interest or functional variants retaining relevant activity (such as, for example, mammalian homologs thereof (e.g., primate or rodent homologs)) or providing factors or molecules indirectly enhancing the expression or activity of proteins or genes of interest. As used herein, “down-regulating” includes disrupting (e.g., knocking-out) genes of interest by, for example, providing RNA interference constructs, or inhibitors or other factors indirectly inhibiting the expression or activity of proteins or genes of interest.

In some embodiments, the present invention provides methods for improving cell phenotypes (e.g., cellular productivity, cell density, cell viability, or cell growth rate) using methods as described herein. In one embodiment, the present invention provides methods for improving cell phenotypes (e.g., cellular productivity, cell density, cell viability, or cell growth rate) by up-regulating or down-regulating one or more genes or proteins selected from Tables 2, 3, 4, 5, 8, 9, 10, 12, 13, 14 and 15. In some embodiments, the present invention provides methods for improving cell lines by up-regulating or down-regulating one or more genes selected from Tables 12 and 13. In some embodiments, the present invention provides methods for improving cell lines by up-regulating or down-regulating one or more genes selected from Table 15.

In some embodiments, the present invention provides methods for evaluating cell culture phenotypes. Inventive methods include steps of (a) detecting a first expression level of at least one control cell-only or test cell-only differentially expressed protein or gene identified in accordance with the present invention at a time point taken during an exponential phase; (b) detecting a second expression level of said at least one control cell-only or test cell-only differentially expressed protein or gene at a time point taken during a lag phase; and (c) comparing the first expression level to the second expression level to evaluate cell phenotypes over time under a cell culture condition.

In some embodiments, the present invention provides methods for evaluating cell culture phenotypes by (a) detecting a first expression level of at least one protein or gene selected from Tables 2, 3, 4, 5, 8, 9, 10, 12, 13, 14 and 15 at a time point taken during an exponential phase; (b) detecting a second expression level of said at least one protein or gene at a time point taken during a lag phase; and (c) comparing the first expression level to the second expression level to evaluate cell phenotypes over time under a cell culture condition.

In some embodiments, the present invention provides engineered cell lines with improved cell phenotypes containing a population of engineered cells, each of which comprises an engineered construct up-regulating or down-regulating one or more differentially expressed proteins or genes identified according to various methods described herein. In particular, the present invention provides engineered cell lines with improved cell phenotypes containing a population of engineered cells, each of which comprises an engineered construct up-regulating or down-regulating one or more control cell-only or test cell-only differentially expressed proteins or genes according to various methods as described herein.

In some embodiments, the present invention provides engineered cell lines with improved cell phenotypes, each of which comprises an engineered construct up-regulating or down-regulating one or more proteins or genes selected from Tables 2, 3, 4, 5, 8, 9, 10, 12, 13, 14 and 15.

In some embodiments, the present invention provides engineered cell lines with improved peak cell density comprising a population of engineered cells, each of which comprises an engineered construct up-regulating or down-regulating one or more proteins or genes selected from Tables 2, 3, 4, 5, 8, 9 and 10.

In some embodiments, engineered constructs in accordance with the present invention are over-expression constructs or interfering RNA constructs.

In some embodiments, the invention provides methods for expression of proteins of interest using engineered cell lines as described herein. Inventive methods include steps of introducing into an engineered cell line described herein a nucleic acid encoding a protein of interest; and harvesting the protein of interest.

Among other things, the invention provides isolated genes or proteins involved with regulating or indicative of cell phenotypes of interest as described herein. The invention also provides genetically engineered expression vectors, host cells, and transgenic animals comprising nucleic acid molecules or proteins in accordance with the invention. The invention additionally provides inhibitory polynucleotides (e.g., antisense and interfering RNAs) to nucleic acid molecules identified herein or nucleic acids encoding proteins identified herein.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

FIG. 1 depicts an exemplary time course analysis between a test cell line and a control cell line. The test cell line maintains a high viability (high cell density) throughout the fed batch, while the control cell line declines in viability relatively early.

FIGS. 2A and 2B show graphical depictions of relative abundance of exemplary process-related spots.

FIGS. 3-1 through 3-48 illustrate sequence data and analysis for individual, differentially-expressed proteins.

FIG. 4 illustrates exemplary processing of miRNA.

FIG. 5 depicts the sequence of an exemplary miRNA, Cgr-mir-21.

FIG. 6 depicts an exemplary 2D gel map image of comparison of parent vs. B19 Day 10 samples. The indicated spots have been identified using MALDI-ToF mass spectrometry except in the cases of spots 3340 and 3349 which were identified using LC-MS/MS.

FIG. 7 depicts an exemplary target validation workflow.

FIG. 8 depicts exemplary overexpression assay outlines.

### DEFINITIONS

Antibody: The term “antibody” as used herein refers to an immunoglobulin molecule or an immunologically active portion of an immunoglobulin molecule, i.e., a molecule that contains an antigen binding site which specifically binds an antigen, such as a Fab or F(ab′)₂ fragment. In certain embodiments, an antibody is a typical natural antibody known to those of ordinary skill in the art, e.g., glycoprotein comprising four polypeptide chains: two heavy chains and two light chains. In certain embodiments, an antibody is a single-chain antibody. For example, in some embodiments, a single-chain antibody comprises a variant of a typical natural antibody wherein two or more members of the heavy and/or light chains have been covalently linked, e.g., through a peptide bond. In certain embodiments, a single-chain antibody is a protein having a two-polypeptide chain structure consisting of a heavy and a light chain, which chains are stabilized, for example, by interchain peptide linkers, which protein has the ability to specifically bind an antigen. In certain embodiments, an antibody is an antibody comprised only of heavy chains such as, for example, those found naturally in members of the Camelidae family, including llamas and camels (see, for example, U.S. Pat. Nos. 6,765,087 by Casterman et al., 6,015,695 by Casterman et al., 6,005,079 and by Casterman et al., each of which is incorporated by reference in its entirety). The terms “monoclonal antibodies” and “monoclonal antibody composition”, as used herein, refer to a population of antibody molecules that contain only one species of an antigen binding site and therefore usually interact with only a single epitope or a particular antigen. Monoclonal antibody compositions thus typically display a single binding affinity for a particular epitope with which they immunoreact. The terms “polyclonal antibodies” and “polyclonal antibody composition” refer to populations of antibody molecules that contain multiple species of antigen binding sites that interact with a particular antigen.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Batch culture: The term “batch culture” as used herein refers to a method of culturing cells in which all the components that will ultimately be used in culturing the cells, including the medium (see definition of “Medium” below) as well as the cells themselves, are provided at the beginning of the culturing process. A batch culture is typically stopped at some point and the cells and/or components in the medium are harvested and optionally purified.

Bioreactor: The term “bioreactor” as used herein refers to any vessel used for the growth of a mammalian cell culture. A bioreactor can be of any size so long as it is useful for the culturing of mammalian cells. Typically, such a bioreactor will be at least 1 liter and may be 10, 100, 250, 500, 1000, 2500, 5000, 8000, 10,000, 12,000 liters or more, or any volume in between. The internal conditions of the bioreactor, including, but not limited to pH, dissolved oxygen and temperature, are typically controlled during the culturing period. A bioreactor can be composed of any material that is suitable for holding mammalian cell cultures suspended in media under the culture conditions of the present invention, including glass, plastic or metal. The term “production bioreactor” as used herein refers to the final bioreactor used in the production of the protein of interest. The volume of the production bioreactor is typically at least 500 liters and may be 1000, 2500, 5000, 8000, 10,000, 12,000 liters or more, or any volume in between. One of ordinary skill in the art will be aware of and will be able to choose suitable bioreactors for use in practicing the present invention.

Cell density and high cell density: The term “cell density” as used herein refers to the number of cells present in a given volume of medium. The term “high cell density” as used herein refers to a cell density that exceeds 5×10⁶/mL, 1×10⁷/mL, 5×10⁷/mL, 1×10⁸/mL, 5×10⁸/mL, 1×10⁹/mL, 5×10⁹/mL, or 1×10¹⁰/mL.

Cellular productivity and sustained high cellular productivity: The term “cellular productivity” as used herein refers to the total amount of recombinantly expressed protein (e.g., polypeptides, antibodies, etc.) produced by a cell per unit time. In some embodiments, cellular productivity is typically expressed in picograms/cell/day or micrograms/million cells/day. The term sustained high cellular productivity as used herein refers to the ability of cells in culture to maintain a high cellular productivity (e.g., more than 10 picograms/cell/day, 20 picograms/cell/day, 30 picograms/cell/day, 40 picograms/cell/day, 50 picograms/cell/day, 60 picograms/cell/day, 70 picograms/cell/day, 80 picograms/cell/day, 90 picograms/cell/day, 100 picograms/cell/day) under a given set of cell culture conditions or experimental variations. In some embodiments, the term “cellular productivity” also refers to the total amount of recombinantly expressed protein (e.g., polypeptides, antibodies, etc.) produced by a mammalian cell culture in a given amount of medium volume. In that case, cellular productivity is typically expressed in milligrams of protein per milliliter of medium (mg/mL) or grams of protein per liter of medium (g/L) and the term sustained high cellular productivity refers to the ability of cells in culture to maintain a high cellular productivity (e.g., more than 5 g/L, 7.5 g/L, 10 g/L, 12.5 g/L, 15 g/L, 17.5 g/L, 20 g/L, 22.5 g/L, 25 g/L) under a given set of cell culture conditions or experimental variations.

Cell growth rate and high cell growth rate: The term “cell growth rate” as used herein refers to the rate of change in cell density expressed in “hr⁻¹” units as defined by the equation: (ln X2−ln X1)/(T2−T1) where X2 is the cell density (expressed in millions of cells per milliliter of culture volume) at time point T2 (in hours) and X1 is the cell density at an earlier time point T1. In some embodiments, the term “high cell growth rate” as used herein refers to a growth rate value that exceeds 0.023 hr⁻¹.

Cell viability and sustained high cell viability: The term “cell viability” as used herein refers to the ability of cells in culture to survive under a given set of culture conditions or experimental variations. The term as used herein also refers to that portion of cells which are alive at a particular time in relation to the total number of cells, living and dead, in the culture at that time. The term “sustained high cell viability” as used herein refers to the ability of cells in culture to maintain a high cell viability (e.g., more than 60%, 65%, 70%, 75%, 80%, 85%, 90%, 95%, 98%, 99% of the total number of cells that are alive) under a given set of cell culture conditions or experimental variations.

Control and test: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested or monitored) is applied or present (e.g., a test cell line or culture with a desirable phenotype). In the second experiment, the “control,” the variable being tested is not applied or present (e.g., a control cell line or culture that does not have the desirable phenotype). In some embodiments, a control is a historical control (i.e., of a test or assay performed previously, or an amount or result that is previously known). In some embodiments, a control is or comprises a printed or otherwise saved record. A control may be a positive control or a negative control.

Culture: The term “cell culture” as used herein refers to a cell population that is suspended in a medium (see definition of “Medium” below) under conditions suitable to survival and/or growth of the cell population. As will be clear to those of ordinary skill in the art, in certain embodiments, these terms as used herein refer to the combination comprising the cell population and the medium in which the population is suspended. In certain embodiments, the cells of the cell culture comprise mammalian cells.

Differential expression profiling: The term “differential expression profiling” as used herein refers to methods of comparing the gene or protein expression levels or patterns of two or more samples (e.g., test samples vs. control samples). In some embodiments, differential expression profiling is used to identify genes, proteins or other components that are differentially expressed. A gene or protein is differentially expressed if the difference in the expression level or pattern between two samples is statistically significant (i.e., the difference is not caused by random variations). In some embodiments, a gene or protein is differentially expressed if the difference in the expression level between two samples is more than 1.2-fold, 1.5-fold, 1.75-fold, 2-fold, 2.25-fold, 2.5-fold, 2.75-fold, or 3-fold.

Fed-batch culture: The term “fed-batch culture” as used herein refers to a method of culturing cells in which additional components are provided to the culture at a time or times subsequent to the beginning of the culture process. Such provided components typically comprise nutritional components for the cells which have been depleted during the culturing process. Additionally or alternatively, such additional components may include supplementary components (see definition of “Supplementary components” below). In certain embodiments, additional components are provided in a feed medium (see definition of “Feed medium” below). A fed-batch culture is typically stopped at some point and the cells and/or components in the medium are harvested and optionally purified.

Feed medium: The term “feed medium” as used herein refers to a solution containing nutrients which nourish growing mammalian cells that is added after the beginning of the cell culture. A feed medium may contain components identical to those provided in the initial cell culture medium. Alternatively, a feed medium may contain one or more additional components beyond those provided in the initial cell culture medium. Additionally or alternatively, a feed medium may lack one or more components that were provided in the initial cell culture medium. In certain embodiments, one or more components of a feed medium are provided at concentrations or levels identical or similar to the concentrations or levels at which those components were provided in the initial cell culture medium. In certain embodiments, one or more components of a feed medium are provided at concentrations or levels different than the concentrations or levels at which those components were provided in the initial cell culture medium.

Fragment: The term “fragment” as used herein refers to a polypeptide that is defined as any discrete portion of a given polypeptide that is unique to or characteristic of that polypeptide. For example, the term as used herein refers to any portion of a given polypeptide that includes at least an established sequence element found in the full-length polypeptide. In certain fragments, the sequence element spans at least 4-5, 10, 15, 20, 25, 30, 35, 40, 45, 50 or more amino acids of the full-length polypeptide. Alternatively or additionally, the term as used herein refers to any discrete portion of a given polypeptide that retains at least a fraction of at least one activity of the full-length polypeptide. In certain embodiments, the fraction of activity retained is at least 10% of the activity of the full-length polypeptide. In certain embodiments, the fraction of activity retained is at least 20%, 30%, 40%, 50%, 60%, 70%, 80% or 90% of the activity of the full-length polypeptide. In certain embodiments, the fraction of activity retained is at least 95%, 96%, 97%, 98% or 99% of the activity of the full-length polypeptide. In certain embodiments, the fragment retains 100% of more of the activity of the full-length polypeptide.

Gene: The term “gene” as used herein refers to any nucleotide sequence, DNA or RNA, at least some portion of which encodes a discrete final product, typically, but not limited to, a polypeptide, which functions in some aspect of cellular metabolism or development. Optionally, the gene comprises not only the coding sequence that encodes the polypeptide or other discrete final product, but also comprises regions preceding and/or following the coding sequence that modulate the basal level of expression (sometimes referred to as “genetic control element”), and/or intervening sequences (“introns”) between individual coding segments (“exons”).

Low ammonium producer: The term “low ammonium producer” as used herein refers to a metabolic characteristic of cells that results in a low net ammonium concentration (brought about through a balance between ammonium production and ammonium depletion) in the culture medium. In some embodiments, the term “low ammonium producer” refers to a metabolic characteristic of cells that results in a net ammonium concentration in the culture medium of <3.0 millimolar.

Low lactate producer: The term “low lactate producer” as used herein refers to a metabolic characteristic of cells that results in a low net lactic acid concentration (brought about through a balance between lactic acid production and lactic acid consumption) in the culture medium. In some embodiments, the term “low lactate producer” refers to a metabolic characteristic of cells that results in a net lactic acid concentration in the culture medium of <3.0 g/L.

Polypeptide: The term “polypeptide” as used herein refers a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Supplementary components: The term “supplementary components” as used herein refers to components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, and/or glucose or other energy source. In certain embodiments, supplementary components may be added to the initial cell culture. In certain embodiments, supplementary components may be added after the beginning of the cell culture.

“Titer”: The term “titer” as used herein refers to the total amount of recombinantly expressed protein (e.g., polypeptides, antibodies) produced by a mammalian cell culture in a given amount of medium volume. Titer is typically expressed in units of milligrams of protein per milliliter of medium or grams of protein per liter.

### DETAILED DESCRIPTION OF THE INVENTION

The present invention provides systems and methods for identifying genes and proteins regulating or indicative of cell culture phenotypes. Among other things, inventive methods of the present invention are based on differential expression profiling analysis using test and control cell lines or cultures that have distinct cell culture phenotypes.

Various aspects of the invention are described in further detail in the following subsections. The use of subsections is not meant to limit the invention. Each subsection may apply to any aspect of the invention. In this application, the use of “or” means “and/or” unless stated otherwise.

### Cell Lines and Cell Culture Phenotypes

The present invention contemplates differential expression profiling analysis and optimization of cell lines derived from a variety of organisms, including, but not limited to, bacteria, plants, fungi, and animals (the latter including, but not limited to, insects and mammals). For example, the present invention may be applied to Escherichia coli, Spodoptera frugiperda, Nicotiana sp., Zea mays, Lemna sp., Saccharomyces sp., Pichia sp., Schizosaccharomyces sp., mammalian cells, including, but not limited to, COS cells, CHO cells, 293 cells, A431 cells, 3T3 cells, CV-1 cells, HeLa cells, L cells, BHK21 cells, HL-60 cells, U937 cells, HEK cells, PerC6 cells, Jurkat cells, normal diploid cells, cell strains derived from in vitro culture of primary tissue, and primary explants. The list of organisms and cell lines are meant only to provide non-limiting examples.

In particular, the present invention contemplates differential expression profiling analysis of industrially relevant cell lines, such as, for example, CHO cells. As non-limiting examples, CHO cells are primary hosts for therapeutic protein productions because CHO cells provide fidelity of folding, processing, and glycosylation. For example, CHO cells are utilized to produce monoclonal antibodies, receptors, and fusion proteins (e.g., Fc fusion proteins). CHO cells are also compatible with deep-tank, serum-free cultures and have excellent safety records.

The present invention provides methods for identifying genes and proteins that influence desired cell culture phenotypes or characteristics, for example, cell phenotypes that enable highly productive fed-batch processes. Such desired cell phenotypes include, but are not limited to, high cell growth rate, high peak cell density, sustained high cell viability, high maximum cellular productivity, sustained high cellular productivity, low ammonium production, and low lactate production. Desired phenotypes or characteristics may be inherent properties of established cell lines that have certain genomic backgrounds. Desired phenotypes or characteristics may also be conferred to cells by growing the cells in different conditions, e.g., temperatures, cell densities, the use of agents such as sodium butyrate, to be in different kinetic phases of growth (e.g., lag phase, exponential growth phase, stationary phase or death phase), and/or to become serum-independent, etc. During the period in which these phenotypes are induced, and/or after these phenotypes are achieved, a pool of target nucleic acid or protein samples can be prepared from relevant cell samples and analyzed with, for example, oligonucleotide arrays or by proteomic assays to determine and identify which genes or proteins demonstrate altered expression in a desirable genomic background or in response to a particular stimulus (e.g., temperature, sodium butyrate), and therefore are potentially involved in conferring the desired phenotype or characteristic.

### Time Course Analysis

Cell phenotypes change over time under cell culture conditions. Without wishing to be bound by any theories, it is contemplated that the change of cell phenotypes may correlate with cell growth kinetics under a particular cell culture condition. For example, in the fed batch culture, cells undergo an initial phase of exponential growth. Typically, after several days, the culture temperature is lowered. Nutrient feeds are added to supplement growth and the cells are maintained for up to 14 days. At this time, the cells enter a lag phase, and in some cases, begin to decline in viability towards the end of the culture.

Inventive methods in accordance with the present invention identify proteins or genes regulating or indicative of cell phenotypes of cell cultures over time by examining the changes in gene or protein expression patterns over time. By observing these changes, we can gain an understanding of how a cell culture dynamically responds to its changing environment. In some embodiments, inventive methods of the present invention include a step of comparing at least one pair of different cell lines that display different growth profiles over a particular cell culture (e.g., fed batch culture) to each other. For example, one cell line (referred to as test cell line) maintains a high viability throughout the fed batch, while another cell line (referred to as control cell line) declines in viability relatively early. Replicate cultures of each cell line grown under similar fed batch conditions are sampled at multiple time points. Each is analyzed in order to characterize how the cells change their expression profiles over time. Differentially expressed proteins or genes are identified in each cell line. The differentially expressed proteins or genes in the test cell line are compared to the differentially expressed proteins or genes in the control cell line to classify the differentially expressed proteins or genes into three groups. The first group includes those that are unique to the test (e.g., high viability) cell line. The second group includes those unique to the control (e.g., low viability) cell line. The third group includes those in common between the two cell lines.

Each of the groups of differentially expressed genes or proteins provides insight into genetic backgrounds of cell lines and culture conditions. Those unique to the test cell line provide information regarding what may contribute to the ability of this cell line to maintain a desirable cell phenotype, for example, high viability. This group (test-only) of differentially expressed proteins or genes can be used to engineer cells to reproduce the desirable phenotype, or as indicate biomarkers to screen for or select the desirable phenotype. Conversely, those unique to the control cell line provide insights into what may contribute to a undesirable cell phenotype, for example, a decline in cell viability. This information can be used to engineer cells to avoid undesirable phenotypes, or as biomarkers to screen for or select against this phenotype. Finally, differentially expressed genes and proteins that are in common between the test and control cell lines provide insights into the process itself, that is, how cells generally respond to a cell culture condition, for example, a fed batch culture system. Therefore, those differentially expressed genes or proteins that are in common between control and test cell lines sometimes are referred to as process-related genes or proteins.

In some embodiments, the change of one or more cell phenotypes of interest over time of a test cell culture is distinct from that of a control cell culture. The test cell line (or test cell culture) and the control cell line (or control cell culture) can be different cell lines with different genetic background or similar cell lines with modified genetic background. For example, a test cell line can be generated by over-expressing a protein, a gene or an inhibitory RNA in a control cell line to induce a desirable cell phenotype. In some embodiments, the test cell culture and control cell culture have identical genetic background, but the test cell culture is cultured in a cell culture condition such that one or more desirable cell phenotypes are induced.

In some embodiments, inventive methods of the present invention include a step of comparing two or more pairs of different cell lines (or cell cultures) that display different growth profiles over time under a particular conditions (e.g., fed batch culture). For example, each pair may include two cell cultures, one displays high viability and the other displays low viability. Comparison of each pair (high viability vs. low viability) classifies differentially expressed proteins or genes into three categories of differentially expressed proteins or genes as described above (e.g., high viability-only, low viability-only, common or process-only). Differentially expressed proteins or genes in each group (for example, high viability-only) from one comparison can be further compared to the differentially expressed proteins or genes in a corresponding group from the comparison of another pair to identify genes commonly associated with high viability from different comparisons.

In some embodiments, samples from a test culture and a control culture can be taken from different time points. All control samples are pooled together and all test samples are pooled together for expression profiling analysis. Gene or protein expression profiles of pooled control samples and pooled test samples are compared against each other to identify differentially expressed genes and proteins.

Thus, the present invention provides methods to identify genes, proteins, and/or associated cellular and molecular pathways that regulate or are indicative of desirable cell phenotypes under any culture conditions of interest. The information provided by the present invention can be used to identify overarching limitations or bottlenecks in any particular culture condition, such as fed batch culture. This type of analysis also enables us to compare across cell culture paradigms or platforms to understand how the cells respond to different environments at the molecular level.

The differentially expressed genes or proteins identified by the present invention are candidate genes or proteins that regulate or are indicative of cell culture phenotypes of interest over time under a cell culture condition. The identified genes and proteins can be further confirmed and validated using methods described herein or known in the art (e.g., expression levels of candidate genes or proteins can be verified by Western blotting or Northern blotting). The identified genes or proteins may also be manipulated to improve relevant cell culture phenotypes of interest.

Inventive methods of the present invention can be used to analyze or optimize cell lines derived from a variety of organisms, including, but not limited to, bacteria, plants, fungi, and animals (the latter including, but not limited to, insects and mammals). For example, the present invention may be applied to Escherichia coli, Spodoptera frugiperda, Nicotiana sp., Zea mays, Lemna sp., Saccharomyces sp., Pichia sp., Schizosaccharomyces sp., mammalian cells, including, but not limited to, COS cells, CHO cells, 293 cells, A431 cells, 3T3 cells, CV-1 cells, HeLa cells, L cells, BHK21 cells, HL-60 cells, U937 cells, HEK cells, PerC6 cells, Jurkat cells, normal diploid cells, cell strains derived from in vitro culture of primary tissue, and primary explants. The list of organisms and cell lines are meant only to provide non-limiting examples.

In particular, the present invention contemplates differential expression profiling analysis of industrially relevant cell lines, such as, for example, CHO cells. CHO cells are a primary host for therapeutic protein production, such as, for example, monoclonal antibody production, receptor productions, and Fc fusion proteins because CHO cells provide fidelity of folding, processing, and glycosylation. CHO cells are also compatible with deep-tank, serum-free culture and have excellent safety records.

### Preparation of Pool of Target Nucleic Acids

In order to conduct gene expression profiling analysis, a pool of target nucleic acids are prepared from a sample derived from a cell line at a particular time point of cell culture. Any biological sample may be used as a source of target nucleic acids. The pool of target nucleic acids can be total RNA, or any nucleic acid derived therefrom, including each of the single strands of cDNA made by reverse transcription of the mRNA, or RNA transcribed from the double-stranded cDNA intermediate. Methods of isolating target nucleic acids for analysis with an oligonucleotide array or other probes, such as phenol-chloroform extraction, ethanol precipitation, magnetic bead separation, or silica-gel affinity purification, are well known to one of skill in the art.

For example, various methods are available for isolating or enriching RNA. These methods include, but are not limited to, RNeasy kits (provided by Qiagen), MasterPure kits (provided by Epicentre Technologies), charge-switch technology (see, e.g., U.S. Published patent application Nos. 2003/0054395 and 2003/0130499), and TRIZOL (provided by Gibco BRL). The RNA isolation protocols provided by Affymetrix can also be employed in the present invention. See, e.g., GeneChip® EXPRESSION ANALYSIS TECHNICAL MANUAL (701021 rev. 3, Affymetrix, Inc. 2002).

Preferably, the pool of target nucleic acids (i.e., mRNA or nucleic acids derived therefrom) should reflect the transcription of gene coding regions. In one example, mRNA is enriched by removing rRNA. Different methods are available for eliminating or reducing the amount of rRNA in a sample. For instance, rRNA can be removed by enzyme digestions. According to the latter method, rRNAs are first amplified using reverse transcriptase and specific primers to produce cDNA. The rRNA is allowed to anneal with the cDNA. The sample is then treated with RNAase H, which specifically digests RNA within an RNA:DNA hybrid.

Target nucleic acids may be amplified before incubation with an oligonucleotide array or other probes. Suitable amplification methods, including, but not limited to, reverse transcription-polymerase chain reaction, ligase chain reaction, self-sustained sequence replication, and in vitro transcription, are well known in the art. It should be noted that oligonucleotide probes are chosen to be complementary to target nucleic acids. Therefore, if an antisense pool of target nucleic acids is provided (as is often the case when target nucleic acids are amplified by in vitro transcription), the oligonucleotide probes should correspond with subsequences of the sense complement. Conversely, if the pool of target nucleic acids is sense, the oligonucleotide array should be complementary (i.e., antisense) to them. Finally, if target nucleic acids are double-stranded, oligonucleotide probes can be sense or antisense.

The present invention involves detecting the hybridization intensity between target nucleic acids and complementary oligonucleotide probes. To accomplish this, target nucleic acids may be attached directly or indirectly with appropriate and detectable labels. Direct labels are detectable labels that are directly attached to or incorporated into target nucleic acids. Indirect labels are attached to polynucleotides after hybridization, often by attaching to a binding moiety that was attached to the target nucleic acids prior to hybridization. Such direct and indirect labels are well known in the art. In a preferred embodiment of the invention, target nucleic acids are detected using the biotin-streptavidin-PE coupling system, where biotin is incorporated into target nucleic acids and hybridization is detected by the binding of streptavidin-PE to biotin.

Target nucleic acids may be labeled before, during or after incubation with an oligonucleotide array. Preferably, the target nucleic acids are labeled before incubation. Labels may be incorporated during the amplification step by using nucleotides that are already labeled (e.g., biotin-coupled dUTP or dCTP) in the reaction. Alternatively, a label may be added directly to the original nucleic acid sample (e.g., mRNA, cDNA) or to the amplification product after the amplification is completed. Means of attaching labels to nucleic acids are well known to those of skill in the art and include, but are not limited to, nick translation, end-labeling, and ligation of target nucleic acids to a nucleic acid linker to join it to a label. Alternatively, several kits specifically designed for isolating and preparing target nucleic acids for microarray analysis are commercially available, including, but not limited to, the GeneChip® IVT Labeling Kit (Affymetrix, Santa Clara, Calif.) and the Bioarray™ High Yield™ RNA Transcript Labeling Kit with Fluorescein-UTP for Nucleic Acid Arrays (Enzo Life Sciences, Inc., Farmingdale, N.Y.).

Polynucleotides can be fragmented before being labeled with detectable moieties. Exemplary methods for fragmentation include, but are not limited to, heat or ion-mediated hydrolysis.

### Oligonucleotide Arrays

Probes suitable for the present invention includes oligonucleotide arrays or other probes that capable of detecting the expression of a plurality of genes (including previously undiscovered genes) by a cell (or cell line), including known cells or cells derived from an unsequenced organism, and to identify genes (including previously undiscovered genes) and related pathways that may be involved with the induction of a particular cell phenotype, e.g., increased and efficient transgene expression.

Oligonucleotide probes used in this invention may be nucleotide polymers or analogs and modified forms thereof such that hybridizing to a pool of target nucleic acids occurs in a sequence specific manner under oligonucleotide array hybridization conditions. As used herein, the term “oligonucleotide array hybridization conditions” refers to the temperature and ionic conditions that are normally used in oligonucleotide array hybridization. In many examples, these conditions include 16-hour hybridization at 45° C., followed by at least three 10-minute washes at room temperature. The hybridization buffer comprises 100 mM MES, 1 M [Na⁺], 20 mM EDTA, and 0.01% Tween 20. The pH of the hybridization buffer can range between 6.5 and 6.7. The wash buffer is 6×SSPET, which contains 0.9 M NaCl, 60 mM NaH2PO4, 6 mM EDTA, and 0.005% Triton X-100. Under more stringent oligonucleotide array hybridization conditions, the wash buffer can contain 100 mM MES, 0.1 M [Na⁺], and 0.01% Tween 20. See also GENECHIP® EXPRESSION ANALYSIS TECHNICAL MANUAL (701021 rev. 3, Affymetrix, Inc. 2002), which is incorporated herein by reference in its entirety.

As is known by one of skill in the art, oligonucleotide probes can be of any length. Preferably, oligonucleotide probes suitable for the invention are 20 to 70 nucleotides in length. Most preferably, suitable oligonucleotide probes are 25 nucleotides in length. In one embodiment, the nucleic acid probes of the present invention have relatively high sequence complexity. In many examples, the probes do not contain long stretches of the same nucleotide. In addition, the probes may be designed such that they do not have a high proportion of G or C residues at the 3′ ends. In another embodiment, the probes do not have a 3′ terminal T residue. Depending on the type of assay or detection to be performed, sequences that are predicted to form hairpins or interstrand structures, such as “primer dimers,” can be either included in or excluded from the probe sequences. In many embodiments, each probe employed in the present invention does not contain any ambiguous base.

Oligonucleotide probes are made to be specific for (e.g., complementary to (i.e., capable of hybridizing to)) a template sequence. Any part of a template sequence can be used to prepare probes. Multiple probes, e.g., 5, 10, 15, 20, 25, 30, or more, can be prepared for each template sequence. These multiple probes may or may not overlap each other. Overlap among different probes may be desirable in some assays. In many embodiments, the probes for a template sequence have low sequence identities with other template sequences, or the complements thereof. For instance, each probe for a template sequence can have no more than 70%, 60%, 50% or less sequence identity with other template sequences, or the complements thereof. This reduces the risk of undesired cross-hybridization. Sequence identity can be determined using methods known in the art. These methods include, but are not limited to, BLASTN, FASTA, and FASTDB. The Genetics Computer Group (GCG) program, which is a suite of programs including BLASTN and FASTA, can also be used. Preferable sequences for template sequences include, but are not limited to, consensus sequences, transgene sequences, and control sequences (i.e., sequences used to control or normalize for variation between experiments, samples, stringency requirements, and target nucleic acid preparations). Additionally, any subsequence of consensus, transgene and control sequences can be used as a template sequence.

In one embodiment, only certain regions (i.e., tiling regions) of consensus, transgene and control sequences are used as template sequences for the oligonucleotide probes used in this invention. One of skill in the art will recognize that protocols that may be used in practicing the invention, e.g., in vitro transcription protocols, often result in a bias toward the 3′-ends of target nucleic acids. Consequently, in one embodiment of the invention, the region of the consensus sequence or transgene sequence closest to the 3′-end of a consensus sequence is most often used as a template for oligonucleotide probes. Generally, if a poly-A signal could be identified, the 1400 nucleotides immediately prior to the end of the consensus or transgene sequences are designated as a tiling region. Alternatively, if a poly-A signal could not be identified, only the last 600 nucleotides of the consensus or transgene sequence are designated as a tiling region. However, it should be noted that the invention is not limited to using only these tiling regions within the consensus, transgene and control sequences as templates for the oligonucleotide probes. Indeed, a tiling region may occur anywhere within the consensus, transgene or control sequences. For example, the tiling region of a control sequence may comprise regions from both the 5′ and 3′-ends of the control sequence. In fact, the entire consensus, transgene or control sequence may be used as a template for oligonucleotide probes.

An oligonucleotide array suitable for the invention may include perfect match probes to a plurality of consensus sequences (i.e., consensus sequences for multi-sequence clusters, and consensus sequences for exemplar sequences) identified as described above. The oligonucleotide array suitable for the invention may also include perfect match probes to both consensus and transgene sequences. It will be apparent to one of skill in the art that inclusion of oligonucleotide probes to transgene sequences will be useful when a cell line is genetically engineered to express a recombinant protein encoded by a transgene sequence, and the purpose of the analysis is to confirm expression of the transgene and determine the level of such expression. In those cases where the transgene is linked in a bicistronic mRNA to a downstream ORF, such as dihydrofolate reductase (DHFR), the level of transgene expression may also be determined from the level of expression of the downstream sequence. In another embodiment of the invention, the oligonucleotide array further comprises control probes that normalize the inherent variation between experiments, samples, stringency requirements, and preparations of target nucleic acids. Exemplary compositions of each of these types of control probes are described in U.S. Pat. No. 6,040,138 and in U.S. Publication No. 20060010513, the teachings of both of which are incorporated herein in their entirety by reference.

It is well known to one of skill in the art that two pools of target nucleic acids individually processed from the same sample can hybridize to two separate but identical oligonucleotide arrays with varying results. The varying results between these arrays are attributed to several factors, such as the intensity of the labeled pool of target nucleic acids and incubation conditions. To control for these variations, normalization control probes can be added to the array. Normalization control probes are oligonucleotides exactly complementary to known nucleic acid sequences spiked into the pool of target nucleic acids. Any oligonucleotide sequence may serve as a normalization control probe. For example, the normalization control probes may be created from a template obtained from an organism other than that from which the cell line being analyzed is derived. In one embodiment, an oligonucleotide array to mammalian sequences will contain normalization oligonucleotide probes to the following genes: bioB, bioC, and bioD from the organism Escherichia coli, cre from the organism Bacteriophage PI, and dap from the organism Bacillus subtilis, or subsequences thereof. The signal intensity received from the normalization control probes are then used to normalize the signal intensities from all other probes in the array. Additionally, when the known nucleic acid sequences are spiked into the pool of target nucleic acids at known and different concentrations for each transcript, a standard curve correlating signal intensity with transcript concentration can be generated, and expression levels for all transcripts represented on the array can be quantified (see, e.g., Hill et al. (2001) Genome Biol. 2(12):research0055.1-0055.13).

Due to the naturally differing metabolic states between cells, expression of specific target nucleic acids varies from sample to sample. In addition, target nucleic acids may be more prone to degradation in one pool compared to another pool. Consequently, in another embodiment of the invention, the oligonucleotide array further comprises oligonucleotide probes that are exactly complementary to constitutively expressed genes, or subsequences thereof, that reflect the metabolic state of a cell. Non-limiting examples of these types of genes are beta-actin, transferrin receptor and glyceraldehyde-3-phosphate dehydrogenase (GAPDH).

In one embodiment of the invention, the pool of target nucleic acids is derived by converting total RNA isolated from the sample into double-stranded cDNA and transcribing the resulting cDNA into complementary RNA (cRNA) using methods described in U.S. Publication No. 20060010513, the teachings of which are incorporated herein in their entirety by reference. The RNA conversion protocol is started at the 3′-end of the RNA transcript, and if the process is not allowed to go to completion (if, for example, the RNA is nicked, etc.) the amount of the 3′-end message compared to the 5′-end message will be greater, resulting in a 3′-bias. Additionally, RNA degradation may start at the 5′-end (Jacobs Anderson et al. (1998) EMBO J. 17: 1497-506). The use of these methods suggests that control probes that measure the quality of the processing and the amount of degradation of the sample preferably should be included in the oligonucleotide array. Examples of such control probes are oligonucleotides exactly complementary to 3′- and 5′-ends of constitutively expressed genes, such as beta-actin, transferrin receptor and GAPDH, as mentioned above. The resulting 3′ to 5′ expression ratio of a constitutively expressed gene is then indicative of the quality of processing and the amount of degradation of the sample; i.e., a 3′ to 5′ ratio greater than three (3) indicates either incomplete processing or high RNA degradation (Auer et al. (2003) Nat. Genet. 35:292-93). Consequently, in a preferred embodiment of the invention, the oligonucleotide array includes control probes that are complementary to the 3′- and 5′-ends of constitutively expressed genes.

The quality of the pools of target nucleic acids is not only reflected in the processing and degradation of the target nucleic acids, but also in the origin of the target nucleic acids. Contaminating sequences, such as genomic DNA, may interfere with well-known quantification protocols. Consequently, in a preferred embodiment of the invention, the array further comprises oligonucleotide probes exactly complementary to bacterial genes, ribosomal RNAs, and/or genomic intergenic regions to provide a means to control for the quality of the sample preparation. These probes control for the possibility that the pool of target nucleic acids is contaminated with bacterial DNA, non-mRNA species, and genomic DNA. Such exemplary control sequences are disclosed in U.S. Publication No. 20060010513, the teaching of which are incorporated herein in their entirety by reference.

In some embodiments of the invention, the oligonucleotide array further comprises control mismatch oligonucleotide probes for each perfect match probe. The mismatch probes control for hybridization specificity. Preferably, mismatch control probes are identical to their corresponding perfect match probes with the exception of one or more substituted bases. More preferably, the substitution(s) occurs at a central location on the probe. For example, where a perfect match probe is 25 oligonucleotides in length, a corresponding mismatch probe will have the identical length and sequence except for a single-base substitution at position 13 (e.g., substitution of a thymine for an adenine, an adenine for a thymine, a cytosine for a guanine, or a guanine for a cytosine). The presence of one or more mismatch bases in the mismatch oligonucleotide probe disallows target nucleic acids that bind to complementary perfect match probes to bind to corresponding mismatch control probes under appropriate conditions. Therefore, mismatch oligonucleotide probes indicate whether the incubation conditions are optimal, i.e., whether the stringency being utilized provides for target nucleic acids binding to only exactly complementary probes present in the array.

For each template, a set of perfect match probes exactly complementary to subsequences of consensus, transgene, and/or control sequences (or tiling regions thereof) may be chosen using a variety of strategies. It is known to one of skill in the art that each template can provide for a potentially large number of probes. As is known, apparent probes are sometimes not suitable for inclusion in the array. This can be due to the existence of similar subsequences in other regions of the genome, which causes probes directed to these subsequences to cross-hybridize and give false signals. Another reason some apparent probes may not be suitable for inclusion in the array is because they may form secondary structures that prevent efficient hybridization. Finally, hybridization of target nucleic acids with (or to) an array comprising a large number of probes requires that each of the probes hybridizes to its specific target nucleic acid sequence under the same incubation conditions.

An oligonucleotide array may comprise one perfect match probe for a consensus, transgene, or control sequence, or may comprise a probeset (i.e., more than one perfect match probe) for a consensus, transgene, or control sequence. For example, an oligonucleotide array may comprise 1, 5, 10, 25, 50, 100, or more than 100 different perfect match probes for a consensus, transgene or control sequence. In a preferred embodiment of the invention, the array comprises at least 11-150 different perfect match oligonucleotide probes exactly complementary to subsequences of each consensus and transgene sequence. In an even more preferred embodiment, only the most optimal probeset for each template is included. The suitability of the probes for hybridization can be evaluated using various computer programs. Suitable programs for this purpose include, but are not limited to, LaserGene (DNAStar), Oligo (National Biosciences, Inc.), MacVector (Kodak/IBI), and the standard programs provided by the GCG. Any method or software program known in the art may be used to prepare probes for the template sequences of the present invention. For example, oligonucleotide probes may be generated by using Array Designer, a software package provided by TeleChem International, Inc (Sunnyvale, Calif.). Another exemplary algorithm for choosing optimal probe sets is described in U.S. Pat. No. 6,040,138, the teachings of which are hereby incorporated by reference. Other suitable means to optimize probesets, which will result in a comparable oligonucleotide array, are well known in the art and may be found in, e.g., Lockhart et al. (1996) Nat. Biotechnol. 14:1675-80 and Mei et al. (2003) Proc. Natl. Acad. Sci. USA 100:11237-42.

The oligonucleotide probes of the present invention can be synthesized using a variety of methods. Examples of these methods include, but are not limited to, the use of automated or high throughput DNA synthesizers, such as those provided by Millipore, GeneMachines, and BioAutomation. In many embodiments, the synthesized probes are substantially free of impurities. In many other embodiments, the probes are substantially free of other contaminants that may hinder the desired functions of the probes. The probes can be purified or concentrated using numerous methods, such as reverse phase chromatography, ethanol precipitation, gel filtration, electrophoresis, or any combination thereof.

More detailed information of making an oligonucleotide array suitable for the present invention and exemplary arrays are disclosed in U.S. Publication No. 20060010513, the disclosures of which are hereby incorporated by reference. As described in U.S. Publication No. 20060010513, a CHO chip microarray suitable for the invention includes 122 array quality control sequences (non-CHO), 732 public hamster sequences, 2835 library-derived CHO sequences, and 22 product/process specific sequences. Additional suitable arrays are described in U.S. Pat. No. 6,040,138, the disclosures of which are incorporated by reference. Exemplary microarrays suitable for the invention include, but are not limited to, Affymetrix Custom CHO chip (M. Melville et al. CCE-IX. 2004) which contain 3567 CHO sequences (partial coverage of the CHO transcriptome).

Incubation of Target Nucleic Acids with an Array to Form a Hybridization Profile

Incubation reactions can be performed in absolute or differential hybridization formats. In the absolute hybridization format, polynucleotides derived from one sample are hybridized to the probes in an oligonucleotide array. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to an oligonucleotide array. The oligonucleotide array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

In the present invention, the incubation conditions should be such that target nucleic acids hybridize only to oligonucleotide probes that have a high degree of complementarity. In a preferred embodiment, this is accomplished by incubating the pool of target nucleic acids with an oligonucleotide array under a low stringency condition to ensure hybridization, and then performing washes at successively higher stringencies until the desired level of hybridization specificity is reached. In other embodiments, target nucleic acids are incubated with an array of the invention under stringent or well-known oligonucleotide array hybridization conditions. In many examples, these oligonucleotide array hybridization conditions include 16-hour hybridization at 45° C., followed by at least three 10-minute washes at room temperature. The hybridization buffer comprises 100 mM MES, 1 M [Na⁺], 20 mM EDTA, and 0.01% Tween 20. The pH of the hybridization buffer can range between 6.5 and 6.7. The wash buffer is 6×SSPET, which contains 0.9 M NaCl, 60 mM NaH₂PO₄, 6 mM EDTA, and 0.005% Triton X-100. Under more stringent oligonucleotide array hybridization conditions, the wash buffer can contain 100 mM MES, 0.1 M [Na⁺], and 0.01% Tween 20. See also GENECHIP® EXPRESSION ANALYSIS TECHNICAL MANUAL (701021 rev. 3, Affymetrix, Inc. 2002), which is incorporated herein by reference in its entirety.

TABLE 1
Stringency Conditions

| | Poly- | Hybrid | Hybridization | |
|---|---|---|---|---|
| Stringency | nucleotide | Length | Temperature and | Wash Temp. |
| Condition | Hybrid | (bp)¹ | Buffer^{H} | and Buffer^{H} |
| A | DNA:DNA | >50 | 65° C.; 1xSSC -or- | 65° C.; |
| | | | 42° C.; 1xSSC, 50% | 0.3xSSC |
| | | | formamide | |
| B | DNA:DNA | <50 | T_{B}*; 1xSSC | T_{B}*; 1xSSC |
| C | DNA:RNA | >50 | 67° C.; 1xSSC -or- | 67° C.; |
| | | | 45° C.; 1xSSC, 50% | 0.3xSSC |
| | | | formamide | |
| D | DNA:RNA | <50 | T_{D}*; 1xSSC | T_{D}*; 1xSSC |
| E | RNA:RNA | >50 | 70° C.; 1xSSC -or- | 70° C.; |
| | | | 50° C.; 1xSSC, 50% | 0.3xSSC |
| | | | formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}*; 2xSSC | T_{L}*; 2xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
^{H}SSPE (1x SSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers.
T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀[Na⁺]) + 0.41(% G + C) − (600/N), where N is the number of bases in the hybrid, and [Na⁺] is the molar concentration of sodium ions in the hybridization buffer ([Na⁺] for 1x SSC = 0.165 M).

### Differential Gene Expression Profiling Analysis

Methods used to detect the hybridization profile of target nucleic acids with oligonucleotide probes are well known in the art. In particular, means of detecting and recording fluorescence of each individual target nucleic acid-oligonucleotide probe hybrid have been well established and are well known in the art, described in, e.g., U.S. Pat. No. 5,631,734, U.S. Publication No. 20060010513, incorporated herein in their entirety by reference. For example, a confocal microscope can be controlled by a computer to automatically detect the hybridization profile of the entire array. Additionally, as a further nonlimiting example, the microscope can be equipped with a phototransducer attached to a data acquisition system to automatically record the fluorescence signal produced by each individual hybrid.

It will be appreciated by one of skill in the art that evaluation of the hybridization profile is dependent on the composition of the array, i.e., which oligonucleotide probes were included for analysis. For example, where the array includes oligonucleotide probes to consensus sequences only, or consensus sequences and transgene sequences only, (i.e., the array does not include control probes to normalize for variation between experiments, samples, stringency requirements, and preparations of target nucleic acids), the hybridization profile is evaluated by measuring the absolute signal intensity of each location on the array. Alternatively, the mean, trimmed mean (i.e., the mean signal intensity of all probes after 2-5% of the probesets with the lowest and highest signal intensities are removed), or median signal intensity of the array may be scaled to a preset target value to generate a scaling factor, which will subsequently be applied to each probeset on the array to generate a normalized expression value for each gene (see, e.g., Affymetrix (2000) Expression Analysis Technical Manual, pp. A5-14). Conversely, where the array further comprises control oligonucleotide probes, the resulting hybridization profile is evaluated by normalizing the absolute signal intensity of each location occupied by a test oligonucleotide probe by means of mathematical manipulations with the absolute signal intensity of each location occupied by a control oligonucleotide probe. Typical normalization strategies are well known in the art, and are included, for example, in U.S. Pat. No. 6,040,138 and Hill et al. (2001) Genome Biol. 2(12):research0055.1-0055.13.

Signals gathered from oligonucleotide arrays can be analyzed using commercially available software, such as those provide by Affymetrix or Agilent Technologies. Controls, such as for scan sensitivity, probe labeling and cDNA or cRNA quantitation, may be included in the hybridization experiments. The array hybridization signals can be scaled or normalized before being subjected to further analysis. For instance, the hybridization signal for each probe can be normalized to take into account variations in hybridization intensities when more than one array is used under similar test conditions. Signals for individual target nucleic acids hybridized with complementary probes can also be normalized using the intensities derived from internal normalization controls contained on each array. In addition, genes with relatively consistent expression levels across the samples can be used to normalize the expression levels of other genes.

To identify genes that confer or correlate with a desired phenotype or characteristic, a gene expression profile of a sample derived from a test cell line is compared to a control profile derived from a control cell line that has a cell culture phenotype of interest distinct from that of the test cell line and differentially expressed genes are identified. For example, methods for identifying the genes and related pathways involved in cellular productivity may include the following: 1) growing a first sample of a first cell line with a particular cellular productivity and growing a second sample of a second cell line with a distinct cellular productivity; 2) isolating, processing, and hybridizing total RNA from the first sample to a first oligonucleotide array; 3) isolating, processing, and hybridizing total RNA from the second sample to a second oligonucleotide array; and 4) comparing the resulting hybridization profiles to identify the sequences that are differentially expressed between the first and second samples. Similar methods can be used to identify genes involved in other phenotypes.

Typically, each cell line was represented by at least three biological replicates. Programs known in the art, e.g., GeneExpress 2000 (Gene Logic, Gaithersburg, Md.), were used to analyze the presence or absence of a target sequence and to determine its relative expression level in one cohort of samples (e.g., cell line or condition or time point) compared to another sample cohort. A probeset called present in all replicate samples was considered for further analysis. Generally, fold-change values of 1.2-fold, 1.5-fold or greater were considered statistically significant if the p-values were less than or equal to 0.05.

The identification of differentially expressed genes that correlate with one or more particular cell phenotypes (e.g., cell growth rate, peak cell density, sustained high cell viability, maximum cellular productivity, sustained high cellular productivity, ammonium production or consumption, lactate production or consumption, etc.) can lead to the discovery of genes and pathways, including those which were previously undiscovered, that regulate or are indicative of the cell phenotypes.

The subsequently identified genes are sequenced and the sequences are blasted against various databases to determine whether they are known genes or unknown genes. If genes are known, pathway analysis can be conducted based on the existing knowledge in the art. Both known and unknown genes are further confirmed or validated by various methods known in the art. For example, the identified genes may be manipulated (e.g., up-regulated or down-regulated) to induce or suppress the particular phenotype by the cells.

More detailed identification and validation steps are further described in the Examples section.

### Differential Protein Expression Profiling Analysis

The present invention also provides methods for identifying differentially expressed proteins by protein expression profiling analysis. Protein expression profiles can be generated by any method permitting the resolution and detection of proteins from a cell culture sample. Methods with higher resolving power are generally preferred, as increased resolution can permit the analysis of greater numbers of individual proteins, increasing the power and usefulness of the profile. A sample can be pre-treated to remove abundant proteins from a sample, such as by immunodepletion, prior to protein resolution and detection, as the presence of an abundant protein may mask more subtle changes in expression of other proteins, particularly for low-abundance proteins. A sample can also be subjected to one or more procedures to reduce the complexity of the sample. For example, chromatography can be used to fractionate a sample; each fraction would have a reduced complexity, facilitating the analysis of the proteins within the fractions.

Useful methods for simultaneously resolving and detecting several proteins include, but are not limited to, array-based methods; mass-spectrometry based methods; and two-dimensional gel electrophoresis based methods. Exemplary specific methods include, but are not limited to, 2-D DIGE (Differential In-Gel Electrophoresis), Typhoon™ variable mode imager, DeCyder™ Differential Analysis Software, Automated gel spot picker, MALDI-TOF analysis, and LC MS/MS analysis.

Protein arrays generally involve a significant number of different protein capture reagents, such as antibodies or antibody variable regions, each immobilized at a different location on a solid support. Such arrays are available, for example, from Sigma-Aldrich as part of their Panorama™ line of arrays. The array is exposed to a protein sample and the capture reagents selectively capture the specific protein targets. The captured proteins are detected by detection of a label. For example, the proteins can be labeled before exposure to the array; detection of a label at a particular location on the array indicates the detection of the corresponding protein. If the array is not saturated, the amount of label detected may correlate with the concentration or amount of the protein in the sample. Captured proteins can also be detected by subsequent exposure to a second capture reagent, which can itself be labeled or otherwise detected, as in a sandwich immunoassay format.

Mass spectrometry-based methods include, for example, matrix-assisted laser desorption/ionization (MALDI), Liquid Chromatography/Mass Spectrometry/Mass Spectrometry (LC-MS/MS) and surface enhanced laser desorption/ionization (SELDI) techniques. For example, a protein profile can be generated using electrospray ionization and MALDI. SELDI, as described, for example, in U.S. Pat. No. 6,225,047, incorporates a retention surface on a mass spectrometry chip. A subset of proteins in a protein sample are retained on the surface, reducing the complexity of the mixture. Subsequent time-of-flight mass spectrometry generates a “fingerprint” of the retained proteins.

In methods involving two-dimensional gel electrophoresis, proteins in a sample are generally separated in a first dimension by isoelectric point and in a second dimension by molecular weight during SDS-PAGE. By virtue of the two dimensions of resolution, hundreds or thousands of proteins can be simultaneously resolved and analyzed. The proteins are detected by application of a stain, such as a silver stain, or by the presence of a label on the proteins, such as a Cy2, Cy3, or Cy5 dye. To identify a protein, a gel spot can be cut out and in-gel tryptic digestion performed. The tryptic digest can be analyzed by mass spectrometry, such as MALDI. The resulting mass spectrum of peptides, the peptide mass fingerprint or PMF, is searched against a sequence database. The PMF is compared to the masses of all theoretical tryptic peptides generated in silico by the search program. Programs such as Prospector, Sequest, and MasCot (Matrix Science, Ltd., London, UK) can be used for the database searching. For example, MasCot produces a statistically-based Mowse score indicates if any matches are significant or not. MS/MS can be used to increase the likelihood of getting a database match. CID-MS/MS (collision induced dissociation of tandem MS) of peptides can be used to give a spectrum of fragment ions that contain information about the amino acid sequence. Adding this information to a peptide mass fingerprint allows Mascot to increase the statistical significance of a match. It is also possible in some cases to identify a protein by submitting only a raw MS/MS spectrum of a single peptide.

A recent improvement in comparisons of protein expression profiles involves the use of a mixture of two or more protein samples, each labeled with a different, spectrally-resolvable, charge- and mass-matched dye, such as Cy3 and Cy5. This improvement, called fluorescent 2-dimensional differential in-gel electrophoresis (DIGE), has the advantage that the test and control protein samples are run in the same gel, facilitating the matching of proteins between the two samples and avoiding complications involving non-identical electrophoresis conditions in different gels. The gels are imaged separately and the resulting images can be overlaid directly without further modification. A third spectrally-resolvable dye, such as Cy2, can be used to label a pool of protein samples to serve as an internal control among different gels run in an experiment. Thus, all detectable proteins are included as an internal standard, facilitating comparisons across different gels.

### Engineering Cell Lines to Improve Cell Phenotypes

As described above, the present invention provides polynucleotide sequences (or subsequences) of genes or polypeptide sequences (or subsequences) of proteins that are differentially expressed in different cell lines or cell cultures with at least one distinct cell phenotype, including distinct change of cell phenotype overtime (see, e.g., Tables 2-15). These sequences are collectively referred to as differential sequences. Differential sequences in accordance with the present invention include purified or isolated sequences referenced to in relevant Tables described herein, or fragments or complements thereof. Differential sequences in accordance with the present invention also include sequence variants having 70-100%, including 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100%, sequence identity to the polynucleotide or polypeptide sequences referenced in Tables 2-15. Suitable variants generally share common structural features with the protein or gene of interest and should retain the activity permitting the improved cellular phenotype.

“Percent (%) nucleic acid sequence identity” with respect to the differential sequences identified herein is defined as the percentage of nucleotides in a candidate sequence that are identical with the nucleotides in relevant differential nucleotide sequences, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology, 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and are established by the program itself, depending upon the composition of the particular sequence, however, the minimum values may be adjusted and are set as indicated above.

“Percent (%) amino acid sequence identity” with respect to relevant differential polypeptide sequences identified herein is defined as the percentage of amino acid residues in a candidate sequence that are identical with the amino acid residues in relevant differential sequences, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and are established by the program itself, depending upon the composition of the particular sequence, however, the minimum values may be adjusted and are set as indicated above.

In addition, differential sequences in accordance with the present invention also include homologs or orthologs from other species (e.g. a rodent homolog; a primate homolog, such as a human homolog; another mammalian homolog; or a more distant homolog retaining sequence conservation sufficient to convey the desired effect on cellular phenotype) corresponding to differential sequences referenced in Tables 2-15. Such homologs or orthologs can be identified using standard homolog searching methods known in the art.

Furthermore, differential sequences in accordance with the invention also include nucleic acids that hybridize under stringent conditions to relevant nucleic acid sequences referenced in Tables 2-15 or homologs or orthologs thereof.

Differential sequences in accordance with the present invention can be manipulated to effect desirable cell phenotypes in CHO or other cell lines. This process is also referred to as rational cell engineering. Desirable cell phenotypes include those phenotypes characterized by increased and/or efficient production of recombinant transgenes or proteins. Such exemplary desirable cell phenotypes include, but are not limited to, increased cell growth rate, high peak cell density, sustained high cell viability, high maximum cellular productivity, sustained high cellular productivity, low ammonium production, and low lactate production, etc.

For example, differential sequences in accordance with the invention can be used to improve cellular productivity. The current productivity of a typical CHO cell line is about 1-3 g Mabs/L or less than 5 g Mabs/L. Engineered CHO cell lines in accordance with the present invention can have significantly increased productivity, for example, >5 g Mabs/L, >10 g Mabs/L, >15 g Mabs/L, >20 g Mabs/L, >25 g Mabs/L, >30 g Mabs/L. This productivity increase is not limited to antibody productions. It is applicable to productions of any proteins, such as fusion proteins (e.g., Fc:receptor fusion molecules), cytokines, coagulation factors, and/or native or endogenous proteins.

Differential sequences in accordance with the present invention can be manipulated using various methods known in the art. For example, differential sequences in accordance with the present invention may be down-regulated or up-regulated in cell lines. In some embodiments, differential sequences in accordance with the present invention may be down-regulated by the use of various inhibitory polynucleotides, such as antisense polynucleotides, ribozymes that bind and/or cleave the target mRNAs, triplex-forming oligonucleotides that target regulatory regions of relevant genes, and short interfering RNA that causes sequence-specific degradation of target mRNA (e.g., Galderisi et al. (1999) J. Cell. Physiol. 181:251-57; Sioud (2001) Curr. Mol. Med. 1:575-88; Knauert and Glazer (2001) Hum. Mol. Genet. 10:2243-51; Bass (2001) Nature 411:428-29).

Inhibitory antisense or ribozyme polynucleotides suitable for the invention can be complementary to an entire coding strand of a gene of interest, or to only a portion thereof. Alternatively, inhibitory polynucleotides can be complementary to a noncoding region of the coding strand of a gene of interest. Inhibitory polynucleotides suitable for the invention can be constructed using chemical synthesis and/or enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Mickelfield (2001) Curr. Med. Chem. 8:1157-70). Alternatively, antisense molecules can be produced biologically using an expression vector into which a polynucleotide of the present invention has been subcloned in an antisense (i.e., reverse) orientation.

In some embodiments, antisense polynucleotide molecules suitable for the invention can be α-anomeric polynucleotide molecules. An α-anomeric polynucleotide molecule forms specific double-stranded hybrids with complementary RNA in which, contrary to the usual β-units, the strands run parallel to each other. Antisense polynucleotide molecules can also comprise a 2′-o-methylribonucleotide or a chimeric RNA-DNA analogue, according to techniques that are known in the art.

In some embodiments, inhibitory triplex-forming oligonucleotides (TFOs) suitable for the present invention bind in the major groove of duplex DNA with high specificity and affinity (Knauert and Glazer, supra). Expression of target genes can be inhibited by targeting TFOs complementary to the regulatory regions of the genes (i.e., the promoter and/or enhancer sequences) to form triple helical structures that prevent transcription of the genes.

In some embodiments, inhibitory polynucleotides are short interfering RNA (siRNA) molecules. These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nat. Med. 9:347-51).

siRNA molecules suitable for the present invention can be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). siRNA molecules can be chemically synthesized (Elbashir et al. (2001) Nature 411:494-98) or produced by in vitro transcription using single-stranded DNA templates (Yu et al., supra). Alternatively, siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

siRNA molecules targeted to differential sequences of the present invention can be designed based on criteria well known in the art (e.g., Elbashir et al. (2001) EMBO J. 20:6877-88). For example, target segments of target mRNAs should begin with AA (preferred), TA, GA, or CA; the GC ratio of the siRNA molecule should be 45-55%; siRNA molecules should not contain three of the same nucleotides in a row; siRNA molecules should not contain seven mixed G/Cs in a row; and target segments should be in the ORF region of the target mRNAs and should be at least 75 bp after the initiation ATG and at least 75 bp before the stop codon. siRNA molecules targeted to the polynucleotides of the present invention can be designed by one of ordinary skill in the art using the aforementioned criteria or other known criteria.

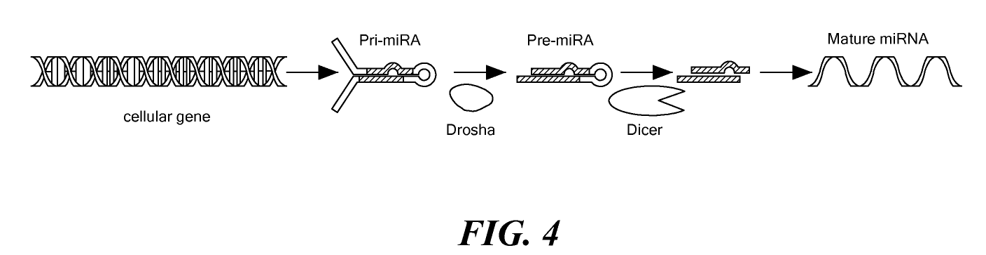

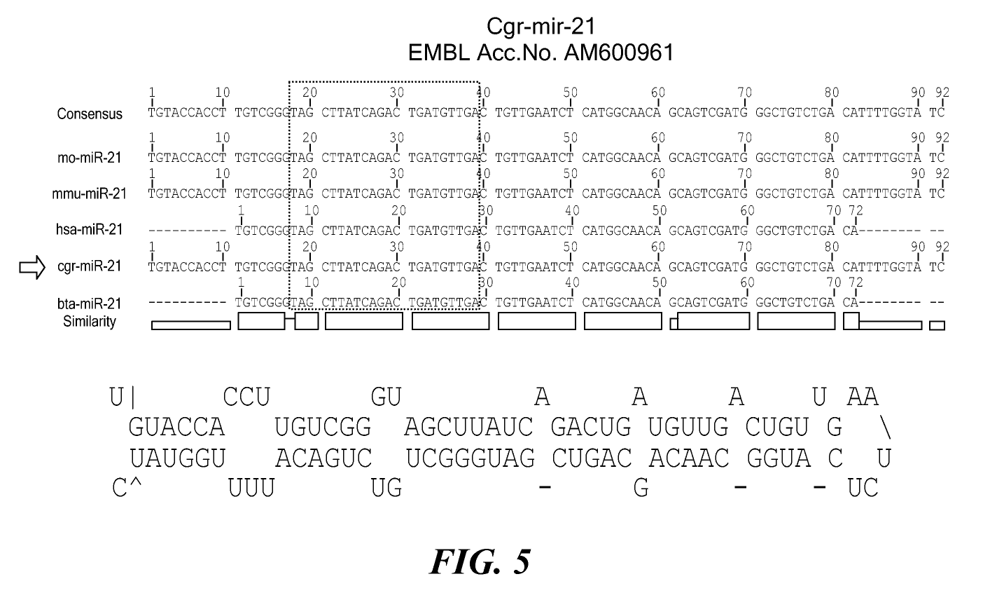

In other embodiments of the invention, inhibitory polynucleotides are microRNA (miRNA) molecules. miRNA are endogenously expressed molecules (typically single-stranded RNA molecules of about 21-23 nucleotides in length), which regulate gene expression at the level of translation. Typically, miRNAs are encoded by genes that are transcribed from DNA but not translated into protein (non-coding RNA). Instead, they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression. FIG. 4 illustrates exemplary processing of miRNA. miRNA are highly conserved and predicted to be responsible for regulating at least about 30% of the genes in a genome. In some embodiments, CHO miRNA can be identified by relying on high human-mouse homology. For example, highly conserved miRNA sequences can be used to screen and identify CHO specific miRNA. CHO specific miRNAs have been cloned. FIG. 5 illustrates the sequence of an exemplary miRNA, Cgr-mir-21.

Down-regulation of relevant differential sequences in accordance with the present invention may also be achieved through the creation of cells whose corresponding endogenous genes have been disrupted through insertion of extraneous polynucleotides sequences (i.e., a knockout cell). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout cells include homologous recombination and are well known in the art (e.g., Wolfer et al. (2002) Trends Neurosci. 25:336-40).

The expression or activity of relevant differential sequences in accordance with the invention may also be up-regulated. Up-regulation includes, but is not limited to, providing exogenous nucleic acids (e.g., an over-expression construct) containing relevant differential sequences in accordance with the invention. For example, isolated polynucleotides corresponding to relevant differential sequences of the present invention may be operably linked to expression control sequences such as, for example, the pMT2 and pED expression vectors, and introduced into cell lines by, for example, transient or stable transfection. General methods of over-expression are well known in the art.

The expression or activity of differentially expressed genes or proteins of the present invention may also be altered by exogenous agents, small molecules, pharmaceutical compounds, or other factors that may be directly or indirectly modulating the activity of the genes or proteins of interest. As a result, these agents, small molecules, pharmaceutical compounds, or other factors may be used to effect desirable cell phenotypes (e.g., increased production of a recombinant transgene, increased cell growth rate, high peak cell density, sustained high cell viability, high maximum cellular productivity, sustained high cellular productivity, low ammonium production, and low lactate production, etc.).

Any combinations of various methods manipulating gene or protein expression or activity described herein are within the scope of the invention. Combinations of genes or proteins affecting different cell phenotypes can be manipulated using methods described herein and are within the scope of the invention.

It should be understood that the above-described embodiments and the following examples are given by way of illustration, not limitation. Various changes and modifications within the scope of the present invention will become apparent to those skilled in the art from the present description.

### EXAMPLES

### Example 1

### Cell Culture and Time Course Analysis

Cells were cultured in serum-free suspension culture in two basic formats, under two basic conditions. One format was small scale, shake flask culture in which cells were cultured in less than 100 ml in a vented tissue culture flask, rotated on an orbiting shaker in a CO₂ incubator. The second format was in bench top bioreactors, 2 L or less working volume, controlled for pH, nutrients, dissolved oxygen, and temperature. The two basic culture conditions were ordinary passage conditions of 37° C., or fed batch culture conditions. In a basic fed batch culture, the cells are grown for a longer period of time, and shifted to a lower temperature in order to prolong cell viability and extend the productive phase of the culture. For example, in the fed batch culture, cells were grown through an initial phase of exponential growth. After several days, the culture temperature was lowered, nutrient feeds were added to supplement growth, and the cells were maintained for up to 14 days. At this time, the cells typically enter a lag phase. In some cases, viability begins to decline towards the end of the culture.

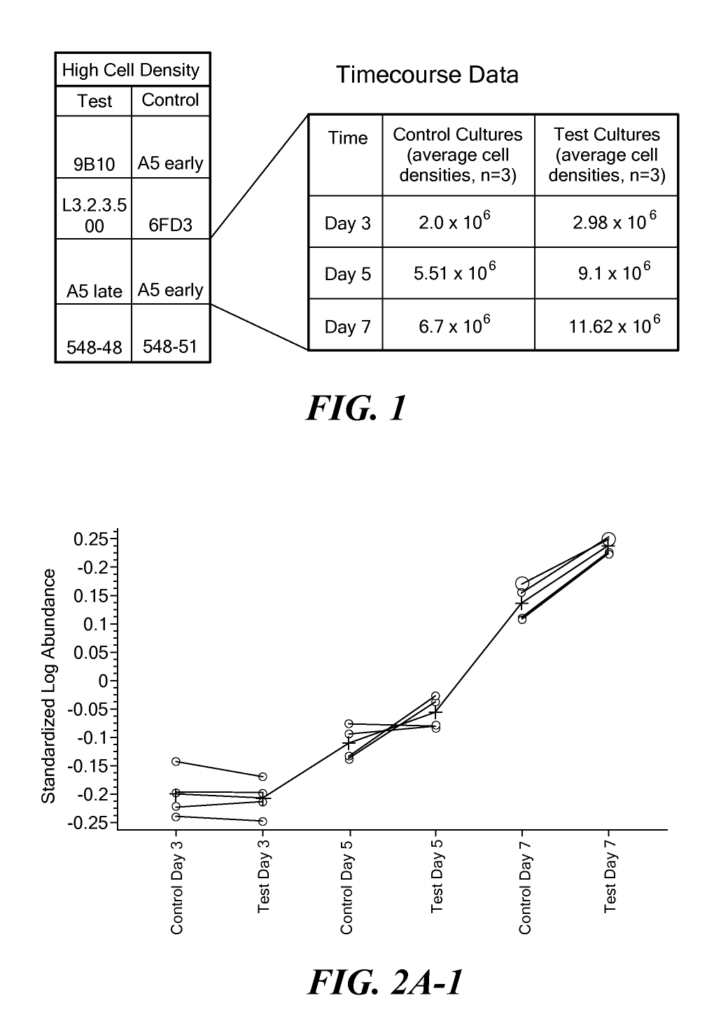

In this experiment, we compared two different cell lines (referred to as test cell line and control cell line) that display different growth profiles over a fed batch culture. As shown in FIG. 1, one cell line (test cell line) maintains a high viability (high cell density) throughout the fed batch, while the other (control cell line) declines in viability relatively early. Replicate culture samples of each cell line grown under similar fed batch conditions were taken at multiple time points. For example, as shown in FIG. 1, samples were taken at day 3, 5, and 7. Average cell densities of each cell line at day 3, 5 and 7, respectively, were also shown FIG. 1. Each sample was analyzed as described below in order to characterize how the cells change their expression profiles over time.

### Example 2

### Detection of Differentially Expressed Proteins

Cells from each sample were harvested and subjected to standard lysis in 7 M urea, 2 M thiourea, 4% CHAPS, 30 mM Tris, 5 mM magnesium acetate at pH 8.5. 150 μg aliquots of the lysates were analyzed by two-dimensional gel electrophoresis to confirm sample quality using 18 cm immobilized pH gradient isoelectric focusing gradient strips, pH 4-7. The strips were rehydrated overnight with 340 μl of buffer per strip. Samples were loaded at the cathodic end of the strip and subjected to 500 V for 1 hour, 1000 V for 1 hour, and 8000 V for 4 hours and stored at −80° C. until the second dimension on 12.5% acrylamide gels. Electrophoresis in the second dimension was performed at 1.5 W per gel for 30 minutes and then a total of 100 W for 5 hours for a Dalt 6 run of 6 large format gels. Proteins were visualized by silver staining to confirm the quality of the proteins in the lysate.

Aliquots of the original lysates were then labeled with fluorescent dyes in preparation for fluorescent 2-dimensional differential in-gel electrophoresis (DIGE). Each comparison of cell cultures was performed four times using duplicate gels for a total of 8 DIGE gels per experiment, using 50 μg each of Cy2-, Cy3-, and Cy5-labeled cell lysates per gel. All cell lysates used in an experiment were pooled and labeled with Cy2 to serve as an internal standard. The control cell lysate was labeled with Cy3 and the test cell lysate is labeled with Cy5. Labeling was performed on ice in the dark for 30 minutes, followed by a 10 minute quenching of the reaction using 10 mM lysine on ice in the dark. The Cy2-, Cy3-, and Cy5-labeled lysates were then pooled and mixed with 2× sample buffer for 15 minutes in the dark on ice.

The samples were applied to immobilized pH gradient isoelectric focusing strips. The strips were rehydrated overnight for about 20 hours. Samples were loaded at the cathodic end of the strip and subjected to 300V/3 hr/G, 600V/3 hr/S&H, 1000V/3 hr/G, 8000V/3 hr/G, 8000V/4 hr/S&H, and 500V/12 hr/S&H. One hour before SDS-PAGE, the strips were subjected to 8000V for one hour. The strips were equilibrated for 15 minutes in SDS buffer+1% DTT and for 15 minutes in SDS buffer+2.5% iodoacetamide. The strips were applied to polyacrylamide gels and overlaid with agarose. Electrophoresis through the gels was performed at 1.5 W/gel at 110° C. for about 18 hours on a Dalt 12 using 12 large format gels. The gels were scanned on a Typhoon™ 9400 scanner with a variable mode imager; cropped; and imported into DeCyder™ software. Differentially regulated proteins were identified using biological variance analysis (BVA). These proteins were matched to a preparative gel loaded with 400 μg of protein and stained with ruthenium. From the preparative gel, an Ettan Spot Picker was used to pick proteins identified by DIGE as differentially regulated. An Ettan Digestor was used to digest the individual proteins with an overnight trypsin incubation. The resulting peptides were analyzed by mass spectrometry. MALDI is used, particularly for highly abundant samples on gels, for peptide mass fingerprinting.

For lower abundance samples, LC-MS/MS using an MDLC LTQ machine is used. Tryptically digested samples from 2D gel spots were resuspended in 20 μL of LC-MS grade water containing 0.1% TFA and analysed by one-dimensional LC-MS using the Ettan™ MDLC system (GE Healthcare) in high-throughput configuration directly connected to a Finnigan™ LTQ™ (Thermo Electron). Samples were concentrated and desalted on RPC trap columns (Zorbax™ 300SB C18, 0.3 mm×5 mm, Agilent Technologies) and the peptides were separated on a nano-RPC column (Zorbax™ 300SB C18, 0.075 mm×100 mm, Agilent Technologies) using a linear acetonitrile gradient from 0-65% Acetonitrile (Riedel-de Haën LC-MS grade) over 60 minutes directly into the LTQ via a 10 μm nanoESI emitter (Presearch FS360-20-10-CE-20). The LTQ ion trap mass spectrometer was used for MS/MS. A scan time of ˜0.15 s (one microscans with a maximum ion injection time of 10 ms) over an m/z range of 300-2000 was used followed by MS/MS analysis of the 3 most abundant peaks from each scan which were then excluded for the next 60 seconds followed by MS/MS of the next three abundant peaks which in turn were excluded for 60 seconds and so on. A “collision energy” setting of 35% was applied for ion fragmentation and dynamic exclusion was used to discriminate against previously analysed ions (data dependent analysis).

All buffers used for nanoLC separations contained 0.1% Formic Acid (Fluke) as the ion pairing reagent. Full scan mass spectra were recorded in profile mode and tandem mass spectra in centric mode. The peptides were identified using the information in the tandem mass spectra by searching against SWISS PROT database using SEQUEST™. An Carr value of >1.5 for singly charged peptides, >2.0 for doubly charged peptide and >2.5 for triply charged peptides was used as statistical cut-off.

### Example 3

### Identification of Differentially Expressed Proteins

The protein expression profiles of replicate samples of the test cell line taken at one time point were compared to the protein expression profiles of replicate samples of the same cell line but taken at a different time point to identify differentially expressed proteins over time (ANOVA analysis). Preferably, samples were taken from distinct growth phases. For example, expression profiles of samples taken from an exponential phase were compared to the expression profiles of samples taken from a lag phase. To be considered as a differentially-expressed protein at in the DeCyder analysis, a protein must have been identified in all sample gels; have demonstrated at least a 1.5-fold up- or down-regulation; and have demonstrated a T-test score less than 0.05. The same analysis was done with the control cell line. In this experiment, the test cell line maintains a high viability throughout the fed batch, while the control cell line declines in viability early.

The differentially expressed proteins in the test cell line were compared to the differentially expressed proteins in the control cell line to classify the differentially expressed proteins into three groups, namely, the test cell-only, the control cell-only and common differentially expressed proteins.

Alternatively, two parallel comparisons were conducted using two pairs of cell lines (test cell line vs. control cell line) (referred to as HCD3 and HCD4, respectively). ANOVA analysis as described above was conducted for each pair at 3 time points (days 3, 5, and 7 for HCD3 and days 3, 6, 9 for HCD4) culture samples. For each of HCD3 and HCD4, the differentially expressed proteins in test or control cells were compared in order to identify those proteins unique to test cells (test-only or test-specific) or control cells (control-only or control-specific), or those in common to both (process-only or process-specific). Each of the test-only, control-only, and process-only protein lists from HCD3 were overlapped with the corresponding list from HCD4 to identify commonly up or down-regulated test-only differentially expressed proteins for both HCD3 and HCD4, commonly up or down-regulated control-only differentially expressed proteins for both HCD3 and HCD4, or commonly up or down-regulated process-only differentially expressed proteins for both HCD3 and HCD4.

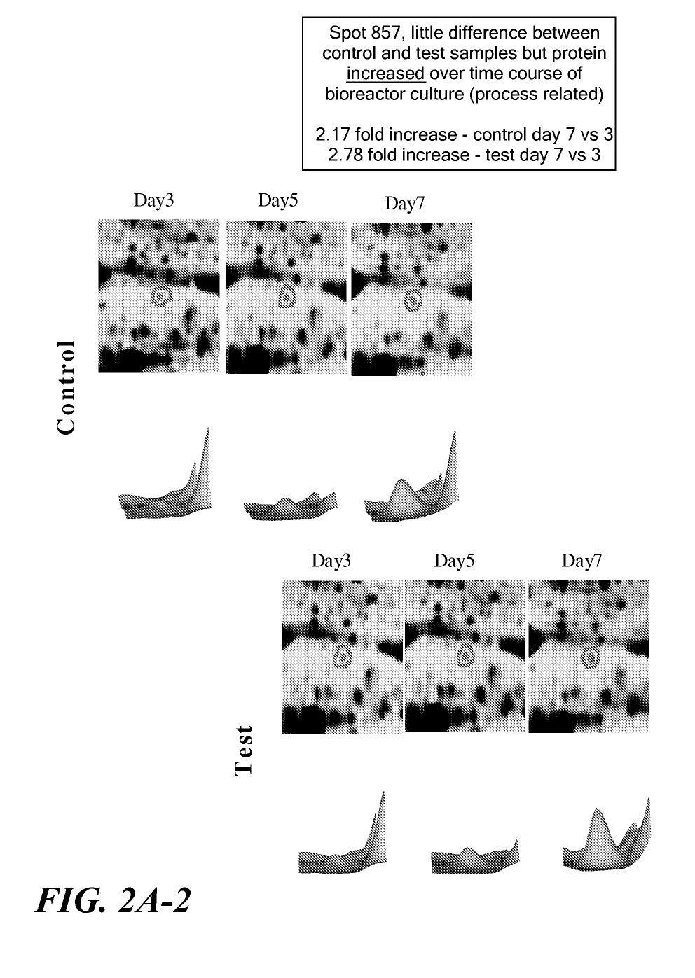

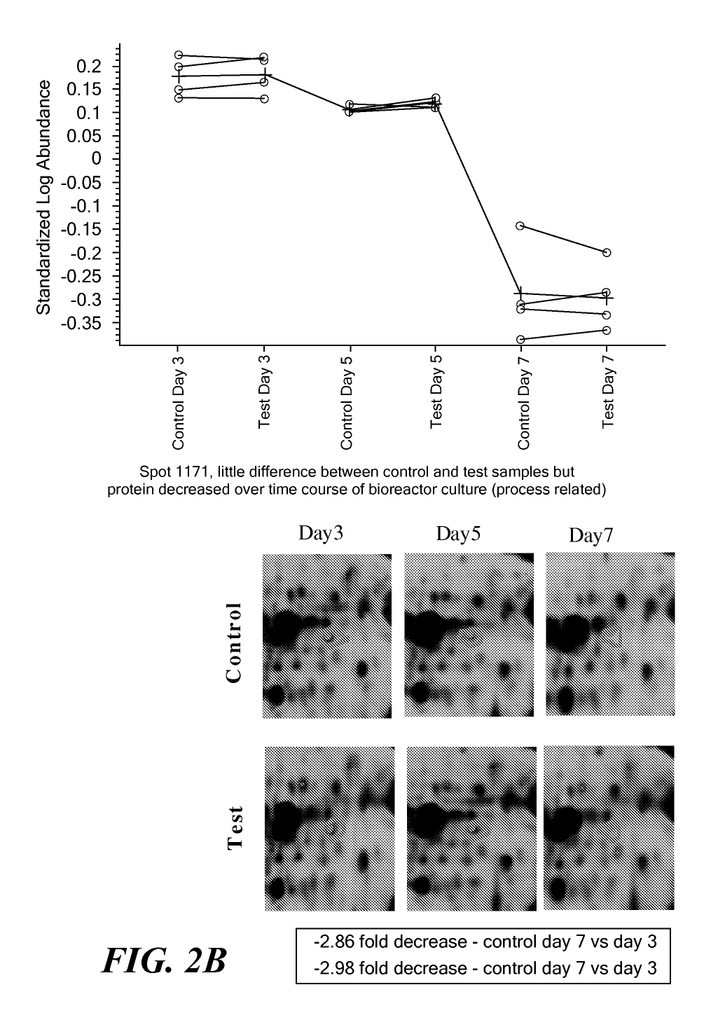

Exemplary control cell-only differentially expressed proteins were shown in Tables 2 and 3. Exemplary test cell-only differentially expressed proteins were shown in Tables 4 and 5. Exemplary common differentially expressed proteins were shown in Tables 6 and 7. Common differentially expressed proteins are process related. Typically, expression levels of those proteins increased over time course of bioreactor culture (process related) but with little difference between control and test samples. Exemplary process related spots were shown in FIGS. 2A and 2B.

For some of the spots listed in the tables, MALDI sequence analysis identified one or two corresponding amino acid sequences. For lower abundance samples, LC-MS/MS using an MDLC LTQ machine is used. The tables provide, for each spot number, the fold difference in protein levels between different time points, labeled as “Fold Change”; proteins whose levels are reduced at a later time point are indicated with a negative sign. The tables also provide the p-value that the differences in expression would be the result of random chance and the protein name and accession number corresponding to any identified amino acid sequence. In the MALDI sequence analysis, the molecular weights of the trypsin fragments were compared to predicted molecular weights of trypsin fragments of known sequences. In some cases, in this sequence analysis and in other peptide sequence analyses included in this application, the detected molecular weights are indicative of detection of a modified form of a peptide, such as where cysteine has been modified with iodacetamide, or where methionine has been partially oxidized. It is understood that this is not necessarily reflective of the initial state of the peptide in the context of the protein in the cell or the cellular milieu. Accordingly, the peptide sequences provided in the sequence listing reflect the unmodified forms of the peptide, and cells engineered to have desirable cellular phenotypes will, in some embodiments, be engineered to regulate genes expressing an amino acid sequence comprising one or more of the peptides.

In the tables, “% coverage” refers to the percentage of the total length of a database sequence for which corresponding trypsin fragments were detected in the experiment. pI and M_{R} refer to the apparent isoelectric point and apparent molecular weight of the protein spot. For some proteins, putative protein functions are also provided in the table.

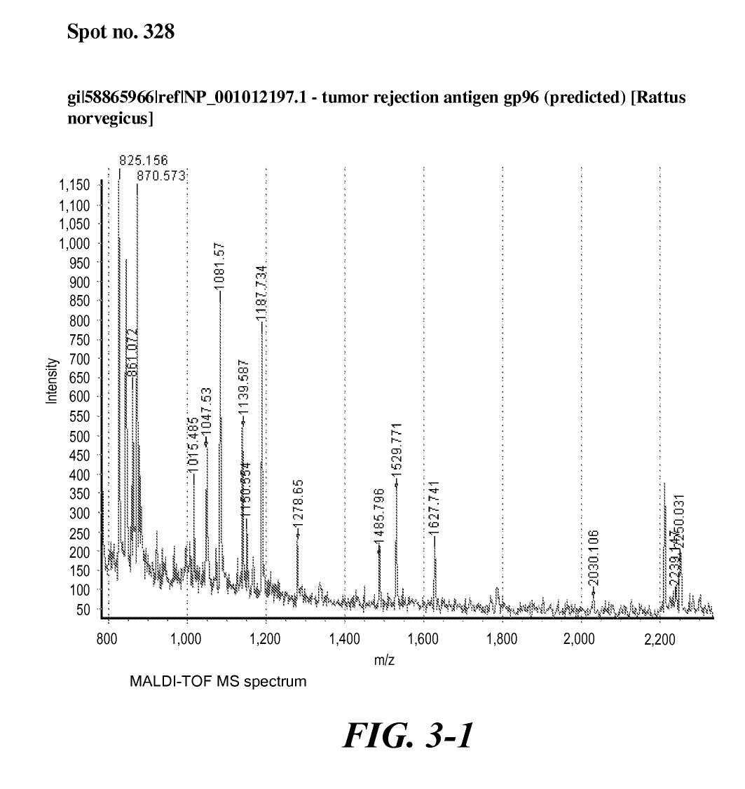

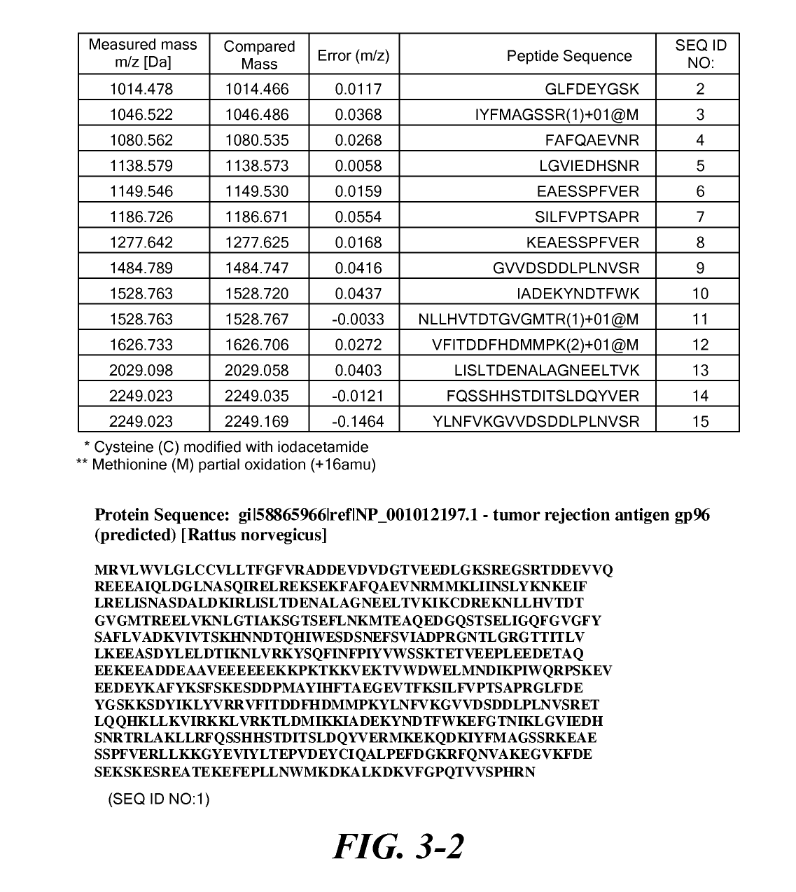

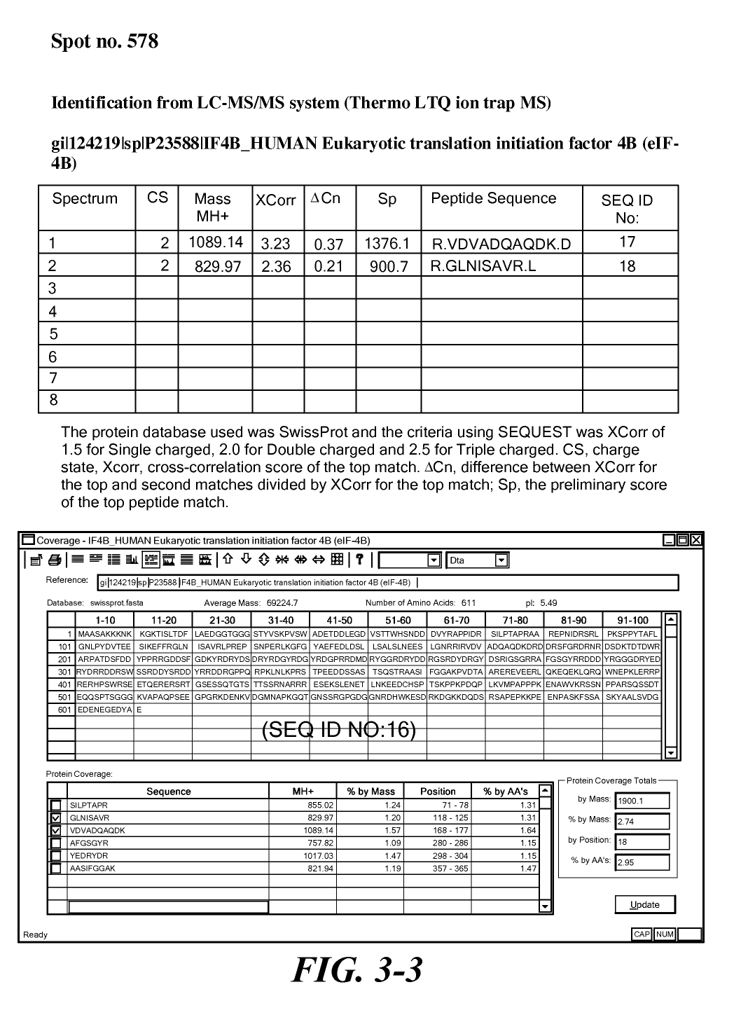

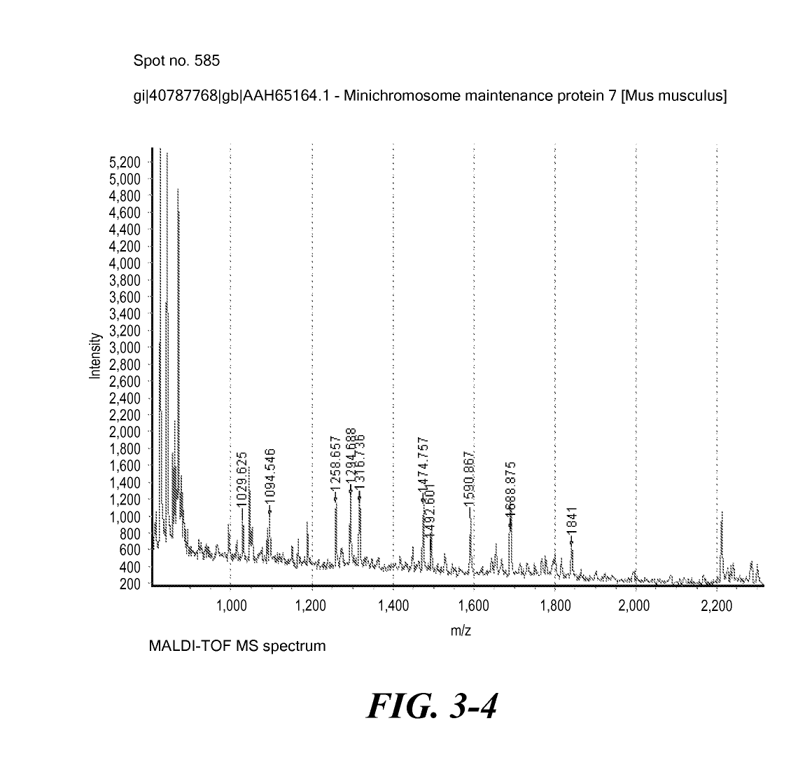

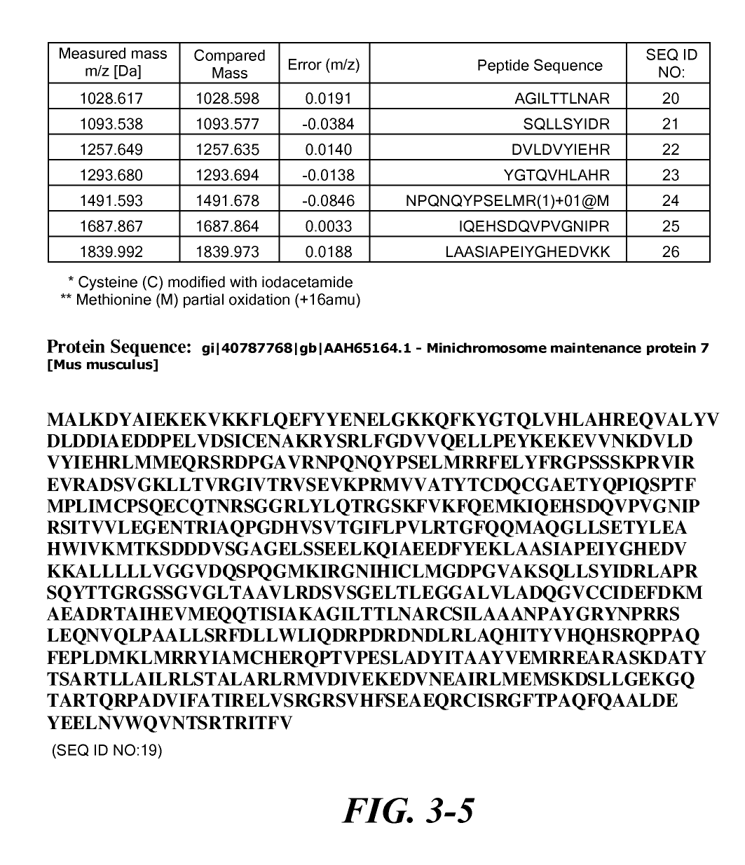

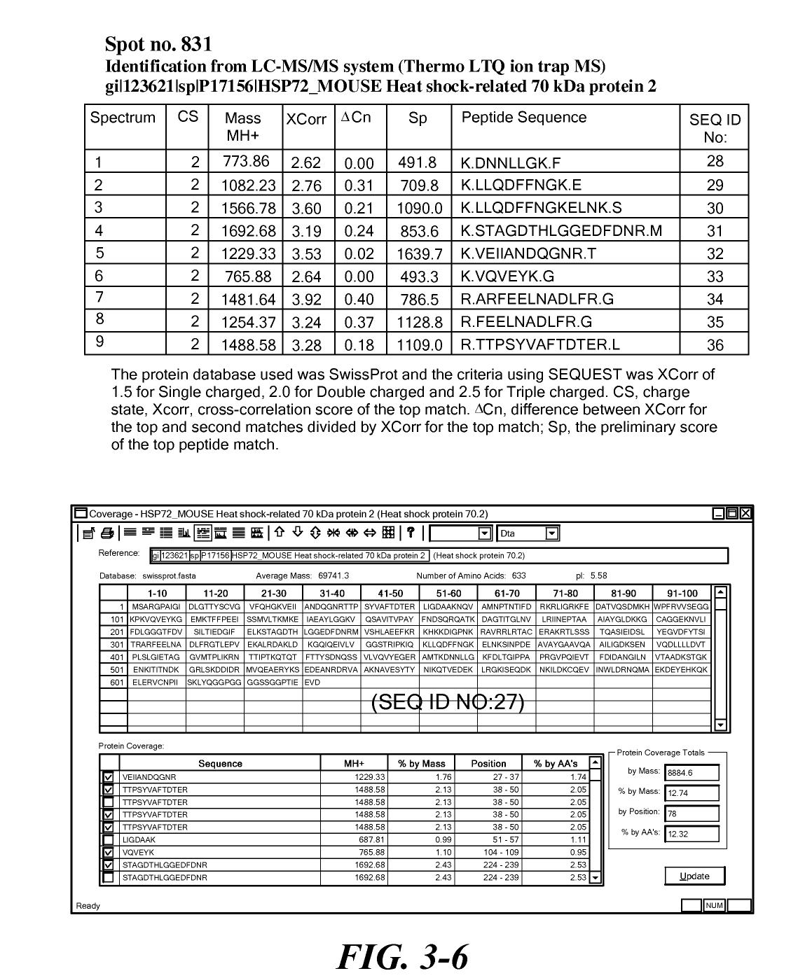

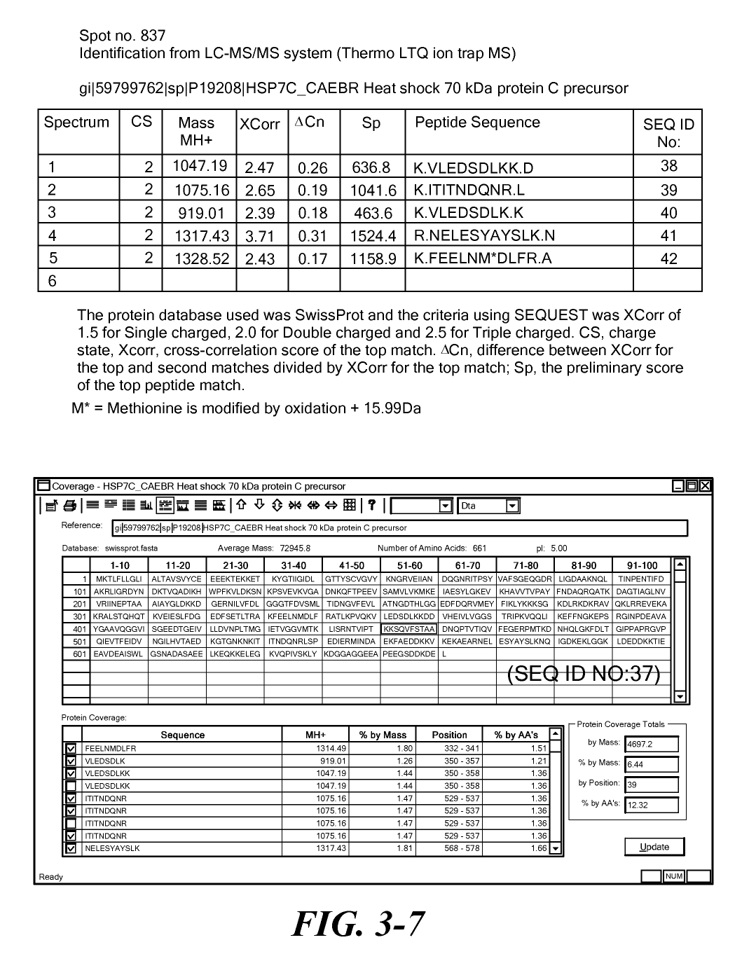

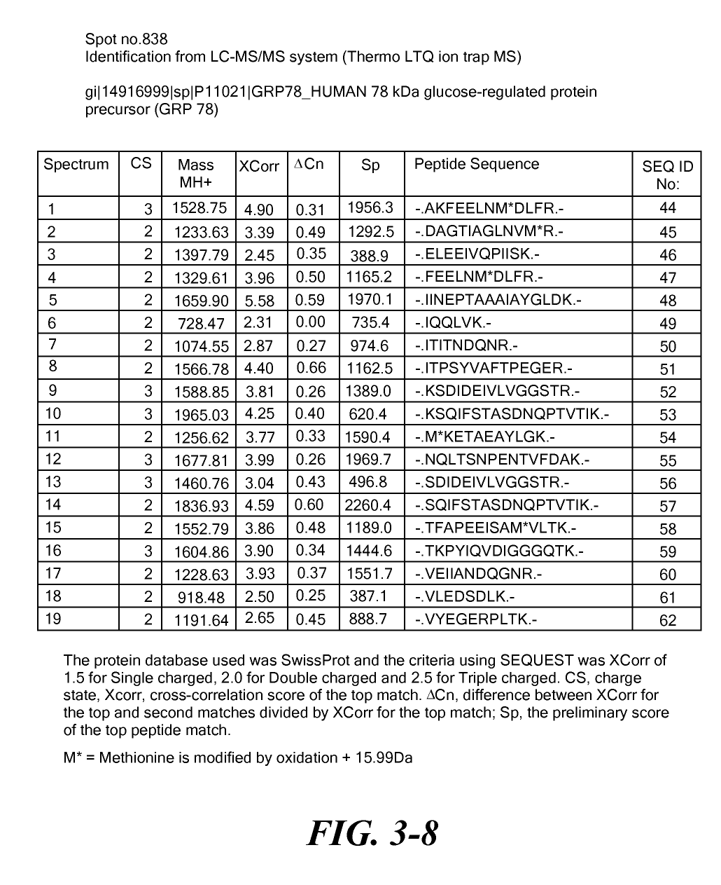

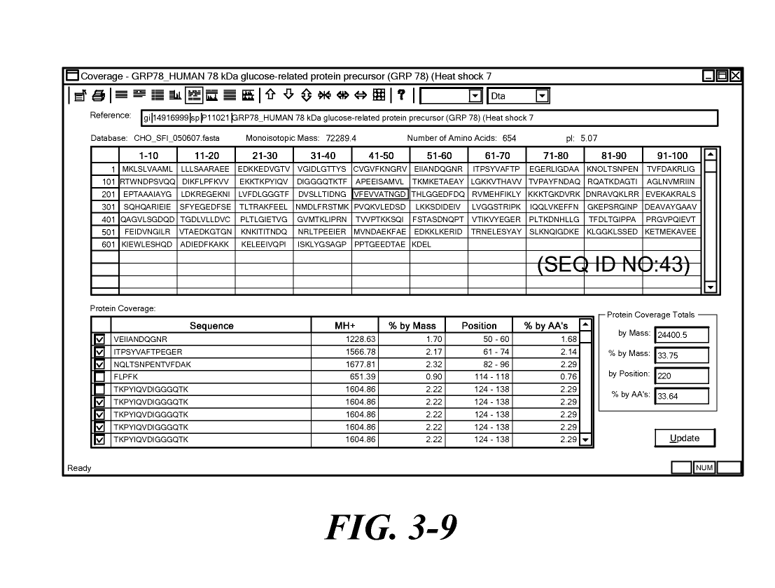

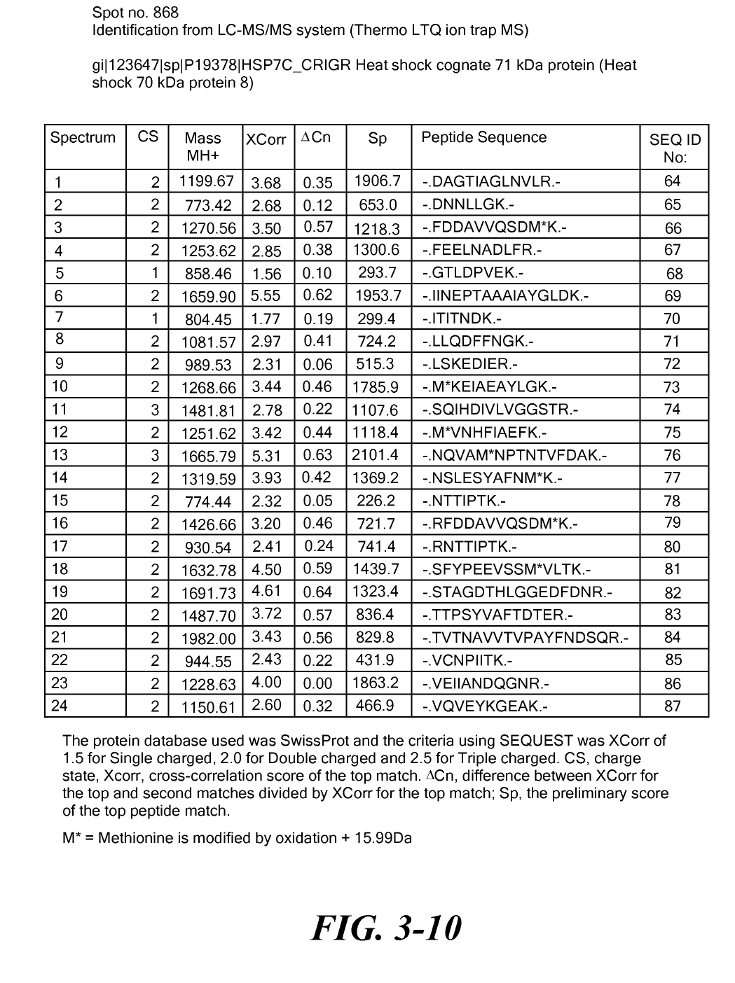

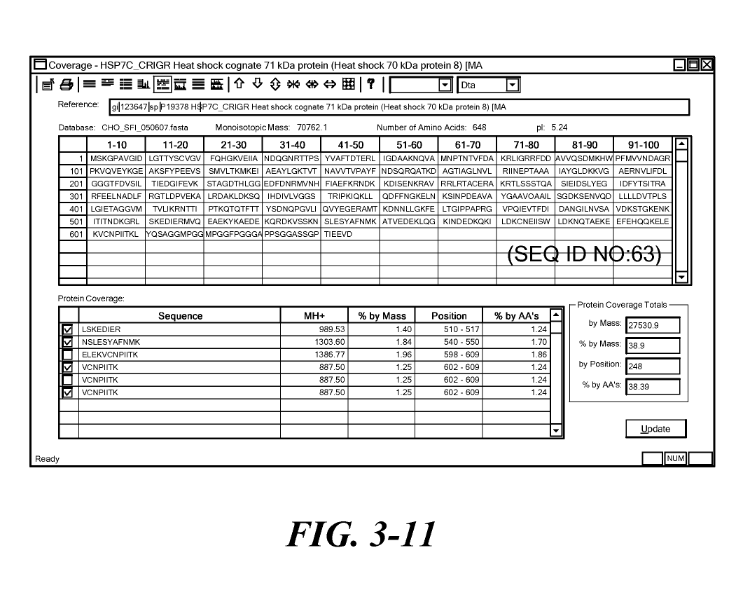

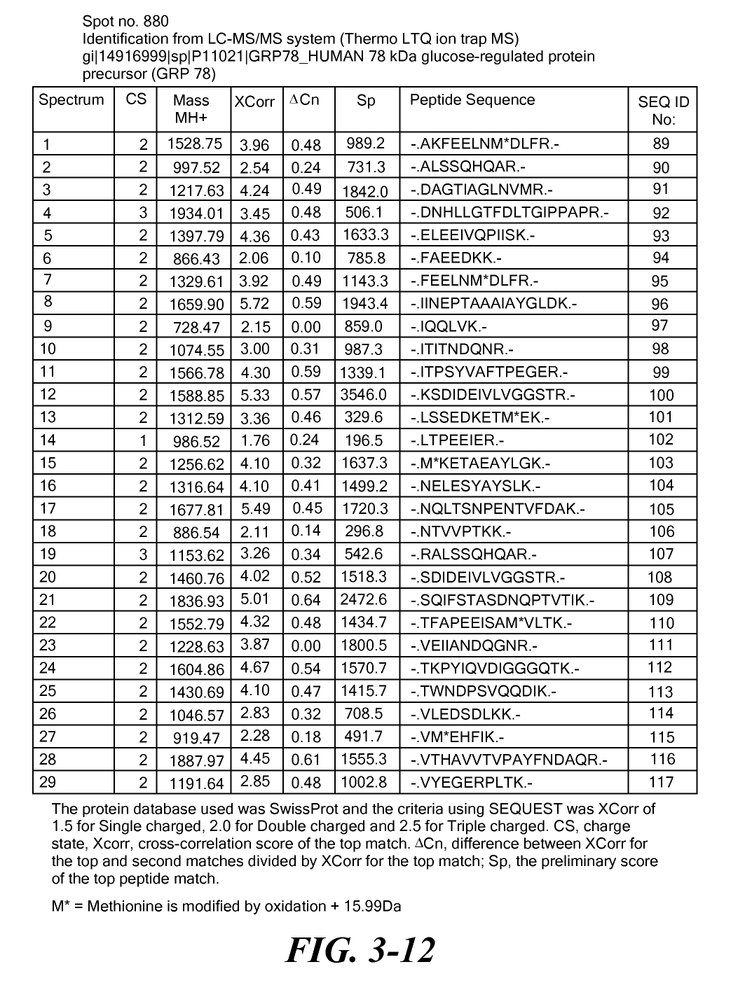

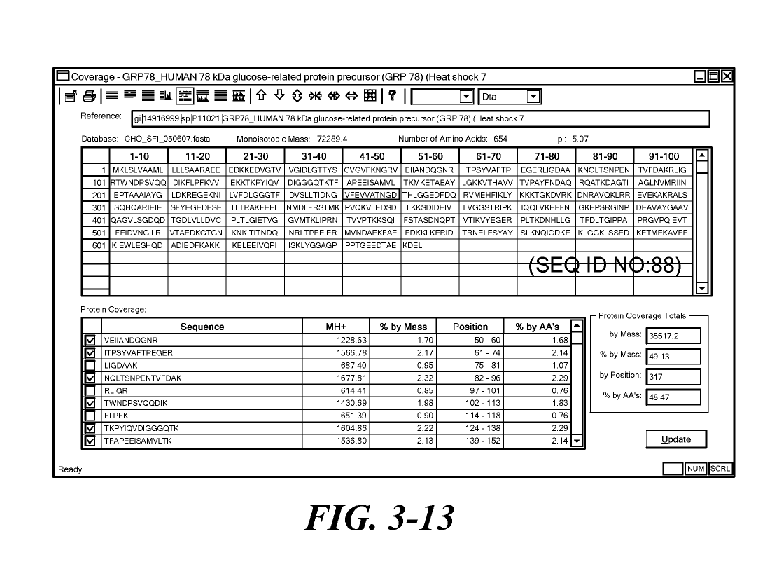

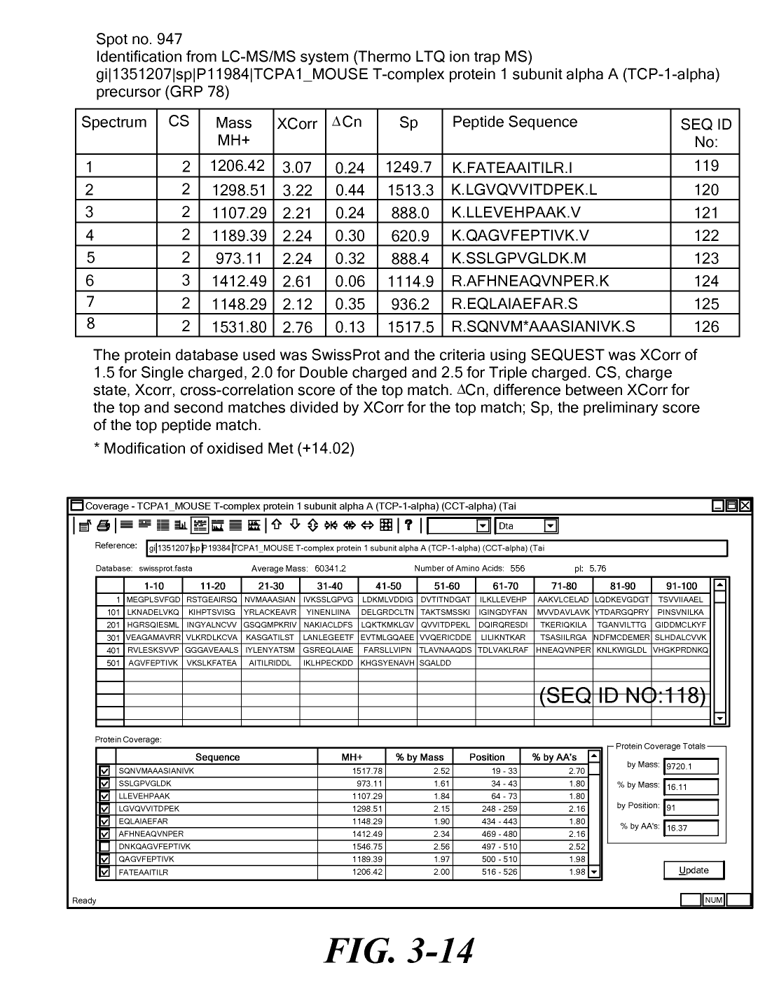

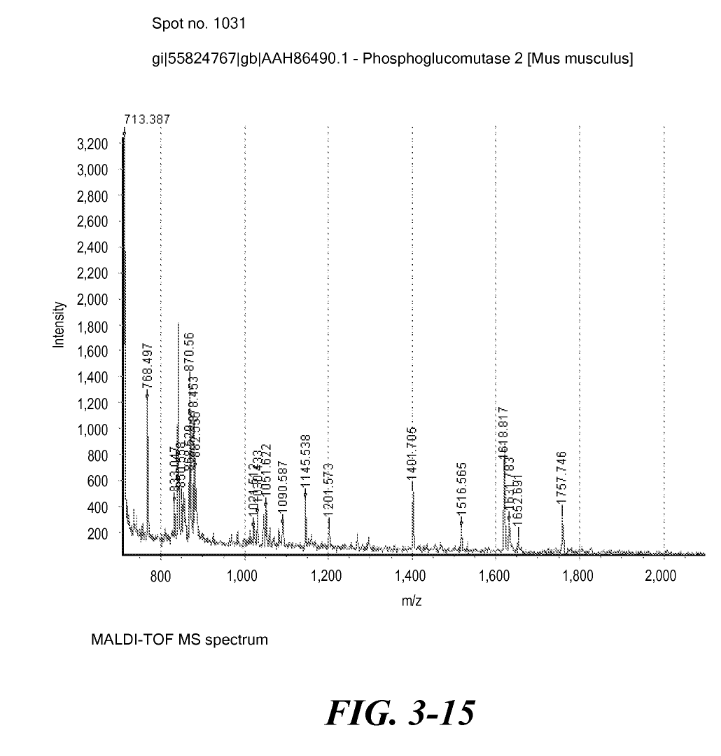

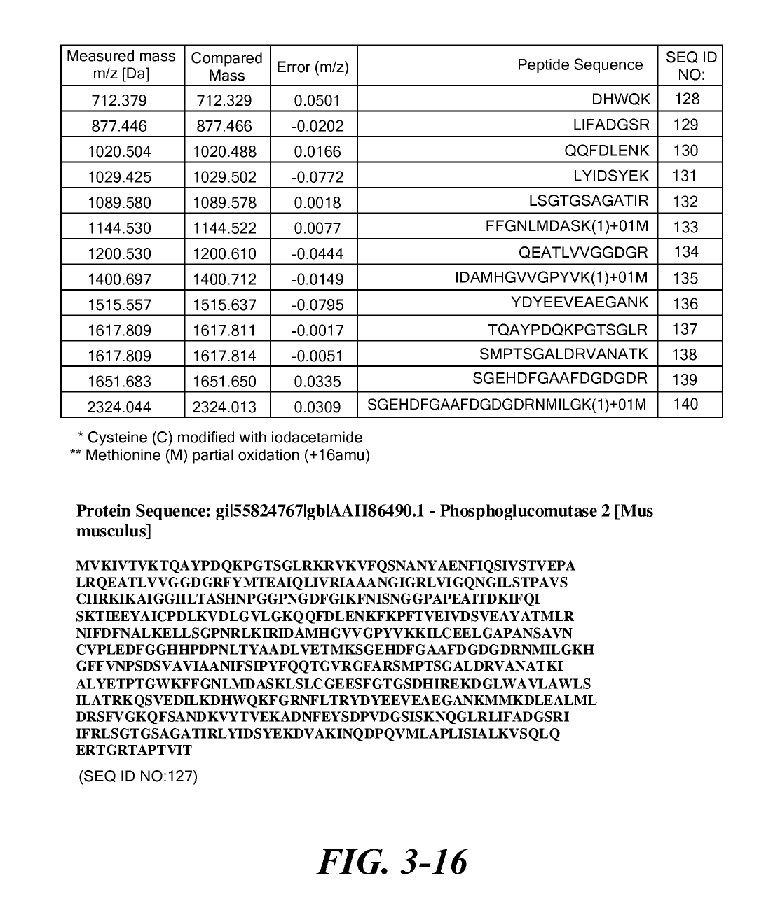

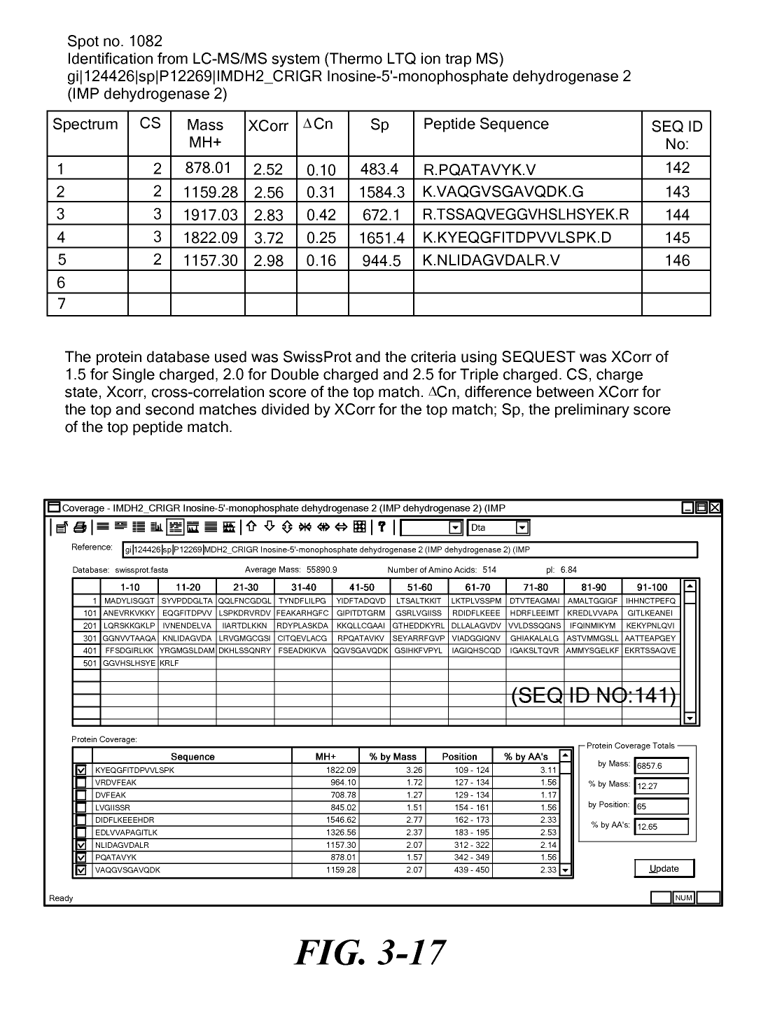

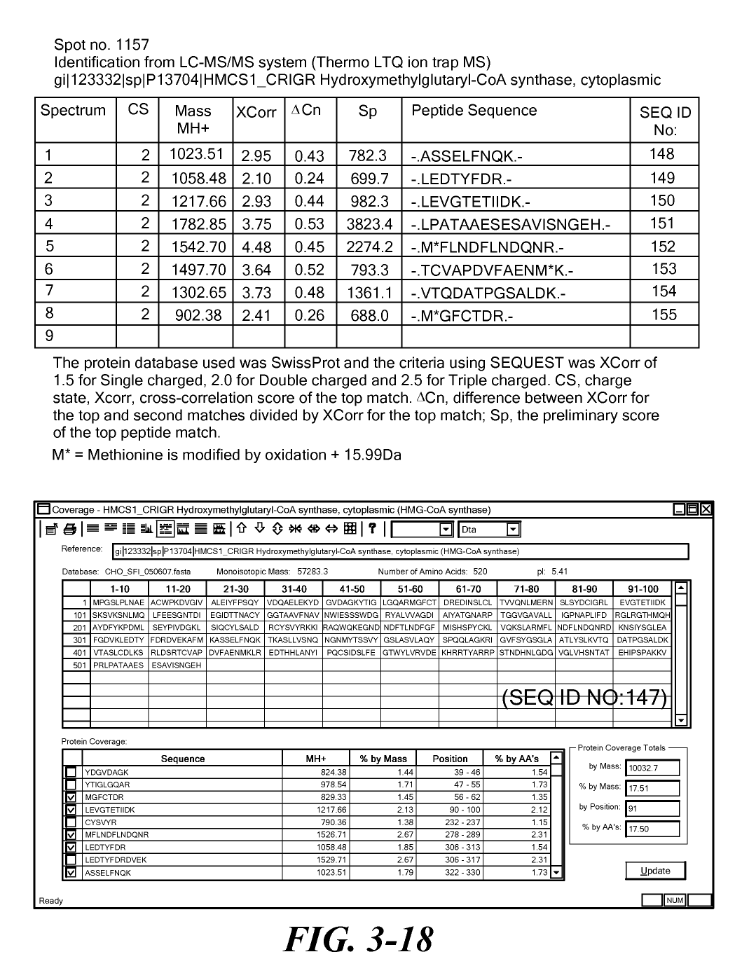

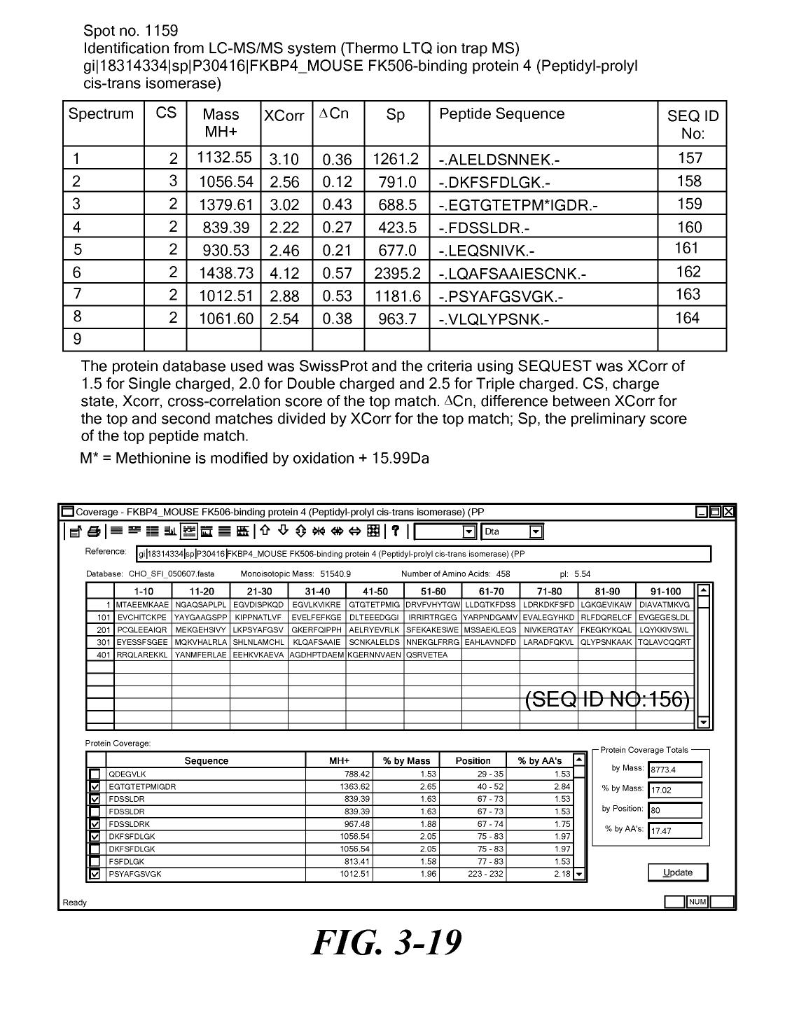

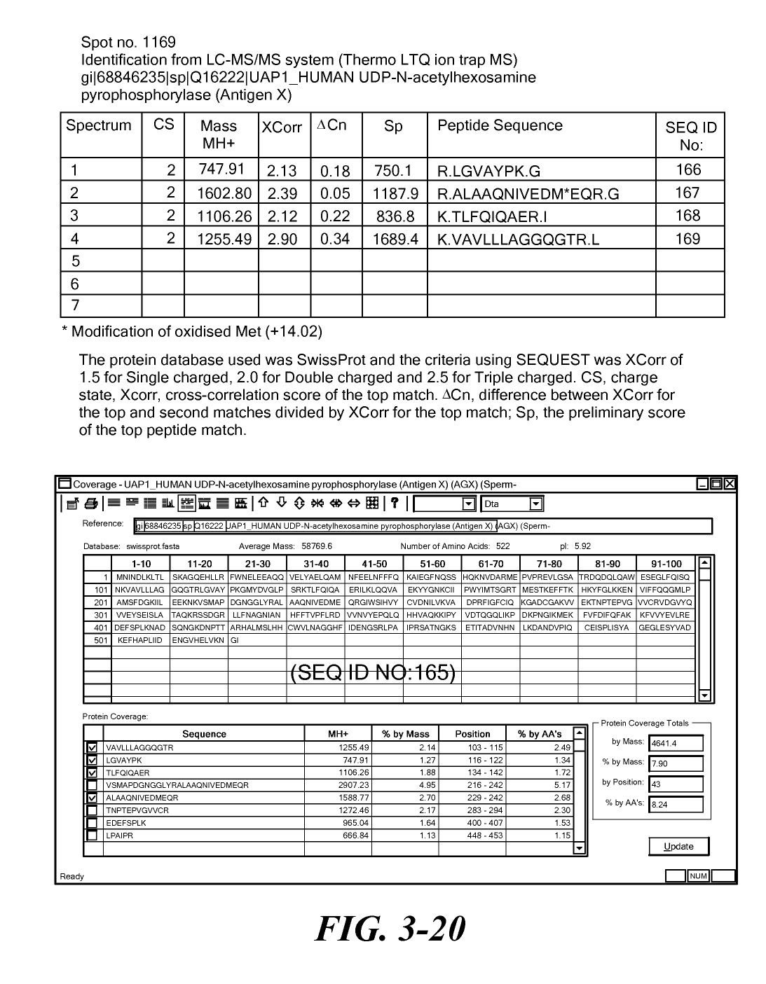

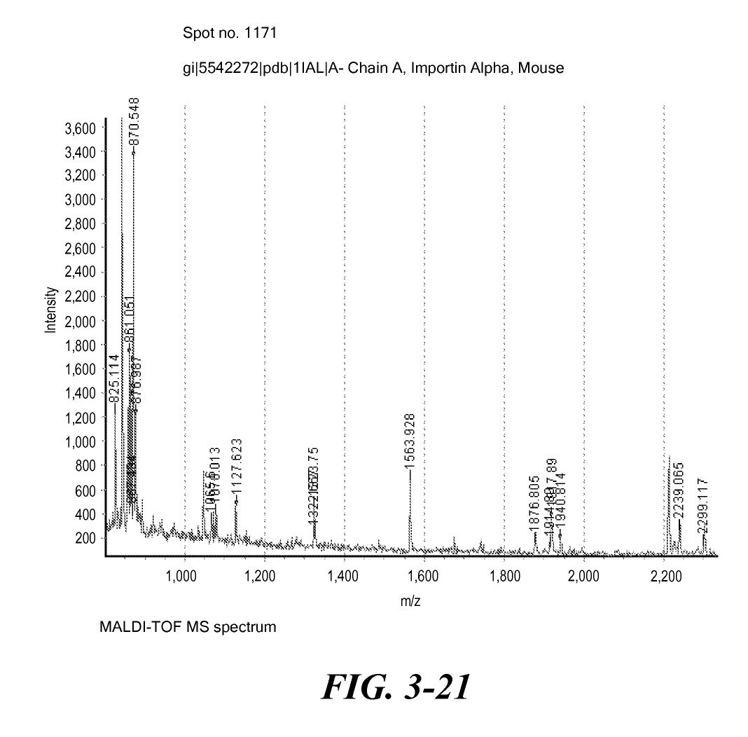

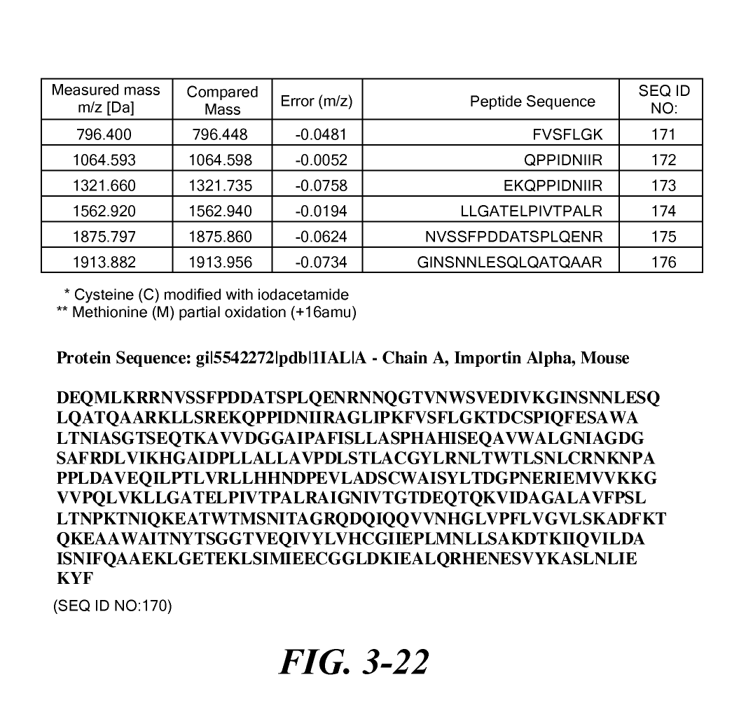

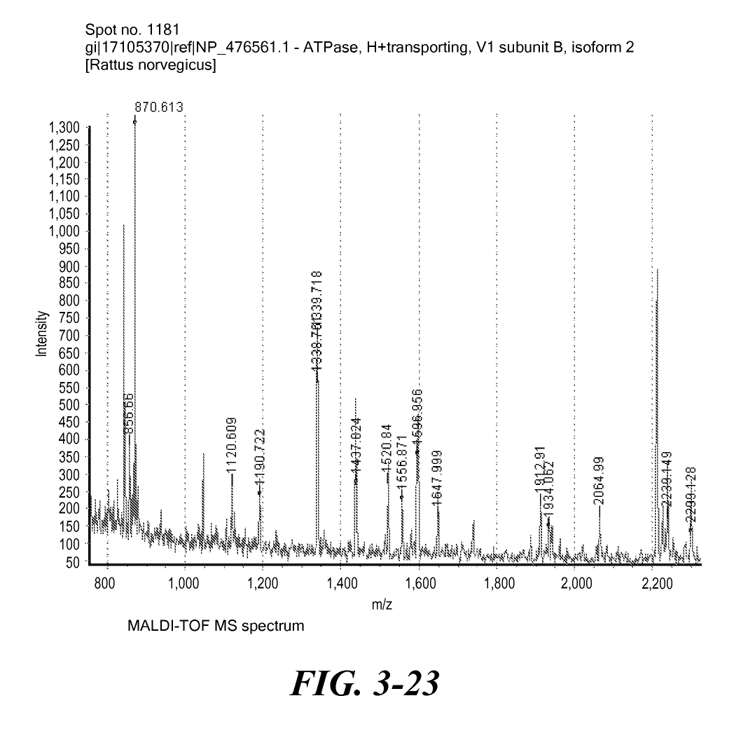

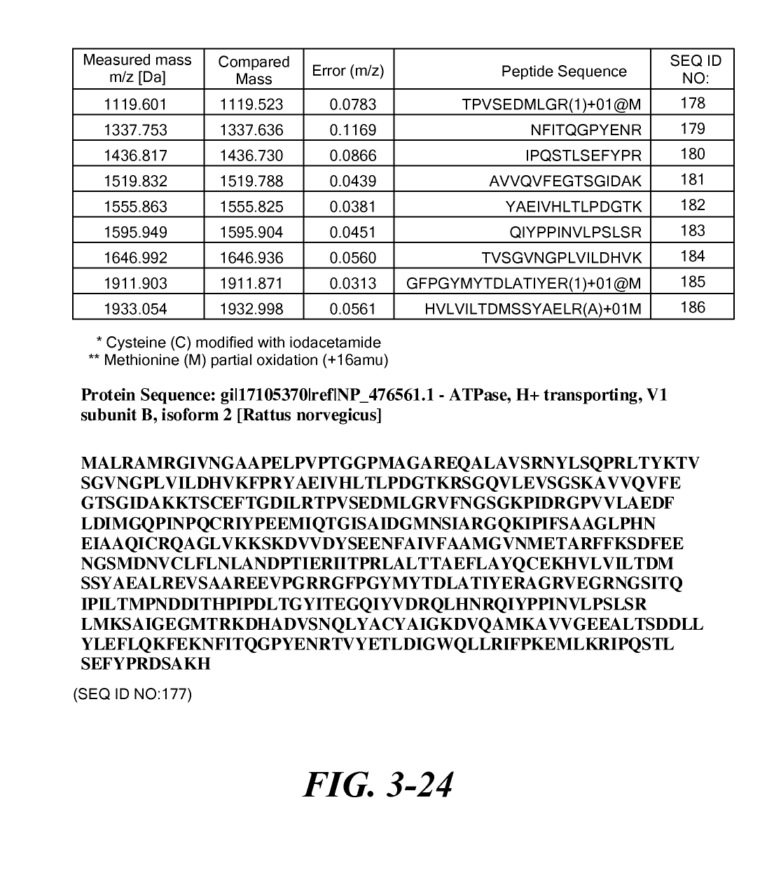

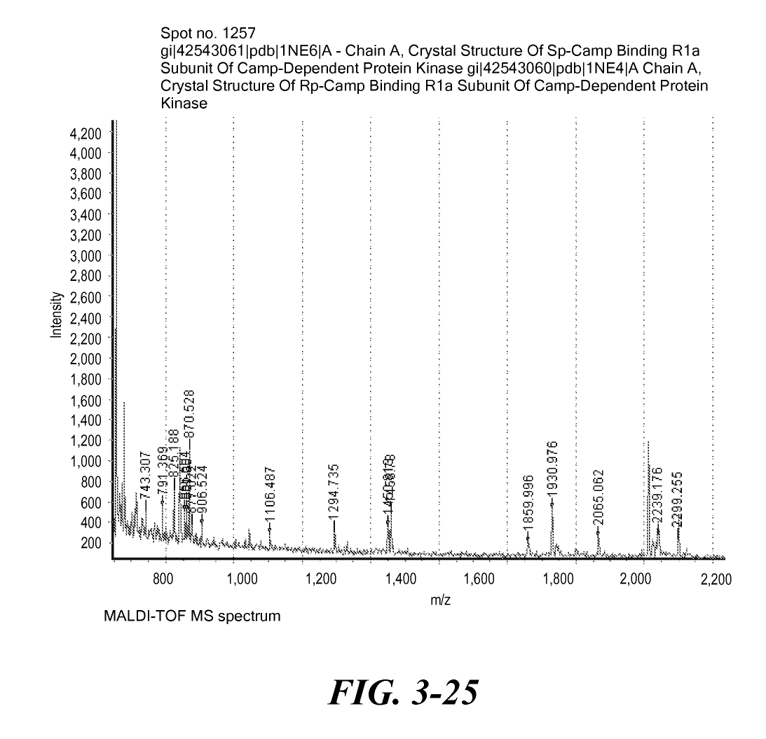

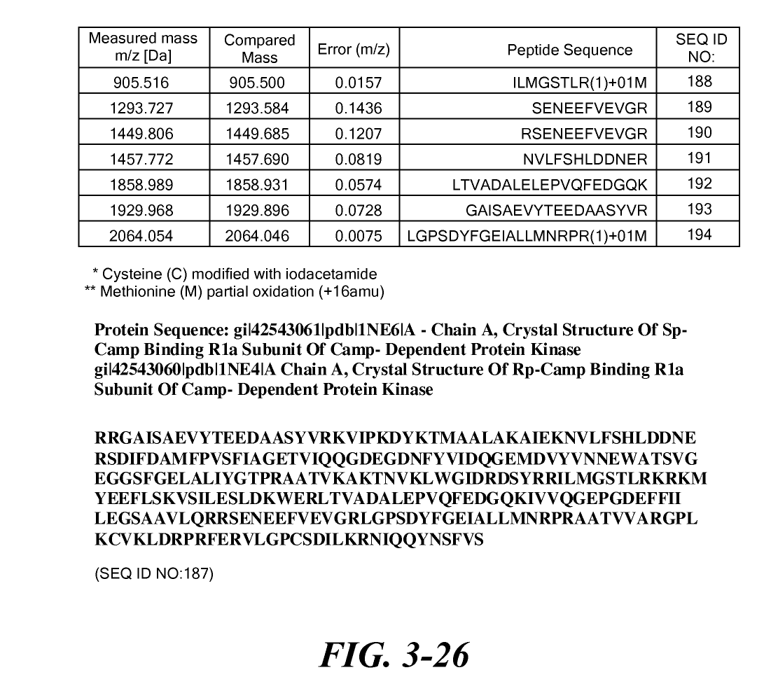

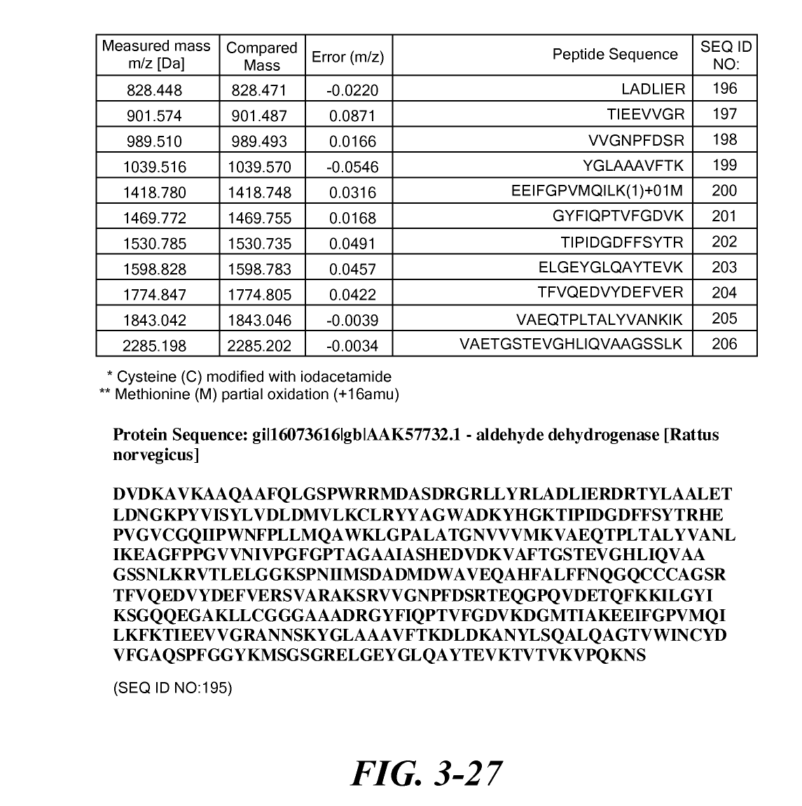

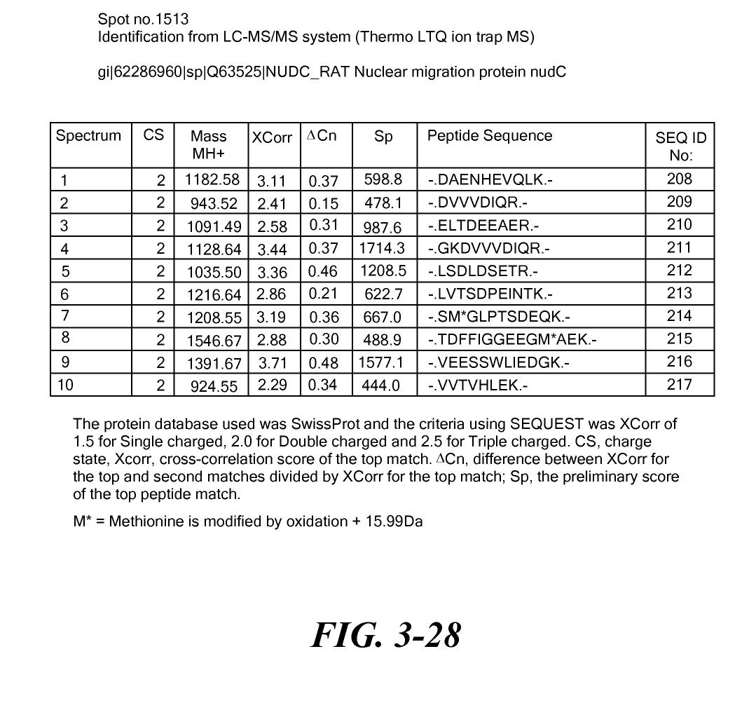

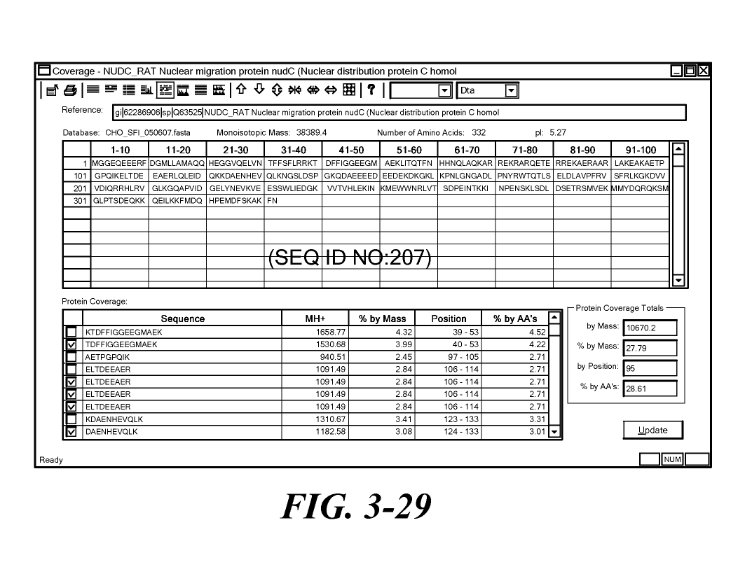

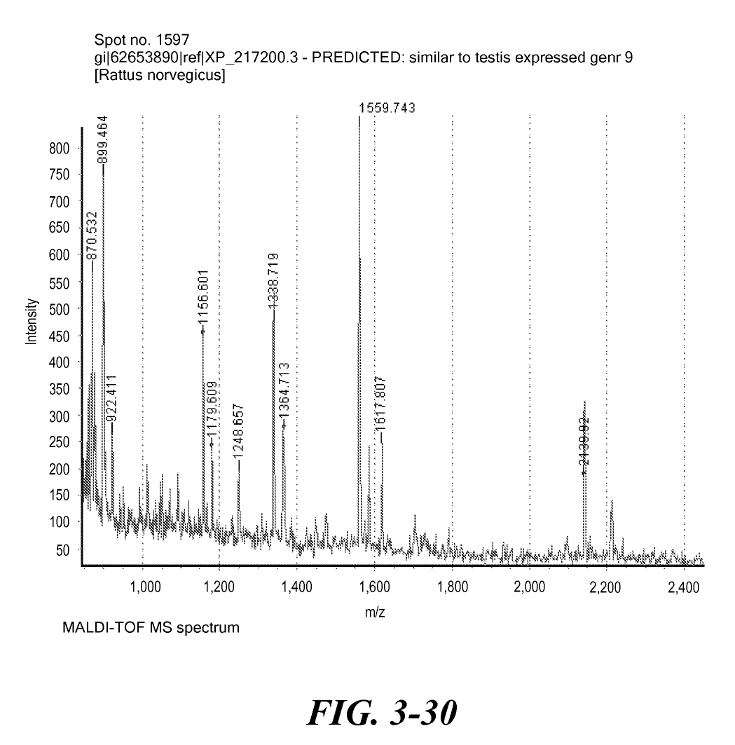

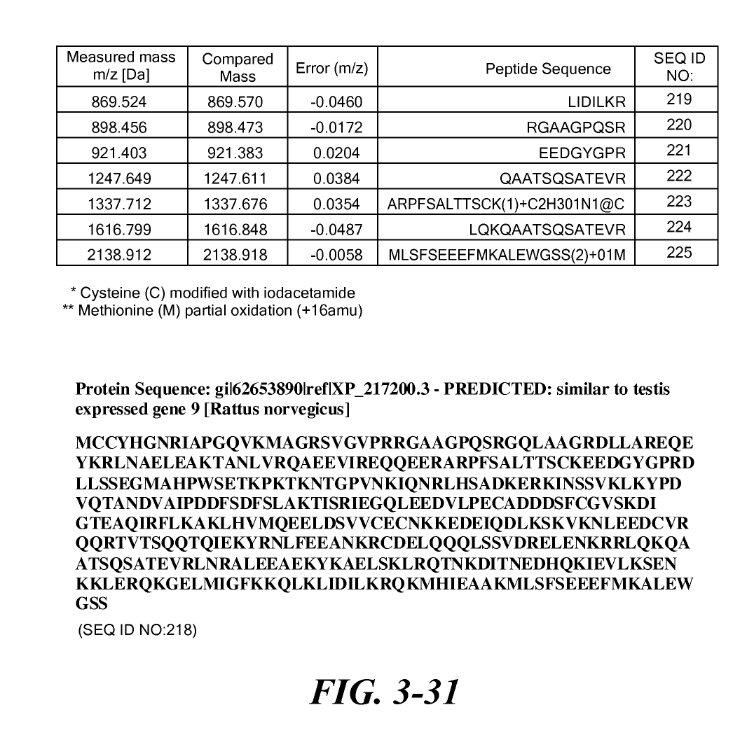

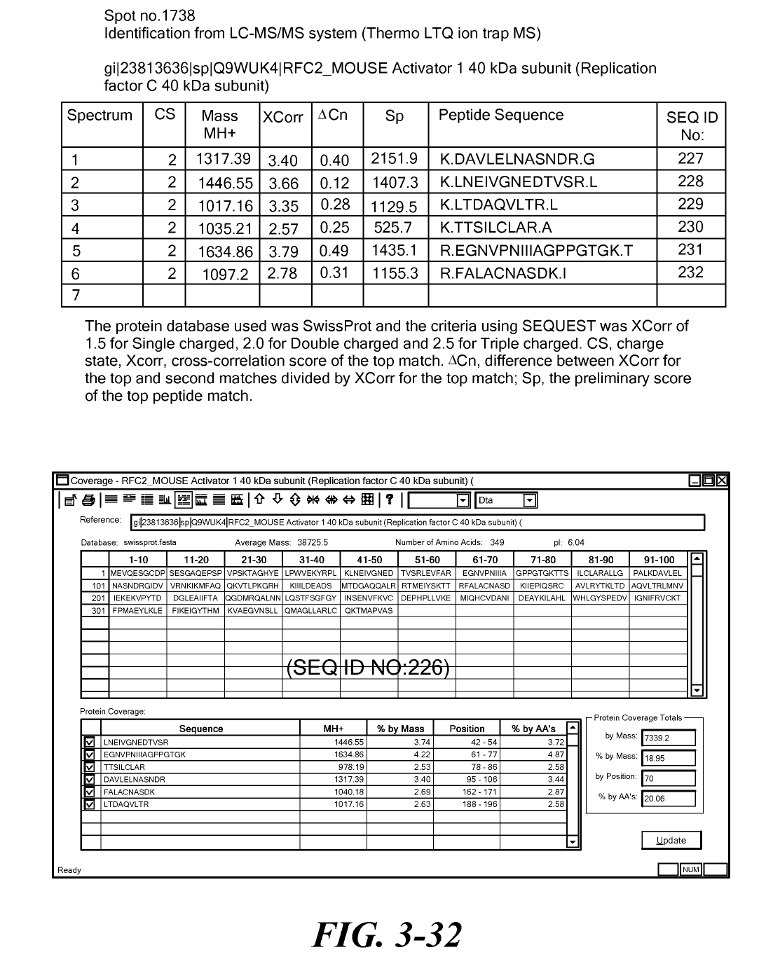

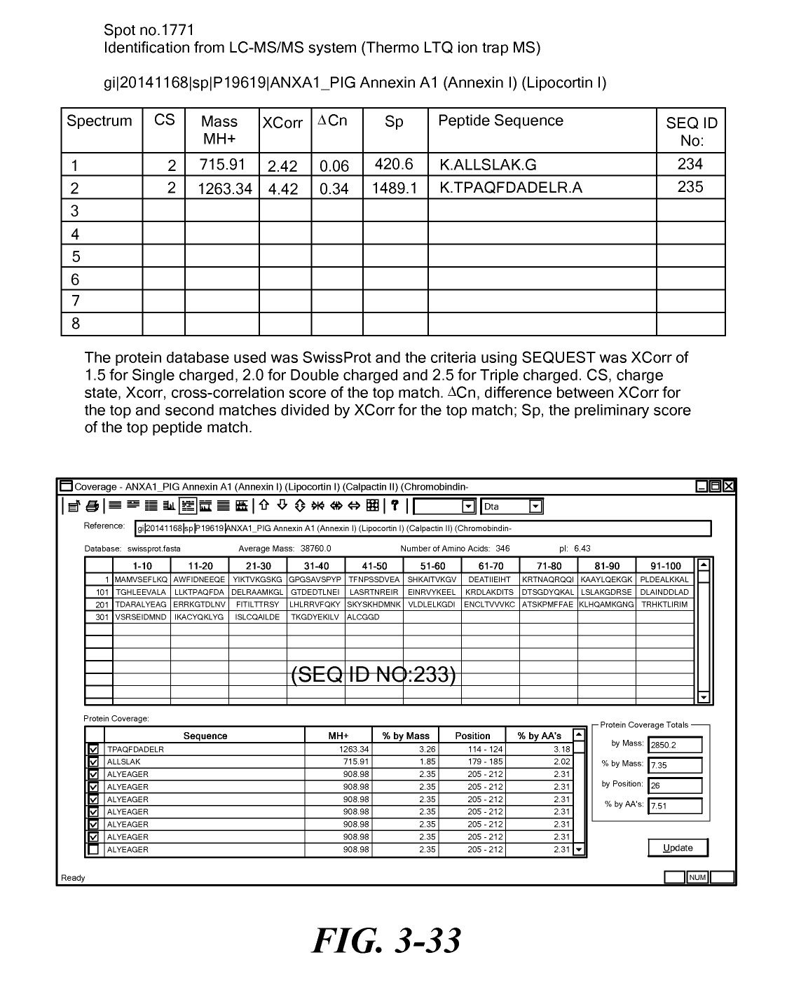

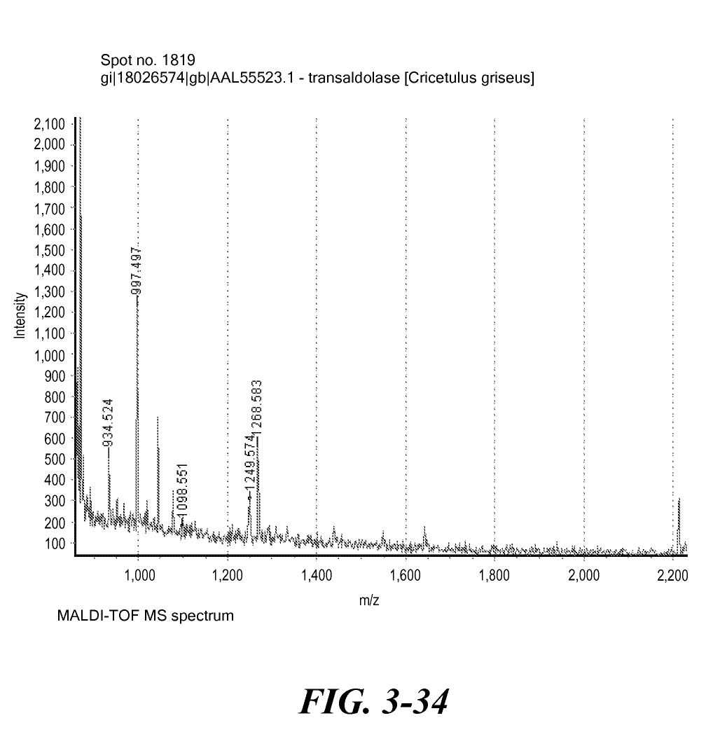

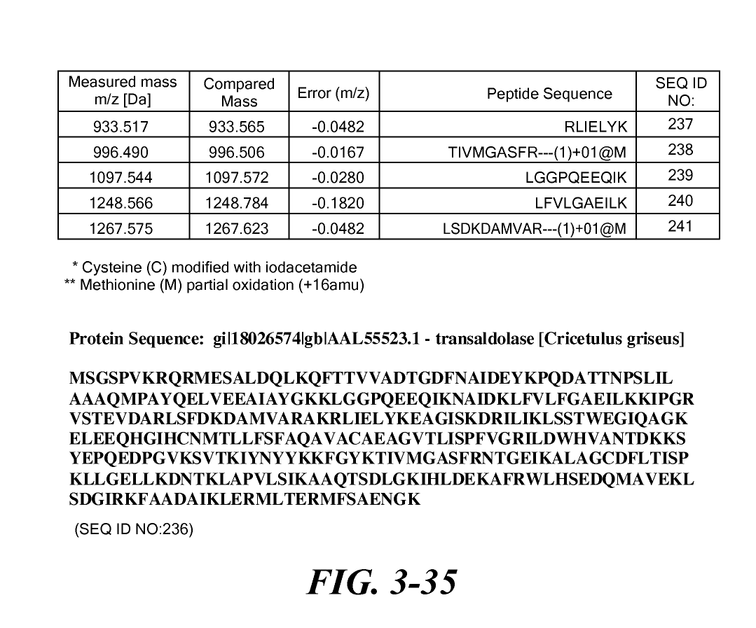

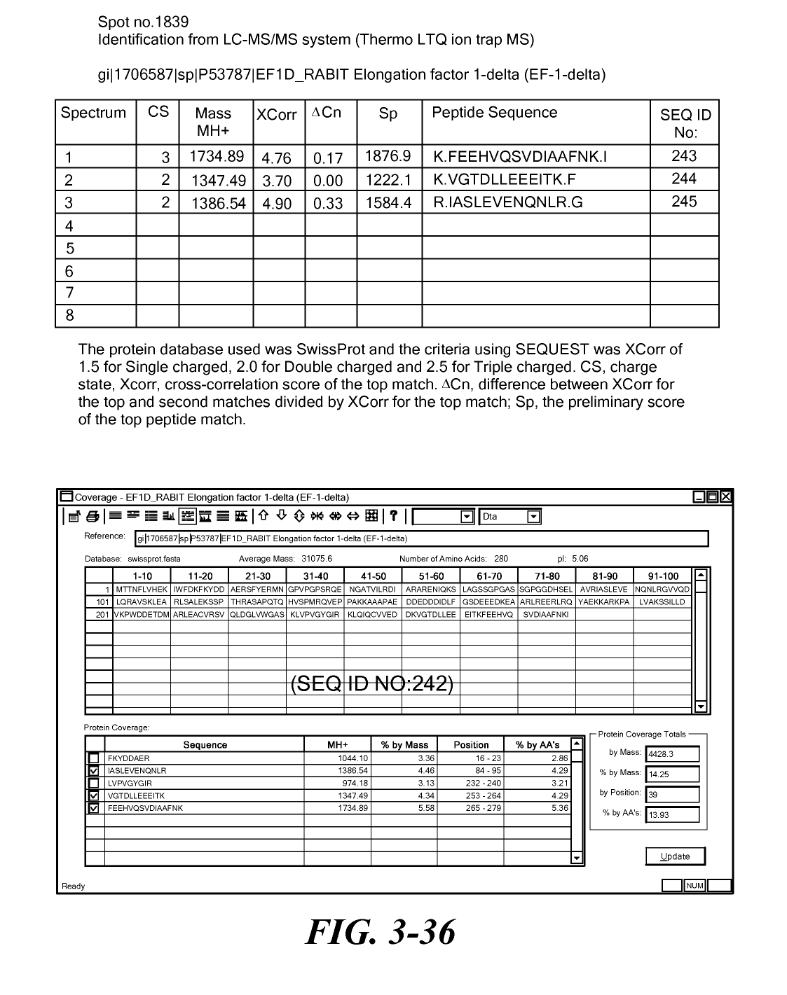

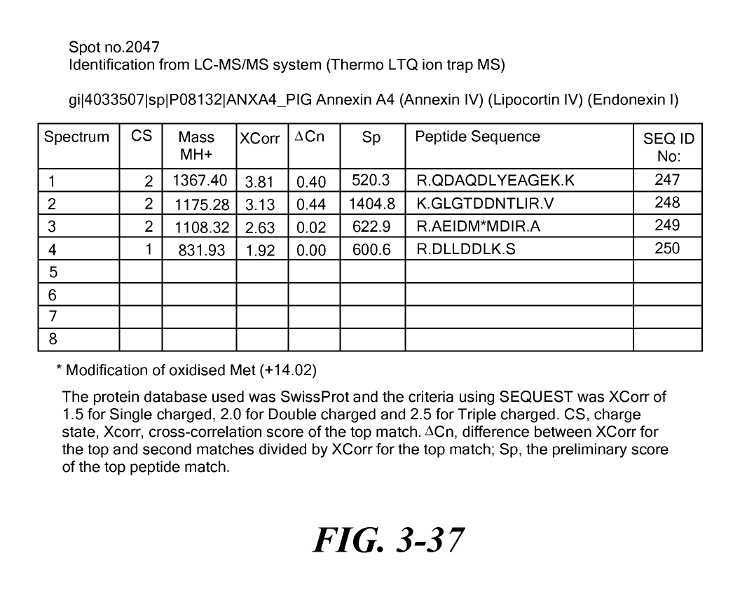

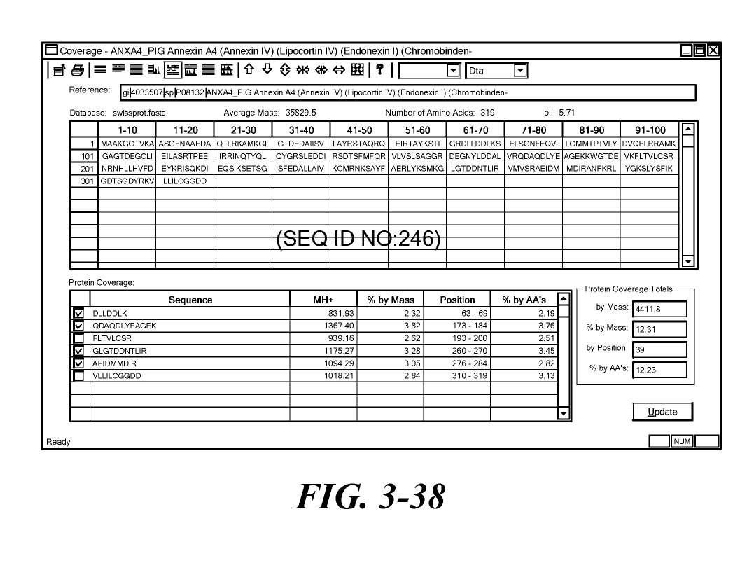

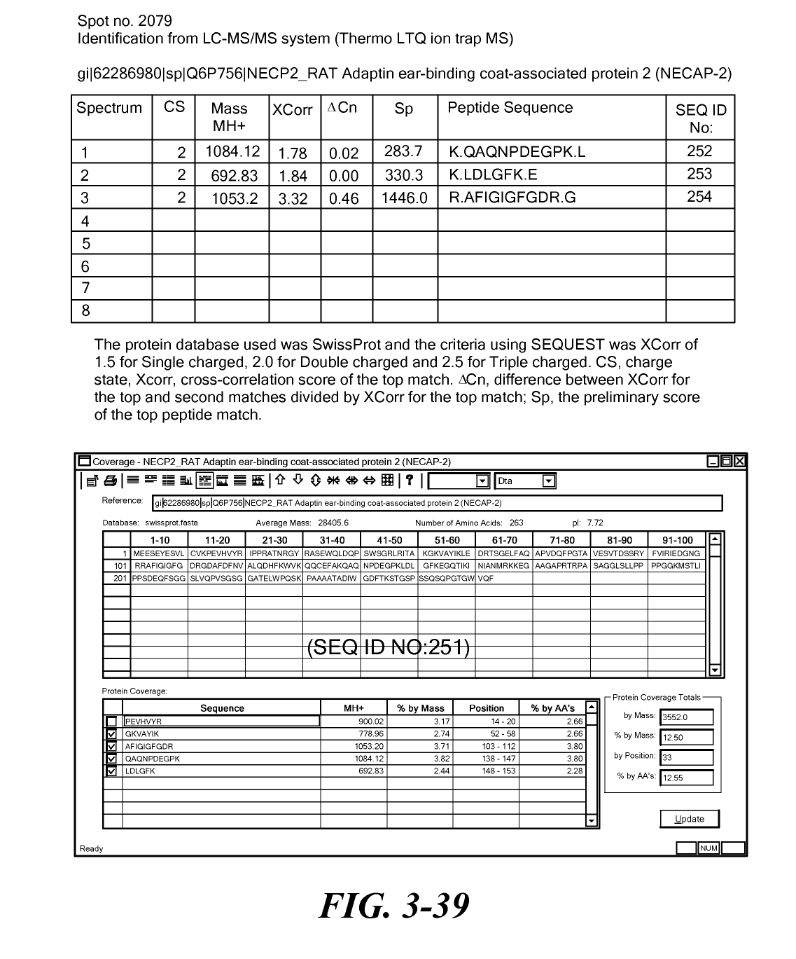

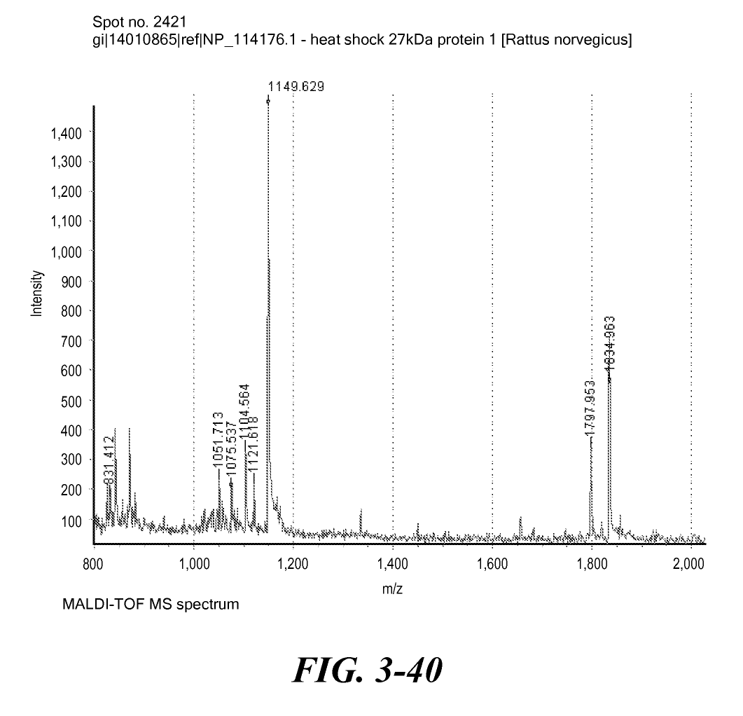

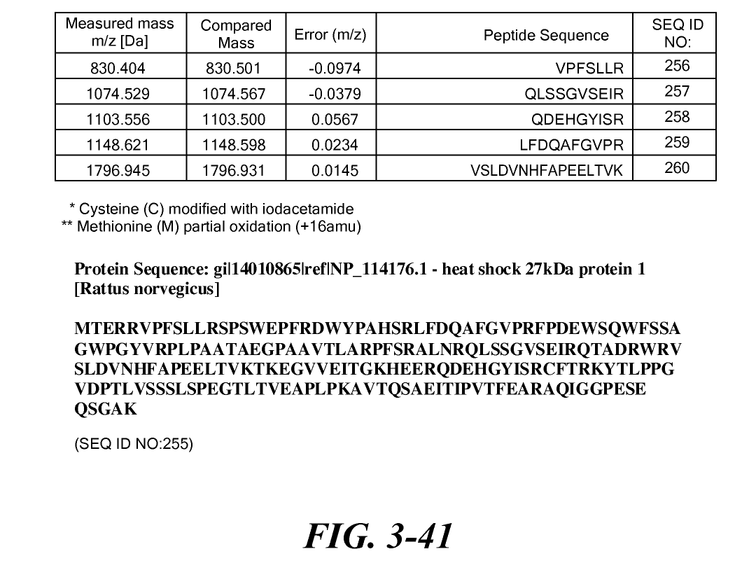

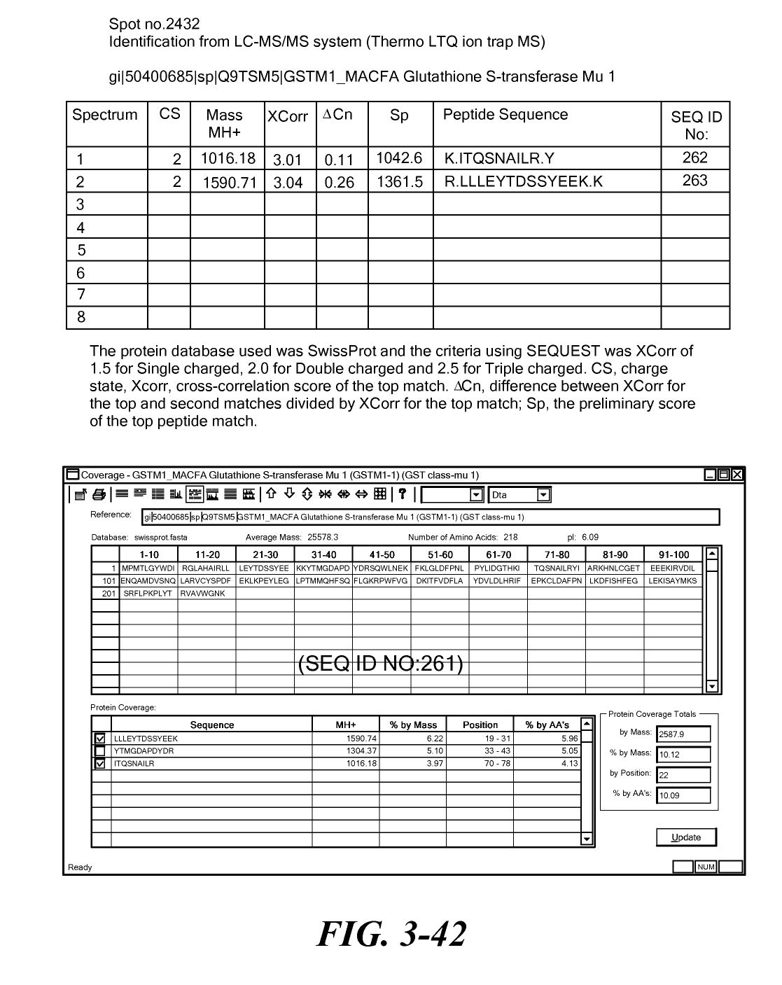

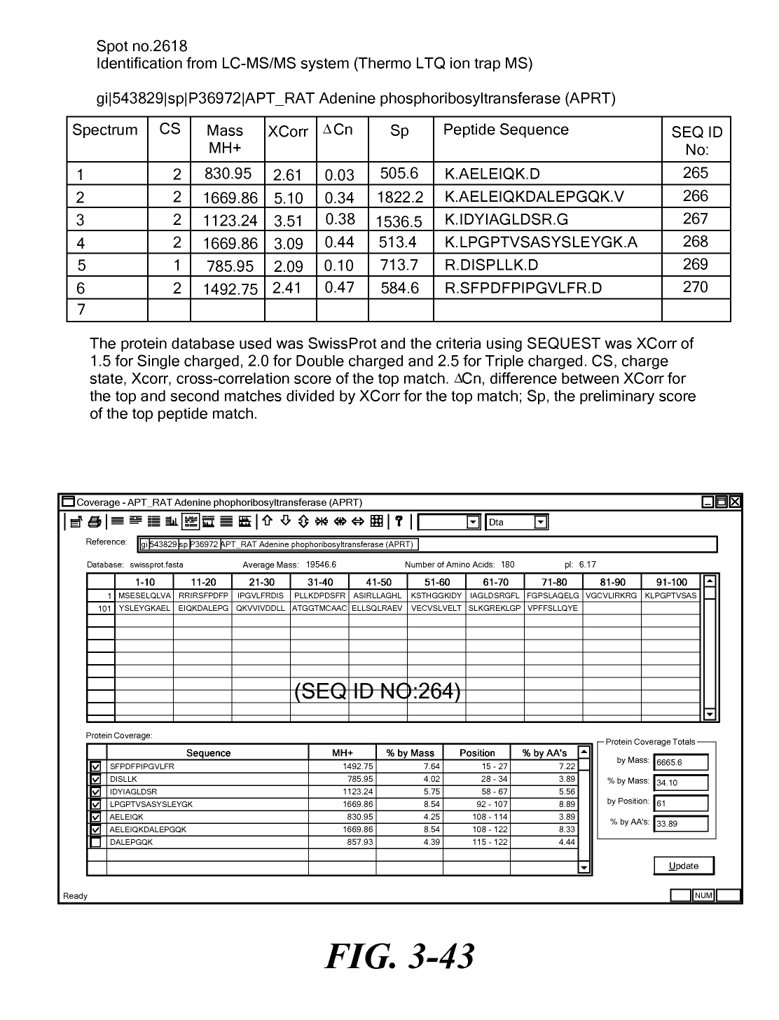

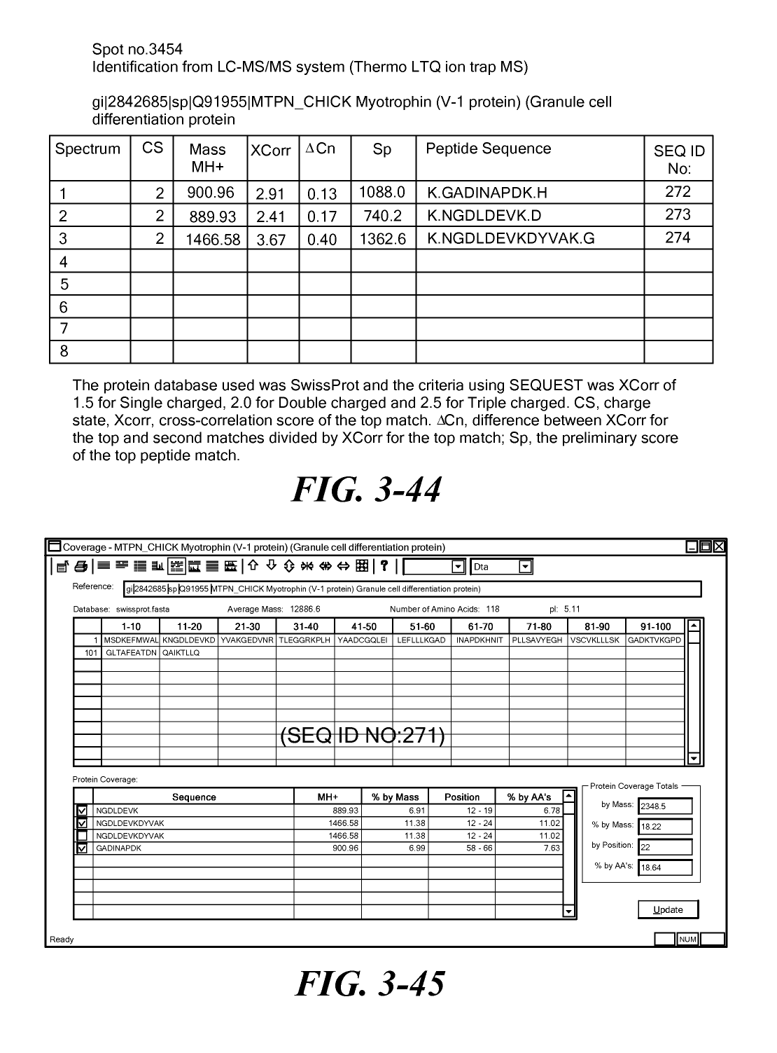

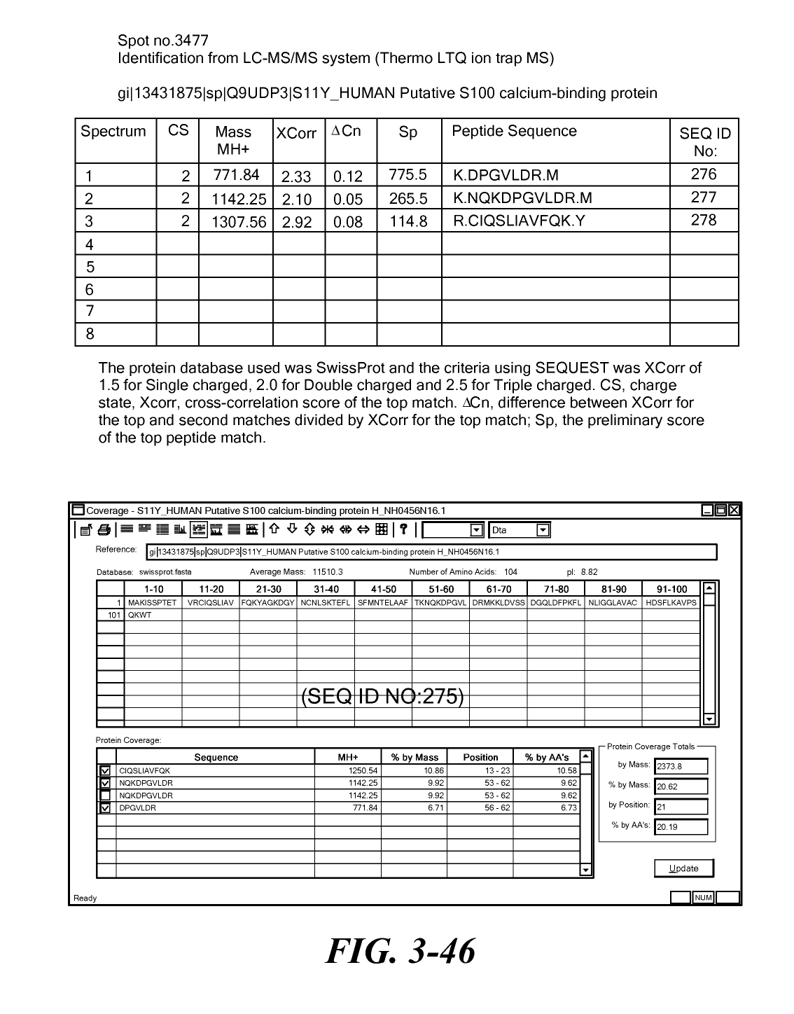

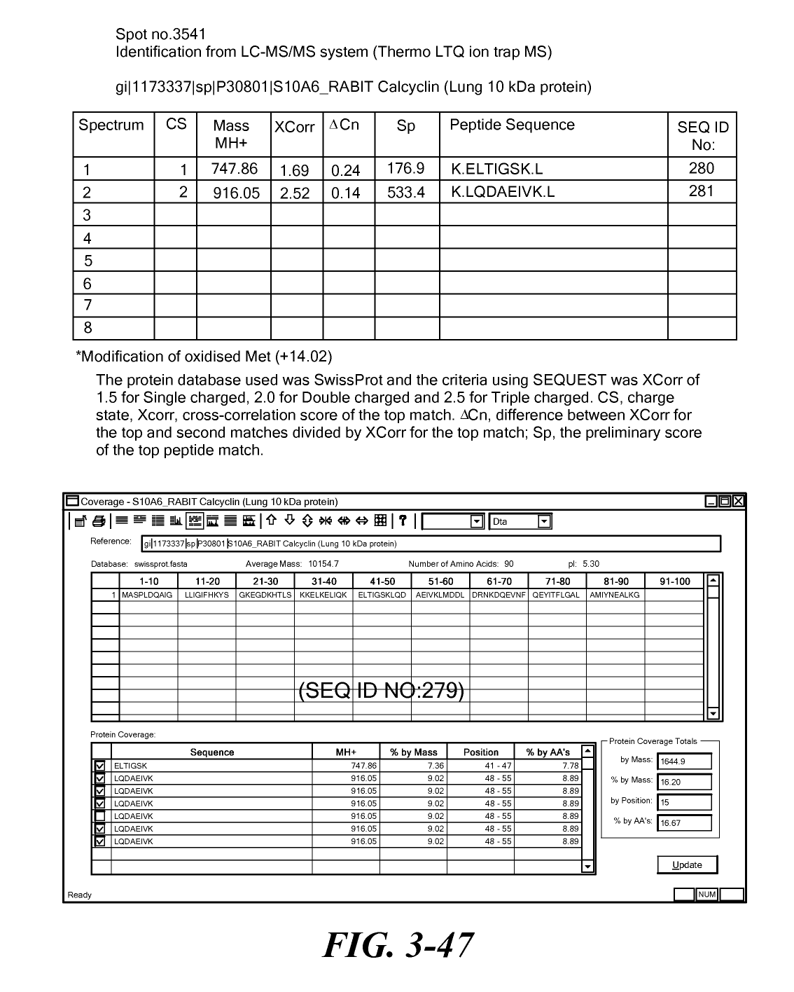

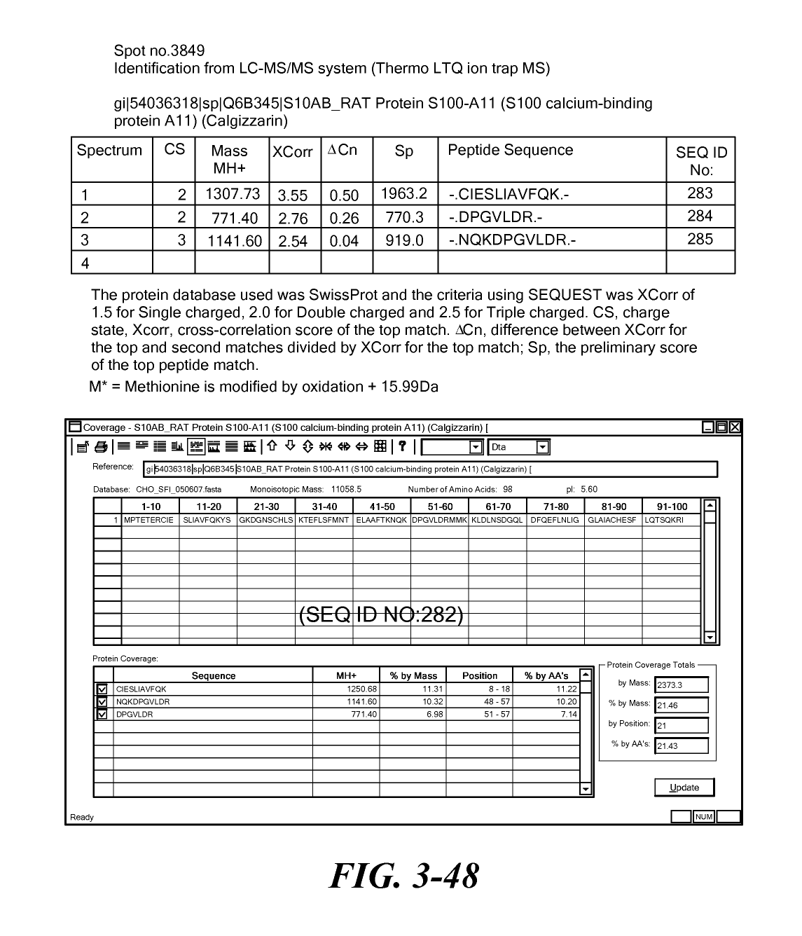

Sequence data for identified proteins are provided in FIGS. 3-1 through 3-118. Each figure provides, for a particular protein spot from the DIGE, the spectrum of molecular weights detected in the tryptic digest; the corresponding protein database match or matches, including the number of peptides matched to the predicted tryptic peptides for the protein database entry, the accession number, name, and species of the protein from the database entry, the percent coverage, the isoelectric point and mass; for each molecular weight matched with a predicted mass of a predicted peptide, the measured mass, the predicted (compared) mass, the difference between the two, and the corresponding peptide sequence; and the full length sequence of the protein from the database entry.

Proteins Unique to Control and Test Samples that Change Over Time of Process
(Filters—1.5 fold up/down regulation, t-test <0.05, 1-way anova <0.05, Decyder software statistical analysis)

TABLE 2
Exemplary Control-only Diferentially Expressed Protiens

| | | | | | | | | | No. |
|---|---|---|---|---|---|---|---|---|---|
| | | | | | | | | | peptides |
| | | | | | | | | | used for |
| | Fold Change | | | | | | | Expectancy | LC- |
| Decyder | (Control Day 7/ | Accession | | Mass Spec | | | | value - | MS/MS |
| Master no. | Control Day 3) | number | Protein Name | Identification | % coverage | pI | Mr | MALDI | ID |
| 2079 | −1.99 | gi\|62286980\| | Adaptin ear- | LC-MS/MS | 12.55 | 7.72 | 28.41 | | 3 |
| | | sp\|Q6P756\| | binding coat- | ID | | | | | |
| | | | associated protein 2 | | | | | | |

TABLE 3
Exemplary Control-only Differentially Expressed Protiens

| | Fold | | | | | | | No. |
|---|---|---|---|---|---|---|---|---|
| | Change | | | | | | | peptides |
| | (Control | | | | | | | used for |
| Decyder | Day 9/ | | | | | | | LC- |
| Master | Control | | | Mass Spec | % | | | MS/MS |
| no. | Day 6) | Accession number | Protein Name | Identification | coverage | pI | Mr | ID |
| 868 | −1.53 | gi\|123647\|sp\|P19378\| | HSP7C_CRIGR | LC-MS/MS ID | 38.91 | 5.24 | 70.8 | 24 |
| | | | Heat shock cognate | | | | | |
| | | | 71 kDa protein (Heat | | | | | |
| | | | shock 70 kDa protein | | | | | |
| | | | 8) | | | | | |
| 1513 | −1.55 | gi\|62286960\|sp\|Q63525\| | NUDC_RAT | LC-MS/MS ID | 27.79 | 5.27 | 38.4 | 9 |
| | | | Nuclear migration | | | | | |
| | | | protein nudC | | | | | |

TABLE 4
Exemplary Test-only Differentially Expressed Protiens

| | Fold | | | | | | | | No. |
|---|---|---|---|---|---|---|---|---|---|
| | Change | | | | | | | | peptides |
| | (Control | | | | | | | | used for |
| | Day 7/ | | | | | | | Expectancy | LC- |
| Decyder | Control | Accession | | Mass Spec | | | | value - | MS/MS |
| Master no. | Day 3) | number | Protein Name | Identification | % coverage | pI | Mr | MALDI | ID |
| 578 | 1.57 | gi\|124219\| | Eukaryotic | LC-MS/MS | 2.95 | 5.49 | 69.22 | | 2 |
| | | sp\|P23588\| | translation initiation | ID | | | | | |
| | | | factor 4B (eIF-4B) | | | | | | |
| 1031 | 1.85 | gi\|55824767\| | Phosphoglucomutase 2 | MALDI ID | 22.4 | 6.1 | 61.65 | 0 | |
| 1771 | 1.55 | gi\|20141168\| | Annexin A1 | LC-MS/MS | 7.51 | 38.76 | 6.43 | | 3 |
| | | sp\|P19619\| | (Annexin I) | ID | | | | | |
| 2432 | 1.52 | gi\|50400685\| | Glutathione S- | LC-MS/MS | 10.09 | 6.09 | 25.59 | | 2 |
| | | sp\|Q9TSM5\| | transferase Mu 1 | ID | | | | | |
| | | | (GSTM1-1) | | | | | | |

TABLE 5
Exemplary Test-only Differentially Expressed Protiens

| | Fold | | | | | | | No. |
|---|---|---|---|---|---|---|---|---|
| | Change | | | | | | | peptides |
| | (Control | | | | | | | used for |
| Decyder | Day 9/ | | | | | | | LC- |
| Master | Control | | | Mass Spec | % | | | MS/MS |
| no. | Day 6) | Accession number | Protein Name | Identification | coverage | pI | Mr | ID |
| 880 | 1.7 | gi\|14916999\|sp\|P11021\| | GRP78_HUMAN 78 kDa | LC-MS/MS | 49.13 | 5.07 | 72.3 | 29 |
| | | | glucose-regulated protein | ID | | | | |
| | | | precursor (GRP 78) | | | | | |

TABLE 6
Exemplary Process-only Differentially Expressed Proteins
Bolded font: Hits from Cricitulus Griseus (CHO)
Process-related proteins (common to control and test over time of process)
(Filters - 1.5 fold up/down regulation, t-test <0.05, 1-way anova <0.05, Decyder software statistical analysis)

| | Fold | | | | | | | | | No. |
|---|---|---|---|---|---|---|---|---|---|---|
| | Change | | | | | | | | | peptides |
| | (Control | | | | | | | | | used for |
| Decyder | Day 7/ | Fold Change | | | | | | | Expectancy | LC- |
| Master | Control | (Test Day 7/Test | Accession | | Mass Spec | | | | value - | MS/MS |
| no. | Day 3) | Day 3) | number | Protein Name | Identification | % coverage | pI | Mr | MALDI | ID |
| 328 | −1.71 | 0 | gi\|58865966 | tumor rejection antigen | MALDI ID | 19.4 | 5 | 74.42 | 0 | |
| | | | | gp96 (predicted) | | | | | | |
| 585 | −1.63 | −1.65 | gi\|40787768\| | Minichromosome | MALDI ID | 11.7 | 6 | 81.82 | 0.004 | |
| | | | | maintenance protein 7 | | | | | | |
| 831 | −1.59 | −1.57 | gi\|123621\| | Heat shock-related 70 kDa | LC-MS/MS | 12.32 | 5.58 | 69.74 | | 9 |
| | | | sp\|P17156 | protein 2 | ID | | | | | |
| 947 | 1.61 | 1.51 | gi\|1351207\| | T-complex protein 1 | LC-MS/MS | 16.37 | 5.76 | 60.34 | | 8 |
| | | | sp\|P11984\| | subunit alpha A (TCP-1- | ID | | | | | |
| | | | | alpha) (CCT-alpha) | | | | | | |
| 1082 | 1.74 | 1.82 | gi\|124426\| | IMDH2—CRIGR | LC-MS/MS | 12.65 | 6.84 | 55.89 | | 5 |
| | | | sp\|P12269\| | Inosine-5′- | ID | | | | | |
| | | | | monophosphate | | | | | | |
| | | | | dehydrogenase 2 (IMP | | | | | | |
| | | | | dehydrogenase 2) | | | | | | |
| 1169 | −1.8 | −1.76 | gi\|68846235\| | UDP-N- | LC-MS/MS | 8.24 | 5.92 | 58.77 | | 4 |
| | | | sp\|Q16222\| | acetylhexosamine | ID | | | | | |
| | | | | pyrophosphorylase | | | | | | |
| | | | | (Antigen X) | | | | | | |
| 1171 | −2.86 | −2.98 | gi\|5542272\| | gChain A, Importin | MALDI ID | 15 | 5.4 | 49.61 | 0.001 | |
| | | | | Alpha, Mouse | | | | | | |
| 1181 | 1.63 | 1.66 | gi\|17105370\| | ATPase, H+ transporting, | MALDI ID | 24.5 | 5.6 | 56.88 | 0.003 | |
| | | | | V1 subunit B, isoform 2 | | | | | | |
| | | | | [Rattus norvegicus] | | | | | | |
| 1257 | 1.6 | 1.54 | gi\|42543061 | Chain A, Crystal | MALDI ID | 30 | 5 | 31.94 | 0.001 | |
| | | | | Structure Of Sp-Camp | | | | | | |
| | | | | Binding R1a Subunit Of | | | | | | |
| | | | | Camp-Dependent Protein | | | | | | |
| | | | | Kinase | | | | | | |
| 1299 | 1.65 | 1.69 | gi\|16073616\| | aldehyde dehydrogenase | MALDI ID | 31.6 | 6.1 | 48.65 | 0.001 | |
| 1597 | 1.91 | 1.59 | gi\|62653890 | PREDICTED: similar to | MALDI ID | 16.9 | 7.1 | 46.48 | 0.005 | |
| | | | | testis expressed gene 9 | | | | | | |
| 1738 | −1.64 | −1.53 | gi\|23813636\| | Activator 1 40 kDa | LC-MS/MS | 20.06 | 6.04 | 38.73 | | 6 |
| | | | sp\|Q9WUK4\| | subunit | ID | | | | | |
| 1819 | −1.87 | −1.68 | gi\|18026574 | transaldolase | MALDI ID | 14.2 | 7 | 37.54 | 0.009 | |
| 1839 | −1.54 | −1.62 | gi\|1706587\| | Elongation factor 1-delta | LC-MS/MS | 13.93 | 5.06 | 31.08 | | 3 |
| | | | sp\|P53787\| | (EF-1-delta) | ID | | | | | |
| 2046 | 1.62 | 1.67 | gi\|4033507\| | Annexin A4 (Annexin | LC-MS/MS | 12.23 | 5.71 | 35.83 | | 4 |
| | | | sp\|P08132\| | IV) (Lipocortin IV) | ID | | | | | |
| | | | | (Endonexin I) | | | | | | |
| 2421 | 1.73 | 1.54 | gi\|14010865\| | heat shock 27 kDa protein 1 | MALDI ID | 25.4 | 6.1 | 22.86 | 0.007 | |
| 2426 | 1.53 | 1.74 | gi\|14010865\| | heat shock 27 kDa protein 1 | MALDI ID | 25.2 | 6.1 | 22.86 | 0.007 | |
| 2618 | 1.86 | 1.88 | gi\|543829\| | adenine | LC-MS/MS | 33.89 | 6.17 | 19.55 | | 6 |
| | | | sp\|P36972 | phosphoribosyltransferase | ID | | | | | |
| 3454 | 3.59 | 2.35 | gi\|2842685\| | Myotrophin (V-1 protein) | LC-MS/MS | 18.64 | 5.11 | 12.89 | | 3 |
| | | | sp\|Q91955 | (Granule cell | ID | | | | | |
| | | | | differentiation protein) | | | | | | |
| 3477 | 1.69 | 1.77 | gi\|13431875\| | Putative S100 calcium- | LC-MS/MS | 20.19 | 8.82 | 11.51 | | 3 |
| | | | sp\|Q9UDP3\| | binding protein | ID | | | | | |
| | | | | H_NH0456N16.1 | | | | | | |
| 3541 | 2.23 | 2 | gi\|1173337\| | Calcyclin (Prolactin | LC-MS/MS | 16.67 | 5.3 | 10.15 | | 2 |
| | | | sp\|P30801 | receptor associated | ID | | | | | |
| | | | | protein) | | | | | | |

TABLE 7
Exemplary Process-only Differentially Expressed Proteins
Process-related proteins (common to control and test over time of process)
(Filters - 1.5 fold up/down regulation, t-test <0.05, 1-way anova <0.05, Decyder software statistical analysis)

| | Fold | Fold | | | | | | | |
|---|---|---|---|---|---|---|---|---|---|
| | Change | Change | | | | | | | |
| | (Control | (Test | | | | | | | |
| Decyder | day 9/ | day 9 | | | | | | | No. peptides |
| Master | Control | Test day | Accession | | Mass Spec | % | | | used for LC- |
| no. | day 6) | 6) | number | Protein Name | Identification | coverage | pI | Mr | MS/MS ID |
| 837 | 1.56 | 1.7 | gi\|59799762\| | HSP7C_CAEBR Heat shock 70 | LC-MS/MS ID | 6.44 | 5.00 | 72.95 | 5 |
| | | | sp\|P19208\| | kDa protein C precursor | | | | | |
| 838 | 1.51 | 1.83 | gi\|14916999\| | GRP78_HUMAN 78 kDa | LC-MS/MS ID | 33.75 | 5.07 | 72.29 | 19 |
| | | | sp\|P11021\| | glucose-regulated protein | | | | | |
| | | | | precursor (GRP 78) | | | | | |
| 1157 | −1.58 | −1.54 | gi\|123332\| | HMCS1_CRIGR | LC-MS/MS ID | 17.51 | 5.41 | 57.28 | 8 |
| | | | sp\|P13704\| | Hydroxymethylglutaryl-CoA | | | | | |
| | | | | synthase, cytoplasmic | | | | | |
| 1159 | −1.63 | −1.65 | gi\|18314334\| | FKBP4_MOUSE FK506- | LC-MS/MS ID | 17.02 | 5.54 | 51.54 | 8 |
| | | | sp\|P30416\| | binding protein 4 (Peptidyl- | | | | | |
| | | | | prolyl cis-trans isomerase) | | | | | |
| 3849 | 1.56 | 1.65 | gi\|54036318\| | S10AB_RAT Protein S100-A11 | LC-MS/MS ID | 21.46 | 5.60 | 11.06 | 3 |
| | | | sp\|Q6B345\| | (S100 calcium-binding protein | | | | | |
| | | | | A11) (Calgizzarin) | | | | | |

### Example 4

### mRNA Expression Profiling

Samples from test cell line or control cell line at multiple time points were taken as described above and ANOVA analysis was conducted. In this experiment, the test cell line maintains a high viability throughout the fed batch, while the control cell line declines in viability early. RNA samples from sample were obtained and analyzed on a microchip containing probes for CHO mRNA sequences as described in U.S. Patent Application Publication US2006/0010513, the complete contents of which are herein incorporated by reference. The hybridization cocktail was spiked with a fragmented cRNA standard to generate a standard curve using labeled, fragmented cRNA of control sequences at known concentrations, permitting normalization of the data and assessment of chip sensitivity and saturation. The scan data were quality controlled using the 3′/5′ ratio of β-actin and GAPDH, the signal intensity and consistency, and the percent present. Generally, data normalization was performed using software tools Affy 5.0 and Genesis 2.0; or dChiP (see Li et al. (2001) Proc. Natl. Acad. Sci. USA 98:31-36 and Li et al. (2001) Genome Biol. 2:0032.1-0032.11) and Genespring. A P_{Value} less than or equal to 0.05 and a fold-change minimum between different time points of 1.2 was required before a gene would be further considered.

The differentially expressed genes in the test cell line were compared to the differentially expressed genes in the control cell line to classify the differentially expressed genes into three groups, namely, the test cell-only, the control cell-only and common differentially expressed genes.

Alternatively, two parallel comparisons were conducted using two pairs of cell lines (test cell line vs. control cell line) (referred to as HCD3 and HCD4, respectively). ANOVA analysis as described above was conducted for each pair at 3 time points (days 3, 5, and 7 for HCD3 and days 3, 6, 9 for HCD4) culture samples. For each of HCD3 and HCD4, the differentially expressed genes in test or control cells were compared in order to identify those genes unique to test cells (test-only or test-specific) or control cells (control-only or control-specific), or those in common to both (process-only or process-specific). Each of the test-only, control-only, and process-only gene lists from HCD3 were overlapped with the corresponding list from HCD4 to identify commonly up or down-regulated test-only differentially expressed genes for both HCD3 and HCD4, commonly up or down-regulated control-only differentially expressed genes for both HCD3 and HCD4, or commonly up or down-regulated process-only differentially expressed genes for both HCD3 and HCD4.

Exemplary differentially expressed genes were shown in different categories in Table 8. Exemplary control cell-only differentially expressed genes were shown in Table 9. Exemplary test cell-only differentially expressed genes were shown in Table 10. Exemplary common differentially expressed genes were shown in Table 11. As discussed above, common differentially expressed genes are process related.

For each nucleic acid, a qualifier name, symbol, and title are provided, as well as whether the nucleic acid is up-regulated or down-regulated in the respective cell lines. For nucleic acids with human or mouse homologs in the Unigene database, the table provides Unigene ID numbers and statistics relating to the comparison, including e-values, percent sequence identities between the CHO sequence and the Unigene databank entries, and percent coverage (“% QC”).

TABLE 8
Exemplary Differentially Expressed Genes

| | | | | Human | | |
|---|---|---|---|---|---|---|
| | FC | Symbol | Title | Unigene ID | eValue | % ID |
| Control Only | | | | | | |
| Up | | | | | | |
| 0 | | | | | | |
| Down | | | | | | |
| WAN013I8X_at | 1.811 | HSPD1 | Heat shock 60 kDa | Hs.595053 | 0 | 90.23291 |
| (SEQ ID NO: 286) | | | protein 1 | | | |
| | | | (chaperonin) | | | |
| WAN008EH7_at | 1.643 | ASNSD1 | Asparagine | Hs.101364 | 8E−39 | 82.15488 |
| (SEQ ID NO: 287) | | | synthetase domain | | | |
| | | | containing 1 | | | |
| WAN013HXI_at | 1.544 | ACAT2 | Acetyl-Coenzyme A | Hs.571037 | 1E−122 | 83.06011 |
| (SEQ ID NO: 288) | | | acetyltransferase 2 | | | |
| | | | (acetoacetyl | | | |
| | | | Coenzyme A | | | |
| | | | thiolase) | | | |
| WAN008D37_at | 1.515 | Eif3s10 | Eukaryotic | #N/A | 3E−66 | 83.73984 |
| (SEQ ID NO: 289) | | | translation initiation | | | |
| | | | factor 3, subunit 10 | | | |
| | | | (theta) | | | |
| Process | | | | | | |
| Up | | | | | | |
| 0 | | | | | | |
| Down | | | | | | |
| AF292400_at | 3.182 | ASK | Activator of S phase | #N/A | 2E−78 | 91.59292 |
| (SEQ ID NO: 290) | | | kinase | | | |
| M80243-rc_at | 1.722 | BIRC5 | Baculoviral IAP | Hs.514527 | 4E−38 | 92.37288 |
| (SEQ ID NO: 291) | | | repeat-containing 5 | | | |
| | | | (survivin) | | | |
| WAN008CYY_at | 3.69 | BUB1B | BUB1 budding | Hs.631699 | 4E−34 | 81.32184 |
| (SEQ ID NO: 292) | | | uninhibited by | | | |
| | | | benzimidazoles 1 | | | |
| | | | homolog beta (yeast) | | | |
| WAN008CVX_at | 5.159 | CDC20 | CDC20 cell division | Hs.524947 | 1E−169 | 90.6639 |
| (SEQ ID NO: 293) | | | cycle 20 homolog | | | |
| | | | (S. cerevisiae) | | | |
| WAN013I9R_at | 4.631 | NA | Cluster includes | #N/A | 1E−104 | 84.573 |
| (SEQ ID NO: 294) | | | Y08202 C. griseus | | | |
| | | | mRNA for RAD51 | | | |
| | | | protein | | | |
| WAN013I5T_at | 8.17 | CCNB1 | Cyclin B1 | Hs.23960 | 1E−93 | 85.30259 |
| (SEQ ID NO: 295) | | | | | | |
| WAN013I8J_at | 5.381 | CCNB2 | Cyclin B2 | Hs.194698 | 1E−173 | 86.9258 |
| (SEQ ID NO: 296) | | | | | | |
| U48852_at | 2.078 | CRELD2 | Cysteine-rich with | Hs.211282 | 1E−109 | 81.76944 |
| (SEQ ID NO: 296) | | | EGF-like domains 2 | | | |
| WAN0088T7_at | 1.667 | Cyp51 | Cytochrome P450, | #N/A | 1E−132 | 86.87873 |
| (SEQ ID NO: 298) | | | family 51 | | | |
| WAN008E8K_at | 1.69 | H2-K1 | Histocompatibility 2, | #N/A | 1E−154 | 92.60204 |
| (SEQ ID NO: 299) | | | K1, K region | | | |
| WAN008EXF_at | 6.014 | KIF11 | Kinesin family | Hs.8878 | 4E−28 | 86.86131 |
| (SEQ ID NO: 300) | | | member 11 | | | |
| X83576_at | 2.459 | KIFC1 | Kinesin family | Hs.436912 | 0 | 86.77111 |
| (SEQ ID NO: 301) | | | member C1 | | | |
| WAN008CX4_at | 7.518 | MCM5 | MCM5 | Hs.517582 | 1E−152 | 87.10247 |
| (SEQ ID NO: 302) | | | minichromosome | | | |
| | | | maintenance | | | |
| | | | deficient 5, cell | | | |
| | | | division cycle 46 | | | |
| | | | (S. cerevisiae) | | | |
| WAN008E7Y_at | 4.301 | MCM7 | MCM7 | Hs.438720 | 1E−96 | 84.5339 |
| (SEQ ID NO: 303) | | | minichromosome | | | |
| | | | maintenance | | | |
| | | | deficient 7 | | | |
| | | | (S. cerevisiae) | | | |
| WAN008DWL_at | 2.879 | NEK2 | NIMA (never in | Hs.153704 | 1E−71 | 87.38739 |
| (SEQ ID NO: 304) | | | mitosis gene a)- | | | |
| | | | related kinase 2 | | | |
| WAN008EML_at | 6.697 | PBK | PDZ binding kinase | Hs.104741 | 5E−52 | 89.50276 |
| (SEQ ID NO: 305) | | | | | | |
| WAN008ELE_at | 2.194 | PSAT1 | Phosphoserine | Hs.494261 | 7E−27 | 93.10345 |
| (SEQ ID NO: 306) | | | aminotransferase 1 | | | |
| WAN013I8E_at | 1.674 | PCNA | Proliferating cell | Hs.147433 | 1E−173 | 90.92784 |
| (SEQ ID NO: 307) | | | nuclear antigen | | | |
| WAN008E3C_at | 3.048 | Ptma | Prothymosin alpha | Hs.459927 | 2E−67 | 93.83886 |
| (SEQ ID NO: 308) | | | | | | |
| WAN008EJV_at | 5.368 | Racgap1 | Rac GTPase- | Hs.645513 | 1E−103 | 86.39618 |
| (SEQ ID NO: 309) | | | activating protein 1 | | | |
| WAN0088U6_at | 10.96 | SPAG5 | Sperm associated | Hs.514033 | 1E−108 | 84.43649 |
| (SEQ ID NO: 310) | | | antigen 5 | | | |
| WAN008CJI_at | 1.961 | SFRS2 | Splicing factor, | Hs.584801 | 3E−81 | 97.23757 |
| (SEQ ID NO: 311) | | | arginine/serine-rich 2 | | | |
| WAN013IAD_at | 12.72 | TOP2A | Topoisomerase | Hs.156346 | 3E−37 | 80.62678 |
| (SEQ ID NO: 312) | | | (DNA) II alpha | | | |
| | | | 170 kDa | | | |
| Test | | | | | | |
| Up | | | | | | |
| 0 | | | | | | |
| Down | | | | | | |
| WAN008DMP_at | 1.572 | EWSR1 | Ewing sarcoma | Hs.374477 | 1E−157 | 90.52863 |
| (SEQ ID NO: 313) | | | breakpoint region 1 | | | |
| WAN008EL3_at | 1.56 | LSM3 | LSM3 homolog, U6 | Hs.111632 | 1E−102 | 90.69767 |
| (SEQ ID NO: 314) | | | small nuclear RNA | | | |
| | | | associated | | | |
| | | | (S. cerevisiae) | | | |
| U10249_at | 1.56 | CDK2A | CDK2-associated | Hs.433201 | 9E−77 | 86.82635 |
| (SEQ ID NO: 315) | | P1 | protein 1 | | | |

| | | Mouse | | | |
|---|---|---|---|---|---|
| | % QC | Unigene ID | eValue | % ID | % QC |
| Control Only | | | | | |
| Up | | | | | |
| 0 | | | | | |
| Down | | | | | |
| WAN013I8X_at | 99.77511 | Mm.1777 | 0 | 93.38843 | 99.77511 |
| (SEQ ID NO: 286) | | | | | |
| WAN008EH7_at | 55 | Mm.285783 | 1E−107 | 86.23025 | 82.03704 |
| (SEQ ID NO: 287) | | | | | |
| WAN013HXI_at | 98.74101 | Mm.229342 | 0 | 88.32117 | 98.56115 |
| (SEQ ID NO: 288) | | | | | |
| WAN008D37_at | 78.51064 | Mm.2238 | 1E−152 | 94.10188 | 79.3617 |
| (SEQ ID NO: 289) | | | | | |
| Process | | | | | |
| Up | | | | | |
| 0 | | | | | |
| Down | | | | | |
| AF292400_at | 17.97932 | #N/A | 1E−152 | 86.44338 | 49.88067 |
| (SEQ ID NO: 290) | | | | | |
| M80243-rc_at | 20.34483 | Mm.8552 | 1E−36 | 93.45794 | 18.44828 |
| (SEQ ID NO: 291) | | | | | |
| WAN008CYY_at | 73.10924 | Mm.29133 | 2E−71 | 84.59384 | 75 |
| (SEQ ID NO: 292) | | | | | |
| WAN008CVX_at | 85.15901 | Mm.289747 | 0 | 92.30769 | 87.27915 |
| (SEQ ID NO: 293) | | | | | |
| WAN013I9R_at | 63.46154 | #N/A | 1E−142 | 88.98072 | 63.46154 |
| (SEQ ID NO: 294) | | | | | |
| WAN013I5T_at | 28.11994 | Mm.260114 | 1E−142 | 87.17949 | 28.44408 |
| (SEQ ID NO: 295) | | | | | |
| WAN013I8J_at | 44.39216 | Mm.22592 | 0 | 90.70946 | 46.43137 |
| (SEQ ID NO: 296) | | | | | |
| U48852_at | 55.13673 | Mm.292567 | 0 | 88.79936 | 91.7221 |
| (SEQ ID NO: 296) | | | | | |
| WAN0088T7_at | 98.05068 | Mm.46044 | 1E−152 | 88.51485 | 98.44055 |
| (SEQ ID NO: 298) | | | | | |
| WAN008E8K_at | 98 | Mm.33263 | 1E−168 | 93.75 | 100 |
| (SEQ ID NO: 299) | | | | | |
| WAN008EXF_at | 27.56539 | Mm.42203 | 3E−24 | 91.86047 | 17.30382 |
| (SEQ ID NO: 300) | | | | | |
| X83576_at | 86.84807 | Mm.335713 | 0 | 90.52369 | 90.92971 |
| (SEQ ID NO: 301) | | | | | |
| WAN008CX4_at | 100 | Mm.5048 | 0 | 91.48936 | 99.64664 |
| (SEQ ID NO: 302) | | | | | |
| WAN008E7Y_at | 88.38951 | Mm.241714 | 1E−171 | 90.98532 | 89.32584 |
| (SEQ ID NO: 303) | | | | | |
| WAN008DWL_at | 58.42105 | Mm.33773 | 1E−152 | 89.13934 | 85.61404 |
| (SEQ ID NO: 304) | | | | | |
| WAN008EML_at | 39.09287 | Mm.24337 | 3E−80 | 89.78102 | 59.17927 |
| (SEQ ID NO: 305) | | | | | |
| WAN008ELE_at | 16.171 | Mm.289936 | 4E−70 | 87.53994 | 58.17844 |
| (SEQ ID NO: 306) | | | | | |
| WAN013I8E_at | 83.19039 | Mm.7141 | 0 | 92.2813 | 100 |
| (SEQ ID NO: 307) | | | | | |
| WAN008E3C_at | 44.98934 | Mm.19187 | 1E−148 | 92.74005 | 91.04478 |
| (SEQ ID NO: 308) | | | | | |
| WAN008EJV_at | 93.31849 | Mm.273804 | 1E−133 | 89.31116 | 93.76392 |
| (SEQ ID NO: 309) | | | | | |
| WAN0088U6_at | 98.07018 | Mm.24250 | 1E−153 | 87.12522 | 99.47368 |
| (SEQ ID NO: 310) | | | | | |
| WAN008CJI_at | 41.70507 | Mm.21841 | 1E−143 | 92.79778 | 83.17972 |
| (SEQ ID NO: 311) | | | | | |
| WAN013IAD_at | 29.52061 | Mm.4237 | 1E−86 | 84.11633 | 37.59462 |
| (SEQ ID NO: 312) | | | | | |
| Test | | | | | |
| Up | | | | | |
| 0 | | | | | |
| Down | | | | | |
| WAN008DMP_at | 94.19087 | Mm.142822 | 0 | 92.98246 | 94.60581 |
| (SEQ ID NO: 313) | | | | | |
| WAN008EL3_at | 65.15152 | Mm.246693 | 1E−165 | 91.77489 | 100 |
| (SEQ ID NO: 314) | | | | | |
| U10249_at | 35.95264 | Mm.390335 | 0 | 93.15353 | 51.88375 |
| (SEQ ID NO: 315) | | | | | |

TABLE 9
Exemplary Control-only Differentially Expressed Genes

| Control Only | FC | Symbol | Title | Human Unigene ID | eValue | % ID | % QC | Mouse Unigene ID | eValue | % ID | % QC |
|---|---|---|---|---|---|---|---|---|---|---|---|
| Up | | | | | | | | | | | |
| WAN008E5I_at | 1.560062 | ADAM10 | ADAM metallopeptidase | Hs.578508 | 0 | 94.37148 | 97.61905 | Mm.3037 | 0 | 96.88645 | 100 |
| (SEQ ID NO: 316) | | | domain 10 | | | | | | | | |
| WAN013I73_at | 1.602564 | NA | Cluster includes X61958 | #N/A | 0 | 0 | 0 | #N/A | 4E−45 | 88.54167 | 42.01313 |
| (SEQ ID NO: 317) | | | C. longicaudatus mRNA for | | | | | | | | |
| | | | thrombin receptor | | | | | | | | |
| AF061256_at | 1.54321 | FOLR1 | Folate receptor 1 (adult) | Hs.73769 | 1E−124 | 83.96825 | 61.76471 | Mm.2135 | 0 | 89.23077 | 76.47059 |
| (SEQ ID NO: 318) | | | | | | | | | | | |
| WAN008EMJ_at | 1.798561 | GPC6 | Glypican 6 | Hs.444329 | 7E−64 | 92.55319 | 37.4502 | Mm.234129 | 3E−58 | 90.37433 | 37.251 |
| (SEQ ID NO: 319) | | | | | | | | | | | |
| M96676_at | 1.589825 | LGALS1 | Lectin, galactoside-binding, | Hs.445351 | 1E−122 | 88.94472 | 100 | Mm.43831 | 1E−131 | 89.94975 | 100 |
| (SEQ ID NO: 320) | | | soluble, 1 (galectin 1) | | | | | | | | |
| WAN008EKF_at | 1.650165 | Lass2 | Longevity assurance homolog 2 | Hs.643565 | 1E−151 | 89.65517 | 98.7234 | Mm.181009 | 0 | 94.20601 | 99.14894 |
| (SEQ ID NO: 321) | | | (S. cerevisiae) | | | | | | | | |
| U21937_at | 1.577287 | Kcnj6 | Potassium inwardly-rectifying | Hs.50927 | 0.000001 | 84.93151 | 13.27273 | Mm.328720 | 2E−19 | 86.36364 | 20 |
| (SEQ ID NO: 322) | | | channel, subfamily J, member 6 | | | | | | | | |
| WAN013I93_at | 1.569859 | PRKACB | Protein kinase, cAMP- | Hs.487325 | 2E−20 | 96.66667 | 13.1291 | Mm.16766 | 6E−72 | 90.16393 | 66.73961 |
| (SEQ ID NO: 323) | | | dependent, catalytic, beta | | | | | | | | |
| WAN013I1G_at | 1.584786 | SLC25A20 | Solute carrier family 25 | Hs.13845 | 1E−137 | 86.70635 | 87.65217 | Mm.29666 | 0 | 92.35412 | 86.43478 |
| (SEQ ID NO: 324) | | | (carnitine/acylcarnitine | | | | | | | | |
| | | | translocase), member 20 | | | | | | | | |
| Down | | | | | | | | | | | |
| L00180_at | 1.998 | Hmgcr | 3-hydroxy-3-methylglutaryl- | Hs.643495 | 5E−35 | 86.30952 | 68.01619 | Mm.316652 | 1E−49 | 90.2439 | 66.39676 |
| (SEQ ID NO: 325) | | | Coenzyme A reductase | | | | | | | | |
| L00330_x_at | 2.186 | Hmgcs1 | 3-hydroxy-3-methylglutaryl- | Hs.397729 | 2E−58 | 90.71038 | 98.91892 | Mm.61526 | 5E−61 | 92 | 94.59459 |
| (SEQ ID NO: 326) | | | Coenzyme A synthase 1 | | | | | | | | |
| WAN013HXI_at | 1.519 | ACAT2 | Acetyl-Coenzyme A | Hs.571037 | 1E−122 | 83.06011 | 98.74101 | Mm.229342 | 0 | 88.32117 | 98.56115 |
| (SEQ ID NO: 288) | | | acetyltransferase 2 (acetoacetyl | | | | | | | | |
| | | | Coenzyme A thiolase) | | | | | | | | |
| WAN0088T2_at | 2.246 | ATF4 | Activating transcription factor 4 | Hs.496487 | 1E−158 | 88.53974 | 97.83002 | Mm.641 | 0 | 91.71375 | 96.0217 |
| (SEQ ID NO: 327) | | | (tax-responsive enhancer | | | | | | | | |
| | | | element B67) | | | | | | | | |
| M27838_s_at | 1.591 | ASNS | Asparagine synthetase | Hs.489207 | 0 | 89.07199 | 100 | Mm.2942 | 0 | 91.84735 | 100 |
| (SEQ ID NO: 328) | | | | | | | | | | | |
| WAN008EH7_at | 1.641 | ASNSD1 | Asparagine synthetase domain | Hs.101364 | 8E−39 | 82.15488 | 55 | Mm.285783 | 1E−107 | 86.23025 | 82.03704 |
| (SEQ ID NO: 287) | | | containing 1 | | | | | | | | |
| WAN0088J7_at | 1.514 | BCLAF1 | BCL2-associated transcription | Hs.486542 | 0 | 94.28571 | 99.11504 | Mm.294783 | 0 | 93.60568 | 99.64602 |
| (SEQ ID NO: 329) | | | factor 1 | | | | | | | | |
| WAN013I3P_at | 1.598 | CAMLG | Calcium modulating ligand | Hs.529846 | 1E−147 | 86.70213 | 99.29577 | #N/A | 1E−172 | 88.6121 | 98.94366 |
| (SEQ ID NO: 330) | | | | | | | | | | | |
| WAN013HZ9_at | 1.732 | CSPG6 | Chondroitin sulfate proteoglycan | #N/A | 0 | 90.15544 | 98.80546 | #N/A | 0 | 92.9432 | 99.14676 |
| (SEQ ID NO: 331) | | | 6 (bamacan) | | | | | | | | |
| WAN008D37_at | 1.515 | Eif3s10 | Eukaryotic translation initiation | #N/A | 3E−66 | 83.73984 | 78.51064 | Mm.2238 | 1E−152 | 94.10188 | 79.3617 |
| (SEQ ID NO: 289) | | | factor 3, subunit 10 (theta) | | | | | | | | |
| WAN008DDZ_at | 2.023 | XPO1 | Exportin 1 (CRM1 homolog, | Hs.370770 | 1E−176 | 89.04847 | 98.40989 | Mm.217547 | 0 | 94.34629 | 100 |
| (SEQ ID NO: 332) | | | yeast) | | | | | | | | |
| WAN008EQP_at | 1.55 | AA536749 | Expressed sequence AA536749 | #N/A | 2E−57 | 84.71338 | 93.17507 | Mm.2402 | 1E−130 | 93.18885 | 95.8457 |
| (SEQ ID NO: 333) | | | | | | | | | | | |
| WAN008CQI_at | 1.763 | GLTSCR2 | Glioma tumor suppressor | Hs.421907 | 1E−113 | 85.94378 | 100 | Mm.277634 | 1E−175 | 91.16466 | 100 |
| (SEQ ID NO: 334) | | | candidate region gene 2 | | | | | | | | |
| WAN008CSC_at | 1.57 | GARS | Glycyl-tRNA synthetase | Hs.404321 | 1E−176 | 89.14591 | 99.29329 | Mm.250004 | 0 | 92.93286 | 100 |
| (SEQ ID NO: 335) | | | | | | | | | | | |
| WAN013I8X_at | 1.544 | HSPD1 | Heat shock 60 kDa protein 1 | Hs.595053 | 0 | 90.23291 | 99.77511 | Mm.1777 | 0 | 93.38843 | 99.77511 |
| (SEQ ID NO: 286) | | | (chaperonin) | | | | | | | | |
| WAN013I4U_at | 1.617 | HMGA1 | High mobility group AT-hook 1 | Hs.518805 | 1E−115 | 93.57143 | 97.9021 | Mm.4438 | 1E−126 | 94.75524 | 100 |
| (SEQ ID NO: 336) | | | | | | | | | | | |
| Y00365_at | 2.1 | HMGB1 | High-mobility group box 1 | Hs.434102 | 1E−104 | 93.97993 | 23.39593 | Mm.207047 | 1E−145 | 88.84058 | 53.99061 |
| (SEQ ID NO: 337) | | | | | | | | | | | |
| D45419_at | 1.641 | HCFC1 | Host cell factor C1 (VP16- | Hs.83634 | 1E−22 | 84.86486 | 32.74336 | Mm.40343 | 1E−123 | 85.99291 | 99.82301 |
| (SEQ ID NO: 338) | | | accessory protein) | | | | | | | | |
| WAN008E3O_at | 1.52 | LINCR | Likely ortholog of mouse lung- | Hs.149219 | 3E−19 | 84.61538 | 38.0117 | Mm.389110 | 3E−76 | 85.33724 | 99.7076 |
| (SEQ ID NO: 339) | | | inducible Neutralized-related | | | | | | | | |
| | | | C3HC4 RING domain protein | | | | | | | | |
| WAN008EV8_at | 1.503 | MTHFD2 | Methylenetetrahydrofolate | Hs.469030 | 7E−50 | 88.35979 | 68.23105 | Mm.443 | 4E−84 | 91.05691 | 88.80866 |
| (SEQ ID NO: 340) | | | dehydrogenase (NADP+ | | | | | | | | |
| | | | dependent) 2, | | | | | | | | |
| | | | methenyltetrahydrofolate | | | | | | | | |
| | | | cyclohydrolase | | | | | | | | |
| WAN008D66_at | 1.679 | Mthfd1 | Methylenetetrahydrofolate | Hs.614936 | 1E−115 | 87.17949 | 90.12605 | Mm.29584 | 1E−129 | 88.06306 | 93.27731 |
| (SEQ ID NO: 341) | | | dehydrogenase (NADP+ | | | | | | | | |
| | | | dependent), | | | | | | | | |
| | | | methenyltetrahydrofolate | | | | | | | | |
| | | | cyclohydrolase, | | | | | | | | |
| | | | formyltetrahydrofolate synthase | | | | | | | | |
| WAN013I09_at | 1.54 | NEDD4 | Neural precursor cell expressed, | Hs.1565 | 4E−90 | 88.67314 | 57.5419 | Mm.279923 | 1E−136 | 89.70588 | 88.6406 |
| (SEQ ID NO: 342) | | | developmentally down-regulated 4 | | | | | | | | |
| WAN008CWD_at | 1.601 | PTCD2 | Pentatricopeptide repeat domain 2 | Hs.126906 | 6E−43 | 84.29752 | 42.53076 | Mm.276502 | 1E−64 | 84.10405 | 60.80844 |
| (SEQ ID NO: 343) | | | | | | | | | | | |
| WAN013I3J_at | 1.775 | PHGDH | Phosphoglycerate | Hs.487296 | 0 | 89.47368 | 99.0689 | Mm.16898 | 0 | 93.64486 | 99.62756 |
| (SEQ ID NO: 344) | | | dehydrogenase | | | | | | | | |
| WAN008EBY_at | 1.612 | PLCG1 | Phospholipase C, gamma 1 | Hs.268177 | 1E−175 | 89.63636 | 99.4575 | Mm.44463 | 0 | 92.5859 | 100 |
| (SEQ ID NO: 345) | | | | | | | | | | | |
| WAN013I0O_at | 1.609 | PAICS | Phosphoribosylaminoimidazole | Hs.518774 | 0 | 89.86254 | 100 | Mm.182931 | 0 | 91.40893 | 100 |
| (SEQ ID NO: 346) | | | carboxylase, | | | | | | | | |
| | | | phosphoribosylaminoimidazole | | | | | | | | |
| | | | succinocarboxamide synthetase | | | | | | | | |
| WAN008E9C_at | 1.544 | Pscd2 | Pleckstrin homology, Sec7 and | Hs.144011 | 1E−171 | 91.83223 | 99.34211 | Mm.272130 | 0 | 96.02649 | 99.34211 |
| (SEQ ID NO: 347) | | | coiled-coil domains 2 | | | | | | | | |
| WAN0088WG_at | 1.745 | PA2G4 | Proliferation-associated 2G4, | Hs.524498 | 0 | 91.31313 | 86.84211 | Mm.4742 | 0 | 94.91228 | 100 |
| (SEQ ID NO: 348) | | | 38 kDa | | | | | | | | |
| WAN008EFI_at | 1.511 | PRSS15 | Protease, serine, 15 | Hs.350265 | 1E−167 | 88.27709 | 98.2548 | Mm.329136 | 0 | 91.44852 | 100 |
| (SEQ ID NO: 349) | | | | | | | | | | | |
| WAN013HYE_at | 1.714 | Psmd11_predicted | Proteasome (prosome, | #N/A | 0 | 92.33577 | 100 | #N/A | 0 | 94.34307 | 100 |
| (SEQ ID NO: 350) | | | macropain) 26S subunit, non- | | | | | | | | |
| | | | ATPase, 11 (predicted) | | | | | | | | |
| WAN013HWW_at | 1.531 | RAN | RAN, member RAS oncogene | Hs.10842 | 1E−150 | 88.33992 | 100 | Mm.297440 | 0 | 96.04743 | 100 |
| (SEQ ID NO: 351) | | | family | | | | | | | | |
| WAN008DUB_at | 1.837 | RDH11 | Retinol dehydrogenase 11 (all- | Hs.226007 | 1E−77 | 84.84043 | 81.38528 | Mm.291799 | 3E−89 | 91.37255 | 55.19481 |
| (SEQ ID NO: 352) | | | trans/9-cis/11-cis) | | | | | | | | |
| WAN008E6V_at | 1.501 | RBM28 | RNA binding motif protein 28 | Hs.274263 | 1E−178 | 89.61039 | 94.23077 | Mm.40802 | 0 | 93.15589 | 91.95804 |
| (SEQ ID NO: 353) | | | | | | | | | | | |
| WAN008DNJ_at | 1.58 | Rbmxrt | RNA binding motif protein, X | #N/A | 1E−131 | 92.19653 | 71.04723 | Mm.24718 | 0 | 94.75891 | 97.94661 |
| (SEQ ID NO: 354) | | | chromosome retrogene | | | | | | | | |
| WAN008EPC_at | 1.594 | SHMT2 | Serine hydroxymethyltransferase | Hs.75069 | 1E−143 | 90.43062 | 99.05213 | Mm.29890 | 1E−142 | 90.68627 | 96.68246 |
| (SEQ ID NO: 355) | | | 2 (mitochondrial) | | | | | | | | |
| WAN013I0Q_at | 1.609 | SERBP1 | SERPINE1 mRNA binding | Hs.530412 | 0 | 93.12169 | 98.95288 | Mm.240490 | 0 | 94.58988 | 100 |
| (SEQ ID NO: 356) | | | protein 1 | | | | | | | | |
| WAN008ETE_x_at | 2.544 | SMC2L1 | SMC2 structural maintenance of | #N/A | 8E−46 | 87.36842 | 96.4467 | #N/A | 5E−67 | 91.37056 | 100 |
| (SEQ ID NO: 357) | | | chromosomes 2-like 1 (yeast) | | | | | | | | |
| WAN008D4B_at | 2.687 | SMC4L1 | SMC4 structural maintenance of | #N/A | 0 | 91.32075 | 99.43715 | #N/A | 0 | 92.83019 | 99.43715 |
| (SEQ ID NO: 358) | | | chromosomes 4-like 1 (yeast) | | | | | | | | |
| WAN0088NR_at | 1.534 | SF3B3 | Splicing factor 3b, subunit 3, | Hs.514435 | 1E−131 | 92.39766 | 81.04265 | Mm.236123 | 1E−151 | 94.97041 | 80.09479 |
| (SEQ ID NO: 359) | | | 130 kDa | | | | | | | | |
| WAN008F2D_at | 1.775 | SFPQ | Splicing factor | Hs.355934 | 4E−80 | 88.15789 | 72.90168 | Mm.257276 | 4E−91 | 90 | 69.54436 |
| (SEQ ID NO: 360) | | | proline/glutamine-rich | | | | | | | | |
| | | | (polypyrimidine tract binding | | | | | | | | |
| | | | protein associated) | | | | | | | | |
| U22819_s_at | 1.615 | SREBF2 | Sterol regulatory element | Hs.443258 | 1E−118 | 89.83516 | 99.45355 | Mm.38016 | 1E−133 | 92.39437 | 96.99454 |
| (SEQ ID NO: 361) | | | binding transcription factor 2 | | | | | | | | |
| WAN0088ZO_x_at | 1.502 | SYNCRIP | Synaptotagmin binding, | Hs.472056 | 1E−50 | 94.61538 | 70.65217 | Mm.32874 | 3E−53 | 95.38462 | 70.65217 |
| (SEQ ID NO: 362) | | | cytoplasmic RNA interacting | | | | | | | | |
| | | | protein | | | | | | | | |
| WAN0088JV_at | 1.651 | TRIB3 | Tribbles homolog 3 (Drosophila) | Hs.516826 | 4E−62 | 81.77778 | 86.37236 | Mm.276018 | 1E−158 | 88.8454 | 98.08061 |
| (SEQ ID NO: 363) | | | | | | | | | | | |
| WAN008DKJ_x_at | 1.735 | Zfp297b | Zinc finger protein 297B | #N/A | 1E−27 | 91.57895 | 90.47619 | Mm.44186 | 2E−24 | 89.69072 | 92.38095 |
| (SEQ ID NO: 364) | | | | | | | | | | | |

TABLE 10
Exemplary Test-Only Differentially Expressed Genes

| Test Only | FC | Symbol | Title | Human Unigene ID | eValue | % ID | % QC | Mouse Unigene ID | eValue | % ID | % QC |
|---|---|---|---|---|---|---|---|---|---|---|---|
| Up | | | | | | | | | | | |
| WAN008EJY_at | 2.12766 | | | | | | | | | | |
| (SEQ ID NO: 365) | | | | | | | | | | | |
| Down | | | | | | | | | | | |
| AJ225170_f_at | 1.664 | NA | AJ225170 Mesocricetus | #N/A | 0 | 0 | 0 | #N/A | 0 | 0 | 0 |
| (SEQ ID NO: 366) | | | auratus aphrodisin gene. | | | | | | | | |
| WAN008D0D_x_at | 1.713 | Cflar | CASP8 and FADD-like | Hs.390736 | 0 | 0 | 0 | Mm.11778 | 3E−25 | 85.27607 | 28.69718 |
| (SEQ ID NO: 367) | | | apoptosis regulator | | | | | | | | |
| AF281019_at | 1.652 | CDC7 | CDC7 cell division cycle 7 (S. cerevisiae) | Hs.533573 | 1E−30 | 90.90909 | 8.86382 | Mm.20842 | 8E−55 | 88.09524 | 16.92184 |
| (SEQ ID NO: 368) | | | | | | | | | | | |
| WAN008E4T_at | 1.74 | CLK1 | CDC-like kinase 1 | Hs.433732 | 1E−74 | 93.33333 | 37.57225 | Mm.1761 | 3E−83 | 85.71429 | 71.48362 |
| (SEQ ID NO: 369) | | | | | | | | | | | |
| U10249_at | 1.721 | CDK2AP1 | CDK2-associated protein 1 | Hs.433201 | 9E−77 | 86.82635 | 35.95264 | Mm.390335 | 0 | 93.15353 | 51.88375 |
| (SEQ ID NO: 315) | | | | | | | | | | | |
| WAN008DJD_at | 1.608 | CDCA2 | Cell division cycle associated 2 | Hs.33366 | 6E−18 | 85.34483 | 22.17973 | Mm.33831 | 5E−82 | 86.3388 | 69.98088 |
| (SEQ ID NO: 370) | | | | | | | | | | | |
| WAN013I2T_at | 2.029 | CBX5 | Chromobox homolog 5 (HP1 | Hs.632724 | 1E−142 | 91.86352 | 72.02268 | Mm.262059 | 1E−168 | 94.75066 | 72.02268 |
| (SEQ ID NO: 371) | | | alpha homolog, Drosophila) | | | | | | | | |
| WAN013I6G_at | 1.572 | NA | Cluster includes M12252 | #N/A | 0 | 0 | 0 | #N/A | 0 | 0 | 0 |
| (SEQ ID NO: 372) | | | Chinese hamster alpha-tubulin I | | | | | | | | |
| | | | mRNA, complete cds. | | | | | | | | |
| WAN013HZA_at | 1.603 | CSE1L | CSE1 chromosome segregation | Hs.90073 | 1E−180 | 88.51351 | 100 | Mm.22417 | 0 | 93.07432 | 100 |
| (SEQ ID NO: 373) | | | 1-like (yeast) | | | | | | | | |
| WAN008EZN_at | 1.994 | DNAJC15 | DnaJ (Hsp40) homolog, | Hs.438830 | 7E−92 | 89.93056 | 49.39966 | Mm.248046 | 1E−116 | 92.66667 | 51.45798 |
| (SEQ ID NO: 374) | | | subfamily C, member 15 | | | | | | | | |
| WAN013HWL_at | 1.85 | EBP | Emopamil binding protein | Hs.632801 | 6E−21 | 84.17266 | 24.86583 | Mm.27183 | 2E−46 | 91.9708 | 24.50805 |
| (SEQ ID NO: 375) | | | (sterol isomerase) | | | | | | | | |
| WAN008CLU_at | 1.885 | Emp1 | Epithelial membrane protein 1 | Hs.436298 | 0 | 0 | 0 | Mm.182785 | 3E−28 | 90.16393 | 21.66963 |
| (SEQ ID NO: 376) | | | | | | | | | | | |
| WAN013HW1_at | 1.764 | Eef1d | Eukaryotic translation | Hs.333388 | 1E−115 | 84.05797 | 99.45946 | Mm.258927 | 0 | 91.24088 | 98.73874 |
| (SEQ ID NO: 377) | | | elongation factor 1 delta | | | | | | | | |
| | | | (guanine nucleotide exchange | | | | | | | | |
| | | | protein) | | | | | | | | |
| WAN008DMP_at | 1.852 | EWSR1 | Ewing sarcoma breakpoint | Hs.374477 | 1E−157 | 90.52863 | 94.19087 | Mm.142822 | 0 | 92.98246 | 94.60581 |
| (SEQ ID NO: 313) | | | region 1 | | | | | | | | |
| WAN013HVQ_x_at | 1.575 | H3f3b | H3 histone, family 3B | Hs.180877 | 0 | 91.82609 | 99.65338 | Mm.18516 | 0 | 95 | 97.05373 |
| (SEQ ID NO: 378) | | | | | | | | | | | |
| WAN0088TK_x_at | 1.566 | HDGF | Hepatoma-derived growth | Hs.506748 | 3E−26 | 86.46617 | 76.43678 | Mm.292208 | 2E−26 | 86.46617 | 76.43678 |
| (SEQ ID NO: 379) | | | factor (high-mobility group | | | | | | | | |
| | | | protein 1-like) | | | | | | | | |
| WAN013I1P_at | 1.765 | HNRPA2B1 | Heterogeneous nuclear | Hs.487774 | 0 | 97.22222 | 90.94737 | Mm.155896 | 0 | 96.52778 | 90.94737 |
| (SEQ ID NO: 380) | | | ribonucleoprotein A2/B1 | | | | | | | | |
| X83575_at | 3.142 | KIF23 | Kinesin family member 23 | Hs.270845 | 1E−177 | 92.47788 | 37.07957 | Mm.259374 | 0 | 91.99372 | 52.25595 |
| (SEQ ID NO: 381) | | | | | | | | | | | |
| U11790_at | 3.323 | KIF2C | Kinesin family member 2C | Hs.69360 | 0 | 89.25714 | 66.43888 | Mm.247651 | 0 | 92.51055 | 71.98178 |
| (SEQ ID NO: 382) | | | | | | | | | | | |
| WAN008D31_at | 1.508 | Lss | Lanosterol synthase | Hs.596543 | 1E−80 | 85.30184 | 68.27957 | Mm.55075 | 1E−150 | 91.52542 | 74.01434 |
| (SEQ ID NO: 383) | | | | | | | | | | | |
| WAN013HZX_at | 1.517 | LTB4DH | Leukotriene B4 12- | Hs.584864 | 2E−95 | 83.67347 | 100 | Mm.34497 | 1E−176 | 90.61224 | 100 |
| (SEQ ID NO: 384) | | | hydroxydehydrogenase | | | | | | | | |
| WAN008EL3_at | 1.74 | LSM3 | LSM3 homolog, U6 small | Hs.111632 | 1E−102 | 90.69767 | 65.15152 | Mm.246693 | 1E−165 | 91.77489 | 100 |
| (SEQ ID NO: 314) | | | nuclear RNA associated (S. cerevisiae) | | | | | | | | |
| WAN0088X5_at | 2.721 | MAD2L1 | MAD2 mitotic arrest deficient- | Hs.591697 | 1E−111 | 87.4092 | 93.01802 | Mm.290830 | 1E−153 | 90.95128 | 97.07207 |
| (SEQ ID NO: 385) | | | like 1 (yeast) | | | | | | | | |
| WAN008D06_at | 1.888 | MCM4 | MCM4 minichromosome | Hs.460184 | 1E−159 | 87.97814 | 98.21109 | Mm.1500 | 0 | 92.98561 | 99.46333 |
| (SEQ ID NO: 386) | | | maintenance deficient 4 (S. cerevisiae) | | | | | | | | |
| J00061_at | 1.994 | MT1 | metallothionein I | #N/A | 3E−46 | 87.70053 | 66.31206 | Mm.192991 | 2E−64 | 91.89189 | 65.60284 |
| (SEQ ID NO: 387) | | | | | | | | | | | |
| WAN008CSN_at | 1.739 | OACT5 | O-acyltransferase (membrane | #N/A | 1E−140 | 87.82961 | 99.79757 | #N/A | 0 | 92.71255 | 100 |
| (SEQ ID NO: 388) | | | bound) domain containing 5 | | | | | | | | |
| WAN013I62_at | 1.56 | ODC1 | Ornithine decarboxylase 1 | Hs.467701 | 1E−178 | 85.99222 | 56.64952 | Mm.34102 | 0 | 91.76788 | 54.44526 |
| (SEQ ID NO: 389) | | | | | | | | | | | |
| AB041733_at | 1.584 | PEX12 | Peroxisomal biogenesis factor | Hs.591190 | 1E−39 | 92.56198 | 9.173616 | Mm.102205 | 4E−75 | 86.98413 | 23.88173 |
| (SEQ ID NO: 390) | | | 12 | | | | | | | | |
| WAN008DUC_at | 1.532 | PHF14 | PHD finger protein 14 | Hs.159918 | 8E−86 | 94.63415 | 99.51456 | Mm.212411 | 4E−74 | 92.19512 | 99.51456 |
| (SEQ ID NO: 391) | | | | | | | | | | | |
| U21937_at | 1.596 | Kcnj6 | Potassium inwardly-rectifying | Hs.50927 | 0.000001 | 84.93151 | 13.27273 | Mm.328720 | 2E−19 | 86.36364 | 20 |
| (SEQ ID NO: 322) | | | channel, subfamily J, member 6 | | | | | | | | |
| WAN008ET0_at | 1.54 | RNASEH2A | Ribonuclease H2, large subunit | Hs.532851 | 1E−111 | 86.28319 | 95.96603 | Mm.182470 | 0 | 92.14437 | 100 |
| (SEQ ID NO: 392) | | | | | | | | | | | |
| WAN008EPM_at | 1.516 | SEPHS1 | Selenophosphate synthetase 1 | Hs.124027 | 0 | 93.53349 | 96.00887 | Mm.34329 | 0 | 95.78714 | 100 |
| (SEQ ID NO: 393) | | | | | | | | | | | |
| M74776_at | 1.6 | SERPINA6 | Serpin peptidase inhibitor, | Hs.532635 | 0.000006 | 88.46154 | 9.42029 | Mm.290079 | 8E−10 | 82.20339 | 21.37681 |
| (SEQ ID NO: 394) | | | clade A (alpha-1 antiproteinase, | | | | | | | | |
| | | | antitrypsin), member 6 | | | | | | | | |
| Y12074_at | 1.944 | SLC35A1 | Solute carrier family 35 (CMP- | Hs.423163 | 1E−171 | 90.07634 | 39.30983 | Mm.281885 | 0 | 91.73291 | 47.1868 |
| (SEQ ID NO: 395) | | | sialic acid transporter), member | | | | | | | | |
| | | | A1 | | | | | | | | |
| AJ245700_at | 1.559 | ST3GAL4 | ST3 beta-galactoside alpha-2,3- | Hs.591947 | 1E−117 | 91.41104 | 55.7265 | Mm.275973 | 1E−131 | 93.25153 | 55.7265 |
| (SEQ ID NO: 396) | | | sialyltransferase 4 | | | | | | | | |
| WAN008EN4_at | 1.516 | SUV39H2 | Suppressor of variegation 3-9 | Hs.554883 | 0.000001 | 92.68293 | 8.991228 | Mm.23483 | 6E−07 | 85.52632 | 16.66667 |
| (SEQ ID NO: 397) | | | homolog 2 (Drosophila) | | | | | | | | |
| X98066_at | 1.582 | TSN | Translin | Hs.75066 | 1E−87 | 91.30435 | 47.02602 | Mm.426637 | 0 | 0 | 0 |
| (SEQ ID NO: 398) | | | | | | | | | | | |
| D86467_at | 2.312 | TM4SF1 | Transmembrane 4 L six family | Hs.351316 | 2E−41 | 86.19048 | 71.91781 | Mm.856 | 2E−40 | 86.74033 | 61.9863 |
| (SEQ ID NO: 399) | | | member 1 | | | | | | | | |
| WAN013I9O_at | 1.923 | TUBB6 | Tubulin, beta 6 | Hs.193491 | 0 | 92.2528 | 71.39738 | Mm.181860 | 0 | 91.74573 | 76.71033 |
| (SEQ ID NO: 400) | | | | | | | | | | | |
| WAN008CS2_at | 1.605 | VKORC1L1 | Vitamin K epoxide reductase | Hs.427232 | 1E−168 | 91.89189 | 96.73203 | Mm.288718 | 0 | 97.28507 | 96.2963 |
| (SEQ ID NO: 401) | | | complex, subunit 1-like 1 | | | | | | | | |

TABLE 11
Exemplary Process-Only Differentially Expressed Genes

| | | | | Human | |
|---|---|---|---|---|---|
| | | | | Unigene | |
| Process Only | FC | Symbol | Title | ID | eValue |
| Up | | | | | |
| AF022945- | 1.72117 | Thbd | Thrombomodulin | Hs.2030 | 0 |
| rc_f_at | | | | | |
| (SEQ ID NO: 402) | | | | | |
| Down | | | | | |
| AF292400_at | 3.372 | ASK | Activator of S phase kinase | #N/A | 2E−78 |
| (SEQ ID NO: 290) | | | | | |
| WAN008EB0_at | 1.586 | ACOT7 | Acyl-CoA thioesterase 7 | Hs.126137 | 1E−49 |
| (SEQ ID NO: 403) | | | | | |
| M80243-rc_at | 2.371 | BIRC5 | Baculoviral IAP repeat- | Hs.514527 | 4E−38 |
| (SEQ ID NO: 291) | | | containing 5 (survivin) | | |
| WAN008CYY_at | 1.899 | BUB1B | BUB1 budding uninhibited by | Hs.631699 | 4E−34 |
| (SEQ ID NO: 292) | | | benzimidazoles 1 homolog beta | | |
| | | | (yeast) | | |
| WAN008CI5_at | 4.869 | CDC20 | CDC20 cell division cycle 20 | Hs.524947 | 1E−105 |
| (SEQ ID NO: 404) | | | homolog (S. cerevisiae) | | |
| WAN013I9R_at | 2.093 | NA | Cluster includes Y08202 | #N/A | 1E−104 |
| (SEQ ID NO: 294) | | | C. griseus mRNA for RAD51 | | |
| | | | protein | | |
| WAN013I5T_at | 4.366 | CCNB1 | Cyclin B1 | Hs.23960 | 1E−93 |
| (SEQ ID NO: 295) | | | | | |
| WAN013I8J_at | 5.868 | CCNB2 | Cyclin B2 | Hs.194698 | 1E−173 |
| (SEQ ID NO: 296) | | | | | |
| U48852_at | 1.853 | CRELD2 | Cysteine-rich with EGF-like | Hs.211282 | 1E−109 |
| (SEQ ID NO: 297) | | | domains 2 | | |
| WAN0088T7_at | 1.888 | Cyp51 | Cytochrome P450, family 51 | #N/A | 1E−132 |
| (SEQ ID NO: 298) | | | | | |
| WAN008E8K_at | 1.747 | H2-K1 | Histocompatibility 2, K1, K region | #N/A | 1E−154 |
| (SEQ ID NO: 299) | | | | | |
| WAN008EXF_at | 4.151 | KIF11 | Kinesin family member 11 | Hs.8878 | 4E−28 |
| (SEQ ID NO: 300) | | | | | |
| X83576_at | 1.796 | KIFC1 | Kinesin family member C1 | Hs.436912 | 0 |
| (SEQ ID NO: 301) | | | | | |
| WAN008CX4_at | 2.332 | MCM5 | MCM5 minichromosome | Hs.517582 | 1E−152 |
| (SEQ ID NO: 302) | | | maintenance deficient 5, cell | | |
| | | | division cycle 46 (S. cerevisiae) | | |
| WAN008DZY_at | 1.945 | MCM7 | MCM7 minichromosome | Hs.438720 | 3E−99 |
| (SEQ ID NO: 405) | | | maintenance deficient 7 | | |
| | | | (S. cerevisiae) | | |
| WAN008DWL_at | 3.059 | NEK2 | NIMA (never in mitosis gene | Hs.153704 | 1E−71 |
| (SEQ ID NO: 304) | | | a)-related kinase 2 | | |
| WAN008EML_at | 2.728 | PBK | PDZ binding kinase | Hs.104741 | 5E−52 |
| (SEQ ID NO: 305) | | | | | |
| WAN008D2Z_at | 1.599 | PHGDH | Phosphoglycerate | Hs.487296 | 1E−160 |
| (SEQ ID NO: 406) | | | dehydrogenase | | |
| WAN013I9V_at | 1.611 | Pgk1 | Phosphoglycerate kinase 1 | Hs.78771 | 0 |
| (SEQ ID NO: 407) | | | | | |
| WAN008ELE_at | 2.063 | PSAT1 | Phosphoserine | Hs.494261 | 7E−27 |
| (SEQ ID NO: 306) | | | aminotransferase 1 | | |
| WAN013I8E_at | 1.554 | PCNA | Proliferating cell nuclear | Hs.147433 | 1E−173 |
| (SEQ ID NO: 307) | | | antigen | | |
| WAN008E3C_at | 2.404 | Ptma | Prothymosin alpha | Hs.459927 | 2E−67 |
| (SEQ ID NO: 308) | | | | | |
| WAN008EJV_at | 3.128 | Racgap1 | Rac GTPase-activating protein 1 | Hs.645513 | 1E−103 |
| (SEQ ID NO: 309) | | | | | |
| WAN0088U6_at | 4.664 | SPAG5 | Sperm associated antigen 5 | Hs.514033 | 1E−108 |
| (SEQ ID NO: 310) | | | | | |
| WAN008CJI_at | 1.579 | SFRS2 | Splicing factor, arginine/serine- | Hs.584801 | 3E−81 |
| (SEQ ID NO: 311) | | | rich 2 | | |
| L00365_at | 3.014 | TK1 | Thymidine kinase 1, soluble | Hs.515122 | 6E−24 |
| (SEQ ID NO: 408) | | | | | |
| WAN013IAD_at | 4.806 | TOP2A | Topoisomerase (DNA) II alpha | Hs.156346 | 3E−37 |
| (SEQ ID NO: 312) | | | 170 kDa | | |

| Process Only | % ID | % QC | Mouse Unigene ID | eValue | % ID | % QC |
|---|---|---|---|---|---|---|
| Up | | | | | | |
| AF022945- | 0 | 0 | Mm.24096 | 1E−13 | 89.55224 | 65.04854 |
| rc_f_at | | | | | | |
| (SEQ ID NO: 402) | | | | | | |
| Down | | | | | | |
| AF292400_at | 91.59292 | 17.97932 | #N/A | 1E−152 | 86.44338 | 49.88067 |
| (SEQ ID NO: 290) | | | | | | |
| WAN008EB0_at | 88.64865 | 50.40872 | Mm.296191 | 3E−70 | 93.04813 | 50.95368 |
| (SEQ ID NO: 403) | | | | | | |
| M80243-rc_at | 92.37288 | 20.34483 | Mm.8552 | 1E−36 | 93.45794 | 18.44828 |
| (SEQ ID NO: 291) | | | | | | |
| WAN008CYY_at | 81.32184 | 73.10924 | Mm.29133 | 2E−71 | 84.59384 | 75 |
| (SEQ ID NO: 292) | | | | | | |
| WAN008CI5_at | 89.21283 | 68.6 | Mm.289747 | 1E−142 | 93.58601 | 68.6 |
| (SEQ ID NO: 404) | | | | | | |
| WAN013I9R_at | 84.573 | 63.46154 | #N/A | 1E−142 | 88.98072 | 63.46154 |
| (SEQ ID NO: 294) | | | | | | |
| WAN013I5T_at | 85.30259 | 28.11994 | Mm.260114 | 1E−110 | 87.17949 | 28.44408 |
| (SEQ ID NO: 295) | | | | | | |
| WAN013I8J_at | 86.9258 | 44.39216 | Mm.22592 | 0 | 90.70946 | 46.43137 |
| (SEQ ID NO: 296) | | | | | | |
| U48852_at | 81.76944 | 55.13673 | Mm.292567 | 0 | 88.79936 | 91.7221 |
| (SEQ ID NO: 297) | | | | | | |
| WAN0088T7_at | 86.87873 | 98.05068 | Mm.46044 | 1E−152 | 88.51485 | 98.44055 |
| (SEQ ID NO: 298) | | | | | | |
| WAN008E8K_at | 92.60204 | 98 | Mm.33263 | 1E−168 | 93.75 | 100 |
| (SEQ ID NO: 299) | | | | | | |
| WAN008EXF_at | 86.86131 | 27.56539 | Mm.42203 | 3E−24 | 91.86047 | 17.30382 |
| (SEQ ID NO: 300) | | | | | | |
| X83576_at | 86.77111 | 86.84807 | Mm.335713 | 0 | 90.52369 | 90.92971 |
| (SEQ ID NO: 301) | | | | | | |
| WAN008CX4_at | 87.10247 | 100 | Mm.5048 | 0 | 91.48936 | 99.64664 |
| (SEQ ID NO: 302) | | | | | | |
| WAN008DZY_at | 88.37209 | 100 | Mm.241714 | 1E−123 | 91.27907 | 100 |
| (SEQ ID NO: 405) | | | | | | |
| WAN008DWL_at | 87.38739 | 58.42105 | Mm.33773 | 1E−152 | 89.13934 | 85.61404 |
| (SEQ ID NO: 304) | | | | | | |
| WAN008EML_at | 89.50276 | 39.09287 | Mm.24337 | 3E−80 | 89.78102 | 59.17927 |
| (SEQ ID NO: 305) | | | | | | |
| WAN008D2Z_at | 88.41121 | 100 | Mm.16898 | 0 | 92.52336 | 100 |
| (SEQ ID NO: 406) | | | | | | |
| WAN013I9V_at | 92.71028 | 42.39303 | Mm.316355 | 0 | 94.82759 | 45.9588 |
| (SEQ ID NO: 407) | | | | | | |
| WAN008ELE_at | 93.10345 | 16.171 | Mm.289936 | 4E−70 | 87.53994 | 58.17844 |
| (SEQ ID NO: 306) | | | | | | |
| WAN013I8E_at | 90.92784 | 83.19039 | Mm.7141 | 0 | 92.2813 | 100 |
| (SEQ ID NO: 307) | | | | | | |
| WAN008E3C_at | 93.83886 | 44.98934 | Mm.19187 | 1E−148 | 92.74005 | 91.04478 |
| (SEQ ID NO: 308) | | | | | | |
| WAN008EJV_at | 86.39618 | 93.31849 | Mm.273804 | 1E−133 | 89.31116 | 93.76392 |
| (SEQ ID NO: 309) | | | | | | |
| WAN0088U6_at | 84.43649 | 98.07018 | Mm.24250 | 1E−153 | 87.12522 | 99.47368 |
| (SEQ ID NO: 310) | | | | | | |
| WAN008CJI_at | 97.23757 | 41.70507 | Mm.21841 | 1E−143 | 92.79778 | 83.17972 |
| (SEQ ID NO: 311) | | | | | | |
| L00365_at | 90.32258 | 70.45455 | Mm.2661 | 4E−48 | 94.4 | 94.69697 |
| (SEQ ID NO: 408) | | | | | | |
| WAN013IAD_at | 80.62678 | 29.52061 | Mm.4237 | 1E−86 | 84.11633 | 37.59462 |
| (SEQ ID NO: 312) | | | | | | |

### Example 5

### Platform Analysis

Four cell lines were analyzed from the Platform Process category that exhibit a “good fed batch phenotype.” These cells grow well, maintain good viability throughout the fedbatch, and exhibit a “metabolic shift” phenotype that is characterized by the ability to consume the metabolic byproducts lactate and ammonium when cultured in fed batch culture. Multiple time points were collected for each cell line grown in fed batch culture. The time points from each cell line were examined by ANOVA analysis to monitor the changes in gene expression over the course of the culture. The gene lists from each cell line were compared, and those that were in common between all 4 cell lines were identified. Exemplary nucleic acid sequences are listed in Tables 12 and 13.

TABLE 12
Platform Analysis

| | | | Human | | | | | | |
|---|---|---|---|---|---|---|---|---|---|
| | | | Unigene | | | | Mouse | | |
| Qualifier List | Symbol | Title | ID | eValue | % ID | % QC | Unigene ID | eValue | % ID |
| AF022941_x_at | Cirbp | Cold inducible RNA | Hs.634522 | 9E−27 | 93.65079 | 69.61326 | Mm.17898 | 1E−52 | 96.26866 |
| (SEQ ID NO: 409) | | binding protein | | | | | | | |
| AF081141_at | CCL2 | Chemokine (C-C motif) | Hs.303649 | 3E−13 | 90.625 | 13.41719 | Mm.290320 | 5E−41 | 91.04478 |
| (SEQ ID NO: 410) | | ligand 2 | | | | | | | |
| AF254572_at | ORC1L | Origin recognition | Hs.17908 | 0 | 85.62005 | 63.06156 | Mm.294154 | 0 | 89.31624 |
| (SEQ ID NO: 411) | | complex, subunit 1-like | | | | | | | |
| | | (yeast) | | | | | | | |
| L00366_x_at | TK1 | Thymidine kinase 1, | Hs.515122 | 4E−18 | 89.87342 | 84.94624 | Mm.2661 | 1E−16 | 88.75 |
| (SEQ ID NO: 412) | | soluble | | | | | | | |
| M12329_at | NA | M12329 Chinese hamster | #N/A | 0 | 93.01676 | 54.28355 | #N/A | 0 | 96.47391 |
| (SEQ ID NO: 413) | | alpha-tubulin III mRNA, | | | | | | | |
| | | complete cds. | | | | | | | |
| M80243-rc_at | BIRC5 | Baculoviral IAP repeat- | Hs.514527 | 4E−38 | 92.37288 | 20.34483 | Mm.8552 | 1E−36 | 93.45794 |
| (SEQ ID NO: 291) | | containing 5 (survivin) | | | | | | | |
| U11790_at | KIF2C | Kinesin family member | Hs.69360 | 0 | 89.25714 | 66.43888 | Mm.247651 | 0 | 92.51055 |
| (SEQ ID NO: 382) | | 2C | | | | | | | |
| U48852_at | CRELD2 | Cysteine-rich with EGF- | Hs.211282 | 1E−109 | 81.76944 | 55.13673 | Mm.292567 | 0 | 88.79936 |
| (SEQ ID NO: 297) | | like domains 2 | | | | | | | |
| WAN0088J9_x_at | NA | WAN0088J9 10595A- | #N/A | 0 | 0 | 0 | #N/A | 0 | 0 |
| (SEQ ID NO: 414) | | E01 | | | | | | | |
| WAN0088K2_at | DUSP16 | Dual specificity | Hs.536535 | 0.00002 | 84.44444 | 15.98579 | Mm.3994 | 4E−21 | 87.5 |
| (SEQ ID NO: 415) | | phosphatase 16 | | | | | | | |
| WAN0088ON_at | ATAD2 | ATPase family, AAA | Hs.370834 | 2E−45 | 83.7037 | 59.08096 | Mm.221758 | 9E−71 | 87.31884 |
| (SEQ ID NO: 416) | | domain containing 2 | | | | | | | |
| WAN0088Q6_at | NA | WAN0088Q6 10595D- | #N/A | 1E−153 | 93.71585 | 63.43154 | #N/A | 0 | 94.43155 |
| (SEQ ID NO: 417) | | A09 | | | | | | | |
| WAN0088S8_at | SLC29A1 | Solute carrier family 29 | Hs.25450 | 3E−35 | 81.35593 | 76.12903 | Mm.29744 | 5E−97 | 86.09756 |
| (SEQ ID NO: 418) | | (nucleoside transporters), | | | | | | | |
| | | member 1 | | | | | | | |
| WAN0088T7_at | Cyp51 | Cytochrome P450, | #N/A | 1E−132 | 86.87873 | 98.05068 | Mm.46044 | 1E−152 | 88.51485 |
| (SEQ ID NO: 298) | | family 51 | | | | | | | |
| WAN0088U6_at | SPAG5 | Sperm associated | Hs.514033 | 1E−108 | 84.43649 | 98.07018 | Mm.24250 | 1E−153 | 87.12522 |
| (SEQ ID NO: 310) | | antigen 5 | | | | | | | |
| WAN0088X5_at | MAD2L1 | MAD2 mitotic arrest | Hs.591697 | 1E−111 | 87.4092 | 93.01802 | Mm.290830 | 1E−153 | 90.95128 |
| (SEQ ID NO: 385) | | deficient-like 1 (yeast) | | | | | | | |
| WAN008906_at | Zfp259 | Zinc finger protein 259 | #N/A | 1E−162 | 90.1354 | 94.86239 | Mm.17519 | 0 | 92.84404 |
| (SEQ ID NO: 419) | | | | | | | | | |
| WAN00893Z_at | NA | WAN00893Z 10599B- | #N/A | 8E−33 | 85.87571 | 30.62284 | #N/A | 3E−77 | 88.25503 |
| (SEQ ID NO: 420) | | D03 | | | | | | | |
| WAN008BNE_x_at | NA | WAN008BNE 11233D- | #N/A | 0 | 0 | 0 | #N/A | 0 | 0 |
| (SEQ ID NO: 421) | | H09 | | | | | | | |
| WAN008BNO_at | 2810025M15Rik | RIKEN cDNA | #N/A | 9E−08 | 83.90805 | 15.90494 | Mm.286863 | 1E−146 | 88.09524 |
| (SEQ ID NO: 422) | | 2810025M15 gene | | | | | | | |
| WAN008BRX_at | RETSAT | Retinol saturase | Hs.440401 | 5E−74 | 83.52403 | 80.18349 | Mm.305108 | 0 | 91.37615 |
| (SEQ ID NO: 423) | | (all-trans- retinol 13, | | | | | | | |
| | | 14-reductase) | | | | | | | |
| WAN008BSS_at | ATAD2 | ATPase family, AAA | Hs.370834 | 7E−51 | 86.74699 | 61.63366 | Mm.221758 | 8E−71 | 91.34615 |
| (SEQ ID NO: 424) | | domain containing 2 | | | | | | | |
| WAN008CI5_at | CDC20 | CDC20 cell division | Hs.524947 | 1E−105 | 89.21283 | 68.6 | Mm.289747 | 1E−142 | 93.58601 |
| (SEQ ID NO: 404) | | cycle 20 homolog | | | | | | | |
| | | (S. cerevisiae) | | | | | | | |
| WAN008CLU_at | Emp1 | Epithelial membrane | Hs.436298 | 0 | 0 | 0 | Mm.182785 | 3E−28 | 90.16393 |
| (SEQ ID NO: 376) | | protein 1 | | | | | | | |
| WAN008CRT_at | ALG14 | Asparagine-linked | Hs.408927 | 4E−47 | 88.39779 | 32.43728 | Mm.269881 | 5E−51 | 88.77005 |
| (SEQ ID NO: 425) | | glycosylation 14 | | | | | | | |
| | | homolog (yeast) | | | | | | | |
| WAN008CS2_at | VKORC1L1 | Vitamin K epoxide | Hs.427232 | 1E−168 | 91.89189 | 96.73203 | Mm.288718 | 0 | 97.28507 |
| (SEQ ID NO: 401) | | reductase complex, | | | | | | | |
| | | subunit 1-like 1 | | | | | | | |
| WAN008CSG_at | Mthfd1 | Methylene- | Hs.614936 | 1E−147 | 86.8705 | 100 | Mm.29584 | 0 | 90.57971 |
| (SEQ ID NO: 426) | | tetrahydrofolate | | | | | | | |
| | | dehydrogenase (NADP+ | | | | | | | |
| | | dependent), | | | | | | | |
| | | methenyltetrahydrofolate | | | | | | | |
| | | cyclohydrolase, | | | | | | | |
| | | formyltetrahydrofolate | | | | | | | |
| | | synthase | | | | | | | |
| WAN008CT2_at | NA | WAN008CT2 10602B- | #N/A | 2E−98 | 92.39544 | 47.0483 | #N/A | 1E−112 | 94.05204 |
| (SEQ ID NO: 427) | | C08 | | | | | | | |
| WAN008CTA_at | NOLC1 | Nucleolar and coiled- | Hs.523238 | 1E−101 | 89.12387 | 59.63964 | Mm.402190 | 3E−28 | 89.90826 |
| (SEQ ID NO: 428) | | body phosphoprotein 1 | | | | | | | |
| WAN008CVX_at | CDC20 | CDC20 cell division | Hs.524947 | 1E−169 | 90.6639 | 85.15901 | Mm.289747 | 0 | 92.30769 |
| (SEQ ID NO: 293) | | cycle 20 homolog | | | | | | | |
| | | (S. cerevisiae) | | | | | | | |
| WAN008CX4_at | MCM5 | MCM5 minichromosome | Hs.517582 | 1E−152 | 87.10247 | 100 | Mm.5048 | 0 | 91.48936 |
| (SEQ ID NO: 302) | | maintenance deficient 5, | | | | | | | |
| | | cell division cycle 46 | | | | | | | |
| | | (S. cerevisiae) | | | | | | | |
| WAN008CXZ_at | UMPS | Uridine monophosphate | Hs.2057 | 1E−135 | 86.13139 | 99.63636 | Mm.13145 | 0 | 91.43898 |
| (SEQ ID NO: 429) | | synthetase (orotate | | | | | | | |
| | | phosphoribosyl | | | | | | | |
| | | transferase and orotidine- | | | | | | | |
| | | 5′-decarboxylase) | | | | | | | |
| WAN008CYY_at | BUB1B | BUB1 budding | Hs.631699 | 4E−34 | 81.32184 | 73.10924 | Mm.29133 | 2E−71 | 84.59384 |
| (SEQ ID NO: 292) | | uninhibited by | | | | | | | |
| | | benzimidazoles 1 | | | | | | | |
| | | homolog beta (yeast) | | | | | | | |
| WAN008CZP_at | NA | WAN008CZP 10604A- | #N/A | 3E−29 | 90.65421 | 21.44289 | #N/A | 7E−50 | 82.69231 |
| (SEQ ID NO: 430) | | A08 | | | | | | | |
| WAN008D06_at | MCM4 | MCM4 minichromosome | Hs.460184 | 1E−159 | 87.97814 | 98.21109 | Mm.1500 | 0 | 92.98561 |
| (SEQ ID NO: 386) | | maintenance deficient 4 | | | | | | | |
| | | (S. cerevisiae) | | | | | | | |
| WAN008D31_at | Lss | Lanosterol synthase | Hs.596543 | 1E−80 | 85.30184 | 68.27957 | Mm.55075 | 1E−150 | 91.52542 |
| (SEQ ID NO: 383) | | | | | | | | | |
| WAN008D7X_at | NA | WAN008D7X 11164B- | #N/A | 4E−16 | 88.23529 | 16.73228 | #N/A | 1E−109 | 91.23377 |
| (SEQ ID NO: 431) | | D06 | | | | | | | |
| WAN008DBR_at | LUC7L | LUC7-like | Hs.16803 | 0 | 93.66197 | 100 | Mm.386921 | 0 | 95.07042 |
| (SEQ ID NO: 432) | | (S. cerevisiae) | | | | | | | |
| WAN008DGK_at | CHAF1A | Chromatin assembly | Hs.79018 | 1E−83 | 91.32231 | 57.89474 | Mm.391010 | 1E−101 | 90.84746 |
| (SEQ ID NO: 433) | | factor 1, subunit A | | | | | | | |
| | | (p150) | | | | | | | |
| WAN008DK1_at | UQCRC1 | Ubiquinol-cytochrome c | Hs.119251 | 3E−69 | 85.66879 | 64.87603 | Mm.335460 | 1E−110 | 91.0828 |
| (SEQ ID NO: 434) | | reductase core protein I | | | | | | | |
| WAN008DMP_at | EWSR1 | Ewing sarcoma | Hs.374477 | 1E−157 | 90.52863 | 94.19087 | Mm.142822 | 0 | 92.98246 |
| (SEQ ID NO: 313) | | breakpoint region 1 | | | | | | | |
| WAN008DO3_at | ACIN1 | Apoptotic chromatin | Hs.124490 | 2E−54 | 89.2562 | 62.85714 | Mm.297078 | 2E−59 | 84.94318 |
| (SEQ ID NO: 435) | | condensation inducer 1 | | | | | | | |
| WAN008DRM_at | EPHX1 | Epoxide hydrolase 1, | Hs.89649 | 9E−85 | 87.98701 | 60.39216 | Mm.9075 | 1E−113 | 91.22257 |
| (SEQ ID NO: 436) | | microsomal (xenobiotic) | | | | | | | |
| WAN008DWL_at | NEK2 | NIMA (never in mitosis | Hs.153704 | 1E−71 | 87.38739 | 58.42105 | Mm.33773 | 1E−152 | 89.13934 |
| (SEQ ID NO: 304) | | gene a)-related kinase 2 | | | | | | | |
| WAN008DXL_at | NA | WAN008DXL 11229A- | #N/A | 5E−58 | 89.74359 | 44.72477 | #N/A | 2E−83 | 94.92386 |
| (SEQ ID NO: 437) | | C02 | | | | | | | |
| WAN008DZY_at | MCM7 | MCM7 minichromosome | Hs.438720 | 3E−99 | 88.37209 | 100 | Mm.241714 | 1E−123 | 91.27907 |
| (SEQ ID NO: 405) | | maintenance deficient 7 | | | | | | | |
| | | (S. cerevisiae) | | | | | | | |
| WAN008E3C_at | Ptma | Prothymosin alpha | Hs.459927 | 2E−67 | 93.83886 | 44.98934 | Mm.19187 | 1E−148 | 92.74005 |
| (SEQ ID NO: 308) | | | | | | | | | |
| WAN008E3O_at | LINCR | Likely ortholog of mouse | Hs.149219 | 3E−19 | 84.61538 | 38.0117 | Mm.389110 | 3E−76 | 85.33724 |
| (SEQ ID NO: 339) | | lung-inducible | | | | | | | |
| | | Neutralized-related | | | | | | | |
| | | C3HC4 RING domain | | | | | | | |
| | | protein | | | | | | | |
| WAN008E4X_at | NA | WAN008E4X 11230A- | #N/A | 0 | 0 | 0 | #N/A | 0 | 0 |
| (SEQ ID NO: 438) | | D06 | | | | | | | |
| WAN008E4Z_at | Nup153 | Nucleoporin 153 | Hs.601591 | 1E−169 | 89.96063 | 92.53188 | Mm.255398 | 0 | 93.75 |
| (SEQ ID NO: 439) | | | | | | | | | |
| WAN008E5L_at | SLC1A5 | Solute carrier family 1 | Hs.631582 | 8E−42 | 84.16667 | 45.62738 | Mm.1056 | 1E−115 | 87.67123 |
| (SEQ ID NO: 440) | | (neutral amino acid | | | | | | | |
| | | transporter), member 5 | | | | | | | |
| WAN008E65_at | ERP29 | Endoplasmic reticulum | Hs.75841 | 1E−164 | 91.04803 | 79.79094 | Mm.154570 | 1E−171 | 90.98532 |
| (SEQ ID NO: 441) | | protein 29 | | | | | | | |
| WAN008E6I_at | NA | WAN008E6I | #N/A | 1E−43 | 88.95706 | 42.22798 | #N/A | 3E−76 | 85.38682 |
| (SEQ ID NO: 442) | | 11230B-F07 | | | | | | | |
| WAN008EED_at | Sc5d | Sterol-C5-desaturase | #N/A | 2E−42 | 85.44601 | 40.72658 | Mm.32700 | 9E−99 | 87.70492 |
| (SEQ ID NO: 443) | | (fungal ERG3, delta-5- | | | | | | | |
| | | desaturase) homolog | | | | | | | |
| | | (S. cerevisae) | | | | | | | |
| WAN008EJV_at | Racgap1 | Rac GTPase-activating | Hs.645513 | 1E−103 | 86.39618 | 93.31849 | Mm.273804 | 1E−133 | 89.31116 |
| (SEQ ID NO: 309) | | protein 1 | | | | | | | |
| WAN008EK5- | NA | WAN008EK5 11232A- | #N/A | 2E−35 | 92.66055 | 26.65037 | #N/A | 6E−44 | 94.78261 |
| rc_f_at | | G08 | | | | | | | |
| (SEQ ID NO: 444) | | | | | | | | | |
| WAN008EML_at | PBK | PDZ binding kinase | Hs.104741 | 5E−52 | 89.50276 | 39.09287 | Mm.24337 | 3E−80 | 89.78102 |
| (SEQ ID NO: 305) | | | | | | | | | |
| WAN008EMN_at | NA | WAN008EMN 11232B- | #N/A | 0 | 0 | 0 | #N/A | 0 | 0 |
| (SEQ ID NO: 445) | | E01 | | | | | | | |
| WAN008EP0_at | NA | WAN008EP0 11232C- | #N/A | 0 | 0 | 0 | #N/A | 0.00003 | 95.12195 |
| (SEQ ID NO: 446) | | B07 | | | | | | | |
| WAN008ET3_at | NA | WAN008ET3 11233A- | #N/A | 3E−47 | 85.65574 | 50.30928 | #N/A | 1E−133 | 89.27739 |
| (SEQ ID NO: 447) | | C09 | | | | | | | |
| WAN008ETA_at | Usp40 | Ubiquitin specific | Hs.96513 | 0 | 0 | 0 | Mm.80484 | 3E−46 | 84.72222 |
| (SEQ ID NO: 448) | | peptidase 40 | | | | | | | |
| WAN008EXF_at | KIF11 | Kinesin family member | Hs.8878 | 4E−28 | 86.86131 | 27.56539 | Mm.42203 | 3E−24 | 91.86047 |
| (SEQ ID NO: 300) | | 11 | | | | | | | |
| WAN008F1A_at | CYC1 | Cytochrome c-1 | Hs.289271 | 1E−124 | 86.82008 | 89.34579 | Mm.29196 | 0 | 92.42424 |
| (SEQ ID NO: 449) | | | | | | | | | |
| WAN013HV4_at | NA | Cluster includes | #N/A | 5E−09 | 97.2973 | 7.07457 | #N/A | 5E−20 | 86.92308 |
| (SEQ ID NO: 450) | | WAN008F09 10599A- | | | | | | | |
| | | D09 | | | | | | | |
| WAN013HVE_at | NARS | Asparaginyl-tRNA | Hs.465224 | 1E−104 | 85.0211 | 79.5302 | Mm.29192 | 0 | 92.22904 |
| (SEQ ID NO: 451) | | synthetase | | | | | | | |
| WAN013HW1_at | Eefld | Eukaryotic translation | Hs.333388 | 1E−115 | 84.05797 | 99.45946 | Mm.258927 | 0 | 91.24088 |
| (SEQ ID NO: 377) | | elongation factor 1 delta | | | | | | | |
| | | (guanine nucleotide | | | | | | | |
| | | exchange protein) | | | | | | | |
| WAN013HW5_at | RPL10A | Ribosomal protein L10a | Hs.546269 | 1E−164 | 89.09465 | 98.98167 | Mm.336955 | 0 | 91.85336 |
| (SEQ ID NO: 452) | | | | | | | | | |
| WAN013HWL_at | EBP | Emopamil binding | Hs.632801 | 6E−21 | 84.17266 | 24.86583 | Mm.27183 | 2E−46 | 91.9708 |
| (SEQ ID NO: 375) | | protein | | | | | | | |
| | | (sterol isomerase) | | | | | | | |
| WAN013HX8_x_at | EIF4A2 | Eukaryotic translation | Hs.518475 | 2E−75 | 94.08602 | 68.50829 | Mm.260084 | 0 | 92.50936 |
| (SEQ ID NO: 453) | | initiation factor 4A, | | | | | | | |
| | | isoform 2 | | | | | | | |
| WAN013HXG_at | NA | Cluster includes | #N/A | 1E−103 | 88.0814 | 62.54545 | #N/A | 1E−118 | 89.14286 |
| (SEQ ID NO: 454) | | WAN008CY6 10604A- | | | | | | | |
| | | H03 | | | | | | | |
| WAN013HZA_at | CSE1L | CSE1 chromosome | Hs.90073 | 1E−180 | 88.51351 | 100 | Mm.22417 | 0 | 93.07432 |
| (SEQ ID NO: 373) | | segregation 1-like (yeast) | | | | | | | |
| WAN013I03_at | RPL8 | Ribosomal protein L8 | Hs.178551 | 1E−166 | 88.00705 | 97.92746 | Mm.30066 | 0 | 92.91883 |
| (SEQ ID NO: 455) | | | | | | | | | |
| WAN013I06_at | NA | Cluster includes | #N/A | 1E−111 | 85.15284 | 84.34622 | #N/A | 1E−143 | 87.71552 |
| (SEQ ID NO: 456) | | WAN008E0Q 11229C- | | | | | | | |
| | | H06 | | | | | | | |
| WAN013I0L_at | SND1 | Staphylococcal nuclease | Hs.122523 | 1E−156 | 87.89683 | 99.40828 | #N/A | 0 | 91.51874 |
| (SEQ ID NO: 457) | | domain containing 1 | | | | | | | |
| WAN013I2L_at | NA | Cluster includes | #N/A | 0 | 0 | 0 | #N/A | 0 | 0 |
| (SEQ ID NO: 458) | | WAN0088QX 10596B- | | | | | | | |
| | | F05 | | | | | | | |
| WAN013I2T_at | CBX5 | Chromobox homolog 5 | Hs.632724 | 1E−142 | 91.86352 | 72.02268 | Mm.262059 | 1E−168 | 94.75066 |
| (SEQ ID NO: 371) | | (HP1 alpha homolog, | | | | | | | |
| | | Drosophila) | | | | | | | |
| WAN013I3N_at | NA | Cluster includes | #N/A | 6E−29 | 87.31343 | 40.36145 | #N/A | 7E−76 | 92.57426 |
| (SEQ ID NO: 459) | | WAN00893W 10599B- | | | | | | | |
| | | D08 | | | | | | | |
| WAN013I5T_at | CCNB1 | Cyclin B1 | Hs.23960 | 1E−93 | 85.30259 | 28.11994 | Mm.260114 | 1E−110 | 87.17949 |
| (SEQ ID NO: 295) | | | | | | | | | |
| WAN013I6G_at | NA | Cluster includes M12252 | #N/A | 0 | 0 | 0 | #N/A | 0 | 0 |
| (SEQ ID NO: 372) | | Chinese hamster alpha- | | | | | | | |
| | | tubulin I mRNA, | | | | | | | |
| | | complete cds. | | | | | | | |
| WAN013I81_at | POLD1 | Polymerase (DNA | Hs.279413 | 0 | 86.30952 | 98.31748 | Mm.16549 | 0 | 91.73372 |
| (SEQ ID NO: 460) | | directed), delta 1, | | | | | | | |
| | | catalytic subunit | | | | | | | |
| | | 125 kDa | | | | | | | |
| WAN013I8D_at | PARP1 | Poly (ADP-ribose) | Hs.177766 | 2E−43 | 85.57692 | 35.01684 | Mm.277779 | 1E−102 | 87.78055 |
| (SEQ ID NO: 461) | | polymerase family, | | | | | | | |
| | | member 1 | | | | | | | |
| WAN013I8J_at | CCNB2 | Cyclin B2 | Hs.194698 | 1E−173 | 86.9258 | 44.39216 | Mm.22592 | 0 | 90.70946 |
| (SEQ ID NO: 296) | | | | | | | | | |
| WAN013I8N_at | IMPDH2 | IMP (inosine | Hs.476231 | 0 | 90.28974 | 95.36968 | Mm.6065 | 0 | 93.18358 |
| (SEQ ID NO: 462) | | monophosphate) | | | | | | | |
| | | dehydrogenase 2 | | | | | | | |
| WAN013I8R_at | Rps2 | Ribosomal protein S2 | Hs.356366 | 0 | 90.22298 | 99.14966 | Mm.157452 | 0 | 95.05119 |
| (SEQ ID NO: 463) | | | | | | | | | |
| WAN013I9O_at | TUBB6 | Tubulin, beta 6 | Hs.193491 | 0 | 92.2528 | 71.39738 | Mm.181860 | 0 | 91.74573 |
| (SEQ ID NO: 400) | | | | | | | | | |
| WAN013I9R_at | NA | Cluster includes Y08202 | #N/A | 1E−104 | 84.573 | 63.46154 | #N/A | 1E−142 | 88.98072 |
| (SEQ ID NO: 294) | | C. griseus mRNA for | | | | | | | |
| | | RAD51 protein | | | | | | | |
| WAN013IAD_at | TOP2A | Topoisomerase (DNA) II | Hs.156346 | 3E−37 | 80.62678 | 29.52061 | Mm.4237 | 1E−86 | 84.11633 |
| (SEQ ID NO: 312) | | alpha 170 kDa | | | | | | | |
| WAN013IAQ- | CDKN1A | Cyclin-dependent kinase | Hs.370771 | 2E−10 | 100 | 14.1129 | Mm.195663 | 1E−31 | 88.88889 |
| rc_x_at | | inhibitor 1A (p21, Cip1) | | | | | | | |
| (SEQ ID NO: 464) | | | | | | | | | |
| X83575_at | KIF23 | Kinesin family member | Hs.270845 | 1E−177 | 92.47788 | 37.07957 | Mm.259374 | 0 | 91.99372 |
| (SEQ ID NO: 381 | | 23 | | | | | | | |
| X83576_at | KIFC1 | Kinesin family member | Hs.436912 | 0 | 86.77111 | 86.84807 | Mm.335713 | 0 | 90.52369 |
| (SEQ ID NO: 301 | | C1 | | | | | | | |

TABLE 13
Platform Process Analysis

| Qualifier List | Symbol | 12A11 d3-d10 | anti5T4 d3-d10 | anti IL-22 2.8 | anti IL- 22 1.19 | Title | Human Unigene ID | eValue | Mouse Unigene ID | eValue |
|---|---|---|---|---|---|---|---|---|---|---|
| WAN008BSN_at | POLR1C | 0.3564015 | 0.5585973 | 0.7136308 | 0.446756 | Polymerase (RNA) I | Hs.584839 | 2E−54 | #N/A | 5E−63 |
| (SEQ ID NO: 465) | | | | | | polypeptide C, 30 kDa | | | | |
| | | | | | | (POLR1C) | | | | |
| WAN008BSS_at | ATAD2 | 0.3468104 | 0.187122 | 0.1709359 | 0.270746 | ATPase family, AAA | Hs.370834 | 7E−51 | Mm.221758 | 8E−71 |
| (SEQ ID NO: 424) | | | | | | domain containing 2 | | | | |
| WAN008CSG_at | Mthfd1 | 0.5305614 | 0.5601446 | 0.6309032 | 0.6230418 | Methylenetetrahydrofolate | Hs.632340 | 1E−147 | Mm.29584 | 0 |
| (SEQ ID NO: 426) | | | | | | dehydrogenase (NADP+ dependent), | | | | |
| | | | | | | methenyltetrahydrofolate | | | | |
| | | | | | | cyclohydrolase, | | | | |
| | | | | | | formyltetrahydrofolate | | | | |
| | | | | | | synthase (Mthfd1), mRNA | | | | |
| WAN008CTZ_at | PGD | 0.8036567 | 0.7430901 | 0.7519262 | 0.7845169 | Phosphogluconate dehydrogenase | Hs.464071 | 1E−105 | Mm.252080 | 1E−153 |
| (SEQ ID NO: 466) | | | | | | | | | | |
| WAN008CVL_x_at | TUBG1 | 0.2894665 | 0.8021069 | 0.6105807 | 0.7480988 | Tubulin, gamma 1 | Hs.279669 | 8E−64 | Mm.142348 | 2E−75 |
| (SEQ ID NO: 467) | | | | | | | | | | |
| WAN008D06_at | MCM4 | 0.5614707 | 0.4896907 | 0.3400396 | 0.6638637 | MCM4 minichromosome | Hs.460184 | 1E−159 | Mm.1500 | 0 |
| (SEQ ID NO: 386) | | | | | | maintenance deficient 4 | | | | |
| | | | | | | (S. cerevisiae) | | | | |
| WAN008D66_at | Mthfd1 | 0.4619552 | 0.517521 | 0.5380162 | 0.4938937 | Methylenetetrahydrofolate | Hs.632340 | 1E−115 | Mm.29584 | 1E−129 |
| (SEQ ID NO: 341) | | | | | | dehydrogenase (NADP+ | | | | |
| | | | | | | dependent), | | | | |
| | | | | | | methenyltetrahydrofolate | | | | |
| | | | | | | cyclohydrolase, | | | | |
| | | | | | | formyltetrahydrofolate | | | | |
| | | | | | | synthase (Mthfd1), | | | | |
| | | | | | | mRNA | | | | |
| WAN008DQC_at | 1110007A13Rik | 0.5689982 | 0.5029418 | 0.5429948 | 0.5160832 | RIKEN cDNA | Hs.124246 | 1E−163 | Mm.97383 | 0 |
| (SEQ ID NO: 468) | | | | | | 1110007A13 gene, | | | | |
| | | | | | | mRNA (cDNA clone | | | | |
| | | | | | | MGC: 28519 | | | | |
| | | | | | | IMAGE: 4191750) | | | | |
| WAN008DTT_at | ATIC | 0.444561 | 0.6791882 | 0.6356441 | 0.6098155 | 5-aminoimidazole-4- | Hs.90280 | 1E−166 | Mm.38010 | 0 |
| (SEQ ID NO: 469) | | | | | | carboxamide | | | | |
| | | | | | | ribonucleotide | | | | |
| | | | | | | formyltransferase/IMP | | | | |
| | | | | | | cyclohydrolase | | | | |
| WAN008DUQ_at | KPNB1 | 0.589151 | 0.6744785 | 0.5598541 | 0.4924376 | Karyopherin (importin) | Hs.532793 | 0 | Mm.251013 | 0 |
| (SEQ ID NO: 470) | | | | | | beta 1 | | | | |
| WAN008DXJ_at | Mm.324279 | 0.6232822 | 0.6457668 | 0.741549 | 0.6234478 | Transcribed locus | #N/A | 1E−113 | Mm.324279 | 0 |
| (SEQ ID NO: 471) | | | | | | | | | | |
| WAN008DYV_at | NOL5A | 0.2796473 | 0.5517918 | 0.8000938 | 0.2160977 | Nucleolar protein 5A | Hs.376064 | 1E−123 | Mm.29363 | 1E−154 |
| (SEQ ID NO: 472) | | | | | | (56 kDa with KKE/D | | | | |
| | | | | | | repeat) | | | | |
| WAN008DZD_at | USP10 | 0.327416 | 0.688104 | 0.796735 | 0.554588 | Ubiquitin specific | Hs.136778 | 1E−151 | Mm.256910 | 0 |
| (SEQ ID NO: 473) | | | | | | peptidase 10 | | | | |
| WAN008E5L_at | Slc1a5 | 0.442547 | 0.437469 | 0.791016 | 0.352119 | Solute carrier family 1 | Hs.631582 | 8E−42 | Mm.1056 | 1E−115 |
| (SEQ ID NO: 440) | | | | | | (neutral amino acid | | | | |
| | | | | | | transporter), member 5, | | | | |
| | | | | | | mRNA (cDNA clone | | | | |
| | | | | | | MGC: 46952 | | | | |
| | | | | | | IMAGE: 4192790) | | | | |
| WAN008EHW_at | OPRS1 | 0.516148 | 0.740823 | 0.768598 | 0.722368 | Opioid receptor, sigma 1 | Hs.522087 | 1E−141 | Mm.29025 | 1E−163 |
| (SEQ ID NO: 474) | | | | | | | | | | |
| WAN008ETA_at | NA | 0.611091 | 0.50109 | 0.583993 | 0.646248 | Ubiquitin specific | #N/A | 0 | Mm.80484 | 3E−46 |
| (SEQ ID NO: 448) | | | | | | peptidase 40 (Usp40) | | | | |
| WAN013HVE_at | NARS | 0.423147 | 0.472965 | 0.637676 | 0.320468 | Asparaginyl-tRNA | Hs.465224 | 1E−104 | Mm.29192 | 0 |
| (SEQ ID NO: 451) | | | | | | synthetase | | | | |
| WAN013HZA_at | CSE1L | 0.35612 | 0.431439 | 0.339859 | 0.428959 | CSE1 chromosome | Hs.90073 | 1E−180 | Mm.22417 | 0 |
| (SEQ ID NO: 373) | | | | | | segregation 1-like (yeast) | | | | |
| WAN013I1P_at | HNRPA2B1 | 0.404557 | 0.420546 | 0.325162 | 0.471038 | Heterogeneous nuclear | Hs.487774 | 0 | Mm.155896 | 0 |
| (SEQ ID NO: 380) | | | | | | ribonucleoprotein A2/B1 | | | | |
| WAN013I3N_at | NA | 0.414224 | 0.581925 | 0.404672 | 0.590106 | Heat shock protein 8 | | 6E−29 | Mm.336743 | 8E−76 |
| (SEQ ID NO: 459) | | | | | | (Hspa8) | | | | |
| WAN013I6N_at | EEF2 | 0.375943 | 0.678406 | 0.733043 | 0.548719 | Eukaryotic translation | Hs.515070 | 0 | Mm.289431 | 0 |
| (SEQ ID NO: 475) | | | | | | elongation factor 2 | | | | |
| WAN013I8N_at | IMPDH2 | 0.342863 | 0.689042 | 0.547075 | 0.41901 | IMP (inosine | Hs.476231 | 0 | Mm.6065 | 0 |
| (SEQ ID NO: 462) | | | | | | monophosphate) | | | | |
| | | | | | | dehydrogenase 2 | | | | |
| WAN013I9E_at | Akr1b8 | 0.62991 | 0.717648 | 0.573995 | 0.799038 | Aldo-keto reductase | Hs.116724 | 1E−136 | Mm.5378 | 0 |
| (SEQ ID NO: 476) | | | | | | family 1, member B10 | | | | |
| | | | | | | (aldose reductase) | | | | |
| | | | | | | (AKR1B10) | | | | |
| WAN013IAD_at | TOP2A | 0.300828 | 0.116559 | 0.121984 | 0.174821 | Topoisomerase (DNA) II | Hs.156346 | 3E−37 | Mm.4237 | 2E−86 |
| (SEQ ID NO: 312) | | | | | | alpha 170 kDa | | | | |
| AF004814_at | NA | 1.743781 | 1.534337 | 1.218754 | 1.443915 | AF004814 Mesocricetus | #N/A | 1E−157 | #N/A | 0 |
| (SEQ ID NO: 477) | | | | | | auratus ubiquitin | | | | |
| | | | | | | conjugating enzyme | | | | |
| | | | | | | (UBC9) mRNA, complete | | | | |
| | | | | | | cds. | | | | |
| AF022941_x_at | Cirbp | 12.32258 | 4.109663 | 4.088415 | 5.81758 | Cold inducible RNA | Hs.501309 | 9E−27 | Mm.17898 | 1E−52 |
| (SEQ ID NO: 409) | | | | | | binding protein (Cirbp), | | | | |
| | | | | | | mRNA | | | | |
| AF022942_at | CIRBP | 3.793483 | 3.737599 | 3.985995 | 3.264907 | Cold inducible RNA | Hs.501309 | 4E−60 | Mm.17898 | 9E−94 |
| (SEQ ID NO: 478) | | | | | | binding protein | | | | |
| AF093673_at | LLN | 2.06764 | 1.400671 | 1.622571 | 4.182745 | layilin | #N/A | 1E−158 | #N/A | 1E−155 |
| (SEQ ID NO: 479) | | | | | | | | | | |
| S74024_at | Xpa | 1.934745 | 1.86472 | 1.815468 | 1.610521 | Xeroderma pigmentosum, | Hs.591907 | 4E−54 | Mm.247036 | 6E−64 |
| (SEQ ID NO: 480) | | | | | | complementation group | | | | |
| | | | | | | A, mRNA (cDNA clone | | | | |
| | | | | | | MGC: 36016 | | | | |
| | | | | | | IMAGE: 4489063) | | | | |
| WAN0088IR_at | Nqo1 | 2.285019 | 1.23648 | 1.480098 | 3.629589 | NAD(P)H dehydrogenase, | Hs.406515 | 1E−42 | Mm.252 | 4E−87 |
| (SEQ ID NO: 481) | | | | | | quinone 1 (Nqo1), mRNA | | | | |
| WAN0088T3_at | ANXA1 | 2.306751 | 2.378823 | 2.276998 | 1.666077 | Annexin A1 | Hs.494173 | 9E−72 | Mm.248360 | 1E−114 |
| (SEQ ID NO: 482) | | | | | | | | | | |
| WAN0088YL_f_at | 2700085E05Rik | 1.619824 | 1.604107 | 1.433464 | 1.664261 | RIKEN cDNA | #N/A | 2E−58 | Mm.249700 | 3E−75 |
| (SEQ ID NO: 483) | | | | | | 2700085E05 gene | | | | |
| | | | | | | (2700085E05Rik), mRNA | | | | |
| WAN008D29_s_at | LGALS3 | 2.388289 | 2.661416 | 2.042812 | 2.590684 | Lectin, galactoside- | Hs.531081 | 3E−41 | Mm.248615 | 5E−79 |
| (SEQ ID NO: 484) | | | | | | binding, soluble, 3 | | | | |
| | | | | | | (galectin 3) | | | | |
| WAN008D85_at | Calm3 | 2.966847 | 1.714363 | 1.242839 | 2.386649 | Calmodulin III (Calm3) | Hs.515487 | 3E−19 | Mm.288630 | 1E−115 |
| (SEQ ID NO: 485) | | | | | | mRNA, 3′ untranslated | | | | |
| | | | | | | region | | | | |
| WAN008DKX_at | NA | 2.467922 | 2.208711 | 1.525653 | 2.845161 | WAN008DKX 11188C- | #N/A | 0 | #N/A | 5E−13 |
| (SEQ ID NO: 486) | | | | | | B06 | | | | |
| WAN008DQD_at | RIT1 | 2.149397 | 1.299586 | 1.903483 | 1.667202 | Ras-like without CAAX1 | Hs.491234 | 1E−136 | Mm.4009 | 1E−146 |
| (SEQ ID NO: 487) | | | | | | | | | | |
| WAN008E7S_at | NA | 1.724905 | 0.587986 | 1.438937 | 2.190444 | WAN008E7S 11230B- | #N/A | 0 | #N/A | |
| (SEQ ID NO: 488) | | | | | | A08 | | | | |
| WAN008EUZ_x_at | NA | 1.314677 | 1.975245 | 1.26652 | 1.266918 | WAN008EUZ 11233C- | #N/A | 2E−21 | #N/A | |
| (SEQ ID NO: 489) | | | | | | D05 | | | | |
| WAN013HUG_at | CDKN2C | 1.804659 | 1.522681 | 1.22055 | 2.164155 | Cyclin-dependent kinase | Hs.525324 | 1E−113 | Mm.1912 | |
| (SEQ ID NO: 490) | | | | | | inhibitor 2C (p18, inhibits | | | | |
| | | | | | | CDK4) | | | | |
| WAN013HUW_at | Arl1 | 1.469845 | 1.703938 | 1.339026 | 1.310857 | ADP-ribosylation factor- | Hs.372616 | 2E−88 | Mm.291247 | |
| (SEQ ID NO: 491) | | | | | | like 1 (Arl1), mRNA | | | | |
| WAN013HX5_at | MGST1 | 1.654933 | 1.444058 | 1.316584 | 2.43629 | Microsomal glutathione | Hs.389700 | 3E−28 | Mm.14796 | |
| (SEQ ID NO: 492) | | | | | | S-transferase 1 | | | | |
| WAN013HXQ_at | ADH5 | 1.860866 | 1.973314 | 1.313058 | 1.679271 | Alcohol dehydrogenase 5 | Hs.78989 | 1E−180 | Mm.3874 | |
| (SEQ ID NO: 493) | | | | | | (class III), chi polypeptide | | | | |
| WAN013HZO_at | Anxa2 | 1.925088 | 2.698972 | 2.096341 | 1.275319 | Annexin A2, mRNA | Hs.511605 | 8E−76 | Mm.238343 | |
| (SEQ ID NO: 494) | | | | | | (cDNA clone MGC: 6547 | | | | |
| | | | | | | IMAGE: 2655513) | | | | |
| WAN013I1X_at | ANXA1 | 3.210728 | 2.885541 | 2.042778 | 1.864157 | Annexin A1 | Hs.494173 | 1E−137 | Mm.248360 | |
| (SEQ ID NO: 495) | | | | | | | | | | |
| WAN013I2Q_at | 1500011L16Rik | 2.657517 | 3.051476 | 3.294782 | 3.393943 | SSU72 RNA polymerase | Hs.30026 | 1E−132 | Mm.294770 | |
| (SEQ ID NO: 496) | | | | | | II CTD phosphatase | | | | |
| | | | | | | homolog (S. cerevisiae) | | | | |
| WAN013I33_at | 2210013O21Rik | 2.664118 | 1.250289 | 1.615807 | 2.311342 | PREDICTED: | #N/A | 2E−88 | Mm.146408 | |
| (SEQ ID NO: 497) | | | | | | hypothetical protein | | | | |
| | | | | | | LOC70123 [Mus | | | | |
| | | | | | | musculus], mRNA | | | | |
| | | | | | | sequence | | | | |
| WAN013I3V_at | APRT | 1.457976 | 1.856872 | 1.73596 | 1.682916 | Adenine | Hs.28914 | 4E−64 | Mm.1786 | |
| (SEQ ID NO: 498) | | | | | | phosphoribosyltransferase | | | | |
| WAN013I4Q_at | GLUL | 1.961117 | 1.741955 | 1.574109 | 2.192446 | Glutamate-ammonia | Hs.518525 | 1E−136 | Mm.210745 | |
| (SEQ ID NO: 499) | | | | | | ligase (glutamine | | | | |
| | | | | | | synthetase) | | | | |
| WAN013I51_at | SAT | 1.963134 | 2.108509 | 1.532499 | 1.473022 | Spermidine/spermine N1- | Hs.28491 | 1E−118 | #N/A | |
| (SEQ ID NO: 500) | | | | | | acetyltransferase | | | | |
| WAN013I66_f_at | Vim | 2.634189 | 2.037839 | 2.226342 | 1.903473 | Vimentin (Vim), mRNA | Hs.533317 | 1E−126 | Mm.268000 | |
| (SEQ ID NO: 501) | | | | | | | | | | |
| WAN013I96_at | MDM2 | 3.30029 | 4.084442 | 2.893163 | 1.519829 | Mdm2, transformed 3T3 | Hs.567303 | 5E−83 | Mm.22670 | 2E−89 |
| (SEQ ID NO: 502) | | | | | | cell double minute 2, p53 | | | | |
| | | | | | | binding protein (mouse) | | | | |
| WAN013I9K_at | | 2.225103 | 1.775629 | 1.982478 | 8.531181 | Glutathione S-transferase | Hs.301961 | 1E−87 | Mm.37199 | 1E−173 |
| (SEQ ID NO: 503) | | | | | | M1 (GSTM1) | | | | |

### Example 6

### Proteomic Analysis of Proteins Associated with Enhanced Survival

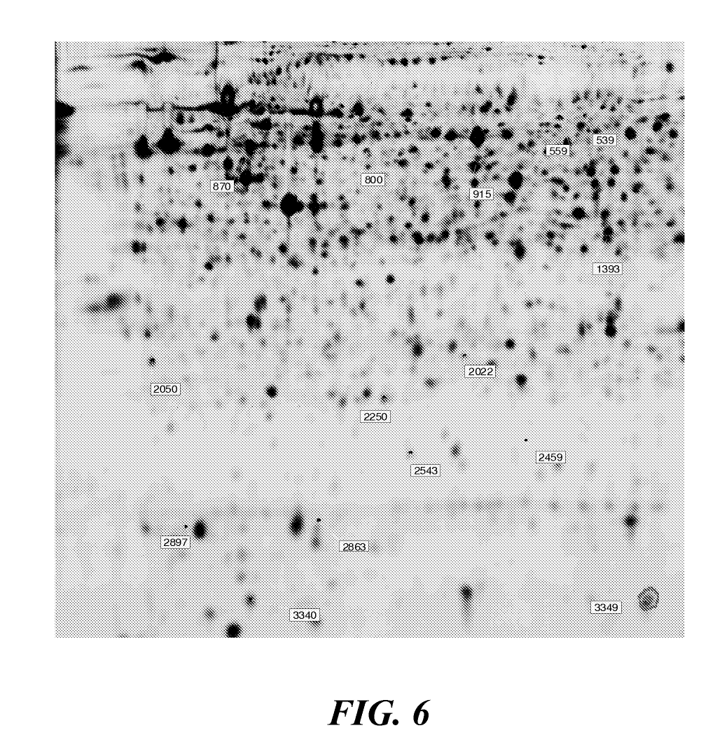

Samples were taken from the parental lineage and the high viability B19 clone fermentations used for the transcriptional profiling, and proteins were isolated for proteomic analysis on 2D gels with a pH range of 4-7. From this analysis, 53 protein spots were considered differentially expressed (p≦0.05 with a fold difference of ≧1.5), of which 29 were upregulated and 24 were downregulated in B19 (FIG. 6), many of which were of low abundance on the gel. Using MALDI-ToF mass spectrometry and LC-MS/MS we were able to identify 15 of the differentially expressed proteins (Table 14). The low number of identified proteins is due in part to the limitations of spot separation resulting from the PI range of the first-dimension, and also in part to the fact that the greater proportion of differentially expressed proteins were of low abundance and therefore difficult to identify (a common limitation of 2D DIGE analysis). Significantly, several proteins identified in the proteomic analysis displayed similar trends of differential expression in the microarray analysis of their cognate coding sequences. For example, in the cases of tumor protein, translationally-controlled 1, Atp5b protein and 3-hydroxy-3 methylglutaryl-Coenzyme A synthase 1 there are consistent expression profiles in both the proteomic and genomic analyses (Table 14). In the case of mitochondrial ribosomal protein (MRPL), which was identified as being downregulated from 2D DIGE analysis. Two mitochondrial ribosomal proteins (MRPL12 and MRPL30) were also similarly downregulated in the transcriptional profiling data, which suggests the data may support one another. In contrast, there are probes for both succinate dehydrogenase (Sdha) and Chaperonin containing TCP1, subunit 3 (gamma) on the WyeHamster2a array, but in each case the transcript levels were essentially unchanged indicating that the altered protein levels may reflect a post transcriptional regulation mechanism.

TABLE 14
Differential gene and protein expression comparing a parental cell line vs. a Bcl-xL expressing derivative (B19).
Where possible, the relevant transcript information is given also.

| | | | | | Microarray Data | | | |
|---|---|---|---|---|---|---|---|---|
| 2D DIGE Data | | | | | Early | | Late | |
| Spot | Protein | ID Method | FC^{a} | Biological Function | FC | P Value | FC | P Value |
| 3340 | S100 calcium-binding protein A13 | LC-MS/MS | −7.5 | cell differentiation | — | — | — | — |
| 870 | Atp5b protein | MALDI ID | −1.8 | ATP biosynthetic process | −1.8 | 3.84E−02 | — | — |
| 2250 | Mitochondrial ribosomal protein | MALDI ID | −1.6 | translation | −1.7 | 3.68E−02^{b} | — | — |
| | | | | | −1.6 | 3.48E−02^{c} | — | — |
| 1393 | Transaldolase | MALDI ID | −1.6 | Carbohydrate metabolic process | | | — | — |
| 2050 | Tumor protein, translationally- | MALDI ID | −1.6 | calcium ion homeostasis | −2.0 | 3.12E−03 | — | — |
| | controlled 1 | | | | | | | |
| 3349 | S100 calcium-binding protein A10 | LC-MS/MS | −1.6 | signal transduction | — | — | — | — |
| 915 | Aldehyde dehydrogenase, | MALDI ID | −1.5 | Carbohydrate metabolic process | — | — | — | — |
| | mitochondrial. | | | | | | | |
| 559 | Sdha protein (succinate | MALDI ID | −1.5 | tricarboxylic acid cycle | # | # | # | # |
| | dehydrogenase) | | | | | | | |
| 800 | HMGCS1 | MALDI ID | +2.9 | Cholesterol biosynthetic process | +2.5 | 9.76E−03 | +2.9 | 5.21E−03 |
| 2459 | prefoldin subunit 2 | MALDI ID | +2.1 | protein folding | — | — | — | — |
| 539 | Chaperonin containing TCP1, | MALDI ID | +1.9 | protein folding | # | # | # | # |
| | subunit 3 (gamma) | | | | | | | |
| 2897 | ATP synthase alpha chain, | MALDI ID | +1.8 | ATP synthesis coupled proton | — | — | — | — |
| | mitochondrial precursor isoform 3 | | | transport | | | | |
| 2543 | stathmin | MALDI ID | +1.7 | intracellular signaling cascade | — | — | — | — |
| 2863 | profilin II | MALDI ID | +1.7 | actin cytoskeleton organization | — | — | — | — |
| | | | | and biogenesis | | | | |
| 2022 | GRP2 (Growth factor receptor- | MALDI ID | +1.5 | Ras protein signal transduction | — | — | — | — |
| | bound protein 2) | | | | | | | |

^{a}(+) Upregulation in B19, ratio is B19/parent; (−) Downregulation in B19, ratio is parent/B19
^{b}WyeHamster2a array data for Mitochondrial ribosomal protein L12 (MRPL12)
^{c}WyeHamster2a array data for Mitochondrial ribosomal protein L30 (MRPL30)
— Not Available on WyeHamster2a array
# Available on WyeHamser2a array but transcript level unchanged.

### Example 7

### Target Validation Screens

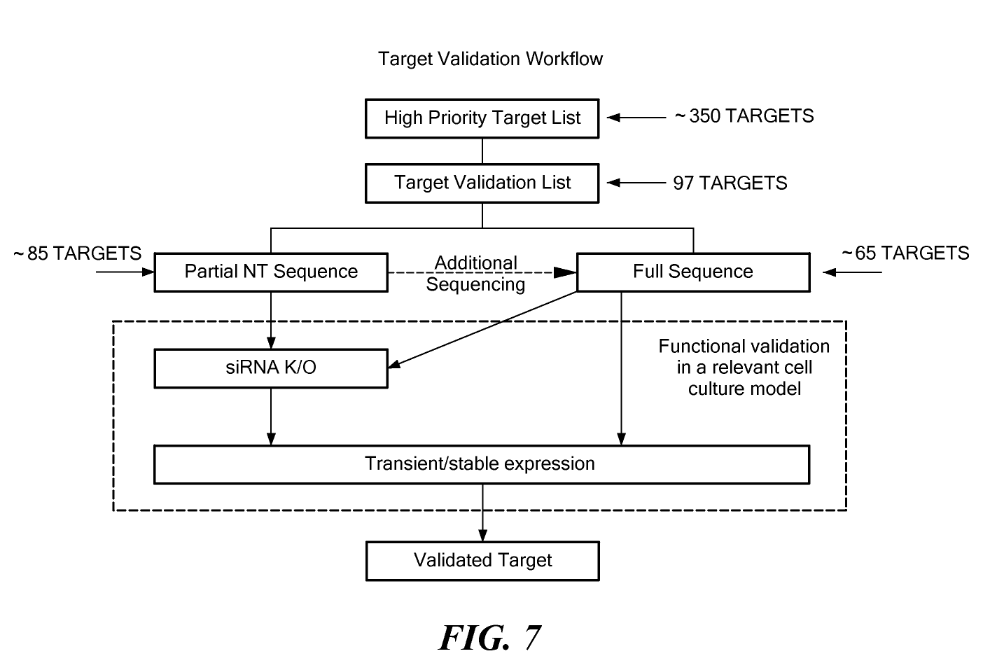

The differentially expressed proteins or genes can be used to engineer cells to improve a cell line. For example, those proteins or genes unique to test cell lines or test cell cultures may be overexpressed to reproduce or further improve desirable cell phenotypes. Conversely, those proteins or genes unique to control cell lines or control cell cultures may be down-regulated to avoid undesirable cell phenotypes. FIG. 7 illustrates an exemplary target validation workflow.

To help prioritizing the targets for validation, target validation screens were designed. Typically, siRNA and transient overexpression assays are used for validation screens. Typically, validation assays are not optimized for any single gene target, but rather for the assay format and the controls that are used. Typically, multiple cell lines are used for both siRNA and transient overexpression experiments to identify targets that have more consistent and/or desirable effect. For example, two cell lines are used for siRNA assays and three cell lines are used for transient expression assays. Typically, multiple time points (e.g., at least 2 for each assay type) are analyzed, because relevant timepoints for determination of knockdown or overexpression effects of different targets may vary for different targets. For example, in siRNA assays, the turnover rate of different protein products may vary, and therefore the relevant effective timepoints of the knockdown vary among different proteins. Likewise, in transient overexpression assays, when the expression of transfected proteins reach an effective level may vary among different protein targets. In addition, at least 2 siRNA molecules are used for each target to avoid an ineffective siRNA molecules.

Typically, primary endpoints measured in both the knockdown and overexpression assays are growth and productivity. To determine the knockdown or overexpression effects, we compare the test cells against appropriate controls in each assay, and calculate the fold change difference with respect to growth and/or productivity of targeted cells versus control cells. Typically, in siRNA assays, suitable controls include untransfected cells, mock transfected cells, cells carrying scrambled RNAs or nonsense RNAs. In overexpression assays, suitable controls include untransfected cells, mock transfected cells, cells carrying empty vectors. Appropriate productivity or growth controls can also be included.

### Example 8

### Target Validation: siRNA

The ability of differentially expressed genes and proteins to affect a cellular phenotype can be first verified by overexpression of nucleic acids inhibiting the expression of relevant genes using methods known in the art. Exemplary methods based on interfering RNA constructs are described below.

Design and Synthesis of siRNA

Typically, candidate targets suitable for siRNA mediated gene knockdown are sequenced, and the sequences are verified. Full-length cDNA sequence information is preferred (although not required) to facilitate siRNAs design. Candidate target sequences are compared to gene sequences available on public or proprietary databases (e.g., BLAST search). Sequences within candidate target genes that overlap with other known sequences (for example, 16-17 contiguous basepairs of homology) are generally not suitable targets for specific siRNA-mediated gene knockdown.

siRNAs may be designed using, for example, online design tools, over secure internet connections, such as the one available on the Ambion® website (http://www.ambion.com/techlib/misc/siRNA_finder.html). Alternatively, custom siRNAs may also be requested from Ambion®, which applies the Cenix algorithm for designing effective siRNAs. Standard format for siRNAs is typically 5 nmol, annealed and with standard purity in plates. Upon receipt, siRNAs are prepared according to the instructions provided by the manufacture and stored at the appropriate temperature (−20° C.)

Spin Tube siRNA Transfection

Two antibody-expressing cell lines were used for siRNA transfections. Cells to be transfected were typically pre-passaged on the day before transfection to ensure that the cells are in logarithmic growth phase.

All spin tubes were labeled in hood. For each target, 8 tubes (e.g., 2 cell lines, 2 siRNA's, all in duplicate) were used. For each experiment, about 100,000 cells in 1 mL total volume were used. For each transfection, 100 μL R1 and 2 μL Mirus TKO reagent were mixed and incubated for 10 minutes at room temperature. Meanwhile, siRNAs were aliquoted into appropriate eppendorf tubes. Start cells spinning to give appropriate seed densities. After 10 minutes incubation, add 102 μL mixture to each siRNA eppendorf tube and incubate transfection mixtures for 15-20 minutes. Resuspend cells in serum free medium to get a final density of 1.0E5 cell/mL. 1.9 mL of cells were transferred to each spin tube. After 15 minutes incubation, siRNA mix (112 μL) was added to each spin tube. The culture was incubated at 37° C. Spin tube cultures were shaken rapidly (˜250 RPM). Samples were taken on day 1 (count), day 3 (count titer, feed), and day 7 (count and titer). Cultures were terminated on day 7.

### 24 Well Suspension Transfections

For each experiment, 100,000 cells (e.g., 3C7 cells) in 1 mL total volume, and 50 nM siRNA were used. To make a mix for 3 reactions, 150 μL R1 and 70 μL Mirus TKO reagent were mixed and incubated for 10 minutes at room temperature. 15 μL of 10 μM siRNA was added and the mix was incubated for 10 minutes at room temperature. 57.3 μL of the mix was transferred into each of 3 wells. 942.7 μL of R5CD1 (containing 100,000 cells) was added and the plate was incubated on rocker at 37° C. for 72 hrs.

Growth and productivity controls were included on each plate. An exemplary productivity control is DHFR (selectable marker on bicistronic mRNA). Treatment with DHFR siRNA reproducibly decreases amount of antibody in the CM-FcIGEN (antibody production control). An exemplary growth control is CHO1 (kinesin) (see Matuliene et al. (2002) Mol. Cell. Biol. 13:1832-45) (typically, about 20-30% growth inhibition was observed with CHO1 treatment). Other standard controls such as no siRNA treatment (transfection reagents only) and non-targeting siRNA treatment (non-specific siRNA) were also included. Plates were then subjected to cell counting (for example, in a 96-well cell counting instrument) to assess growth and to, for example, an automated 96-well titer assay, to assess productivity. Exemplary results are shown in Table 15.

Genes whose modulation, singly or in combination, are sufficient to modify useful cellular phenotypes were thereby validated and such changes can be engineered, singly or in combination, into a mammalian cell line to modify its properties.

### Example 9

### Target Validation: Overexpression

The ability of differentially expressed genes and proteins to affect a cellular phenotype can also be verified by overexpression. In these experiments, specific targets were introduced into CHO cells by transient transfections and then the impact of over-expression on cellular growth and productivity were monitored.

Lipofectamine 2000 reagent (Invitrogen), serum-free base media, serum-free feed media and 24-well non-tissue culture plates (standing order set up from VWR) were used. Three cell lines were used for the overexpression assessment. Banks of each cell line have been created for the overexpression assay, and are stored in liquid nitrogen for long term storage. New vials are thawed out every 4-6 weeks. Cells are transfected on day 3 of a 3-day/4-day passage.

Basic Transfection protocol (per transfection): Count cells using standard methods. Viability should be in the upper nineties. Need 8*e5 cells in 900 ul, so adjust the final cell concentration to 9*e5 cells/ml. Need approximately 30 ml of cell suspension for each assay plate per cell line. Spin down cell culture at 1000 rpm for 7 minutes, and resuspend in fresh base medium.

Dilute DNAs (vector only, controls, and test genes) in fresh base medium. Prepare master mix for each depending on the number of transfections. Each cell line has 3 wells as replicates. For testing 3 cell lines, prepare master mix for 9+1=10 transfections. Mastermix was prepared as follows: 1 μg of DNA was diluted in base medium and adjusted to total volume of 50 ul)*number of transfections.

Dilute L2000 in base medium (2 μl in 50 μl). Label each 24-well plate. Tilt the plate on the edge of the lid, and pipette 50 μl of the diluted L2000 at the bottom of each well. Add 50 μl of the relevant diluted DNA master mix to each well. Carefully pipette up and down 3-4 times to mix. Pipette tips were changed when switching between different DNAs. The mixtures were incubate for 20 minutes at room-temperature. During this time, spin down the cells that had been counted, and resuspend in fresh base medium. Pipette 900 μl of cell-suspension into each well. Mix gently by pipetting up and down 3-4 times. Incubate at 37° C. on the orbital shaker at the marked speed.

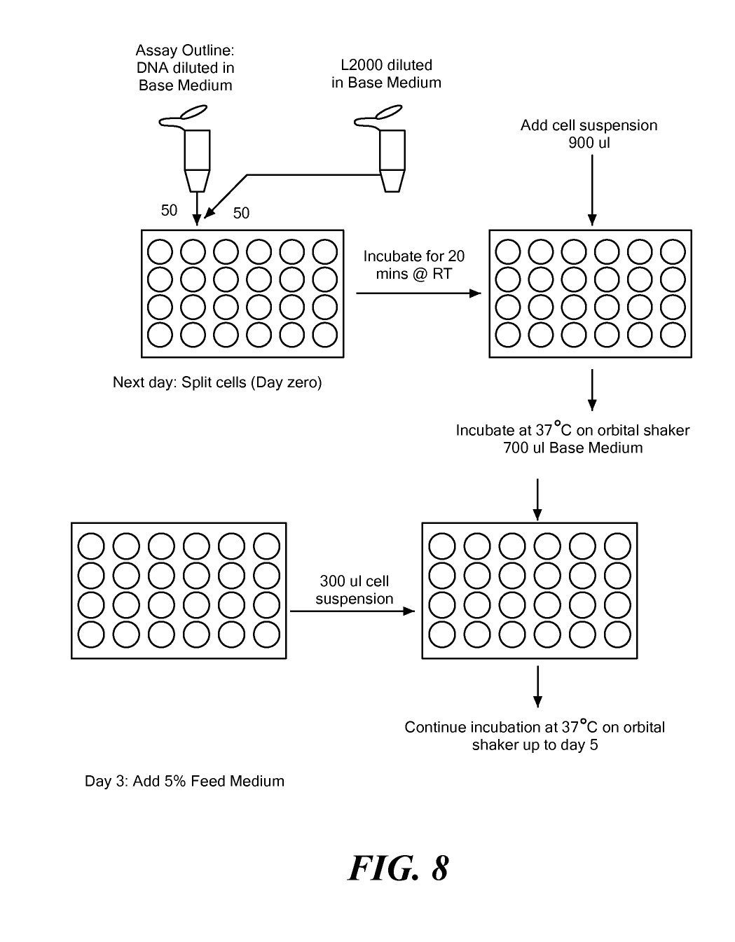

On the next day, pull out 300 μl of cell suspension from each well and transfer to corresponding well in a fresh 24 well non-tc plate. Normally, two 1000 μl pipettes were used for this step. One to pipette the cell suspension up and down 3 to 4 times to ensure proper mixing (important because the cells settle down very quickly), then go back to pull out the 300 μl with the other pipette. Add 700 ul of fresh base medium to each well and mix. This day was designated as day zero. Count cells and repeat count on day 3. Then add 50 μl of feed media (5%). Count cells on day 5, then spin down the cell suspension from each well and collect conditioned media. Send for titer assay. Exemplary overexpression assays are outlined in FIG. 8.

Growth and productivity controls are typically used for overexpression assays. For example, positive growth/viability control used in this experiment included Ha-Ras and Bcl-xL. Negative growth control used included p27. Other suitable growth and productivity controls are known in the art and can be used for overexpression assays. Additional standard controls such as no nucleic acid control (transfection reagents only) were also included. Exemplary results are shown in Table 15.

### Example 10

### Engineering Cell Lines to Improve Cell Phenotypes Based on the Verified Target Genes

The verified target genes are used to effect a cell phenotype, particularly a phenotype characterized by increased and efficient production of a recombinant transgene, increased cell growth rate, high peak cell density, sustained high cell viability, high maximum cellular productivity, sustained high cellular productivity, low ammonium production, and low lactate production, etc. Exemplary target genes are disclosed above, for example, in Tables 2 through 13.

Standard cell engineering methods are used to modify target genes to effect desired cell phenotypes. As discussed above, target genes are modified to achieve desired CHO cell phenotypes by interfering RNA, conventional gene knockout or overexpression methods. Typically, knockout methods or stable transfection methods with overexpression constructs are used to engineer modified CHO cell lines. Other suitable methods are discussed in the general description section and known in the art.

TABLE 15

| | | | siRNA | | | | | | | | Overexpression | | | | | | | | | | | |
|---|---|---|---|---|---|---|---|---|---|---|---|---|---|---|---|---|---|---|---|---|---|---|
| | | | Growth | | | | Productivity | | | | Growth | | | | | | Productivity | | | | | |
| | | | 1.14 | 5C10 | 1.14 | 5C10 | 1.14 | 5C10 | 1.14 | 5C10 | 1.14 | 2B6 | 3B12 | 1.14 | 2B6 | 3B12 | 1.14 | 2B6 | 3B12 | 1.14 | 2B6 | 3B13 |
| | Name | | | | | | | | | | | | | | | | | | | | | |
| ID | (Symbol) | siRNA ID | D3 | D3 | D7 | D7 | D3 | D3 | D7 | D7 | D3 | D3 | D3 | D5 | D5 | D5 | D3 | D3 | D3 | D5 | D5 | D5 |
| WAN008D2Q_at | Eukaryotic translation | 289077 | 0.97 | 0.74 | 0.94 | 0.64 | 0.87 | 0.78 | 0.93 | 0.81 | 1.24 | 1.34 | 1.11 | 0.91 | 1.24 | 1.26 | 0.67 | 0.87 | 0.63 | 0.90 | 0.82 | 0.74 |
| | initiation factor 4B | 289078 | 0.74 | 0.97 | 0.64 | 1.06 | 0.89 | 0.74 | 0.95 | 0.86 | | | | | | | | | | | | |
| | (Eif4b) | | | | | | | | | | | | | | | | | | | | | |
| WAN013I8K_at | Cluster includes | 289073 | 0.46 | 0.25 | 0.39 | 0.41 | 0.62 | 1.27 | 0.68 | 0.76 | | | | | | | | | | | | |
| | D29972 Cricetulus | 289074 | 0.65 | 0.39 | 0.81 | 0.87 | 0.64 | 0.88 | 0.74 | 0.67 | | | | | | | | | | | | |
| | griseus mitochondrial | | | | | | | | | | | | | | | | | | | | | |
| | DNA, D-loop region. | | | | | | | | | | | | | | | | | | | | | |
| X51747_at | Heat shock 27 kDa | 289081 | 0.97 | 0.72 | 0.71 | 0.84 | 0.46 | 1.02 | 0.59 | 0.85 | | | | | | | | | | | | |
| | protein 1 | 289082 | 0.74 | 0.57 | 0.47 | 0.60 | 0.83 | 1.57 | 0.84 | 0.94 | | | | | | | | | | | | |
| | (HSPB1) | | | | | | | | | | | | | | | | | | | | | |
| U48852_at | U48852 Cricetulus | 289088 | 0.96 | 0.54 | 0.72 | 0.67 | 0.60 | 1.01 | 0.84 | 0.89 | 0.62 | 1.31 | 1.17 | 0.78 | 1.22 | 1.32 | 1.39 | 0.87 | 0.72 | 1.03 | 0.77 | 0.60 |
| | griseus HT protein | 289089 | 1.26 | 0.87 | 0.77 | 0.79 | 0.50 | 0.83 | 0.80 | 0.74 | | | | | | | | | | | | |
| | mRNA, complete cds. | | | | | | | | | | | | | | | | | | | | | |
| | (HT) | | | | | | | | | | | | | | | | | | | | | |
| WAN008DJ9_at | Solute carrier family 1 | 292184 | 1.25 | 0.90 | 1.03 | 0.86 | 0.88 | 1.61 | 0.81 | 1.09 | 1.05 | 1.10 | 0.93 | 0.91 | 1.21 | 0.92 | 1.04 | 0.86 | 1.23 | 1.12 | 0.85 | 1.06 |
| | (glutamate/neutral | 292185 | 1.20 | 0.99 | 0.99 | 0.99 | 0.65 | 1.33 | 0.80 | 0.99 | | | | | | | | | | | | |
| | amino acid | | | | | | | | | | | | | | | | | | | | | |
| | transporter), member 4 | | | | | | | | | | | | | | | | | | | | | |
| | (SLC1A4) | | | | | | | | | | | | | | | | | | | | | |
| gi\|15100179 | malate dehydrogenase | 292186 | 1.18 | 1.06 | 0.99 | 1.17 | 0.95 | 1.26 | 0.84 | 0.97 | 0.98 | 0.93 | 0.92 | 0.79 | 1.12 | 0.83 | 1.06 | 0.90 | 1.18 | 1.24 | 0.78 | 1.15 |
| | (soluble) | 292187 | 1.45 | 1.04 | 1.11 | 0.91 | 0.82 | 1.20 | 0.86 | 0.87 | | | | | | | | | | | | |
| WAN0088T2_at | Activating transcription | 292188 | 0.80 | 1.05 | 0.95 | 0.88 | 1.15 | 0.69 | 1.00 | 0.83 | | | | | | | | | | | | |
| | factor 4 (tax- | 292189 | 0.94 | 1.06 | 0.98 | 0.96 | 1.39 | 0.73 | 0.71 | 0.80 | | | | | | | | | | | | |
| | responsive enhancer | | | | | | | | | | | | | | | | | | | | | |
| | element B67) | | | | | | | | | | | | | | | | | | | | | |
| | (ATF4) | | | | | | | | | | | | | | | | | | | | | |
| gi\|73968066 | heat shock 70 kDa | 293861 | 1.32 | 1.05 | 1.31 | 1.15 | 0.72 | 0.90 | 0.96 | 0.80 | 1.16 | 0.90 | 1.08 | 0.96 | 1.03 | 0.99 | 0.86 | 0.90 | 0.86 | 1.02 | 0.85 | 0.95 |
| | protein 5 (glucose- | 293862 | 1.00 | 1.01 | 0.71 | 1.03 | 0.76 | 0.84 | 1.09 | 0.64 | | | | | | | | | | | | |
| | regulated protein) or | | | | | | | | | | | | | | | | | | | | | |
| | dnaK-type molecular | | | | | | | | | | | | | | | | | | | | | |
| | chaperone GRP78 | | | | | | | | | | | | | | | | | | | | | |
| | precursor - Chinese | | | | | | | | | | | | | | | | | | | | | |
| | hamster (Bip) | | | | | | | | | | | | | | | | | | | | | |
| gi\|73993723 | PREDICTED: similar | 294375 | 1.93 | 0.97 | 0.34 | 1.17 | 0.42 | 1.01 | 1.11 | 1.09 | 0.81 | 0.86 | 0.94 | 0.44 | 0.77 | 0.72 | 1.23 | 1.05 | 1.03 | 2.37 | 1.18 | 1.34 |
| | to Serine/threonine | 294376 | 2.68 | 1.07 | 2.01 | 1.48 | 0.41 | 0.96 | 1.06 | 1.08 | | | | | | | | | | | | |
| | protein phosphatase | 293393 | 0.80 | 1.07 | 0.71 | 0.90 | 0.99 | 0.81 | 1.67 | 0.93 | | | | | | | | | | | | |
| | 2A, 55 kDa regulatory | 293394 | 0.99 | 0.98 | 1.40 | 1.01 | 0.91 | 0.81 | 0.85 | 0.98 | | | | | | | | | | | | |
| | subunit B, alpha | | | | | | | | | | | | | | | | | | | | | |
| | isoform (PP2A, | | | | | | | | | | | | | | | | | | | | | |
| | subunit B, B-alpha | | | | | | | | | | | | | | | | | | | | | |
| | isoform) (PP2A, | | | | | | | | | | | | | | | | | | | | | |
| | subunit B, B55-alpha | | | | | | | | | | | | | | | | | | | | | |
| | isoform) (PP2A, | | | | | | | | | | | | | | | | | | | | | |
| | subunit B, PR55-alpha | | | | | | | | | | | | | | | | | | | | | |
| | isoform) (PP2A, | | | | | | | | | | | | | | | | | | | | | |
| | subunit B, R2-alpha | | | | | | | | | | | | | | | | | | | | | |
| | isoform) . . . isoform 9 | | | | | | | | | | | | | | | | | | | | | |
| | (PP2r2A) | | | | | | | | | | | | | | | | | | | | | |
| WAN008EA0_at | Valosin-containing | 293401 | 0.65 | 0.46 | 0.72 | 0.51 | 1.15 | 1.01 | 1.74 | 1.25 | 2.17 | 1.09 | 1.06 | 1.33 | 1.26 | 1.35 | 0.69 | 0.97 | 0.74 | 1.00 | 0.77 | 0.66 |
| | protein | 293402 | 0.62 | 0.37 | 0.63 | 0.37 | 1.08 | 1.18 | 1.98 | 1.18 | | | | | | | | | | | | |
| | (VCP) | 293403 | 0.50 | 0.34 | 0.52 | 0.30 | 1.18 | 1.12 | 1.44 | 1.01 | | | | | | | | | | | | |
| | | 293404 | 0.48 | 0.38 | 0.40 | 0.31 | 1.02 | 1.24 | 1.31 | 0.97 | | | | | | | | | | | | |
| WAN0088JV_at | Tribbles homolog 3 | 293857 | 0.81 | 0.94 | 0.66 | 0.88 | 0.96 | 0.94 | 1.21 | 0.83 | 1.70 | 1.55 | 1.25 | 1.38 | 1.26 | 1.24 | 0.84 | 0.93 | 0.70 | 1.05 | 0.79 | 0.79 |
| | (Drosophila) | 293858 | 0.86 | 0.86 | 0.56 | 0.70 | 0.85 | 1.10 | 1.25 | 1.03 | | | | | | | | | | | | |
| | (TRIB3) | | | | | | | | | | | | | | | | | | | | | |
| WAN008CQP_at | Apoptosis | 295199 | 0.53 | 0.61 | 0.67 | 0.45 | 1.33 | 1.08 | 0.99 | 1.11 | | | | | | | | | | | | |
| | antagonizing | 295200 | 0.38 | 0.48 | 0.34 | 0.33 | 0.88 | 1.14 | 0.96 | 1.45 | | | | | | | | | | | | |
| | transcription factor | | | | | | | | | | | | | | | | | | | | | |
| | (AATF) | | | | | | | | | | | | | | | | | | | | | |
| M27838_s_at | Asparagine synthetase | 295223 | | | | | | | | | | | | | | | | | | | | |
| | (ASNS) | 295224 | | | | | | | | | | | | | | | | | | | | |
| gi\|21618633 | HMG-CoA Synthase | 293859 | 1.06 | 1.07 | 0.85 | 0.99 | 0.77 | 0.88 | 1.04 | 0.89 | | | | | | | | | | | | |
| | (HMGCS) | 293860 | 1.11 | 1.11 | 0.53 | 0.78 | 0.90 | 0.89 | 1.09 | 0.82 | | | | | | | | | | | | |
| gi\|54114937 | Eno1 protein (enolase | 294947 | 0.96 | 1.08 | 1.40 | 1.68 | 0.78 | 1.62 | 0.79 | 0.86 | 0.95 | 0.99 | 1.07 | 1.00 | 1.06 | 0.82 | 1.05 | 0.86 | 1.02 | 0.93 | 0.83 | 1.17 |
| | 1) | 294948 | 0.91 | 1.03 | 1.38 | 1.37 | 0.89 | 1.76 | 0.75 | 0.85 | | | | | | | | | | | | |
| | (Eno1) | | | | | | | | | | | | | | | | | | | | | |
| gi\|10442752 | eukaryotic translation | 294925 | 1.00 | 1.30 | 1.16 | 0.97 | 0.68 | 0.56 | 0.67 | 0.71 | | | | | | | | | | | | |
| | elongation factor 1- | 294926 | 0.71 | 0.88 | 0.76 | 0.56 | 0.95 | 0.82 | 1.02 | 1.17 | | | | | | | | | | | | |
| | delta | | | | | | | | | | | | | | | | | | | | | |
| | (EEF1D) | | | | | | | | | | | | | | | | | | | | | |
| gi\|52353955 | 3-phosphoglycerate | | | | | | | | | | 0.73 | 0.76 | 0.87 | 0.60 | 0.59 | 0.89 | 1.20 | 1.10 | 1.05 | 1.42 | 1.49 | 1.04 |
| | dehydrogenase | | | | | | | | | | | | | | | | | | | | | |
| | (pdgh) | | | | | | | | | | | | | | | | | | | | | |
| WAN013I8V_at | Nucleolin | | | | | | | | | | 0.93 | 1.00 | 0.95 | 0.87 | 1.07 | 0.87 | 1.06 | 0.92 | 1.00 | 1.10 | 0.90 | 1.13 |
| | (NCL) | | | | | | | | | | | | | | | | | | | | | |
| WAN008EOB_at | Nucleolar protein 1, | 294373 | 1.37 | 0.88 | 1.11 | 0.86 | 0.58 | 0.93 | 0.92 | 0.96 | 3.58 | 1.59 | 1.30 | 1.52 | 1.40 | 1.38 | 0.67 | 0.81 | 0.73 | 1.13 | 0.68 | 0.71 |
| | 120 kDa | | | | | | | | | | | | | | | | | | | | | |
| | (NOL1) | | | | | | | | | | | | | | | | | | | | | |
| | | 294374 | 1.45 | 0.61 | 0.69 | 0.45 | 0.42 | 1.16 | 0.98 | 1.11 | | | | | | | | | | | | |
| | | 293395 | 0.95 | 1.08 | 1.38 | 1.29 | 0.93 | 0.78 | 0.81 | 0.91 | | | | | | | | | | | | |
| | | 293396 | 0.95 | 1.13 | 1.33 | 1.13 | 1.01 | 0.72 | 0.91 | 0.87 | | | | | | | | | | | | |
| WAN008EJ7_at | Eukaryotic translation | 294937 | 1.31 | 1.33 | 1.65 | 1.55 | 0.76 | 0.73 | 0.67 | 0.71 | 0.94 | 0.95 | 1.02 | 0.89 | 1.02 | 0.93 | 1.02 | 1.04 | 0.92 | 1.05 | 0.94 | 1.01 |
| | initiation factor 5A | 294938 | 1.37 | 1.16 | 1.35 | 1.22 | 0.62 | 0.70 | 0.65 | 0.77 | | | | | | | | | | | | |
| | (EIF5A) | | | | | | | | | | | | | | | | | | | | | |
| gi\|21704020 | NADH dehydrogenase | 294933 | 1.03 | 1.15 | 0.96 | 1.10 | 0.66 | 0.77 | 0.74 | 0.80 | 1.16 | 0.92 | 1.04 | 1.08 | 1.06 | 0.87 | 0.91 | 1.00 | 0.99 | 0.91 | 0.89 | 1.12 |
| | (ubiquinone) Fe—S | 294934 | 1.20 | 1.04 | 1.13 | 1.02 | 0.70 | 0.79 | 0.65 | 0.76 | | | | | | | | | | | | |
| | protein 1 | | | | | | | | | | | | | | | | | | | | | |
| | (Nduf1) | | | | | | | | | | | | | | | | | | | | | |
| WAN013HUC_at | Superoxide dismutase | | | | | | | | | | 0.92 | 0.88 | 0.90 | 0.39 | 0.89 | 0.71 | 1.02 | 0.92 | 1.10 | 2.41 | 1.01 | 1.17 |
| | 1, soluble | | | | | | | | | | | | | | | | | | | | | |
| | (amyotrophic lateral | | | | | | | | | | | | | | | | | | | | | |
| | sclerosis 1 (adult)) | | | | | | | | | | | | | | | | | | | | | |
| | (SOD1) | | | | | | | | | | | | | | | | | | | | | |
| AF022942_at | Cold inducible RNA | 294965 | 1.24 | 1.21 | 1.29 | 0.94 | 0.94 | 0.81 | 0.77 | 0.98 | | | | | | | | | | | | |
| | binding protein | 294966 | 1.28 | 1.22 | 1.51 | 1.33 | 0.87 | 0.76 | 0.72 | 0.86 | | | | | | | | | | | | |
| | (Cirbp) | | | | | | | | | | | | | | | | | | | | | |
| gi\|2833344\| | Gelsolin (Actin- | 294963 | 1.50 | 1.39 | 1.95 | 1.604 | 0.84 | 0.77 | 0.67 | 0.88 | | | | | | | | | | | | |
| sp\|Q28372\| | depolymerizing factor) | 294964 | 1.01 | 0.93 | 1.24 | 0.913 | 0.90 | 0.79 | 0.65 | 1.36 | | | | | | | | | | | | |
| | (gelsolin) | | | | | | | | | | | | | | | | | | | | | |
| gi\|14010837 | NSFL1 (p97) cofactor | 295207 | | | | | | | | | 0.81 | 1.04 | 0.92 | 0.96 | 1.04 | 0.99 | 1.34 | 0.96 | 1.20 | 1.11 | 0.94 | 1.13 |
| | (p47) | 295208 | | | | | | | | | | | | | | | | | | | | |
| | (NSFL1) | | | | | | | | | | | | | | | | | | | | | |
| WAN013I9G_at | Solute carrier family 3 | 294377 | 2.21 | 1.34 | 1.21 | 1.18 | 0.41 | 0.73 | 1.01 | 0.97 | 1.04 | 1.14 | 1.32 | 1.57 | 1.25 | 1.52 | 0.91 | 1.00 | 0.99 | 0.91 | 0.89 | 1.12 |
| | (activators of dibasic | 294378 | 3.14 | 1.43 | 2.37 | 1.58 | 0.42 | 0.82 | 1.03 | 1.22 | | | | | | | | | | | | |
| | and neutral amino acid | | | | | | | | | | | | | | | | | | | | | |
| | transport), member 2 | | | | | | | | | | | | | | | | | | | | | |
| | (SLC3A2) | | | | | | | | | | | | | | | | | | | | | |
| WAN013HZJ_at | YY1 transcription | 295185 | Down | 0.46 | 0.33 | 0.44 | NE | 1.13 | 1.06 | 0.81 | | | | | | | | | | | | |
| | factor | 295186 | | 0.72 | 0.76 | 0.76 | | 0.96 | 0.86 | 0.90 | | | | | | | | | | | | |
| | (YY1) | | | | | | | | | | | | | | | | | | | | | |
| WAN008F1L_at | Max interacting protein 1 | | | | | | | | | | | | | | | | | | | | | |
| | (Mxi1) | | | | | | | | | | | | | | | | | | | | | |
| WAN0088XH_at | Homocysteine- | 294954 | 0.97 | 1.03 | 1.21 | 1.20 | 0.77 | 0.76 | 0.75 | 0.96 | 0.98 | 0.97 | 1.02 | 1.03 | 1.03 | 0.96 | 0.98 | 0.88 | 0.92 | 0.91 | 0.89 | 0.97 |
| | inducible, endoplasmic | 294955 | 1.03 | 1.04 | 1.40 | 1.20 | 0.80 | 0.84 | 0.75 | 0.94 | | | | | | | | | | | | |
| | reticulum stress- | | | | | | | | | | | | | | | | | | | | | |
| | inducible, ubiquitin-like | | | | | | | | | | | | | | | | | | | | | |
| | domain member 1 | | | | | | | | | | | | | | | | | | | | | |
| | (HERPUD1) | | | | | | | | | | | | | | | | | | | | | |
| WAN008CX9_at | Interferon-stimulated | 295201 | 0.63 | 0.48 | 0.71 | 0.41 | 1.58 | 1.12 | 1.12 | 1.09 | 0.97 | 0.93 | 0.97 | 0.88 | 1.01 | 0.95 | 1.01 | 0.98 | 0.98 | 1.10 | 0.95 | 1.00 |
| | transcription factor 3, | 295202 | 0.44 | 0.38 | 0.47 | 0.30 | 0.92 | 1.14 | 1.06 | 1.23 | | | | | | | | | | | | |
| | gamma 48 kDa | | | | | | | | | | | | | | | | | | | | | |
| | (ISGF3G) | | | | | | | | | | | | | | | | | | | | | |
| gi\|13097417 | FK506 binding protein 4 | 294951 | 1.33 | 1.53 | 0.86 | 1.73 | 0.89 | 0.73 | 0.71 | 0.80 | | | | | | | | | | | | |
| | | 294952 | 1.26 | 1.38 | 1.62 | 1.60 | 0.85 | 0.74 | 0.68 | 0.71 | | | | | | | | | | | | |
| WAN008E5L_at | Solute carrier family 1 | 295234 | | | | | | | | | | | | | | | | | | | | |
| | (neutral amino acid | 295235 | | | | | | | | | | | | | | | | | | | | |
| | transporter), member 5 | | | | | | | | | | | | | | | | | | | | | |
| | (SLC1A5) | | | | | | | | | | | | | | | | | | | | | |
| gi\|73921733\| | Prefoldin 5 | | | | | | | | | | | | | | | | | | | | | |
| sp\|Q5RAY0\| | (PFDN5) | | | | | | | | | | | | | | | | | | | | | |
| WAN008DRQ_x_at | Succinate | 294909 | 1.07 | 0.88 | 0.92 | 0.97 | 1.03 | 0.92 | 0.90 | 0.92 | | | | | | | | | | | | |
| | dehydrogenase | 294910 | 1.02 | 0.98 | 1.09 | 1.06 | 0.94 | 0.85 | 0.68 | 0.70 | | | | | | | | | | | | |
| | complex, subunit A, | | | | | | | | | | | | | | | | | | | | | |
| | flavoprotein (Fp) | | | | | | | | | | | | | | | | | | | | | |
| | (Sdha) | | | | | | | | | | | | | | | | | | | | | |
| WAN008E2Q_at | G1 to S phase | 295221 | | | | | | | | | | | | | | | | | | | | |
| | transition 1 | 295222 | | | | | | | | | | | | | | | | | | | | |
| | (GSPT1) | | | | | | | | | | | | | | | | | | | | | |
| WAN013I15_at | Succinate-CoA ligase, | 292190 | 1.03 | 1.13 | 0.93 | 1.12 | 0.98 | 0.71 | 0.99 | 0.81 | | | | | | | | | | | | |
| | GDP-forming, beta | 292191 | 1.10 | 0.90 | 1.09 | 1.07 | 0.73 | 1.01 | 1.00 | 0.84 | | | | | | | | | | | | |
| | subunit | | | | | | | | | | | | | | | | | | | | | |
| | (SUCLG2) | | | | | | | | | | | | | | | | | | | | | |
| WAN013HUG_at | Cyclin-dependent | 294921 | 1.05 | 1.48 | 1.27 | 0.63 | 0.64 | 0.67 | 0.59 | 1.82 | | | | | | | | | | | | |
| | kinase inhibitor 2C | 294922 | 0.69 | 0.95 | 0.77 | 1.15 | 1.17 | 1.04 | 1.48 | 1.25 | | | | | | | | | | | | |
| | (p18, inhibits CDK4) | | | | | | | | | | | | | | | | | | | | | |
| | (CDKN2C) | | | | | | | | | | | | | | | | | | | | | |
| WAN008EX2_x_at | Interferon-related | | | | | | | | | | 1.13 | 1.11 | 1.14 | 1.01 | 1.12 | 0.94 | 1.02 | 0.98 | 0.91 | 1.02 | 0.89 | 1.35 |
| | developmental | | | | | | | | | | | | | | | | | | | | | |
| | regulator 1 | | | | | | | | | | | | | | | | | | | | | |
| | (IFRD1) | | | | | | | | | | | | | | | | | | | | | |
| WAN008D16_at | Protein inhibitor of | 295215 | | | | | | | | | | | | | | | | | | | | |
| | activated STAT, 1 | 295216 | | | | | | | | | | | | | | | | | | | | |
| | (PIAS1) | | | | | | | | | | | | | | | | | | | | | |
| WAN008DXT_at | Succinate-CoA ligase, | 294955 | 1.51 | 1.38 | 1.72 | 1.41 | 0.90 | 0.73 | 0.73 | 0.87 | | | | | | | | | | | | |
| | ADP-forming, beta | 294956 | 1.50 | 1.48 | 1.57 | 1.57 | 0.81 | 0.66 | 0.69 | 0.75 | | | | | | | | | | | | |
| | subunit | | | | | | | | | | | | | | | | | | | | | |
| | (SUCLA2) | | | | | | | | | | | | | | | | | | | | | |
| gi\|34853001 | PREDICTED: similar | | | | | | | | | | 1.91 | 0.97 | 1.20 | 1.14 | 1.14 | 1.22 | 0.72 | 0.80 | 0.87 | 0.92 | 0.83 | 0.80 |
| | to UDP-N- | | | | | | | | | | | | | | | | | | | | | |
| | acteylglucosamine | | | | | | | | | | | | | | | | | | | | | |
| | pyrophosphorylase 1- | | | | | | | | | | | | | | | | | | | | | |
| | like 1 | | | | | | | | | | | | | | | | | | | | | |
| | (UAP) | | | | | | | | | | | | | | | | | | | | | |
| WAN008CX2- | MAF1 homolog (S. cerevisiae) | 294919 | 1.03 | 1.41 | 1.22 | 1.36 | 0.87 | 0.59 | 0.89 | 0.64 | 0.94 | 0.97 | 1.02 | 0.99 | 1.05 | 0.90 | 1.02 | 0.95 | 0.95 | 1.00 | 0.94 | 1.12 |
| rc_at | (MAF1) | 294920 | 0.89 | 0.82 | 1.01 | 0.83 | 1.33 | 1.58 | 1.29 | 0.96 | | | | | | | | | | | | |
| WAN008DMJ_at | NGFI-A binding | 295209 | | | | | | | | | | | | | | | | | | | | |
| | protein 2 (EGR1 | 295210 | | | | | | | | | | | | | | | | | | | | |
| | binding protein 2) | | | | | | | | | | | | | | | | | | | | | |
| | (NAB2) | | | | | | | | | | | | | | | | | | | | | |
| WAN008DMI_at | Acyl-CoA synthetase | 294935 | 1.63 | 1.44 | 1.82 | 1.52 | 0.70 | 0.79 | 0.65 | 0.76 | | | | | | | | | | | | |
| | long-chain family | 294936 | 1.78 | 1.45 | 1.97 | 1.53 | 0.68 | 0.82 | 0.77 | 0.81 | | | | | | | | | | | | |
| | member 5 | | | | | | | | | | | | | | | | | | | | | |
| | (ACSL5) | | | | | | | | | | | | | | | | | | | | | |
| WAN008D2S | BPY2 Interacting | 295227 | | | | | | | | | | | | | | | | | | | | |
| | protein 1 | 295228 | | | | | | | | | | | | | | | | | | | | |
| | (BPY2IP1) | | | | | | | | | | | | | | | | | | | | | |
| WAN013I9K_at | Glutathione S- | | | | | | | | | | 1.68 | 0.98 | 1.17 | 1.12 | 1.06 | 1.27 | 0.73 | 0.73 | 0.79 | 0.88 | 0.88 | 0.74 |
| | transferase, mu 1 | | | | | | | | | | | | | | | | | | | | | |
| | (Gstm1) | | | | | | | | | | | | | | | | | | | | | |
| WAN013I6C_at | Solute carrier family | | | | | | | | | | | | | | | | | | | | | |
| | 16 (monocarboxylic | | | | | | | | | | | | | | | | | | | | | |
| | acid transporters), | | | | | | | | | | | | | | | | | | | | | |
| | member 1 | | | | | | | | | | | | | | | | | | | | | |
| | (SLC16A1) | | | | | | | | | | | | | | | | | | | | | |
| WAN008DK1_at | Ubiquinol-cytochrome | 295517 | | | | | | | | | | | | | | | | | | | | |
| | c reductase core | 295518 | | | | | | | | | | | | | | | | | | | | |
| | protein I | | | | | | | | | | | | | | | | | | | | | |
| | (UQCRC1) | | | | | | | | | | | | | | | | | | | | | |
| WAN008E8M_at | Hydroxyacyl- | 294939 | 1.28 | 1.18 | 1.45 | 1.27 | 0.65 | 0.74 | 0.83 | 1.11 | | | | | | | | | | | | |
| | Coenzyme A | 294940 | 1.26 | 1.20 | 1.74 | 2.01 | 0.47 | 0.51 | 0.63 | 0.73 | | | | | | | | | | | | |
| | dehydrogenase/3- | | | | | | | | | | | | | | | | | | | | | |
| | ketoacyl-Coenzyme A | | | | | | | | | | | | | | | | | | | | | |
| | thiolase/enoyl- | | | | | | | | | | | | | | | | | | | | | |
| | Coenzyme A | | | | | | | | | | | | | | | | | | | | | |
| | hydratase (trifunctional | | | | | | | | | | | | | | | | | | | | | |
| | protein), beta subunit | | | | | | | | | | | | | | | | | | | | | |
| | (HADHB) | | | | | | | | | | | | | | | | | | | | | |
| Y11149_at | Thyrotrophic | | | | | | | | | | | | | | | | | | | | | |
| | embryonic factor | | | | | | | | | | | | | | | | | | | | | |
| | (TEF) | | | | | | | | | | | | | | | | | | | | | |
| WAN0088OY_x_at | Heterogeneous | 295191 | | 2.25 | 0.80 | 0.92 | Down | 0.37 | 0.98 | 0.93 | | | | | | | | | | | | |
| | nuclear | 295192 | | 1.80 | 0.66 | 0.75 | | 0.47 | 1.02 | 1.03 | | | | | | | | | | | | |
| | ribonucleoprotein F | | | | | | | | | | | | | | | | | | | | | |
| | (Hnrpf) | | | | | | | | | | | | | | | | | | | | | |
| WAN008DZF | Expressed sequence | 293380 | 0.86 | 0.92 | 0.78 | 0.94 | 0.85 | 0.78 | 0.94 | 1.04 | | | | | | | | | | | | |
| | (AL033326) | 293381 | 0.91 | 1.04 | 0.95 | 1.01 | 0.88 | 0.70 | 0.85 | 1.06 | | | | | | | | | | | | |
| AF056934_at | APEX nuclease | 295197 | 0.56 | 0.67 | 0.73 | 0.51 | 0.99 | 1.03 | 0.81 | 1.24 | | | | | | | | | | | | |
| | (multifunctional DNA | 295198 | 0.50 | 0.66 | 0.69 | 0.49 | 1.23 | 1.10 | 1.14 | 1.92 | | | | | | | | | | | | |
| | repair enzyme) 1 | | | | | | | | | | | | | | | | | | | | | |
| | (APEX1) | | | | | | | | | | | | | | | | | | | | | |
| WAN0088S8_at | Solute carrier family | 294957 | 1.28 | 1.45 | 1.45 | 1.50 | 1.79 | 0.63 | 0.66 | 0.76 | 0.93 | 0.98 | 1.29 | 1.27 | 1.05 | 1.38 | 1.02 | 0.92 | 0.81 | 0.77 | 0.98 | 0.75 |
| | 29 (nucleoside | 294958 | 1.44 | 1.42 | 0.90 | 1.50 | 0.87 | 0.70 | 0.64 | 0.810 | | | | | | | | | | | | |
| | transporters), member 1 | | | | | | | | | | | | | | | | | | | | | |
| | (SLC29A1) | | | | | | | | | | | | | | | | | | | | | |
| WAN008CTZ_at | Phosphogluconate | 294917 | 0.78 | 0.97 | 0.90 | 0.98 | 1.33 | 1.20 | 0.66 | 1.36 | | | | | | | | | | | | |
| | dehydrogenase | 294918 | 1.09 | 1.34 | 1.11 | 1.35 | 0.90 | 0.88 | 1.09 | 0.96 | | | | | | | | | | | | |
| | (PGD) | 295211 | | | | | | | | | | | | | | | | | | | | |
| | | 295212 | | | | | | | | | | | | | | | | | | | | |
| K00924_at | Vimentin | 293391 | 1.08 | 1.09 | 0.85 | 1.08 | 0.85 | 0.85 | 1.38 | 1.10 | | | | | | | | | | | | |
| | (Vim) | 293392 | 0.43 | 0.99 | 0.35 | 0.85 | 1.03 | 0.83 | 1.61 | 0.99 | | | | | | | | | | | | |
| WAN008D6J_at | High mobility group | | | | | | | | | | | | | | | | | | | | | |
| | AT-hook 2 | | | | | | | | | | | | | | | | | | | | | |
| | (HMGA2) | | | | | | | | | | | | | | | | | | | | | |
| WAN013I20_x_at | V-maf | | | | | | | | | | | | | | | | | | | | | |
| | musculoaponeurotic | | | | | | | | | | | | | | | | | | | | | |
| | fibrosarcoma | | | | | | | | | | | | | | | | | | | | | |
| | oncogene homolog G | | | | | | | | | | | | | | | | | | | | | |
| | (avian) | | | | | | | | | | | | | | | | | | | | | |
| | (MAFG) | | | | | | | | | | | | | | | | | | | | | |
| WAN008END_at | SCY1-like 1 (S. cerevisiae) | 295195 | 0.85 | 0.95 | 0.84 | 0.71 | 0.93 | 1.01 | 1.17 | 1.20 | | | | | | | | | | | | |
| | (SCYL1) | 295196 | 0.54 | 0.54 | 0.60 | 0.59 | 0.54 | 0.59 | 0.87 | 0.64 | | | | | | | | | | | | |
| U22819_s_at | Sterol regulatory | 295213 | | | | | | | | | | | | | | | | | | | | |
| | element binding | 295214 | | | | | | | | | | | | | | | | | | | | |
| | transcription factor 2 | | | | | | | | | | | | | | | | | | | | | |
| | (SREBF2) | | | | | | | | | | | | | | | | | | | | | |
| WAN013I2L_at | solute carrier family 7 | | | | | | | | | | | | | | | | | | | | | |
| | (cationic amino acid | | | | | | | | | | | | | | | | | | | | | |
| | transporter, y+ | | | | | | | | | | | | | | | | | | | | | |
| | system), member 5 | | | | | | | | | | | | | | | | | | | | | |
| | (SLC7A5) | | | | | | | | | | | | | | | | | | | | | |
| WAN008E65_at | Endoplasmic reticulum | 294905 | 0.81 | 0.85 | 0.80 | 1.36 | 0.96 | 0.88 | 0.95 | 0.96 | | | | | | | | | | | | |
| | protein 29 | 294906 | 0.81 | 0.88 | 0.89 | 1.37 | 1.01 | 0.93 | 0.94 | 0.91 | | | | | | | | | | | | |
| | (ERP29) | | | | | | | | | | | | | | | | | | | | | |
| WAN008ERP_at | Leprecan-like 1 | | | | | | | | | | | | | | | | | | | | | |
| | (LEPREL1) | | | | | | | | | | | | | | | | | | | | | |
| AF081141_at | Chemokine (C-C | 294949 | 1.35 | 1.28 | 1.47 | 1.20 | 0.85 | 0.86 | 0.87 | 0.99 | 0.97 | 0.98 | 1.02 | 1.12 | 0.95 | 0.99 | 1.04 | 0.95 | 0.98 | 0.86 | 0.96 | 1.00 |
| | motif) ligand 2 or | 294950 | 1.23 | 1.49 | 1.27 | 1.55 | 0.80 | 0.62 | 0.84 | 0.79 | | | | | | | | | | | | |
| | Monocyte | 289094 | 0.99 | 1.03 | 1.04 | 1.08 | 0.97 | 0.86 | 1.00 | 0.84 | | | | | | | | | | | | |
| | Chemoattractant | 289095 | 1.51 | 1.06 | 1.11 | 1.06 | 0.60 | 1.06 | 0.86 | 0.89 | | | | | | | | | | | | |
| | Protein 1 | | | | | | | | | | | | | | | | | | | | | |
| | (CCL2) | | | | | | | | | | | | | | | | | | | | | |
| WAN008DGZ_at | Solute carrier family 7, | | | | | | | | | | | | | | | | | | | | | |
| | member 6 opposite | | | | | | | | | | | | | | | | | | | | | |
| | strand | | | | | | | | | | | | | | | | | | | | | |
| | (SLC7A6OS) | | | | | | | | | | | | | | | | | | | | | |
| U42430_at | CD36 antigen | 294381 | 1.95 | 0.91 | 1.58 | 1.37 | 0.54 | 1.16 | 0.96 | 1.02 | 1.09 | 1.11 | 1.24 | 1.26 | 1.02 | 1.19 | 1.05 | 1.00 | 0.97 | 0.86 | 1.04 | 0.97 |
| | (collagen type I | 294382 | 2.48 | 1.12 | 1.58 | 1.50 | 0.49 | 0.93 | 0.86 | 0.86 | | | | | | | | | | | | |
| | receptor, | | | | | | | | | | | | | | | | | | | | | |
| | thrombospondin | | | | | | | | | | | | | | | | | | | | | |
| | receptor) | | | | | | | | | | | | | | | | | | | | | |
| | (CD36) | | | | | | | | | | | | | | | | | | | | | |
| WAN013I8N_at | IMP (inosine | 294941 | 1.13 | 1.27 | 1.46 | 1.46 | 0.82 | 0.52 | 0.79 | 0.94 | 1.07 | 0.91 | 1.00 | 1.04 | 0.97 | 0.98 | 0.92 | 0.92 | 0.96 | 0.89 | 0.89 | 1.00 |
| | monophosphate) | 294942 | 1.34 | 1.45 | 1.32 | 1.38 | 0.76 | 0.55 | 0.85 | 1.02 | | | | | | | | | | | | |
| | dehydrogenase 2 | | | | | | | | | | | | | | | | | | | | | |
| | (IMPDH2) | | | | | | | | | | | | | | | | | | | | | |
| WAN013I3K_at | Isocitrate | 295181 | | 0.53 | 0.44 | 0.50 | | 1.04 | 1.08 | 0.83 | | | | | | | | | | | | |
| | dehydrogenase 1 | 295182 | | 0.58 | 0.48 | 0.65 | | 1.12 | 1.05 | 0.93 | | | | | | | | | | | | |
| | (NADP+), soluble | | | | | | | | | | | | | | | | | | | | | |
| | (IDH1) | | | | | | | | | | | | | | | | | | | | | |
| WAN008DNJ_at | RNA binding motif | 294915 | 0.68 | 0.87 | 0.76 | 0.90 | 1.55 | 1.20 | 1.53 | 1.31 | | | | | | | | | | | | |
| | protein, X | 294916 | 1.01 | 1.24 | 1.10 | 1.17 | 0.62 | 0.56 | 0.71 | 0.68 | | | | | | | | | | | | |
| | chromosome | | | | | | | | | | | | | | | | | | | | | |
| | retrogene | | | | | | | | | | | | | | | | | | | | | |
| | (Rbmxrt) | | | | | | | | | | | | | | | | | | | | | |
| WAN008DIE_at | Retinoic acid induced | 295183 | | 0.59 | 0.59 | 0.59 | | 1.13 | 1.10 | 1.00 | | | | | | | | | | | | |
| | 14 | 295184 | | 0.49 | 0.40 | 0.54 | | 1.12 | 1.02 | 0.79 | | | | | | | | | | | | |
| | (RAI14) | | | | | | | | | | | | | | | | | | | | | |
| S74024_at | Xeroderma | | | | | | | | | | | | | | | | | | | | | |
| | pigmentosum, complementation | | | | | | | | | | | | | | | | | | | | | |
| | group A | | | | | | | | | | | | | | | | | | | | | |
| | (XPA) | | | | | | | | | | | | | | | | | | | | | |
| gi\|381964 | actin-related protein | 293382 | 0.80 | 1.01 | 0.76 | 0.92 | 0.84 | 0.69 | 0.88 | 1.02 | | | | | | | | | | | | |
| | | 293383 | 1.14 | 1.08 | 0.84 | 0.96 | 0.73 | 0.80 | 1.47 | 1.28 | | | | | | | | | | | | |
| AF120325_f_at | Tubulin, beta 2B | 294943 | 1.29 | 1.32 | 1.07 | 1.06 | 0.70 | 0.78 | 0.77 | 0.93 | | | | | | | | | | | | |
| | (TUBB2B) | 294944 | 1.35 | 1.55 | 1.25 | 1.32 | 0.81 | 0.93 | 0.81 | 1.08 | | | | | | | | | | | | |
| WAN008D6O_at | Spermatid perinuclear | 295187 | | 1.76 | 0.69 | 0.86 | | 0.44 | 0.91 | 0.85 | | | | | | | | | | | | |
| | RNA binding protein | | | 2.16 | 0.53 | 0.56 | | 0.39 | 0.84 | 1.43 | | | | | | | | | | | | |
| | (STRBP) | | | | | | | | | | | | | | | | | | | | | |
| WAN008DGD_at | Amyloid beta (A4) | | | | | | | | | | | | | | | | | | | | | |
| | precursor-like protein | | | | | | | | | | | | | | | | | | | | | |
| | 2 (Aplp2) | | | | | | | | | | | | | | | | | | | | | |
| WAN013HUI_at | huntingtin interacting | 294927 | 1.11 | 0.94 | 1.02 | 1.07 | 0.65 | 0.82 | 0.94 | 0.88 | 1.06 | 0.91 | 1.03 | 0.98 | 0.93 | 0.90 | 1.00 | 0.99 | 1.02 | 0.98 | 0.95 | 1.11 |
| | protein-2 | 294928 | 1.15 | 1.05 | 1.02 | 0.78 | 0.74 | 0.80 | 0.90 | 0.90 | | | | | | | | | | | | |
| | (HIP2) | | | | | | | | | | | | | | | | | | | | | |
| WAN008DMP_at | Ewing sarcoma | 294913 | 1.18 | 0.93 | 1.26 | 1.08 | 0.97 | 0.83 | 0.66 | 0.81 | | | | | | | | | | | | |
| | breakpoint region 1 | 294914 | 1.02 | | 0.95 | | 1.08 | | 0.92 | | | | | | | | | | | | | |
| | (EWSR1) | | | | | | | | | | | | | | | | | | | | | |
| WAN008DS9_at | Cofilin 2 (muscle) | 294959 | 1.50 | 1.30 | 1.06 | 1.42 | 0.79 | 0.75 | 0.61 | 0.85 | 0.89 | 1.01 | 1.17 | 1.12 | 1.05 | 1.32 | 1.32 | 1.12 | 1.05 | 1.00 | 1.05 | 0.94 |
| | (CFL2) | 294960 | 1.32 | 1.34 | 1.75 | 1.41 | 0.96 | 0.78 | 0.72 | 0.93 | | | | | | | | | | | | |
| WAN008D6R | Transmembrane | 294953 | 1.33 | 1.73 | 1.60 | 1.75 | 0.88 | 0.68 | 0.73 | 0.77 | 1.05 | 0.93 | 0.98 | 1.00 | 1.10 | 0.86 | 0.93 | 0.93 | 0.97 | 0.97 | 0.87 | 0.73 |
| | EMP24 protein | 294954 | 1.37 | 1.81 | 1.90 | 1.95 | 0.88 | 0.68 | 0.69 | 0.79 | | | | | | | | | | | | |
| | transporter | | | | | | | | | | | | | | | | | | | | | |
| | (TMED4) | | | | | | | | | | | | | | | | | | | | | |
| WAN013I6J_s_at | Carbamoyl-phosphate | 295205 | | | | | | | | | | | | | | | | | | | | |
| | synthetase 2, | 295206 | | | | | | | | | | | | | | | | | | | | |
| | aspartate | | | | | | | | | | | | | | | | | | | | | |
| | transcarbamylase, and | | | | | | | | | | | | | | | | | | | | | |
| | dihydroorotase | | | | | | | | | | | | | | | | | | | | | |
| | (CAD) | | | | | | | | | | | | | | | | | | | | | |
| WAN013IAB_x_at | Tumor protein p53 (Li- | 293863 | 0.76 | 0.77 | 0.66 | 0.86 | 0.78 | 1.03 | 1.02 | 0.71 | | | | | | | | | | | | |
| | Fraumeni syndrome) | 293864 | 0.84 | 1.01 | 0.85 | 1.20 | 0.84 | 0.85 | 1.00 | 0.62 | | | | | | | | | | | | |
| | (TP53) | | | | | | | | | | | | | | | | | | | | | |
| WAN008ERQ_at | ARP6 actin-related | 294907 | 1.18 | 1.22 | 1.27 | 1.85 | 0.87 | 0.81 | 0.68 | 0.88 | 1.71 | 1.09 | 1.24 | 1.04 | 1.01 | 1.19 | 0.74 | 0.77 | 0.82 | 0.99 | 0.92 | 0.85 |
| | protein 6 homolog | 294908 | 1.12 | 0.96 | 1.24 | 0.72 | 0.93 | 0.97 | 0.70 | 0.77 | | | | | | | | | | | | |
| | (yeast) | | | | | | | | | | | | | | | | | | | | | |
| | (ACTR6) | | | | | | | | | | | | | | | | | | | | | |
| X53074_f_at | Hypoxanthine | 294923 | 1.00 | 1.33 | 1.27 | 1.45 | 0.90 | 0.62 | 0.92 | 0.76 | | | | | | | | | | | | |
| | phosphoribosyltransferase | 294924 | 1.08 | 1.18 | 1.12 | 1.25 | 0.91 | 0.90 | 1.17 | 0.76 | | | | | | | | | | | | |
| | 1 (Lesch-Nyhan | 294911 | 0.84 | 0.94 | 0.86 | 1.06 | 1.10 | 0.81 | 0.91 | 0.88 | | | | | | | | | | | | |
| | syndrome) | 294912 | 1.22 | 1.01 | 1.32 | 1.32 | 0.95 | 0.77 | 0.66 | 0.85 | | | | | | | | | | | | |
| | (HPRT1) | | | | | | | | | | | | | | | | | | | | | |
| WAN008BSG_x_at | Translocation | 294961 | 1.28 | 1.15 | 1.73 | 0.94 | 0.97 | 0.87 | 0.67 | 1.17 | | | | | | | | | | | | |
| | associated membrane | 294962 | 1.37 | 1.06 | 1.40 | 0.95 | 0.83 | 0.92 | 0.74 | 1.04 | | | | | | | | | | | | |
| | protein 1 | | | | | | | | | | | | | | | | | | | | | |
| | (TRAM1) | | | | | | | | | | | | | | | | | | | | | |
| WAN008EC4_at | Hippocampus | 295193 | 0.54 | 0.48 | 0.72 | 0.34 | 0.62 | 0.65 | 0.91 | 1.17 | | | | | | | | | | | | |
| | abundant transcript 1 | 295194 | 0.57 | 0.62 | 0.59 | 0.36 | 0.91 | 0.96 | 1.13 | 1.40 | | | | | | | | | | | | |
| | (HIAT1) | | | | | | | | | | | | | | | | | | | | | |
| M12329_at | M12329 Chinese | 294931 | 1.01 | 0.81 | 0.83 | 0.83 | 0.75 | 0.97 | 1.00 | 0.90 | 1.06 | 0.98 | 0.99 | 0.95 | 1.06 | 1.02 | 0.93 | 0.95 | 0.99 | 0.95 | 0.86 | 0.98 |
| | hamster alpha-tubulin | 294932 | 1.11 | 0.78 | 1.04 | 0.75 | 0.65 | 0.84 | 0.76 | 1.03 | | | | | | | | | | | | |
| | III mRNA, complete | | | | | | | | | | | | | | | | | | | | | |
| | cds. | | | | | | | | | | | | | | | | | | | | | |
| AF221841_at | Peroxiredoxin 1 | 294967 | 1.40 | 1.40 | 1.60 | 1.43 | 0.89 | 0.83 | 0.74 | 0.90 | 1.00 | 1.00 | 1.05 | 1.04 | 0.92 | 0.93 | 1.04 | 1.00 | 0.95 | 0.98 | 0.99 | 1.04 |
| | (Prdx1) | 294968 | 1.44 | 1.52 | 2.40 | 1.55 | 0.82 | 0.00 | 0.56 | 0.80 | | | | | | | | | | | | |
| WAN008CXC_at | ATPase, H+ | | | | | | | | | | 1.90 | 1.21 | 1.23 | 1.04 | 1.05 | 1.15 | 0.78 | 0.77 | 0.79 | 0.97 | 0.97 | 0.83 |
| | transporting, | | | | | | | | | | | | | | | | | | | | | |
| | lysosomal V0 subunit | | | | | | | | | | | | | | | | | | | | | |
| | a isoform 1 | | | | | | | | | | | | | | | | | | | | | |
| | (ATP6V0A1) | | | | | | | | | | | | | | | | | | | | | |
| WAN0088X2_at | Progressive external | 294929 | 1.28 | 1.06 | 1.19 | 1.17 | 0.63 | 0.69 | 0.66 | 0.59 | | | | | | | | | | | | |
| | ophthalmoplegia 1 | 294930 | 1.38 | 1.34 | 1.34 | 1.27 | 0.61 | 0.69 | 0.76 | 0.69 | | | | | | | | | | | | |
| | (PEO1) | | | | | | | | | | | | | | | | | | | | | |
| M29238_at | DNA-damage- | | | | | | | | | | 0.49 | 0.67 | 0.54 | 0.70 | 0.93 | 0.76 | 1.40 | 1.06 | 1.35 | 1.17 | 0.73 | 1.02 |
| | inducible transcript 3 | | | | | | | | | | | | | | | | | | | | | |
| | (DDIT3) | | | | | | | | | | | | | | | | | | | | | |
| WAN0088ZJ_at | Solute carrier family 4 | | | | | | | | | | | | | | | | | | | | | |
| | (anion exchanger), | | | | | | | | | | | | | | | | | | | | | |
| | member 2 | | | | | | | | | | | | | | | | | | | | | |
| | (Slc4a2) | | | | | | | | | | | | | | | | | | | | | |
| U62588_x_at | Syndecan 1 | | | | | | | | | | | | | | | | | | | | | |
| | (SDC1) | | | | | | | | | | | | | | | | | | | | | |
| WAN013I3P_at | Calcium modulating | 295219 | | | | | | | | | | | | | | | | | | | | |
| | ligand | 295220 | | | | | | | | | | | | | | | | | | | | |
| | (CAMLG) | | | | | | | | | | | | | | | | | | | | | |
| M76730_at | Procollagen, type V, | 295203 | 0.52 | 0.60 | 0.56 | 0.67 | 1.12 | 1.41 | 0.85 | 0.91 | | | | | | | | | | | | |
| | alpha 1 | 295204 | 0.48 | 0.47 | 0.65 | 0.36 | 0.78 | 1.04 | 0.68 | 1.09 | | | | | | | | | | | | |
| | (Col5a1) | | | | | | | | | | | | | | | | | | | | | |
| WAN008EMQ_at | Karyopherin alpha 3 | 295289 | | 1.73 | 0.62 | 0.87 | | 0.49 | 0.98 | 0.96 | | | | | | | | | | | | |
| | (importin alpha 4) | 295290 | | 3.22 | 0.88 | 0.89 | | 0.24 | 0.92 | 0.83 | | | | | | | | | | | | |
| | (KPNA3) | | | | | | | | | | | | | | | | | | | | | |

### Equivalents and Scope

Those skilled in the art will recognize, or be able to ascertain using no more than routine experimentation, many equivalents to the specific embodiments, described herein. The scope of the present invention is not intended to be limited to the above Description, but rather is as set forth in the appended claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any cell type; any neuronal cell system; any reporter of synaptic vesicle cycling; any electrical stimulation system; any imaging system; any synaptic vesicle cycling assay; any synaptic vesicle cycle modulator; any working memory modulator; any disorder associated with working memory; any method of use; etc.) can be excluded from any one or more claims, for any reason, whether or not related to the existence of prior art.

### INCORPORATION BY REFERENCE

All sequence accession numbers, publications and patent documents cited in this application are incorporated by reference in their entirety for all purposes to the same extent as if the contents of each individual publication or patent document was incorporated herein.

What is claimed is:

1. A method of identifying a protein or gene regulating or indicative of a cell phenotype of interest, the method comprising:
obtaining a first control sample from a control cell culture at a first time point and generating a first control expression profile of the first control sample;
obtaining a second control sample from the control cell culture at a second time point and generating a second control expression profile of the second control sample;
comparing the first control expression profile to the second control expression profile to identify one or more differentially expressed proteins or genes in the control cell culture;
obtaining a first test sample from a test cell culture at a first time point and generating a first test expression profile of the first test sample;
obtaining a second test sample from the test cell culture at a second time point and generating a second test expression profile of the second test sample;
comparing the first test expression profile to the second test expression profile to identify one or more differentially expressed proteins or genes in the test cell culture; and
comparing the one or more differentially expressed proteins or genes in the control cell culture to the one or more differentially expressed proteins or genes in the test cell culture to classify the one or more differentially expressed proteins or genes into control cell-only, test cell-only, and common differentially expressed proteins or genes;
wherein the cell phenotype of interest or a change of the cell phenotype of interest over time in the test cell culture is distinct from that in the control cell culture.

2. The method of claim 1, wherein the test and control cell culture comprise Chinese hamster ovary (CHO) cells.

3. The method of claim 1, wherein the cell phenotype is selected from the group consisting of cell growth rate, cellular productivity, peak cell density, sustained cell viability, rate of ammonia production or consumption, rate of lactate production or consumption and combinations thereof.

4. The method of claim 3, wherein the cell phenotype is maximum cellular productivity.

5. The method of claim 3, wherein the cell phenotype is sustained cell viability.

6. The method of claim 3, wherein the cell phenotype is peak cell density.

7. The method of claim 3, wherein the cell phenotype is cell growth rate.

8. The method of claim 1, wherein the expression profile is a protein expression profile.

9. The method of claim 8, wherein the protein expression profile is generated by fluorescent two-dimensional differential in-gel electrophoresis.

10. The method of claim 1, wherein the expression profile is a gene expression profile.

11. The method of claim 1, wherein the first time point is taken during an exponential growth phase and the second time point is taken during a lag phase.

12. The method of claim 1, wherein the test and control cell cultures are grown under a fed batch condition.

13. A method for improving a cell line, the method comprising up-regulating or down-regulating one or more control cell-only or test cell-only differentially expressed proteins or genes identified according to the method of claim 1.

14. A method for improving cellular productivity of a cell line, the method comprising up-regulating or down-regulating one or more control cell-only or test cell-only differentially expressed proteins or genes identified according to the method of claim 4.

15. A method for improving cell growth rate of a cell line, the method comprising up-regulating or down-regulating one or more control cell-only or test cell-only differentially expressed proteins or genes identified according to the method of claim 7.

16. A method for increasing peak cell density of a cell line, the method comprising up-regulating or down-regulating one or more control cell-only or test cell-only differentially expressed proteins or genes identified according to the method of claim 6.

17. A method for increasing peak cell density of a cell line, the method comprising up-regulating or down-regulating one or more control-only or test-only genes or proteins selected from Tables 2, 3, 4, 5, 8, 9, and 10.

18. A method for increasing sustained cell viability of a cell line, the method comprising up-regulating or down-regulating one or more control cell-only or test cell-only differentially expressed proteins or genes identified according to the method of claim 5.

19. A method for improving a cell line, the method comprising up-regulating or down-regulating one or more genes selected from Tables 12 and 13.

20. A method evaluating a cell phenotype of a cell culture, the method comprising:
detecting a first expression level of at least one control cell-only or test cell-only differentially expressed protein or gene identified according to claim 1 at a time point taken during an exponential phase;
detecting a second expression level of said at least one control cell-only or test cell-only differentially expressed protein or gene at a time point taken during a lag phase; and
comparing the first expression level to the second expression level to evaluate the cell phenotype of the cell culture.

21. A method of evaluating a cell phenotype of a cell culture, the method comprising:
detecting a first expression level of at least one protein or gene selected from Tables 2, 3, 4, 5, 8, 9, 10, 12, 13, 14 and 15 at a time point taken during an exponential phase;
detecting a second expression level of said at least one protein or gene at a time point taken during a lag phase; and
comparing the first expression level to the second expression level to evaluate the cell phenotypes of the cell culture.

23. An engineered cell line with an improved cell phenotype comprising a population of engineered cells, each of which comprising an engineered construct up-regulating or down-regulating one or more control cell-only or test cell-only differentially expressed proteins or genes identified according to claim 1.

24. An engineered cell line with an improved cell phenotype comprising a population of engineered cells, each of which comprising an engineered construct up-regulating or down-regulating one or more proteins or genes selected from Tables 2, 3, 4, 5, 8, 9, 10, 12, 13, 14 and 15.

25. The engineered cell line of claim 24, wherein the engineered construct is an over-expression construct.

26. The engineered cell line of claim 24, wherein the engineered construct is an interfering RNA construct.

27. An engineered cell line with an improved peak cell density comprising a population of engineered cells, each of which comprising an engineered construct up-regulating or down-regulating one or more genes or proteins selected from Tables 2, 3, 4, 5, 8, 9, and 10.

28. The engineered cell line of claim 27, wherein the engineered construct is an over-expression construct.

29. The engineered cell line of claim 28, wherein the engineered construct is an interfering RNA construct.


SEQUENCE LISTING (503 sequences)

SEQ ID NO: 1 - protein, 643 aa, Rattus norvegicus
MRVLWVLGLCCVLLTFGFVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALAGNEELTVKIKCDREKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVRKYSQFINFPIYVWSSKTETVEEPLEEDETAQEEKEEADDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADEKYNDTFWKEFGTNIKLGVIEDHSNRTRLAKLLRFQSSHHSTDITSLDQYVERMKEKQDKIYFMAGSSRKEAESSPFVERLLKKGYEVIYLTEPVDEYCIQALPEFDGKRFQNVAKEGVKFDESEKSKESREATEKEFEPLLNWMKDKALKDKVFGPQTVVSPHRN

SEQ ID NO: 2 - protein, 9 aa, Cricetulus griseus
GLFDEYGSK

SEQ ID NO: 3 - protein, 9 aa, Cricetulus griseus
IYFMAGSSR

SEQ ID NO: 4 - protein, 9 aa, Cricetulus griseus
FAFQAEVNR

SEQ ID NO: 5 - protein, 10 aa, Cricetulus griseus
LGVIEDHSNR

SEQ ID NO: 6 - protein, 10 aa, Cricetulus griseus
EAESSPFVER

SEQ ID NO: 7 - protein, 11 aa, Cricetulus griseus
SILFVPTSAPR

SEQ ID NO: 8 - protein, 11 aa, Cricetulus griseus
KEAESSPFVER

SEQ ID NO: 9 - protein, 14 aa, Cricetulus griseus
GVVDSDDLPLNVSR

SEQ ID NO: 10 - protein, 12 aa, Cricetulus griseus
IADEKYNDTFWK

SEQ ID NO: 11 - protein, 14 aa, Cricetulus griseus
NLLHVTDTGVGMTR

SEQ ID NO: 12 - protein, 13 aa, Cricetulus griseus
VFITDDFHDMMPK

SEQ ID NO: 13 - protein, 19 aa, Cricetulus griseus
LISLTDENALAGNEELTVK

SEQ ID NO: 14 - protein, 19 aa, Cricetulus griseus
FQSSHHSTDITSLDQYVER

SEQ ID NO: 15 - protein, 20 aa, Cricetulus griseus
YLNFVKGVVDSDDLPLNVSR

SEQ ID NO: 16 - protein, 611 aa, Homo sapiens
MAASAKKKNKKGKTISLTDFLAEDGGTGGGSTYVSKPVSWADETDDLEGDVSTTWHSNDDDVYRAPPIDRSILPTAPRAAREPNIDRSRLPKSPPYTAFLGNLPYDVTEESIKEFFRGLNISAVRLPREPSNPERLKGFGYAEFEDLDSLLSALSLNEESLGNRRIRVDVADQAQDKDRDDRSFGRDRNRDSDKTDTDWRARPATDSFDDYPPRRGDDSFGDKYRDRYDSDRYRDGYRDGYRDGPRRDMDRYGGRDRYDDRGSRDYDRGYDSRIGSGRRAFGSGYRRDDDYRGGGDRYEDRYDRRDDRSWSSRDDYSRDDYRRDDRGPPQRPKLNLKPRSTPEEDDSSASTSQSTRAASIFGGAKPVDTAAREREVEERLQKEQEKLQRQWNEPKLERRPRERHPSWRSEETQERERSRTGSESSQTGTSTTSSRNARRRESEKSLENETLNKEEDCHSPTSKPPKPDQPLKVMPAPPPKENAWVKRSSNPPARSQSSDTEQQSPTSGGGKVAPAQPSEEGPGRKDENKVDGMNAPKGQTGNSSRGPGDGGNRDHWKESDRKDGKKDQDSRSAPEPKKPEENPASKFSSASKYAALSVDGEDENEGEDYAE

SEQ ID NO: 17 - protein, 10 aa, Cricetulus griseus
VDVADQAQDK

SEQ ID NO: 18 - protein, 8 aa, Cricetulus griseus
GLNISAVR

SEQ ID NO: 19 - protein, 719 aa, Mus musculus
MALKDYAIEKEKVKKFLQEFYYENELGKKQFKYGTQLVHLAHREQVALYVDLDDIAEDDPELVDSICENAKRYSRLFGDVVQELLPEYKEKEVVNKDVLDVYIEHRLMMEQRSRDPGAVRNPQNQYPSELMRRFELYFRGPSSSKPRVIREVRADSVGKLLTVRGIVTRVSEVKPRMVVATYTCDQCGAETYQPIQSPTFMPLIMCPSQECQTNRSGGRLYLQTRGSKFVKFQEMKIQEHSDQVPVGNIPRSITVVLEGENTRIAQPGDHVSVTGIFLPVLRTGFQQMAQGLLSETYLEAHWIVKMTKSDDDVSGAGELSSEELKQIAEEDFYEKLAASIAPEIYGHEDVKKALLLLLVGGVDQSPQGMKIRGNIHICLMGDPGVAKSQLLSYIDRLAPRSQYTTGRGSSGVGLTAAVLRDSVSGELTLEGGALVLADQGVCCIDEFDKMAEADRTAIHEVMEQQTISIAKAGILTTLNARCSILAAANPAYGRYNPRRSLEQNVQLPAALLSRFDLLWLIQDRPDRDNDLRLAQHITYVHQHSRQPPAQFEPLDMKLMRRYIAMCHERQPTVPESLADYITAAYVEMRREARASKDATYTSARTLLAILRLSTALARLRMVDIVEKEDVNEAIRLMEMSKDSLLGEKGQTARTQRPADVIFATIRELVSRGRSVHFSEAEQRCISRGFTPAQFQAALDEYEELNVWQVNTSRTRITFV

SEQ ID NO: 20 - protein, 10 aa, Cricetulus griseus
AGILTTLNAR

SEQ ID NO: 21 - protein, 9 aa, Cricetulus griseus
SQLLSYIDR

SEQ ID NO: 22 - protein, 10 aa, Cricetulus griseus
DVLDVYIEHR

SEQ ID NO: 23 - protein, 11 aa, Cricetulus griseus
YGTQLVHLAHR

SEQ ID NO: 24 - protein, 12 aa, Cricetulus griseus
NPQNQYPSELMR

SEQ ID NO: 25 - protein, 15 aa, Cricetulus griseus
IQEHSDQVPVGNIPR

SEQ ID NO: 26 - protein, 17 aa, Cricetulus griseus
LAASIAPEIYGHEDVKK

SEQ ID NO: 27 - protein, 633 aa, Mus musculus
MSARGPAIGIDLGTTYSCVGVFQHGKVEIIANDQGNRTTPSYVAFTDTERLIGDAAKNQVAMNPTNTIFDRKRLIGRKFEDATVQSDMKHWPFRVVSEGGKPKVQVEYKGEMKTFFPEEISSMVLTKMKEIAEAYLGGKVQSAVITVPAYFNDSQRQATKDAGTITGLNVLRIINEPTAAAIAYGLDKKGCAGGEKNVLIFDLGGGTFDVSILTIEDGIFELKSTAGDTHLGGEDFDNRMVSHLAEEFKRKHKKDIGPNKRAVRRLRTACERAKRTLSSSTQASIEIDSLYEGVDFYTSITRARFEELNADLFRGTLEPVEKALRDAKLDKGQIQEIVLVGGSTRIPKIQKLLQDFFNGKELNKSINPDEAVAYGAAVQAAILIGDKSENVQDLLLLDVTPLSLGIETAGGVMTPLIKRNTTIPTKQTQTFTTYSDNQSSVLVQVYEGERAMTKDNNLLGKFDLTGIPPAPRGVPQIEVTFDIDANGILNVTAADKSTGKENKITITNDKGRLSKDDIDRMVQEAERYKSEDEANRDRVAAKNAVESYTYNIKQTVEDEKLRGKISEQDKNKILDKCQEVINWLDRNQMAEKDEYEHKQKELERVCNPIISKLYQGGPGGGGSSGGPTIEEVD

SEQ ID NO: 28 - protein, 7 aa, Cricetulus griseus
DNNLLGK

SEQ ID NO: 29 - protein, 9 aa, Cricetulus griseus
LLQDFFNGK

SEQ ID NO: 30 - protein, 13 aa, Cricetulus griseus
LLQDFFNGKELNK

SEQ ID NO: 31 - protein, 16 aa, Cricetulus griseus
STAGDTHLGGEDFDNR

SEQ ID NO: 32 - protein, 11 aa, Cricetulus griseus
VEIIANDQGNR

SEQ ID NO: 33 - protein, 6 aa, Cricetulus griseus
VQVEYK

SEQ ID NO: 34 - protein, 12 aa, Cricetulus griseus
ARFEELNADLFR

SEQ ID NO: 35 - protein, 10 aa, Cricetulus griseus
FEELNADLFR

SEQ ID NO: 36 - protein, 13 aa, Cricetulus griseus
TTPSYVAFTDTER

SEQ ID NO: 37 - protein, 661 aa, Caenorhabditis briggsae
MKTLFLLGLIALTAVSVYCEEEEKTEKKETKYGTIIGIDLGTTYSCVGVYKNGRVEIIANDQGNRITPSYVAFSGEQGDRLIGDAAKNQLTINPENTIFDAKRLIGRDYNDKTVQADIKHWPFKVLDKSNKPSVEVKVGADNKQFTPEEVSAMVLVKMKEIAESYLGKEVKHAVVTVPAYFNDAQRQATKDAGTIAGLNVVRIINEPTAAAIAYGLDKKDGERNILVFDLGGGTFDVSMLTIDNGVFEVLATNGDTHLGGEDFDQRVMEYFIKLYKKKSGKDLRKDKRAVQKLRREVEKAKRALSTQHQTKVEIESLFDGEDFSETLTRAKFEELNMDLFRATLKPVQKVLEDSDLKKDDVHEIVLVGGSTRIPKVQQLIKEFFNGKEPSRGINPDEAVAYGAAVQGGVISGEEDTGEIVLLDVNPLTMGIETVGGVMTKLISRNTVIPTKKSQVFSTAADNQPTVTIQVFEGERPMTKDNHQLGKFDLTGIPPAPRGVPQIEVTFEIDVNGILHVTAEDKGTGNKNKITITNDQNRLSPEDIERMINDAEKFAEDDKKVKEKAEARNELESYAYSLKNQIGDKEKLGGKLDEDDKKTIEEAVDEAISWLGSNADASAEELKEQKKELEGKVQPIVSKLYKDGGAGGEEAPEEGSDDKDEL

SEQ ID NO: 38 - protein, 9 aa, Cricetulus griseus
VLEDSDLKK

SEQ ID NO: 39 - protein, 9 aa, Cricetulus griseus
ITITNDQNR

SEQ ID NO: 40 - protein, 8 aa, Cricetulus griseus
VLEDSDLK

SEQ ID NO: 41 - protein, 11 aa, Cricetulus griseus
NELESYAYSLK

SEQ ID NO: 42 - protein, 10 aa, Cricetulus griseus
FEELNMDLFR

SEQ ID NO: 43 - protein, 654 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKRALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 44 - protein, 12 aa, Cricetulus griseus
AKFEELNMDLFR

SEQ ID NO: 45 - protein, 12 aa, Cricetulus griseus
DAGTIAGLNVMR

SEQ ID NO: 46 - protein, 12 aa, Cricetulus griseus
ELEEIVQPIISK

SEQ ID NO: 47 - protein, 10 aa, Cricetulus griseus
FEELNMDLFR

SEQ ID NO: 48 - protein, 16 aa, Cricetulus griseus
IINEPTAAAIAYGLDK

SEQ ID NO: 49 - protein, 6 aa, Cricetulus griseus
IQQLVK

SEQ ID NO: 50 - protein, 9 aa, Cricetulus griseus
ITITNDQNR

SEQ ID NO: 51 - protein, 14 aa, Cricetulus griseus
ITPSYVAFTPEGER

SEQ ID NO: 52 - protein, 15 aa, Cricetulus griseus
KSDIDEIVLVGGSTR

SEQ ID NO: 53 - protein, 18 aa, Cricetulus griseus
KSQIFSTASDNQPTVTIK

SEQ ID NO: 54 - protein, 11 aa, Cricetulus griseus
MKETAEAYLGK

SEQ ID NO: 55 - protein, 15 aa, Cricetulus griseus
NQLTSNPENTVFDAK

SEQ ID NO: 56 - protein, 14 aa, Cricetulus griseus
SDIDEIVLVGGSTR

SEQ ID NO: 57 - protein, 17 aa, Cricetulus griseus
SQIFSTASDNQPTVTIK

SEQ ID NO: 58 - protein, 14 aa, Cricetulus griseus
TFAPEEISAMVLTK

SEQ ID NO: 59 - protein, 15 aa, Cricetulus griseus
TKPYIQVDIGGGQTK

SEQ ID NO: 60 - protein, 11 aa, Cricetulus griseus
VEIIANDQGNR

SEQ ID NO: 61 - protein, 8 aa, Cricetulus griseus
VLEDSDLK

SEQ ID NO: 62 - protein, 10 aa, Cricetulus griseus
VYEGERPLTK

SEQ ID NO: 63 - protein, 646 aa, Cricetulus griseus
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGEAKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRNDKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIISWLDKNQTAEKEEFEHQQKELEKVCNPIITKLYQSAGGMPGGMPGGFPGGGAPPSGGASSGPTIEEVD

SEQ ID NO: 64 - protein, 12 aa, Cricetulus griseus
DAGTIAGLNVLR

SEQ ID NO: 65 - protein, 7 aa, Cricetulus griseus
DNNLLGK

SEQ ID NO: 66 - protein, 11 aa, Cricetulus griseus
FDDAVVQSDMK

SEQ ID NO: 67 - protein, 10 aa, Cricetulus griseus
FEELNADLFR

SEQ ID NO: 68 - protein, 8 aa, Cricetulus griseus
GTLDPVEK

SEQ ID NO: 69 - protein, 16 aa, Cricetulus griseus
IINEPTAAAIAYGLDK

SEQ ID NO: 70 - protein, 7 aa, Cricetulus griseus
ITITNDK

SEQ ID NO: 71 - protein, 9 aa, Cricetulus griseus
LLQDFFNGK

SEQ ID NO: 72 - protein, 8 aa, Cricetulus griseus
LSKEDIER

SEQ ID NO: 73 - protein, 11 aa, Cricetulus griseus
MKEIAEAYLGK

SEQ ID NO: 74 - protein, 14 aa, Cricetulus griseus
SQIHDIVLVGGSTR

SEQ ID NO: 75 - protein, 10 aa, Cricetulus griseus
MVNHFIAEFK

SEQ ID NO: 76 - protein, 15 aa, Cricetulus griseus
NQVAMNPTNTVFDAK

SEQ ID NO: 77 - protein, 11 aa, Cricetulus griseus
NSLESYAFNMK

SEQ ID NO: 78 - protein, 7 aa, Cricetulus griseus
NTTIPTK

SEQ ID NO: 79 - protein, 12 aa, Cricetulus griseus
RFDDAVVQSDMK

SEQ ID NO: 80 - protein, 8 aa, Cricetulus griseus
RNTTIPTK

SEQ ID NO: 81 - protein, 14 aa, Cricetulus griseus
SFYPEEVSSMVLTK

SEQ ID NO: 82 - protein, 16 aa, Cricetulus griseus
STAGDTHLGGEDFDNR

SEQ ID NO: 83 - protein, 13 aa, Cricetulus griseus
TTPSYVAFTDTER

SEQ ID NO: 84 - protein, 18 aa, Cricetulus griseus
TVTNAVVTVPAYFNDSQR

SEQ ID NO: 85 - protein, 8 aa, Cricetulus griseus
VCNPIITK

SEQ ID NO: 86 - protein, 11 aa, Cricetulus griseus
VEIIANDQGNR

SEQ ID NO: 87 - protein, 10 aa, Cricetulus griseus
VQVEYKGEAK

SEQ ID NO: 88 - protein, 654 aa, Homo sapiens; identical to SEQ ID NO: 43

SEQ ID NO: 89 - protein, 12 aa, Cricetulus griseus
AKFEELNMDLFR

SEQ ID NO: 90 - protein, 9 aa, Cricetulus griseus
ALSSQHQAR

SEQ ID NO: 91 - protein, 12 aa, Cricetulus griseus
DAGTIAGLNVMR

SEQ ID NO: 92 - protein, 18 aa, Cricetulus griseus
DNHLLGTFDLTGIPPAPR

SEQ ID NO: 93 - protein, 12 aa, Cricetulus griseus
ELEEIVQPIISK

SEQ ID NO: 94 - protein, 7 aa, Cricetulus griseus
FAEEDKK

SEQ ID NO: 95 - protein, 10 aa, Cricetulus griseus
FEELNMDLFR

SEQ ID NO: 96 - protein, 16 aa, Cricetulus griseus
IINEPTAAAIAYGLDK

SEQ ID NO: 97 - protein, 6 aa, Cricetulus griseus
IQQLVK

SEQ ID NO: 98 - protein, 9 aa, Cricetulus griseus
ITITNDQNR

SEQ ID NO: 99 - protein, 14 aa, Cricetulus griseus
ITPSYVAFTPEGER

SEQ ID NO: 100 - protein, 15 aa, Cricetulus griseus
KSDIDEIVLVGGSTR

SEQ ID NO: 101 - protein, 11 aa, Cricetulus griseus
LSSEDKETMEK

SEQ ID NO: 102 - protein, 8 aa, Cricetulus griseus
LTPEEIER

SEQ ID NO: 103 - protein, 11 aa, Cricetulus griseus
MKETAEAYLGK

SEQ ID NO: 104 - protein, 11 aa, Cricetulus griseus
NELESYAYSLK

SEQ ID NO: 105 - protein, 15 aa, Cricetulus griseus
NQLTSNPENTVFDAK

SEQ ID NO: 106 - protein, 8 aa, Cricetulus griseus
NTVVPTKK

SEQ ID NO: 107 - protein, 10 aa, Cricetulus griseus
RALSSQHQAR

SEQ ID NO: 108 - protein, 14 aa, Cricetulus griseus
SDIDEIVLVGGSTR

SEQ ID NO: 109 - protein, 17 aa, Cricetulus griseus
SQIFSTASDNQPTVTIK

SEQ ID NO: 110 - protein, 14 aa, Cricetulus griseus
TFAPEEISAMVLTK

SEQ ID NO: 111 - protein, 11 aa, Cricetulus griseus
VEIIANDQGNR

SEQ ID NO: 112 - protein, 15 aa, Cricetulus griseus
TKPYIQVDIGGGQTK

SEQ ID NO: 113 - protein, 12 aa, Cricetulus griseus
TWNDPSVQQDIK

SEQ ID NO: 114 - protein, 9 aa, Cricetulus griseus
VLEDSDLKK

SEQ ID NO: 115 - protein, 7 aa, Cricetulus griseus
VMEHFIK

SEQ ID NO: 116 - protein, 17 aa, Cricetulus griseus
VTHAVVTVPAYFNDAQR

SEQ ID NO: 117 - protein, 10 aa, Cricetulus griseus
VYEGERPLTK

SEQ ID NO: 118 - protein, 556 aa, Mus musculus
MEGPLSVFGDRSTGEAIRSQNVMAAASIANIVKSSLGPVGLDKMLVDDIGDVTITNDGATILKLLEVEHPAAKVLCELADLQDKEVGDGTTSVVIIAAELLKNADELVKQKIHPTSVISGYRLACKEAVRYINENLIINADELGRDCLTNTAKTSMSSKIIGINGDYFANMVVDAVLAVKYTDARGQPRYPINSVNILKAHGRSQIESMLINGYALNCVVGSQGMPKRIVNAKIACLDFSLQKTKMKLGVQVVITDPEKLDQIRQRESDITKERIQKILATGANVILTTGGIDDMCLKYFVEAGAMAVRRVLKRDLKCVAKASGATILSTLANLEGEETFEVTMLGQAEEVVQERICDDELILIKNTKARTSASIILRGANDFMCDEMERSLHDALCVVKRVLESKSVVPGGGAVEAALSIYLENYATSMGSREQLAIAEFARSLLVIPNTLAVNAAQDSTDLVAKLRAFHNEAQVNPERKNLKWIGLDLVHGKPRDNKQAGVFEPTIVKVKSLKFATEAAITILRIDDLIKLHPECKDDKHGSYENAVHSGALDD

SEQ ID NO: 119 - protein, 11 aa, Cricetulus griseus
FATEAAITILR

SEQ ID NO: 120 - protein, 12 aa, Cricetulus griseus
LGVQVVITDPEK

SEQ ID NO: 121 - protein, 10 aa, Cricetulus griseus
LLEVEHPAAK

SEQ ID NO: 122 - protein, 11 aa, Cricetulus griseus
QAGVFEPTIVK

SEQ ID NO: 123 - protein, 10 aa, Cricetulus griseus
SSLGPVGLDK

SEQ ID NO: 124 - protein, 12 aa, Cricetulus griseus
AFHNEAQVNPER

SEQ ID NO: 125 - protein, 10 aa, Cricetulus griseus
EQLAIAEFAR

SEQ ID NO: 126 - protein, 15 aa, Cricetulus griseus
SQNVMAAASIANIVK

SEQ ID NO: 127 - protein, 562 aa, Mus musculus
MVKIVTVKTQAYPDQKPGTSGLRKRVKVFQSNANYAENFIQSIVSTVEPALRQEATLVVGGDGRFYMTEAIQLIVRIAAANGIGRLVIGQNGILSTPAVSCIIRKIKAIGGIILTASHNPGGPNGDFGIKFNISNGGPAPEAITDKIFQISKTIEEYAICPDLKVDLGVLGKQQFDLENKFKPFTVEIVDSVEAYATMLRNIFDFNALKELLSGPNRLKIRIDAMHGVVGPYVKKILCEELGAPANSAVNCVPLEDFGGHHPDPNLTYAADLVETMKSGEHDFGAAFDGDGDRNMILGKHGFFVNPSDSVAVIAANIFSIPYFQQTGVRGFARSMPTSGALDRVANATKIALYETPTGWKFFGNLMDASKLSLCGEESFGTGSDHIREKDGLWAVLAWLSILATRKQSVEDILKDHWQKFGRNFLTRYDYEEVEAEGANKMMKDLEALMLDRSFVGKQFSANDKVYTVEKADNFEYSDPVDGSISKNQGLRLIFADGSRIIFRLSGTGSAGATIRLYIDSYEKDVAKINQDPQVMLAPLISIALKVSQLQERTGRTAPTVIT

SEQ ID NO: 128 - protein, 5 aa, Cricetulus griseus
DHWQK

SEQ ID NO: 129 - protein, 8 aa, Cricetulus griseus
LIFADGSR

SEQ ID NO: 130 - protein, 8 aa, Cricetulus griseus
QQFDLENK

SEQ ID NO: 131 - protein, 8 aa, Cricetulus griseus
LYIDSYEK

SEQ ID NO: 132 - protein, 12 aa, Cricetulus griseus
LSGTGSAGATIR

SEQ ID NO: 133 - protein, 10 aa, Cricetulus griseus
FFGNLMDASK

SEQ ID NO: 134 - protein, 12 aa, Cricetulus griseus
QEATLVVGGDGR

SEQ ID NO: 135 - protein, 13 aa, Cricetulus griseus
IDAMHGVVGPYVK

SEQ ID NO: 136 - protein, 13 aa, Cricetulus griseus
YDYEEVEAEGANK

SEQ ID NO: 137 - protein, 15 aa, Cricetulus griseus
TQAYPDQKPGTSGLR

SEQ ID NO: 138 - protein, 16 aa, Cricetulus griseus
SMPTSGALDRVANATK

SEQ ID NO: 139 - protein, 16 aa, Cricetulus griseus
SGEHDFGAAFDGDGDR

SEQ ID NO: 140 - protein, 22 aa, Cricetulus griseus
SGEHDFGAAFDGDGDRNMILGK

SEQ ID NO: 141 - protein, 514 aa, Cricetulus griseus
MADYLISGGTSYVPDDGLTAQQLFNCGDGLTYNDFLILPGYIDFTADQVDLTSALTKKITLKTPLVSSPMDTVTEAGMAIAMALTGGIGFIHHNCTPEFQANEVRKVKKYEQGFITDPVVLSPKDRVRDVFEAKARHGFCGIPITDTGRMGSRLVGIISSRDIDFLKEEEHDRFLEEIMTKREDLVVAPAGITLKEANEILQRSKKGKLPIVNENDELVAIIARTDLKKNRDYPLASKDAKKQLLCGAAIGTHEDDKYRLDLLALAGVDVVVLDSSQGNSIFQINMIKYMKEKYPNLQVIGGNVVTAAQAKNLIDAGVDALRVGMGCGSICITQEVLACGRPQATAVYKVSEYARRFGVPVIADGGIQNVGHIAKALALGASTVMMGSLLAATTEAPGEYFFSDGIRLKKYRGMGSLDAMDKHLSSQNRYFSEADKIKVAQGVSGAVQDKGSIHKFVPYLIAGIQHSCQDIGAKSLTQVRAMMYSGELKFEKRTSSAQVEGGVHSLHSYEKRLF

SEQ ID NO: 142 - protein, 8 aa, Cricetulus griseus
PQATAVYK

SEQ ID NO: 143 - protein, 12 aa, Cricetulus griseus
VAQGVSGAVQDK

SEQ ID NO: 144 - protein, 18 aa, Cricetulus griseus
TSSAQVEGGVHSLHSYEK

SEQ ID NO: 145 - protein, 16 aa, Cricetulus griseus
KYEQGFITDPVVLSPK

SEQ ID NO: 146 - protein, 11 aa, Cricetulus griseus
NLIDAGVDALR

SEQ ID NO: 147 - protein, 520 aa, Cricetulus griseus
MPGSLPLNAEACWPKDVGIVALEIYFPSQYVDQAELEKYDGVDAGKYTIGLGQARMGFCTDREDINSLCLTVVQNLMERNSLSYDCIGRLEVGTETIIDKSKSVKSNLMQLFEESGNTDIEGIDTTNACYGGTAAVFNAVNWIESSSWDGRYALVVAGDIAIYATGNARPTGGVGAVALLIGPNAPLIFDRGLRGTHMQHAYDFYKPDMLSEYPIVDGKLSIQCYLSALDRCYSVYRKKIRAQWQKEGNDNDFTLNDFGFMISHSPYCKLVQKSLARMFLNDFLNDQNRDKNSIYSGLEAFGDVKLEDTYFDRDVEKAFMKASSELFNQKTKASLLVSNQNGNMYTSSVYGSLASVLAQYSPQQLAGKRIGVFSYGSGLAATLYSLKVTQDATPGSALDKVTASLCDLKSRLDSRTCVAPDVFAENMKLREDTHHLANYIPQCSIDSLFEGTWYLVRVDEKHRRTYARRPSTNDHNLGDGVGLVHSNTATEHIPSPAKKVPRLPATAAESESAVISNGEH

SEQ ID NO: 148 - protein, 9 aa, Cricetulus griseus
ASSELFNQK

SEQ ID NO: 149 - protein, 8 aa, Cricetulus griseus
LEDTYFDR

SEQ ID NO: 150 - protein, 11 aa, Cricetulus griseus
LEVGTETIIDK

SEQ ID NO: 151 - protein, 18 aa, Cricetulus griseus
LPATAAESESAVISNGEH

SEQ ID NO: 152 - protein, 12 aa, Cricetulus griseus
MFLNDFLNDQNR

SEQ ID NO: 153 - protein, 13 aa, Cricetulus griseus
TCVAPDVFAENMK

SEQ ID NO: 154 - protein, 13 aa, Cricetulus griseus
VTQDATPGSALDK

SEQ ID NO: 155 - protein, 7 aa, Cricetulus griseus
MGFCTDR

SEQ ID NO: 156 - protein, 458 aa, Mus musculus
MTAEEMKAAENGAQSAPLPLEGVDISPKQDEGVLKVIKREGTGTETPMIGDRVFVHYTGWLLDGTKFDSSLDRKDKFSFDLGKGEVIKAWDIAVATMKVGEVCHITCKPEYAYGAAGSPPKIPPNATLVFEVELFEFKGEDLTEEEDGGIIRRIRTRGEGYARPNDGAMVEVALEGYHKDRLFDQRELCFEVGEGESLDLPCGLEEAIQRMEKGEHSIVYLKPSYAFGSVGKERFQIPPHAELRYEVRLKSFEKAKESWEMSSAEKLEQSNIVKERGTAYFKEGKYKQALLQYKKIVSWLEYESSFSGEEMQKVHALRLASHLNLAMCHLKLQAFSAAIESCNKALELDSNNEKGLFRRGEAHLAVNDFDLARADFQKVLQLYPSNKAAKTQLAVCQQRTRRQLAREKKLYANMFERLAEEEHKVKAEVAAGDHPTDAEMKGERNNVAENQSRVETEA

SEQ ID NO: 157 - protein, 10 aa, Cricetulus griseus
ALELDSNNEK

SEQ ID NO: 158 - protein, 9 aa, Cricetulus griseus
DKFSFDLGK

SEQ ID NO: 159 - protein, 13 aa, Cricetulus griseus
EGTGTETPMIGDR

SEQ ID NO: 160 - protein, 7 aa, Cricetulus griseus
FDSSLDR

SEQ ID NO: 161 - protein, 8 aa, Cricetulus griseus
LEQSNIVK

SEQ ID NO: 162 - protein, 13 aa, Cricetulus griseus
LQAFSAAIESCNK

SEQ ID NO: 163 - protein, 10 aa, Cricetulus griseus
PSYAFGSVGK

SEQ ID NO: 164 - protein, 9 aa, Cricetulus griseus
VLQLYPSNK

SEQ ID NO: 165 - protein, 522 aa, Homo sapiens
MNINDLKLTLSKAGQEHLLRFWNELEEAQQVELYAELQAMNFEELNFFFQKAIEGFNQSSHQKNVDARMEPVPREVLGSATRDQDQLQAWESEGLFQISQNKVAVLLLAGGQGTRLGVAYPKGMYDVGLPSRKTLFQIQAERILKLQQVAEKYYGNKCIIPWYIMTSGRTMESTKEFFTKHKYFGLKKENVIFFQQGMLPAMSFDGKIILEEKNKVSMAPDGNGGLYRALAAQNIVEDMEQRGIWSIHVYCVDNILVKVADPRFIGFCIQKGADCGAKVVEKTNPTEPVGVVCRVDGVYQVVEYSEISLATAQKRSSDGRLLFNAGNIANHFFTVPFLRDVVNVYEPQLQHHVAQKKIPYVDTQGQLIKPDKPNGIKMEKFVFDIFQFAKKFVVYEVLREDEFSPLKNADSQNGKDNPTTARHALMSLHHCWVLNAGGHFIDENGSRLPAIPRSATNGKSETITADVNHNLKDANDVPIQCEISPLISYAGEGLESYVADKEFHAPLIIDENGVHELVKNGI

SEQ ID NO: 166 - protein, 7 aa, Cricetulus griseus
LGVAYPK

SEQ ID NO: 167 - protein, 14 aa, Cricetulus griseus
ALAAQNIVEDMEQR

SEQ ID NO: 168 - protein, 9 aa, Cricetulus griseus
TLFQIQAER

SEQ ID NO: 169 - protein, 13 aa, Cricetulus griseus
VAVLLLAGGQGTR

SEQ ID NO: 170 - protein, 453 aa, Mus musculus
DEQMLKRRNVSSFPDDATSPLQENRNNQGTVNWSVEDIVKGINSNNLESQLQATQAARKLLSREKQPPIDNIIRAGLIPKFVSFLGKTDCSPIQFESAWALTNIASGTSEQTKAVVDGGAIPAFISLLASPHAHISEQAVWALGNIAGDGSAFRDLVIKHGAIDPLLALLAVPDLSTLACGYLRNLTWTLSNLCRNKNPAPPLDAVEQILPTLVRLLHHNDPEVLADSCWAISYLTDGPNERIEMVVKKGVVPQLVKLLGATELPIVTPALRAIGNIVTGTDEQTQKVIDAGALAVFPSLLTNPKTNIQKEATWTMSNITAGRQDQIQQVVNHGLVPFLVGVLSKADFKTQKEAAWAITNYTSGGTVEQIVYLVHCGIIEPLMNLLSAKDTKIIQVILDAISNIFQAAEKLGETEKLSIMIEECGGLDKIEALQRHENESVYKASLNLIEKYF

SEQ ID NO: 171 - protein, 7 aa, Cricetulus griseus
FVSFLGK

SEQ ID NO: 172 - protein, 9 aa, Cricetulus griseus
QPPIDNIIR

SEQ ID NO: 173 - protein, 11 aa, Cricetulus griseus
EKQPPIDNIIR

SEQ ID NO: 174 - protein, 15 aa, Cricetulus griseus
LLGATELPIVTPALR

SEQ ID NO: 175 - protein, 17 aa, Cricetulus griseus
NVSSFPDDATSPLQENR

SEQ ID NO: 176 - protein, 18 aa, Cricetulus griseus
GINSNNLESQLQATQAAR

SEQ ID NO: 177 - protein, 511 aa, Rattus norvegicus
MALRAMRGIVNGAAPELPVPTGGPMAGAREQALAVSRNYLSQPRLTYKTVSGVNGPLVILDHVKFPRYAEIVHLTLPDGTKRSGQVLEVSGSKAVVQVFEGTSGIDAKKTSCEFTGDILRTPVSEDMLGRVFNGSGKPIDRGPVVLAEDFLDIMGQPINPQCRIYPEEMIQTGISAIDGMNSIARGQKIPIFSAAGLPHNEIAAQICRQAGLVKKSKDVVDYSEENFAIVFAAMGVNMETARFFKSDFEENGSMDNVCLFLNLANDPTIERIITPRLALTTAEFLAYQCEKHVLVILTDMSSYAEALREVSAAREEVPGRRGFPGYMYTDLATIYERAGRVEGRNGSITQIPILTMPNDDITHPIPDLTGYITEGQIYVDRQLHNRQIYPPINVLPSLSRLMKSAIGEGMTRKDHADVSNQLYACYAIGKDVQAMKAVVGEEALTSDDLLYLEFLQKFEKNFITQGPYENRTVYETLDIGWQLLRIFPKEMLKRIPQSTLSEFYPRDSAKH

SEQ ID NO: 178 - protein, 10 aa, Cricetulus griseus
TPVSEDMLGR

SEQ ID NO: 179 - protein, 11 aa, Cricetulus griseus
NFITQGPYENR

SEQ ID NO: 180 - protein, 12 aa, Cricetulus griseus
IPQSTLSEFYPR

SEQ ID NO: 181 - protein, 15 aa, Cricetulus griseus
AVVQVFEGTSGIDAK

SEQ ID NO: 182 - protein, 14 aa, Cricetulus griseus
YAEIVHLTLPDGTK

SEQ ID NO: 183 - protein, 14 aa, Cricetulus griseus
QIYPPINVLPSLSR

SEQ ID NO: 184 - protein, 16 aa, Cricetulus griseus
TVSGVNGPLVILDHVK

SEQ ID NO: 185 - protein, 16 aa, Cricetulus griseus
GFPGYMYTDLATIYER

SEQ ID NO: 186 - protein, 16 aa, Cricetulus griseus
HVLVILTDMSSYAELR

SEQ ID NO: 187 - protein, 283 aa, Bos taurus
RRGAISAEVYTEEDAASYVRKVIPKDYKTMAALAKAIEKNVLFSHLDDNERSDIFDAMFPVSFIAGETVIQQGDEGDNFYVIDQGEMDVYVNNEWATSVGEGGSFGELALIYGTPRAATVKAKTNVKLWGIDRDSYRRILMGSTLRKRKMYEEFLSKVSILESLDKWERLTVADALEPVQFEDGQKIVVQGEPGDEFFIILEGSAAVLQRRSENEEFVEVGRLGPSDYFGEIALLMNRPRAATVVARGPLKCVKLDRPRFERVLGPCSDILKRNIQQYNSFVS

SEQ ID NO: 188 - protein, 8 aa, Cricetulus griseus
ILMGSTLR

SEQ ID NO: 189 - protein, 11 aa, Cricetulus griseus
SENEEFVEVGR

SEQ ID NO: 190 - protein, 12 aa, Cricetulus griseus
RSENEEFVEVGR

SEQ ID NO: 191 - protein, 12 aa, Cricetulus griseus
NVLFSHLDDNER

SEQ ID NO: 192 - protein, 17 aa, Cricetulus griseus
LTVADALEPVQFEDGQK

SEQ ID NO: 193 - protein, 18 aa, Cricetulus griseus
GAISAEVYTEEDAASYVR

SEQ ID NO: 194 - protein, 18 aa, Cricetulus griseus
LGPSDYFGEIALLMNRPR

SEQ ID NO: 195 - protein, 443 aa, Rattus norvegicus
DVDKAVKAAQAAFQLGSPWRRMDASDRGRLLYRLADLIERDRTYLAALETLDNGKPYVISYLVDLDMVLKCLRYYAGWADKYHGKTIPIDGDFFSYTRHEPVGVCGQIIPWNFPLLMQAWKLGPALATGNVVVMKVAEQTPLTALYVANLIKEAGFPPGVVNIVPGFGPTAGAAIASHEDVDKVAFTGSTEVGHLIQVAAGSSNLKRVTLELGGKSPNIIMSDADMDWAVEQAHFALFFNQGQCCCAGSRTFVQEDVYDEFVERSVARAKSRVVGNPFDSRTEQGPQVDETQFKKILGYIKSGQQEGAKLLCGGGAAADRGYFIQPTVFGDVKDGMTIAKEEIFGPVMQILKFKTIEEVVGRANNSKYGLAAAVFTKDLDKANYLSQALQAGTVWINCYDVFGAQSPFGGYKMSGSGRELGEYGLQAYTEVKTVTVKVPQKNS

SEQ ID NO: 196 - protein, 7 aa, Cricetulus griseus
LADLIER

SEQ ID NO: 197 - protein, 8 aa, Cricetulus griseus
TIEEVVGR

SEQ ID NO: 198 - protein, 9 aa, Cricetulus griseus
VVGNPFDSR

SEQ ID NO: 199 - protein, 10 aa, Cricetulus griseus
YGLAAAVFTK

SEQ ID NO: 200 - protein, 12 aa, Cricetulus griseus
EEIFGPVMQILK

SEQ ID NO: 201 - protein, 13 aa, Cricetulus griseus
GYFIQPTVFGDVK

SEQ ID NO: 202 - protein, 13 aa, Cricetulus griseus
TIPIDGDFFSYTR

SEQ ID NO: 203 - protein, 14 aa, Cricetulus griseus
ELGEYGLQAYTEVK

SEQ ID NO: 204 - protein, 14 aa, Cricetulus griseus
TFVQEDVYDEFVER

SEQ ID NO: 205 - protein, 17 aa, Cricetulus griseus
VAEQTPLTALYVANKIK

SEQ ID NO: 206 - protein, 22 aa, Cricetulus griseus
VAETGSTEVGHLIQVAAGSSLK

SEQ ID NO: 207 - protein, 332 aa, Rattus norvegicus
MGGEQEEERFDGMLLAMAQQHEGGVQELVNTFFSFLRRKTDFFIGGEEGMAEKLITQTFNHHNQLAQKARREKRARQETERREKAERAARLAKEAKAETPGPQIKELTDEEAERLQLEIDQKKDAENHEVQLKNGSLDSPGKQDAEEEEDEEDEKDKGKLKPNLGNGADLPNYRWTQTLSELDLAVPFRVSFRLKGKDVVVDIQRRHLRVGLKGQAPVIDGELYNEVKVEESSWLIEDGKVVTVHLEKINKMEWWNRLVTSDPEINTKKINPENSKLSDLDSETRSMVEKMMYDQRQKSMGLPTSDEQKKQEILKKFMDQHPEMDFSKAKFN

SEQ ID NO: 208 - protein, 10 aa, Cricetulus griseus
DAENHEVQLK

SEQ ID NO: 209 - protein, 8 aa, Cricetulus griseus
DVVVDIQR

SEQ ID NO: 210 - protein, 9 aa, Cricetulus griseus
ELTDEEAER

SEQ ID NO: 211 - protein, 10 aa, Cricetulus griseus
GKDVVVDIQR

SEQ ID NO: 212 - protein, 9 aa, Cricetulus griseus
LSDLDSETR

SEQ ID NO: 213 - protein, 11 aa, Cricetulus griseus
LVTSDPEINTK

SEQ ID NO: 214 - protein, 11 aa, Cricetulus griseus
SMGLPTSDEQK

SEQ ID NO: 215 - protein, 14 aa, Cricetulus griseus
TDFFIGGEEGMAEK

SEQ ID NO: 216 - protein, 12 aa, Cricetulus griseus
VEESSWLIEDGK

SEQ ID NO: 217 - protein, 8 aa, Cricetulus griseus
VVTVHLEK

SEQ ID NO: 218 - protein, 403 aa, Rattus norvegicus
MCCYHGNRIAPGQVKMAGRSVGVPRRGAAGPQSRGQLAAGRDLLAREQEYKRLNAELEAKTANLVRQAEEVIREQQEERARPFSALTTSCKEEDGYGPRDLLSSEGMAHPWSETKPKTKNTGPVNKIQNRLHSADKERKINSSVKLKYPDVQTANDVAIPDDFSDFSLAKTISRIEGQLEEDVLPECADDDSFCGVSKDIGTEAQIRFLKAKLHVMQEELDSVVCECNKKEDEIQDLKSKVKNLEEDCVRQQRTVTSQQTQIEKYRNLFEEANKRCDELQQQLSSVDRELENKRRLQKQAATSQSATEVRLNRALEEAEKYKAELSKLRQTNKDITNEDHQKIEVLKSENKKLERQKGELMIGFKKQLKLIDILKRQKMHIEAAKMLSFSEEEFMKALEWGSS

SEQ ID NO: 219 - protein, 7 aa, Cricetulus griseus
LIDILKR

SEQ ID NO: 220 - protein, 9 aa, Cricetulus griseus
RGAAGPQSR

SEQ ID NO: 221 - protein, 8 aa, Cricetulus griseus
EEDGYGPR

SEQ ID NO: 222 - protein, 12 aa, Cricetulus griseus
QAATSQSATEVR

SEQ ID NO: 223 - protein, 12 aa, Cricetulus griseus
ARPFSALTTSCK

SEQ ID NO: 224 - protein, 15 aa, Cricetulus griseus
LQKQAATSQSATEVR

SEQ ID NO: 225 - protein, 18 aa, Cricetulus griseus
MLSFSEEEFMKALEWGSS

SEQ ID NO: 226 - protein, 349 aa, Mus musculus
MEVQESGCDPSESGAQEPSPVPSKTAGHYELPWVEKYRPLKLNEIVGNEDTVSRLEVFAREGNVPNIIIAGPPGTGKTTSILCLARALLGPALKDAVLELNASNDRGIDVVRNKIKMFAQQKVTLPKGRHKIIILDEADSMTDGAQQALRRTMEIYSKTTRFALACNASDKIIEPIQSRCAVLRYTKLTDAQVLTRLMNVIEKEKVPYTDDGLEAIIFTAQGDMRQALNNLQSTFSGFGYINSENVFKVCDEPHPLLVKEMIQHCVDANIDEAYKILAHLWHLGYSPEDVIGNIFRVCKTFPMAEYLKLEFIKEIGYTHMKVAEGVNSLLQMAGLLARLCQKTMAPVAS

SEQ ID NO: 227 - protein, 12 aa, Cricetulus griseus
DAVLELNASNDR

SEQ ID NO: 228 - protein, 13 aa, Cricetulus griseus
LNEIVGNEDTVSR

SEQ ID NO: 229 - protein, 9 aa, Cricetulus griseus
LTDAQVLTR

SEQ ID NO: 230 - protein, 9 aa, Cricetulus griseus
TTSILCLAR

SEQ ID NO: 231 - protein, 17 aa, Cricetulus griseus
EGNVPNIIIAGPPGTGK

SEQ ID NO: 232 - protein, 10 aa, Cricetulus griseus
FALACNASDK

SEQ ID NO: 233 - protein, 346 aa, Sus scrofa
MAMVSEFLKQAWFIDNEEQEYIKTVKGSKGGPGSAVSPYPTFNPSSDVEASHKAITVKGVDEATIIEIHTKRTNAQRQQIKAAYLQEKGKPLDEALKKALTGHLEEVALALLKTPAQFDADELRAAMKGLGTDEDTLNEILASRTNREIREINRVYKEELKRDLAKDITSDTSGDYQKALLSLAKGDRSEDLAINDDLADTDARALYEAGERRKGTDLNVFITILTTRSYLHLRRVFQKYSKYSKHDMNKVLDLELKGDIENCLTVVVKCATSKPMFFAEKLHQAMKGNGTRHKTLIRIMVSRSEIDMNDIKACYQKLYGISLCQAILDETKGDYEKILVALCGGD

SEQ ID NO: 234 - protein, 7 aa, Cricetulus griseus
ALLSLAK

SEQ ID NO: 235 - protein, 11 aa, Cricetulus griseus
TPAQFDADELR

SEQ ID NO: 236 - protein, 337 aa, Cricetulus griseus
MSGSPVKRQRMESALDQLKQFTTVVADTGDFNAIDEYKPQDATTNPSLILAAAQMPAYQELVEEAIAYGKKLGGPQEEQIKNAIDKLFVLFGAEILKKIPGRVSTEVDARLSFDKDAMVARAKRLIELYKEAGISKDRILIKLSSTWEGIQAGKELEEQHGIHCNMTLLFSFAQAVACAEAGVTLISPFVGRILDWHVANTDKKSYEPQEDPGVKSVTKIYNYYKKFGYKTIVMGASFRNTGEIKALAGCDFLTISPKLLGELLKDNTKLAPVLSIKAAQTSDLGKIHLDEKAFRWLHSEDQMAVEKLSDGIRKFAADAIKLERMLTERMFSAENGK

SEQ ID NO: 237 - protein, 7 aa, Cricetulus griseus
RLIELYK

SEQ ID NO: 238 - protein, 9 aa, Cricetulus griseus
TIVMGASFR

SEQ ID NO: 239 - protein, 10 aa, Cricetulus griseus
LGGPQEEQIK

SEQ ID NO: 240 - protein, 10 aa, Cricetulus griseus
LFVLGAEILK

SEQ ID NO: 241 - protein, 10 aa, Cricetulus griseus
LSDKDAMVAR

SEQ ID NO: 242 - protein, 280 aa, Oryctolagus cuniculus
MTTNFLVHEKIWFDKFKYDDAERSFYERMNGPVPGPSRQENGATVILRDIARARENIQKSLAGSSGPGASSGPGGDHSELAVRIASLEVENQNLRGVVQDLQRAVSKLEARLSALEKSSPTHRASAPQTQHVSPMRQVEPPAKKAAAPAEDDEDDDIDLFGSDEEEDKEAARLREERLRQYAEKKARKPALVAKSSILLDVKPWDDETDMARLEACVRSVQLDGLVWGASKLVPVGYGIRKLQIQCVVEDDKVGTDLLEEEITKFEEHVQSVDIAAFNKI

SEQ ID NO: 243 - protein, 15 aa, Cricetulus griseus
FEEHVQSVDIAAFNK

SEQ ID NO: 244 - protein, 12 aa, Cricetulus griseus
VGTDLLEEEITK

SEQ ID NO: 245 - protein, 12 aa, Cricetulus griseus
IASLEVENQNLR

SEQ ID NO: 246 - protein, 319 aa, Sus scrofa
MAAKGGTVKAASGFNAAEDAQTLRKAMKGLGTDEDAIISVLAYRSTAQRQEIRTAYKSTIGRDLLDDLKSELSGNFEQVILGMMTPTVLYDVQELRRAMKGAGTDEGCLIEILASRTPEEIRRINQTYQLQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAERLYKSMKGLGTDDNTLIRVMVSRAEIDMMDIRANFKRLYGKSLYSFIKGDTSGDYRKVLLILCGGDD

SEQ ID NO: 247 - protein, 12 aa, Cricetulus griseus
QDAQDLYEAGEK

SEQ ID NO: 248 - protein, 11 aa, Cricetulus griseus
GLGTDDNTLIR

SEQ ID NO: 249 - protein, 9 aa, Cricetulus griseus
AEIDMMDIR

SEQ ID NO: 250 - protein, 7 aa, Cricetulus griseus
DLLDDLK

SEQ ID NO: 251 - protein, 263 aa, Rattus norvegicus
MEESEYESVLCVKPEVHVYRIPPRATNRGYRASEWQLDQPSWSGRLRITAKGKVAYIKLEDRTSGELFAQAPVDQFPGTAVESVTDSSRYFVIRIEDGNGRRAFIGIGFGDRGDAFDFNVALQDHFKWVKQQCEFAKQAQNPDEGPKLDLGFKEGQTIKINIANMRKKEGAAGAPRTRPASAGGLSLLPPPPGGKMSTLIPPSGEQFSGGSLVQPVSGSGGATELWPQSKPAAAATADIWGDFTKSTGSPSSQSQPGTGWVQF

SEQ ID NO: 252 - protein, 10 aa, Cricetulus griseus
QAQNPDEGPK

SEQ ID NO: 253 - protein, 6 aa, Cricetulus griseus
LDLGFK

SEQ ID NO: 254 - protein, 10 aa, Cricetulus griseus
AFIGIGFGDR

SEQ ID NO: 255 - protein, 205 aa, Rattus norvegicus
MTERRVPFSLLRSPSWEPFRDWYPAHSRLFDQAFGVPRFPDEWSQWFSSAGWPGYVRPLPAATAEGPAAVTLARPFSRALNRQLSSGVSEIRQTADRWRVSLDVNHFAPEELTVKTKEGVVEITGKHEERQDEHGYISRCFTRKYTLPPGVDPTLVSSSLSPEGTLTVEAPLPKAVTQSAEITIPVTFEARAQIGGPESEQSGAK

SEQ ID NO: 256 - protein, 7 aa, Cricetulus griseus
VPFSLLR

SEQ ID NO: 257 - protein, 10 aa, Cricetulus griseus
QLSSGVSEIR

SEQ ID NO: 258 - protein, 9 aa, Cricetulus griseus
QDEHGYISR

SEQ ID NO: 259 - protein, 10 aa, Cricetulus griseus
LFDQAFGVPR

SEQ ID NO: 260 - protein, 16 aa, Cricetulus griseus
VSLDVNHFAPEELTVK

SEQ ID NO: 261 - protein, 218 aa, Macaca fascicularis
MPMTLGYWDIRGLAHAIRLLLEYTDSSYEEKKYTMGDAPDYDRSQWLNEKFKLGLDFPNLPYLIDGTHKITQSNAILRYIARKHNLCGETEEEKIRVDILENQAMDVSNQLARVCYSPDFEKLKPEYLEGLPTMMQHFSQFLGKRPWFVGDKITFVDFLAYDVLDLHRIFEPKCLDAFPNLKDFISHFEGLEKISAYMKSSRFLPKPLYTRVAVWGNK

SEQ ID NO: 262 - protein, 9 aa, Cricetulus griseus
ITQSNAILR

SEQ ID NO: 263 - protein, 13 aa, Cricetulus griseus
LLLEYTDSSYEEK

SEQ ID NO: 264 - protein, 180 aa, Rattus norvegicus
MSESELQLVARRIRSFPDFPIPGVLFRDISPLLKDPDSFRASIRLLAGHLKSTHGGKIDYIAGLDSRGFLFGPSLAQELGVGCVLIRKRGKLPGPTVSASYSLEYGKAELEIQKDALEPGQKVVIVDDLLATGGTMCAACELLSQLRAEVVECVSLVELTSLKGREKLGPVPFFSLLQYE

SEQ ID NO: 265 - protein, 7 aa, Cricetulus griseus
AELEIQK

SEQ ID NO: 266 - protein, 15 aa, Cricetulus griseus
AELEIQKDALEPGQK

SEQ ID NO: 267 - protein, 10 aa, Cricetulus griseus
IDYIAGLDSR

SEQ ID NO: 268 - protein, 16 aa, Cricetulus griseus
LPGPTVSASYSLEYGK

SEQ ID NO: 269 - protein, 7 aa, Cricetulus griseus
DISPLLK

SEQ ID NO: 270 - protein, 13 aa, Cricetulus griseus
SFPDFPIPGVLFR

SEQ ID NO: 271 - protein, 118 aa, Gallus gallus
MSDKEFMWALKNGDLDEVKDYVAKGEDVNRTLEGGRKPLHYAADCGQLEILEFLLLKGADINAPDKHNITPLLSAVYEGHVSCVKLLLSKGADKTVKGPDGLTAFEATDNQAIKTLLQ

SEQ ID NO: 272 - protein, 9 aa, Cricetulus griseus
GADINAPDK

SEQ ID NO: 273 - protein, 8 aa, Cricetulus griseus
NGDLDEVK

SEQ ID NO: 274 - protein, 13 aa, Cricetulus griseus
NGDLDEVKDYVAK

SEQ ID NO: 275 - protein, 104 aa, Homo sapiens
MAKISSPTETVRCIQSLIAVFQKYAGKDGYNCNLSKTEFLSFMNTELAAFTKNQKDPGVLDRMKKLDVSSDGQLDFPKFLNLIGGLAVACHDSFLKAVPSQKWT

SEQ ID NO: 276 - protein, 7 aa, Cricetulus griseus
DPGVLDR

SEQ ID NO: 277 - protein, 10 aa, Cricetulus griseus
NQKDPGVLDR

SEQ ID NO: 278 - protein, 11 aa, Cricetulus griseus
CIQSLIAVFQK

SEQ ID NO: 279 - protein, 90 aa, Oryctolagus cuniculus
MASPLDQAIGLLIGIFHKYSGKEGDKHTLSKKELKELIQKELTIGSKLQDAEIVKLMDDLDRNKDQEVNFQEYITFLGALAMIYNEALKG

SEQ ID NO: 280 - protein, 7 aa, Cricetulus griseus
ELTIGSK

SEQ ID NO: 281 - protein, 8 aa, Cricetulus griseus
LQDAEIVK

SEQ ID NO: 282 - protein, 98 aa, Rattus norvegicus
MPTETERCIESLIAVFQKYSGKDGNSCHLSKTEFLSFMNTELAAFTKNQKDPGVLDRMMKKLDLNSDGQLDFQEFLNLIGGLAIACHESFLQTSQKRI

SEQ ID NO: 283 - protein, 11 aa, Cricetulus griseus
CIESLIAVFQK

SEQ ID NO: 284 - protein, 7 aa, Cricetulus griseus
DPGVLDR

SEQ ID NO: 285 - protein, 10 aa, Cricetulus griseus
NQKDPGVLDR

SEQ ID NO: 286 - DNA, 2211 bp, Cricetulus griseus
  misc_feature: Qualifier WAN013I8X_at
ggcctgcttcgcctcgtgccctcccgccgccgccccgcagaaatgcttcgactacccacagtccttcgccagatgagaccagtgtcccgggcgctggctcctcatctcactcgggcctatgccaaagatgtaaaatttggtgcagatgctcgagccttaatgcttcaaggtgtagaccttttagcggatgctgtagctgttacaatggggccaawgggaagaacagtgattattgaacagagttggggaagtcccaaagtaacaaaagatggggtcactgttgcaaaggcaattgatctaaaggataaatacaaaaatatcggagctaaacttgktcaagatgttgccaataacacaaatgaagakgctggggatggcrctaccactgctactgytctggcacgttctattgccaaggaaggctttgagaagatcagcaaaggggctaatccagtagaaatccggagaggtgtgatgttggctgttgatgctgtaattgctgaactgaaaaaacaatctaaacctgtgacaacccctgaagaaattgctcaggttgctacaatttctgcgaatggagacaaagacattggaaacatcatctctgatgcaatgaaaaaggttggaaggaaaggcgtcatcacagtgaaggatggaaaaaccctgaatgatgagttagaaattattgaaggcatgaagtttgacagaggatatatttccccgtattttattaacacatcaaaaggtcaaaaatgtgaattccaagatgcctatgttctgttgagtgaaaagaaaatttctagtgtccagtccattgtacctgctcttgaaattgctaatgctcatcgtaagcccttggtcattattgctgaagatgttgatggagaagctctaagcacactggttttgaacaggctaaaagttggtcttcaggtggtagcagtcaaagctccaggttttggggacaataggaagaatcagcttaaagatatggctattgctactggtggtgcggtgtttggagaagaggggctaaatctaaatcttgaagatgttcaagctcatgacttgggaaaagttggagaggtcattgtcaccaaagatgacgccatgcttttgaaaggaaagggtgagaaagctcagattgagaaacgaattcaagaaatcactgagcagctagaaatcacaactagtgaatatgaaaaggaaaaactaaacgaaagactcgctaaactttcagatggagtagcagtgttgaaggttggaggaacaagtgatgttgaagtgaatgagaagaaagacagagttacagatgctctcaatgctacaagagcagctgttgaagaaggcattgttttaggagggggctgtgctctgcttcgatgcatcccagccttggattccttaaagccttctaatgaagatcagaaaataggtatagaaattattaaaagagcactcaaaattcctgcaatgacaattgctaagaatgcaggtgttgaaggatctttgatagttgagaaaattctgcagagttcctcggaaattggttatgatgctatgctcggagattttgtgaacatggtggaaaagggaatcattgatccaacaaaggttgtaagaactgctttactggatgctgctggggtggcctccttgctaactacagcagaagctgtagtgaccgaaattcctaaagaagagaaggaccctggaatgggcgcaatgggtggaatgggagggggtatgggaggtggcatgttctaattcctagaatagtgctttgcccttatcaatgaactgtgacaggaagctcaatgcaggttcctcggtgataacttaagagaagtcacctgaagaaaatgaagagaaggctggctgatcactataaccatcagttactggttcccattgacaatacataatggtttactgctgtcattgtccatgcctacagataatttattttgtatttttgaataaagacatttgtacattcctgatactgggtgcaagagccatataccagtgtcctgctttcaacttaaatcattgaggcagttctactattctgttacagtcaggattctagtgcttgcgtgaccagatgagttcagaagcagcctttctgtggagagtgagaataattgtgtacaaagtagagaagtatccaattatgtgacaacttttgtgtaataaaattttgtttaaaag

SEQ ID NO: 287 - DNA, 600 bp, Cricetulus griseus
  misc_feature: Qualifier WAN008EH7_at
gtcgacccacgcgtccgggcggctcaccggcgtgcatccccgtgggctcaccggcgtgcgacccggcgtggagatgccccgaagtggggtacgagcagactatcgcgccgcgccggtgcccccctacaagtcggatgcacgcacggaggcccttaacagcaagattaaagaacaaaaaattcttgtggatgaactttctaacctgaagaagaatcggaaagtatataagcagcaacagaacagcagcgtatttttacttgcagatggaacagaaatgttttccaaaagcaaaaagacattagatgaactaagaaaacaataccaagcattagaagaaaacacagagacaaccgaagtcaagacgcagtcaccctgatttcacataaccatgtgtggcatttgctgctctgtaggcttctctgatgaacacttcagtaaagaattgaaagaggatttgctgtgcaatcttagacgactgggccccaataacagcaagcagctgttaaaatctggtgttcactatcatcgtttgttttccggtcatgttctttattagaaatgtgttttgactgcccatcctgtggaagatgaaagtggcaa

SEQ ID NO: 288 - DNA, 621 bp, Cricetulus griseus
  misc_feature: Qualifier WAN013HXI_at
ttacctcagtggcagacggctcctggctgacggctggcaggaagcakggcgtcgggatgagtgcaggctcggaccccgtggttatcgtctctgcggcgcggaccgccatargctccttywwtggtgccctgtctactgtgcctgkcsacgacctcgggacaactgtcatcaaagaagtcctgcakagggccaaggtggccccasaagadgtgtccgakgtcatattwggacacgttttgacggcaggttgtgggcagaatcctactcgacaggccagtgtgggtgcakggatcccctactcagttccagcatggakctgccakatgatctgyggctcawgcctcaaggccgtgtgtcttgcagcccagtccatagccataggtgactccakcattgtggtggcaggaggcatggagaacatgagcaaggcccctcactyggctcacctgakagcakgagtcaagatgggtgakatgccgctagctgacagcatactgtgtgatggtcttacagatgcgtttcacaactatcatatgggcatcacagctgagaatgtggccaaaaaatggcaagtgagtagagaagcccaggacaaggttgccgttgtgtcacaaatcaggg

SEQ ID NO: 289 - DNA, 600 bp, Cricetulus griseus
  misc_feature: Qualifier WAN008D37_at
gtcgacccacgcgtccgcggacgcgtgggcggacgcgtgggttcagaaggtacatggtaggaaagggccagaagctgactctgagtggaggagaggcccgccagagaaggagtggagacgaggagagactcgggatgatgagagacctcacaggagagatgaagaccggctgagacgcttgggggatgatgaagagagagaatcttctcttagaccagatgatgatcgaatccccaggcgtggcctggatgatgacagagggcctaggcgtggtcccgatgaagatagattttctcgtagaggaacagatgatgaccgtccatcctggcgtaatgcagatgacgacaggcctcccaggcgaattggtgatgatgataggggaagctggcgtcacacagatgatgacagaccacccagacggggactggatgatgacagaccactgagacggggactggatgatgacagaccactgagacggcgactggatgatgacagaggaagctggcgcacagcagatgatgacagaggacctanacgtgggatggatgatgagcgngggccgagaagaggaggtgctgatgatgaacggtcatcctg

SEQ ID NO: 290 - DNA, 2459 bp, Cricetulus griseus
  misc_feature: Qualifier AF292400_at
gcacgaggcgccttggagccggatccggccccggaaacccgacctgcagacgcggtacctctactgcgtagaggccgtagctggcggaaggagagaggcggccgtcctgtcaacaggcgggggaagccgtgctttcgcggctgcccggtgcgacactttctccggacccagcatgtaggtgccgggcgactgccatgaactccggagccatgaggatccacagtaaaggacatttccagggtggaatccaagtcaaaaatgaaaaaaacagaccatctctgaaatctctgaaaactgataacaggccagaaaaatccaaatgtaagccactttgggaaaaagtattttaccttgacttaccttctgtcaccatatctgaaaaacttcaaaaggacattaaggatctgggagggcgagttgaagaatttctcagcaaagatatcagttatcttgtttcaaataaaaaggaagcaaagtatgcacaaacactgggtcaagtttctcctgtaccaagtccagagtctgcatatactgcagaaaccacctcacctcatcccagccatgatggaagttcctttaagtctccagacagagtatgtttgagccgagggaagttattagctgaaaaagctgtcaaggatcatgattttattcctgctaatagtatattatcaaatgccttaacatggggagtaaagattcttcatattgatgacattagatactacattgaacagaagaagaaagagttatgttcactcaagaaatcaagtacttctgtgagggattcgggaaaaaaagctggcactacaatacaaaaggcgagaacagggagactcaaaaagccttttttaaaggttgaagatgtgaaccggtcttacaggccattctaccttcagctgaccagtgtgccttctataaactatgctacgcacaagccttgcagcccatttgacatagagaagccatccagcgtccaaaagcaagctcagcctaaacccagacctaatacggatggtgataaatgtggtggaaccccagttcaactccaattgaaggaaaagagaaaaaaaggatattgtgaatgttgcttacagaaatacgaagatcttgaaactcaccttttgagtgagaaacacaaaaacttcgcacagagtaaccagtatcaagttgttgatgatattgtatctaagttagtttttgattttgtggaatatgagagagacacacctaaaaagaaaagaataaaatacagcgttggctccttttcttctgtttctgcaaatgtcctgaaaaacactgaaccaaaggagaagctacagttggagcccattttccagaaggacatggtagaaagtaatggacaactgcctgaggagatttttcagtgtgaagatatccagtgtgaagatatccagaaacctgaacaaaggcttgtgttggcttcagaacccatgtcctactcttcaacgggattgaaaggacgtgatgagaaagcagccagcatgttaaatgccagtgaacctgacataaaacagaagtttacacagctacctccatgtaaaaatgaacaggaaggaattcttgatgtttctgaacataaattaattattaatagaaatgatttagaacagagggttggtgactctgtaggtgtgccacggtcatgtgtacaagtttctcatctcagtccagaaaatagtctaccccaaccaaaattaacagcagatactacacatttttcagcaaaagatttacaagaaaaggaccttcattttgtgtttggtcatgattctgatctggtgacattaaatacatcaaaagaacagctaacagtgaaggccggaacgccatcctgtggtcctcagcagcccaatgagtgtgacacggagaatacagataacttgccttgtggtaagatccagcggaaagtgagactgttattagggcagaagaaaaaaaatgtggacccaagtgctgaattggataaaaaaagaactgagtttcttcctatgtgtgaagacagaacttgtgggtcaccagtacagtctctactggacctttttcaaacaagcggggagaaatcagattttttggggtttaccagctacactgaaaacagtgggctatgtgatgttttagatgtttgggaggatgaaaattcaagtagtctgctctcaacatttttctcttccccttcagcttctacattcattggattttagtatttaatgtgtttttcagaagtgacagttttattcttggaatttttataaatacatatataattcttaggagtctgctttttggttttggtttgttctttgccagctttgttgctagaaccatatgtaaatactaagatgatgtttataattttttacagaattgagtagctgttacaaattatacaataaaatcttgtgatttattttacatnnnnnnnnnnnnnnnnnnnn

SEQ ID NO: 291 - DNA, 779 bp, Cricetulus griseus
  misc_feature: Qualifier M80243-rc_at
ggatccgacacccacaccggtggcagaaatgaagacatgcatgtggcatattagccaaaacaaacaaacacctgaatcttaaaatatctcaggcaggccaggccgtggtggcgtacaccacnnnnnnnnnnnnngnnnnnnnnnnnnnncgaaaatctctgagtttnnnnnnnnnnnnnnnnnnnnnnaaagttcttgaacaaccagagctacatagagaaacccatcccaaacaaacaaaaatcaaaacaaatacctcaggcaaaaccatgcaagctggcagcactagggaaaccgaggcagatatggagaactcaagactagtcttaggtatataacaagttcaaggacagcctggaacacgtaacgagttaggaagaccatgctctcctactaaattcaaatcgttttccccccaactccagttcaacccagtaagttcccattggaaccggggcaccagggacttacatggggttgtcatctggctcccagccttccagctccttaaagcagaaaaaacactgggccaagtcaggctcgttctcggtagggcagtggatgaagccggcttccgccatctgaaaatgacagcacagctcgtgagcagaaccaaccgtctggttttgattctgtcactctctggggtcgttccgggccggcctctctggggacggagggaagaggtcgccctgtctggccctgtctaccgggccggggcctgacgcctgacaacagccgtagcgaaggctgggggattagtggggtgccctggtcga

SEQ ID NO: 292 - DNA, 574 bp, Cricetulus griseus
  misc_feature: Qualifier WAN008CYY_at
atacgactcactatagggcgaattgggtaccgggccccccctcgaggtcgacccacgcgtccggccacgcgtccgctttaccagccataaacattttgcaagaacagcctgactctaaaggtcccagcattcctttctcgatttttgatgagtttctttcagacgaaaaggacaaaagtcctgctgcagattctccacaggctctcaaagcccagcgaagaccccttgcagttctcaaaactgcagaaatcattgcctcaaatgaggacatgtctcctgatgtttgtgatgagcttacaggaattgatcctttgggtgaagatgccaccataccaagtttcaggaacgtaaccattggtcctaaccctgaggacacttgttactttgttagagcgcctcggtttgcatctactcctttccatgagataccgtccttgcagggcatcccttccgatcccgagagactgttgcaggaagaggatctggatgggaagaccactgaggttcatcacactgcgctggccactatttataaccagaccctcagcatcaagaagctgagcccaattattga

SEQ ID NO: 293 - DNA, 599 bp, Cricetulus griseus
  misc_feature: Qualifier WAN008CVX_at
gtcgacccacgcgtgcgaacagactgaaagtgctctacagccagaaagccacgcctgggtccagccggaagacttgcagatatattccttcgctaccagacaggatacttgatgcccctgaaatccgaaatgactactacttgaaccttgttgattggagctctggaaatgtactggctgtggcactggacaacagtgtgtacttatggaatgctggttctggtgacatcctgcagctgttgcaaatggagcagcctggggactacatatcctctgtggcctggatcaaagagggcaactacctggctgtgggcaccagtaacgctgaagtgcagctctgggatgtgcagcagcagaaacggcttcggaacatggccagtcactcggctcgagtaagctctctaagttggaacagctatattctgtccagtgggtcacgttctggccacattcaccaccatgatgttcgggtagcagaacaccatgtagccacactgagtggccatagccaggaagtatgtgggttgcgctgggccccatatggacggcatctggcaagcggtggcaatgataacgtggtcaatgtgtggcccagtggt

SEQ ID NO: 294 - DNA, 1373 bp, Cricetulus griseus
  misc_feature: Qualifier WAN013I9R_at
aaatatgacctacggaatcacccagcttccaagtcgtcgggcaagtccgtcaggctcccagcggaagtggctacaggcggggccgagaaaggcgcgatgacgttgcagtgatgcgggcgtagccgtctgtgagcgtttggcgggatttctgaaagtcgcgcgtccctgggcgaagtgcgtgcagacgggttcctgttcaaagccgtgttttccggaaacgcagctgggaaggctaaaatttgctcctgttcttcacagaagaccggacagaggagccggtgacggtggttcttgtggcgtgagcagacccggccgccaaaggagcgcgcggcccacggcggctgcggaccgtaatggctatgcagatgcagcttgaagcaaatgcagatacttcagtggaagaagaaagctttggtccacaacctatttcacggttagagcaatgtggcataagtgccaatgatgtgaagaaattagaagaagctggtttccatacggtggaggctgttgcttatgcaccaaagaaggaactcataaatattaagggaattagtgaagccaaagcagacaaaattctggctgaggccgctaaattagttccaatgggtttcaccactgcaactgaatttcaccaaaggcgttcagaaatcatacagattactactggctccaaagagcttgacaaactgcttcaaggtggaattgagactggatctatcacagagatgtttggagaattccgaactgggaagacacagatctgtcatacattggctgtaacatgccagcttcccattgatcgtggtggaggtgaaggaaaggccatgtacattgacaccgagggtacgtttaggccagaacggctgctagcagtggctgagaggtatggtctgtctggcagcgatgtcctagataatgtagcatatgctcgagggttcaacacagaccaccaaacccagctcctttatcaannnnnnnnnnnnnnnntagaatccagatatgcactgcttattgtagacagtgctactgccctctacagaacagactactcaggtcgaggagagctttcagccaggcaaatgcatttgkmcwsakktctgaggawgctkctsmgmcwkgcwratgmrtttggwgtwkcwgwggwwrtcaccaaccaggtagtagcccaagtggatggagcagccatgttcactgcagatcccaaaaaacccattggaggaaacatcattgcccatgcatcaacaaccaggctgtacctgaggaaaggaagaggggagaccagaatctgcaaagtctatgactctccctgtctccctgaagctgaagccatgtttgccattaatgcagatggagtaggagatgccaaggactga

SEQ ID NO: 295 - DNA, 2352 bp, Cricetulus griseus
  misc_feature: Qualifier WAN013I5T_at
cnnnnnnnnnnnnnnnnnnnngtaggatccgggtaccatgggcggaggaacggctgttggtgtctggccggwggccmgcyagagrcccggggttttgctggtcgccgagcctsttcttcaggaggagccatggcgctcagrgtcactaggaacacgaaacttaacacagaaaataaggccaaggtcagcatgrcaggcgccaagsstgtgcctgtgrcrmttgctgcrgcctccaagcccgggctgagaccragaaccgctctaggrgatattggtaacaaagtcagcgarcaggcacaggcaagastgcctctgaaaaaggaactaaaaacytcagttactggaaaagtttcagctaaaatcccrccaccaaaacctcwggagaaggttcctcctgtgagtgarccagaggtggaacttgctgagmctgaacmtgarcctgaacctgttatggasgaaaagctttctcctgaacctatyttggttgataatccctctccaagcccaatggaaacatctggatgtgcccctgcagaagaatacctgtgycaggctttctctgatgtaattcttgccgtgagcgatgtggatnnnnnnnnnnnnnnnnnnccaaacctctgtagygaatatgtgaaagacatatatgcytatctcagacarctggaggaagarcartcagttagaccaaratayctactgggycgtgaagtcactggaaacatgagagccatcctaattgactggctgatacaggttcaaatgaaattyaggytgytgcargagacyatgtacatgactgtytccattattgaycggttcatgcaggataaytgtgtrcccaagaagatgctgcagctagttggtgtgacwgccatgtttattgcmagcaaatatgaagaaatgtaccctccagaaataggtgacttcgcttttgtgactaayaatacttacactaagcaccaaatcagacaratggaaatgaagattytaagagttctaaacttyagtttgggtcgccctctgcctctgcacttcctccgtagarcstctaagattggagaggttgacgttgaacagcatactttggccaaatacctcatggagctcactwtgttggactatgacatggtssactttgctccttctcannnnnnnnctggagctttttgcttagcactgaaaatycttgacaayggtgaatggacaccraccctgcagcactacctgtcctayactgaagaatcccttctgcctgtcatgcagcacctggctaagaatgtagtcatggtgaaccrtggccttacaaagcacatgactatcaagaayaagtatgcaacatccaagcatgctaagatcagcactctggcacagctgaattgtacactagttcagaatttgtccaaggctgtgkcaaaggcrtaactcaagtggaccacwacactacatcgscaaatgyrattggcaccatgtgccrcctgtacatagtatmcagtgtttacttgctcttcaataaaggytgttttacttttcatagcttacctcatwtgrwwsyyggwtsytctgactctaggataacaagggttgtctaaaacatttaggattatttttgaaactgcttttagtttaacagagaatccaagtaatgtacgtaattgtagcctatatgtttatgtacttccttctttaatgtgtcctggtcatatcttttaagtcatcctgcaaagccctctgcctagctcttgtttaaactgtcaatttaccagcttgtccttagttcccttttctatttcttcaggtggttgctgtccttcactgtgtcttgagctatggactttcagatctgaaccccagttttcttgtgaattatttatttgttaattggcttgggaaatagcatgttttaaaattaaaggtataaaaagaatttgccccttaatctcaatgtcctatataaatgtaatcatgcatatgttgtcaaggagatatggactcatttttttcaacaatattccttttaatgcctatattgcatttcctaagtgtacatttcatactgtatgtaatgtattcagtgatcctttacaatacattcaaattgcattctctcattgcctctaaacaatactgctaaaatgttgttttatgccttgcttggctgtatgcatagtagctttcccagggggtatatgctttaaattctgatgaagccagatgagacttttggttgtgggcactataccaaggtgccatttcctctatctttccactgtcttatgacacttaactggcaaaaatcctgtctctgtttgttttaatttatacatctgatgtcaagttgaataaaatttatta

SEQ ID NO: 296 - DNA, 1403 bp, Cricetulus griseus
  misc_feature: Qualifier WAN013I8J_at
ggcggccgcgcgagatcctgagtckcccaacggcgcyrcggactcrggtggcwgygctgagtccgtmcgtcccagcgcggccctcatggcgctgctccgacgcccgacggtgtccagtgatttgaagamtattgacacaggagttaatcctaaagctaagagccatgtgacyatycggcgggcagttttagaagaaattggaaataaagttagaascmgagcagcyccagtggctaagaaacctcagaacaccaaaataccagttcagcccaccaaagtgacamatgtcaacaaacagccraaacctacagcctctgtgaaacckgtacaratggagacmctggctccmaaggatccttctcctgcccctgaggatgtctccatgaaggaagagaacctctgccaagctttctctgatgctttgctctgcaagatygaggatatagataacgaagactgggagaaccctcagctctgcagygactacgtgaaggacatctaccagtatctcaggcagcttgaggttttgcagtcyatcaacccacayttcttagatggaagagatataaatggacgtatgcgtgccatcctggtggactggctggtccaagtycattccaarkttaggcttytgcargaaactctttacatgtgcattgccatcatggaccggttcctacaggcgcagccggtctnnnnnnnnnnnnnnnnnnnnnnnnnnnnnnnnnnnnnnnnnnnnnnnnnnnnnnncgaggagatgttttctccaaatatcgaagactttgtttatatcacagacaatgcttacaccagttcccaaatccgagaaatggagactctgattttgaaagagctaaaatttgagttgggtcggcctttgccgctccacttcttaaggcgagcctcaaaggctggggaggtggacgtggaacaacacacgctagccaagtatctgatggagctaacgctcatcgactatgacatggtgcactatcacccttctcaggtcgcagcggctgcttcctgcctttctcagaaggtgctgggccagggcaaatggaatttaaagcagcagtattacaccggatacatggagactgaagtactggaagtcatgcagcacatggccaagaacgtggtgaaagtcaacgaaaacctcaccaagttcatcgctgtcaagaacaagtatgccagcagcagactcctgaagatcagcacgatcccacagctgaactccaaaaccatcaaagaccttgcctcccctctgatgggcaggctctaggtggctggctcccgccatgcacatctgattttgtacatactcctgtagtttacttcacaaaacctcttctcagactaatctcctaattgtgtattgaggaaaaaataaagctattgcggcc

SEQ ID NO: 297 - DNA, 1399 bp, Cricetulus griseus
  misc_feature: Qualifier U48852_at
gtagccgggggaacggccggcgcgcttgccggtgggcggaggcgagactccacagcagttctctgccggtcgcccgcgagtgcacccgccatgcacctgccgcccgctgccgcagtcgggctgctnnnnnnnnnnnnnnnnnntcccgcgcgcgtggcctcccggaagccgacaatgtgccagaggtgccgggcgctggtggacaagttcaaccaggggatggccaacacggccaggaagaatttcggcggcggcaacacggcgtgggaggagaagagtctgtccaagtacgaattcagtgagattcggctcctggagattatggagggcctgtgtgacagcaacgactttgaatgcaaccaactcttggaacagcatgaggagcagctagaggcctggtggcagacactgaagaaggagtgccctaacctatttgagtggttctgtgtacacacactgaaagcatgctgtcttccaggcacctatgggccagactgtcaggaatgccagggtgggtctcagaggccttgtagcgggaatggccactgcgacggagatggcagcagacagggcgacgggtcctgccagtgtcacgtaggatacaaggggccgctgtgtatcgactgcatggatggctacttcagcttgctgaggaacgagacccacagcttctgcacagcctgtgatgagtcctgcaagacatgctcaggtccaaccaacaaaggctgtgtggagtgcgaagtgggctggacacgtgtggaggatgcctgtgtggatgttgacgagtgtgcagcagagaccccaccctgcagcaatgtacagtactgtgaaaatgtcaacggctcctacacatgtgaagagtgtgattctacctgtgtgggctgcacaggaaaaggcccagccaattgtaaagagtgtatctctggctacagcaagcagaaaggagagtgtgcagatatagatgaatgctcattagaaacaaaggtgtgtaagaaggaaaatgagaactgctacaatactccagggagctttgtctgcgtgtgtccggaaggtttcgaggaagacagaagatgcttgtgtacagacagcagaaggcgaagtggcagaggaaagtcccacacagccaccctcccatgaggatttgtgacgggcatccaggttcagaagctggactctcacccttttaagttattgagaggacatcctatagaaaatgtggcccatggacatcaaccccatttctccaggaagttttggaggaagaagctgcctgctttgaaacagtagatactcacttggccctttaaaacgctgcatttcttggtggttcttaaacagattcgtatattttgatactgttctttataataaaattgatcattgaaggtcaccaggaaca

SEQ ID NO: 298 - DNA, 600 bp, Cricetulus griseus
  misc_feature: Qualifier WAN0088T7_at
gtcgacccacgcgtccgccagaattggggagaaagtggagaaaggaatgtgtttgaggcgctttctgagctcataattttaacagctagccattgtttacacggaaaggaaattagaagtcaactcaatgagaaggtggcccagctgtatgcagatttggatggaggttttagccatgctgcctggctgttgccaggttggctgcctctgcctatcttcagacgcagggacagagcccatcgggaaatcaagaatattttctacaaagccatcaagaaacgcaggcagtcagaagaaaaagttgatgacattctccaaacgttactagactccacatacaaggatgggaaccctctgacagatgacgaaatagcagggatgctcatcggcctgctactcgcagggcagcacacctcctccaccaccagtgcctggatgggcttcttcttggccagagataaaccacttcaagaaaagtgcttcttagaacagaaagcagtgtgttgcgaggacctgcctcccttaacttatgagcagctgaaggatctgaatttacttgatcgatgcataaaagaaacattaagactgagacctccaata

SEQ ID NO: 299 - DNA, 441 bp, Cricetulus griseus
  misc_feature: Qualifier WAN008E8K_at
gtcgacccacgcgtccggagaatgatgtggacaatgagctcttggactatgaagacgacgaggtggagacagcagcaggtggagatgggacggaagctcctgccaagaaagacgtgaagggctcctacgtctccatccacagctccggctttcgagacttcctgctcaagccagagttgctccgggccatcgtggactgtggctttgagcatccatcagaggtccagcatgaatgcatcccacaggccattctggggatggatgtcctgtgccaggccaagtcaggcatggggaagacagcagtgtttgtcctggccacactgcagcagctggagccaatcactgggcaggtgtctgtgctggtgatgtgtcacactagggagttggcttttcagatcagcaaggaatacgagcgcttctcaaagtacatgccgaatgtca

SEQ ID NO: 300 - DNA, 527 bp, Cricetulus griseus
  misc_feature: Qualifier WAN008EXF_at
gtcgacccacgcgtccgctcttcaaatggcggtgttccgtttttccagcataaaaagccacatggaaaggacaaagaaaacagaggcattaacccagtggagaagtctaaagtggaagagacttcagagtactctgtcacaaaaagcagattgccacttcgagcctcaaatgtgtaattaatctgagggctaaggttttcttttcttaaaaagtaaaacctgaaacttcataatttgaacttttatacagatttcaatgggatagactatgtcaagacatcacagtaactagttgaattgagtcctttttaatcatccttatagtttaagcatgtagtaaatttggtttcatttggaatttatattatgtgtgtttctagcttatataaagtagctcttctgtaatgcgttgttttcttgtatattattaaataccaaatatatggaaattgttcttccaagcttggacttaaatggatgaatgtcatctcccttcctgtcttgctaggcctgagttctgtatgtat

SEQ ID NO: 301 - DNA, 2312 bp, Cricetulus griseus
  misc_feature: Qualifier X83576_at
cgggggtgcggtgctatcccggctccagaccacttcgcggaggctctgctcgggctgcgtcgcgcaggctcgcagaggacgacggccccgggtcgctggccccggaccccttccttcccttgcagcccgtcccggccgagcacagtggacatgcaggcgcagaggccccctttgttggaagtgaagaggaactttgagctgaagacaaccctggtcaaggcctcttccgcccgcctttcagggagcaggctcaagaggggccctgaccagatgaaggaagccctagagcctgcaaagaaacggacacgaggcctgggtgcagtgaccaaaattgacacatcccgctccaaaggaccactcctgagctcactgtcgcagccgcagggccccactgcagctcagaaaggccctaagaagacaggacctcgtggttgcactgccgttggatcagtgctgaagaaccagaagctggctcccactgctcctgcccagaagcctgtcaggaagaggcctggtaagcgtcctgactgggacttaaagggtcagttgtgtgacctcactgaagagctgaaatgctaccgggagaagactcaaaagctggaccaggagaaccaagggcttcaggagcaacttaaagaagcccaggagcaggccgcagccctggggacagagcggaacaccctggaaggggagctggccagtgtgcggacgcaagcggagcagtgccagcagaaactggaggctctgtgtgcccgtgtcttggagctggaggaatggctgggcaccaaggaaaacctgattcaggagctccagaaagagcagctggaattgcaggaagagcggaaggcattggccacccgactggaagagcaagagaggaggttacaggcctcagaagccgctctgctaagcaacgaatcggaggtggtgtgtctgcggcagaagaccgcagcccaggtgaccttactggctgagcaaggtgaccggctccacgggctagagatggagcgccgccgactccacaaccagttgcaggaacttaagggcaatatccgggtattctgccgggtgcgccctgttcttgcaggggaacctactccatcgcctggctttctcctgtttccccatggccctgctggaccctctgaccccccaacccgccttagcctttcacggtctgatgatcggcgttcgaccctgaccagggcgcctgcacccactacgcgccacgatttctcctttgaccgggtgttcccaccaggaagcaaacaggaggaagtgtttgaggagatttccatgctggtccagtcggcactggacggctaccctgtgtgcatctttgcctacggacagaccggcagtggcaagaccttcactatggagggcaggcctgggggagacccccagttggaggggctgatccctcggcgcatgcggcatctcttctctgtggctcaggagatgagtggccagggctggacctacagttttgtggcaagttacgtagagatctacaatgagaccgtccgagacctgctggccactgggacccggaaggggcaaggcgagtgtgagattcgtcgagcaagaccaggcagtgaggagcttactgtcaccaatgcacgctatgttcctgtttcctgtgagagagaggtggaggctctgctccatttggcccaccagaatcgagctgtggcccgcactgcccagaatgagagatcatctcgaagtcacagtgtgttccagctgcagatttctggagagcatgctgctcggggcctgcagtgtgtcgctccccttaaccttgtggacctggctgggagtgagcggctagaccctggtttaaccttaggccctggggaacgtgatcgccttcgggaaacacagtccattaatagcagtctgtctaccctgggactggtcataatggccctgagcaataaggagtcccacgtgccttaccgaaacagcaagcttacctacctgctgcagaactcgctgggtggcagtgccaagatgctcatgtttgtgaacatttcccctctggaagagaatgtctctgagtctctcaattcactacgttttgcttccaaggtgaaccagtgtgttatcggtactgctcaggctaataagaagtgaagatggccagagcctggttccgctgccgctgggttgattgtgtctctctctgtgtgtactgggggggagaggggggtgagagatacgcttttattgggtggacatcatttgtacccaactatcaaataaagaaattttgttgtagannnnnnnnnnnnnnnnnnnnnnnnnnn

SEQ ID NO: 302 - DNA, 600 bp, Cricetulus griseus
  misc_feature: Qualifier WAN008CX4_at
gtcgacccacgcgtgcggtacatgtgagtgcactgacacagacacaggctgtggagggggagattgaactagccaagatgaagaagttcattgcttactgccgagcgaggtgtggccctcggctgtctgcaaaggcagcagagaaactgaagaaccgctatatcatcatgcggagtggggcccgtcagcacgagagggacagtgatcgccgttccagcatccccatcacagtgcggcagctggaggctattgtgcgcattgctgaggcccttagtaagatgaaactgcaaccctttgccaccgaggctgatgtggaggaggcactcangttattccaggtgtgcacactggatgctgctttgtctggcaatctgtcaggggcggagggcttcactacccatgaggaccaagagatgctgagccgtatcgagaagcaactcaagcgccgctttgccattggctcccangtgtctgaacacagcattgttcatgacttcaccaagcagaagtatccagagcatgccatnccgaaaggtgctgcanctcatgctgcgcaggggagaggtccaacaccgcatgcagcgcaaggtgctctaccgg

SEQ ID NO: 303 - DNA, 600 bp, Cricetulus griseus
  misc_feature: Qualifier WAN008E7Y_at
gtcgacccacgcgtccgcccacgcgtccgggctgcatcgcgccgcgcgcttgctctgcgcttccccggagtgaggcttctcggctcgccggctcccgtcgcaggtccccggcagcgatggcgctcaaggactacgcgatcgagaaagaaaaagttagaaaattcctgcaagagttttattatgatgatgaactcggaaagaagcagttcaagtatggaacccagttggttcatctggctcatcgggaacaagtggcattgtacgtggacctagatgatgttgctgaagatgaccctgagctggtcgactcaatttgtgagaatgccaaacgctactcaaagctgtttgctgatgttgtacaagagcttctgcctgagtacaaggagaaggaggtggtgaataaagatgttctggatgtttacattgagcaccgactgatgatggagcagcggagcagagaccctggggcagtccggaaccctcaaaatcaatatccttctgagctgatgcgtagatttgagttgtatttccaaggcccaagtagcagcaagcctcgagtgatccgagaagtgcgggctgattctgtggggaaattggtga

SEQ ID NO: 304 - DNA, 576 bp, Cricetulus griseus
  misc_feature: Qualifier WAN008DWL_at
actgcaggacagagagcgtgcgctcagagctcgagaggacagtctggagcagaaggagcgtgaactttgtattcgagagagactagcagaggacaaactggccagagctgaaagtttggtgaagaactacagcttgctgaaggagcagaggttcctgtgcctggctagtgacccagaccttgatcatccatccttagtaatgaggaagaaggttcatttccgtggggaaagcaaagagaacaccacaaggagtgagaattctgagagccagcttgtcaaatccaagtgcaaggacctgaagaagaggcttcatgctgcccagctgcgggctcaggccctgtcggatattgaaaagaactaccagctgaagagcaggcagatcctgggcatgcgctaggccagcaaggcatggagctgggccagtggtgggtactgccagcccactagagatttgtactcgactgctgtagttttgtgtgtctggttctgagctttgcctttgtgtgcagtgagcatgacgctgggattgtttgtgctctttttcagcagcaagtctacaataaggacatgtctccc

SEQ ID NO: 305 - DNA, 496 bp, Cricetulus griseus
  misc_feature: Qualifier WAN008EML_at
gtcgacccacgcgtccgcgcgccctaaacgctacggcggttactgcgagtgttggcctgcgctgttcgagcacttcccggagcagctgtccagacagctttgagacagctcggcagttctgacctagtaccacccggcgcggagcaacctttgaaaacgcgcgcgcctaaacgctgcgacggttacagcgagtgtcagcgggagctgttcctgcatcttccggcctgggtgctcacacagctttgagccagctcgtcacttcacctttttacttgatcaagggtgtgactgtggacctaaagtgattgctacaatggaaggaatcaataatttcaagacaccaaacaaattatctgaaaaaaggaaatctgtattatgttcgactccatgtgtaactatccctgcctctccatttatgcagaagcttggctttgggactggggtcaatgtttacctaatgaaaagatccccacgtgggttgtctcattctccttga

SEQ ID NO: 306 - DNA, 576 bp, Cricetulus griseus
  misc_feature: Qualifier WAN008ELE_at
tcgacccacgcgtccgggtcagtgggaggcatccgtgcttctctgtataatgctgtcacaactgaagacgttgagaagctggctgccttcatgaagaattttttggagatgcatcagctgtgaacatgactagctatgctctgcctgtgagcagcacgcagagttcagaataacttctggttggctacaaaaacttaacagacatggatttttttcccccaaacacatgcttgttacagattctttttgaaagaataatgactaaacatccagggctctgcagggcggatgagactaccagcttatgaccaccttcattttgacttgacctggaagcagtttcaaagctttcctgttgcttttctaacgaatccagcctggcatgtctctgcccctgtctctaggtggattgcagtcctagccttggactttgttctgctgattccatttagttattttttgggttcacaggagcataccccaaggaggttggggtaccatgttctggatctgacagcagtgagtttgctgggttctgcagctattggttaaagtcaacagtgactcttaagcatt

SEQ ID NO: 307 - DNA, 1001 bp, Cricetulus griseus
  misc_feature: Qualifier WAN013I8E_at
attggtagtcgctccctgcgggtcctcgctgccacttctgcatcgcgagccgggagaccttcgcggccggccctctctcctgtcacagtctctagtatctagtcgtcgcgactccgccassatgttygaggcwcgcctggttcagggctccatcctgaagaaggtgttggaggctctcaaagacctcatcaacgaggcctgctgggacatcagctcgggcggcgtgaacctgcagagcatggactcgtcgcacgtctccttggtgcagctcactctgcgctccgaaggcttcgacacataccgctgcgaccgcaacctagccatgggcgtgaacctcaccagcatgtccaaaatactaaaatgtgctggtaatgaagacatcattacactaagggctgaagataatgccgatactttagcactcgtatttgaagcaccaaatcaagagaaagtttcagactatgaaatgaagttaatggacttggatgttgagcaacttggaatccctgaacaggagtacagctgtgtggtaaagatgccttctggtgaatttgcgcgcatatgccgagaccttagccatattggagatgctgttgtaatatcctgtgcaaaggatggggtgaagttttctgcaagtggagagcttggaaatggaaacattaagttgtcacaaacaagtaatgttgataaagaggaggaagctgtaaccatagaaatgaatgagccagttcagctaacttttgcactaaggtacctgaactttttcacaaaagctactccactgtctcctacagtaacactcagtatgtctacagatgtaccccttgttgtagagtataaaatttctgacatgggacacttaaagtattatttggctcccaagattgaagatgaagaaggatcttaggcgttgctagaatctgagaaaaccaagcctttgagaactgcttctgagatgccagcatgttttgaagtcttttctgtcaccaagtttgtacctg

SEQ ID NO: 308 - DNA, 526 bp, Cricetulus griseus
  misc_feature: Qualifier WAN008E3C_at
agccatctttgcattgttcccgggtcctcctcctcctccgcggcggactccggcagctctctcgccagagtcctcgagctccagttctcgcctccctcgctgcagcgcatcaccggcgtgccccaccatgtcagacgcggccgtggacaccagctccgagatcaccaccaaggacttgaaggagaagaaggaagttgtggaggagacagagaatggaagagacgcacctgccaatggaaatgctaaagaggatggagatgaagatgaggaagctgaggctcctacgggcaagcgggtagctgaagatgatgaggatgatgatgttgacaccaagaagcagaagaccgatgaggatgactagacagcagaaaggaaaagctaaacttaaggccaccgtgacctattcaccctccacttcccgtctcagaatttaaacgtggtcaccttcgagtagaggagcaggccccgcccttagcgggcagtgccacccacagatgacatgcgctctccaccaccccaccaaaac

SEQ ID NO: 309 - DNA, 529 bp, Cricetulus griseus
  misc_feature: Qualifier WAN008EJV_at
gtcgacccacgcgtccgattttatcagatatcagctttgacaagactgatgaatcactggagccaagaactgagacagacaattcaggcacacctcacaataatggaggcatgcgcctacatgactttgtctcaaagacggttataaagcctgaatcttgtgttccctgtggaaagcgcatcaaatttggcaagctgtctctcaaatgtcgagactgccgtttggtctctcatccagaatgtcgggatcgatgtccccttccctgcatttctcccctggtgggaactccagttaagattggagagggaatgttggcagattttgtgtcccagacttctccaatgattcctgctattgttgtcagctgtgtgaatgagatagagcagcgaggcctgactgaggcaggcttatacaggatctcaggctgtgaccgaacagtaaaagagctgaaagagagattcctcaaagtgaaaactgtacccctcctcagcagagtggatgacatccatgccatctgtagcctcctgaa

SEQ ID NO: 310 - DNA, 600 bp, Cricetulus griseus
  misc_feature: Qualifier WAN0088U6_at
gtcgacccacgcgtccgcaacgatttcctttcagcctacagagaccccaggactggagaagagctcatcagaaatgagtactatgttactagagcttcagagcctgtgttctctgctgcaggagtctaaagaagaagccattggggccctgcagagaaaaatctgtgaattgcagactagactgcaggaccaagaggaagagcatcaggaagccctgaaggcaaaggaggcagacatagagaagctgaaccaggccttgtgcttatgctataagaatgaaaaggagctccaggaagtgatacagaagcagaacgagaagatcttagggcagatagacaagagtggtgagctcataagtctcaaggaggaggtgacccagctcacacgtttactgcggcatgcagagacagagacgaaagtgctccaggaagctctggagggccagctagaccccagctgtcagctgatggctactaactggatccaggaaaaagtctctctctcacaggaggtggacaagctgagggccatgttcctggagatgaaaaacgagaaagcaaaactggtgatcaagtatcagagccataggaacattcttga

SEQ ID NO: 311 - DNA, 600 bp, Cricetulus griseus
  misc_feature: Qualifier WAN008CJI_at
gtcgacccacgcgtccgctccaagtccagatccgcgcggaggtccaagtccaagtcgtcgtccgtctccagatcccgctcgagatccagatccaggtcgagatccagaagtcctccccccgtgtccaagagggaatccaagtcccggtcgcgctcgaagagcccccccaagtctccagaggaagaaggcgcggtgtcctcttaagaaaatgatggatcgacaagcagcttaacagaggactttgagggaaaggaccagaaactcagtccatcggtccatggaagagtccttggaacaagcaactggctattgaaaaggttattttgtaacatttgtctaactttttacttgttttaatctgcctcaggtggcaaacttcattttatgtgccattttgttgctgttattcaaatttcttgtaatttagtgaggtgagcgacttcagatttcattattggattggatatttgaggtaaaatttcatttttgtttatagtgctgactttttttgtttgaaattaaacagattggtaacctaaaaaaaaaaaaaagggggggcccgccaccgggggggaccctccacctttggttcccttttgg

SEQ ID NO: 312 - DNA, 5996 bp, Cricetulus griseus
  misc_feature: Qualifier WAN013IAD_at
gaattcccattcctctagctgcctacataaggactaagggaggatttctgtaattttaacgaatctgagataaagaaagaacatccgtttctacatcattttattcaggcggagagctgagaagagctgaggaaacggctttgcacttgcaaaacgccttctctcctcagagttttcggatcacatgggcctttctcattggccagatttcctgtaagtctctgctacaggaatagactattggtctatcctgaagactctcccgcctcctttacctaattggttcattcgaacaggccgccttttcaatgggtatgaataaaagaccgtccacgattggttactctaaacagagctaatgagagcgactcggtgattggttcctctgatttgggcgggctcggcgaaggttcaaatgaaactagggaagccatcctaaccgacgtgcgtctgtggaggagcgctctggtcgcgggtcgcctcgcgtttcccactgatttagtcgctttcgggactcacgagcccctacaggaccgtcaccatggagttgtcgccgctgcagsctryarmtgmrratrkgcmmrkgarswaraagaaaaatgaagatgccaagaaaaggctgtctatygaaagaatctatcagaaaaaaacacaattggaacatatattgctccgcccagatacctacattggctctgtggaattagttacccagcaaatgtgggtttacgatgaagatgttggcattaactacagggaagtaacttttgttcctggtttgtataaaatctttgatgagattctagttaatgctgcagacaacaaacaaagggacccaaaaatgtcttgtattagagtcacaattgatccagaaaataatttrattagtatatggaataatggaaaaggaattcctgttgttgaacacaaagttgagaaaatgtatgtcccagctctcatatttggacagctcttaacctctagtaactatgatgatgatgaaaagaaagtaacaggtggtcgaaatggttatggagccaaactgtgtaacatattcagcaccagatttactgtggaaactgccagtaaagaatacaagaaaatgttcaaacagacatggatggataacatggggagagctggtgacatggaactcaagccctttaatggagaagattatacatgtattaccttccagcctgatttatcaaagtttaagatgcaaagccttgacaaagatattgttgcactgatggttagaagggcatatgatattgctggttccactaaagatgtcaaagttttccttaatggaaataagctgccagtaaaagggttccgcagctatgtggatatgtatttgaaggataaattagatgaaactggtaacgcactgaaagtagtacatgaacaagtaaacccaaggtgggaagtgtgcttaacaatgagtgaaaaaggctttcaacaaattagctttgtcaatagcattgctacttctaagggtggcagacatgttgattatgtagctgatcagattgtgagtaaacttgttgatgtggtgaagaagaagaacaagggtggtgttgcagtgaaagcccaccaggtgaaaaatcacatgtggatttttgtaaatgccttgattgaaaacccaasctttgactctcagacaaaagaaaacatgactttacaggccaagagctttggatcaacatgtcaattaagtgaaaaattcatcaaagctgcaattggctgtggtattgtcgaaagcatactaaactgggtgaagtttaaggcccaaatccagttaaacaagaaatgttcagctgtaaaacataacagaatcaagggaatccccaaacttgatgatgccaacgatgcagggagtcgaaactccactgaatgtacacttatcctaactgagggagactcagccaaaacactggcagtttcaggtcttggtgttgttggaagagataaatatggggtatttcctcttrgaggaaaaatactcaatgtgcgagaagcttctcataaacagatcatggaaaatgctgaaattaacaatatcatcaagattgtgggtcttcagtataagaaaaactatgaagatgaagattcattgaaaactcttcgttatgggaagataatgattatgacagatcaggatcaagatggttcccatatcaaaggcttgctgatcaattttatccatcacaactggccctctcttctgcgtcatcgttttctagaggaatttattactcccattgtaaaggtgtctaaaaacaagcaagaattggcattctatagtcttcctgagtttgaagaatggaagagttctactccaaatcataaaaaatggaaagtcaaatattacaaaggtttgggcaccagcacatcaaaggaagctaaagaatattttgcagatatgaaaagacatcgtattcagttcaaatactctggacctgaagatgatgctgcaatcagcctggcctttagtaagaaacaggtcgatgatcgaaaggaatggttgactcatttcatggaagatagacggcagcggaagttacttggccttcctgaggattatttgtatggacaaaccactacttacctgacgtataatgacttcataaacaaggaacttattctgttctcaaattctgataatgaaagatctatcccatctatggtagatggtttgaaaccaggtcagagaaaggttttgtttacttgtttcaaacggaatgacaagcgagaagtgaaggttgcccagttagctgggtcagtggsagaaatgtcctcttatcaccatggtgagatgtcactgatgatgaccattatcaatttggctcagaattttgtgggtagtaataatttgaaccttttgcagcccattggtcagtttggtaccaggctgcatggtggcaaggattcagctagtcctcgatacatctttactatgctcagccctttgactcggttgttatttccaccaaaagatgatcacacattaaagtttttatatgatgacaatcagcgtgttgaacctgaatggtacattcctattattcccatggtgctgataaatggcgctgagggaattggtaccgggtggtcctgcaaaayccccaactttgatatccgtgaagtcgtaaataatatcagacgactgttggatggagaagagcctctgcccatgctcccaagttacaaaaatttcaaaggtactattgaagagctggcttctaatcagtatgtgattaatggagaagtagctattctaaattccacaaccattgaaatctcagagcttcccatcagaacgtggactcagacttacaaagaacaggttctagaacccatgttgaatggtactgagaagacaccccctcttataacagactatagggaataycacacagacaccactgtcaagtttgtcataaagatgactgaagaaaaattagcagaggcagagagagtgggactacannnnnnnnnnnnnnnnnngactagtctcacttgcaactctatggtgctttttgaccatgtaggttgcttaaagaaatatgacactgtgttggatattctaaaagacttctttgagctyaggcttaaatattatggattaagaaaagaatggcttctgggaatgcttggtgcagaatctgctaaactgaataatcaagctcgttttatattagagaaaatagatggcaaaataatcattgaaaataaacctaaaaaagagctaattaaagttctaattcagagaggatatgattcagatcctgtaaaagcctggaaagaagctcagcaaaaggtcccagatgaagaagaaaatgaagaaagtgacaatgaaaacagtgactctgtagcagaatctggaccgactttcaattaccttcttgatatgcccctttggtatctaaccaaagaaaagaaagatgaactgtgcaaacaaagaaatgaaaaggaacaagagctcaacacattaaagaacaagagtccatcagatttgtggaaggaggatttggcagtttttattgaagaactggaggttgttgaagccaaggaaaagcaagatgaacaagtaggacttcctggaaaaggggggaaagcaaaagggaagaaagcacaaatgtctgaagtcttgccttctcctcayggaaaaagagttattccccaagtgaccatggagatgaaagcagaagcagagaagaaaattaggaagaaaattaagagtgaaaacgttgagggcactcctactgagaatggtctggaactaggaagcttaaaacaaagaatagagaagaagcagaagaaagagccaggtgccatgacaaagaagcagactacattggcttttaagccaatcaaaaaagggaaaaagagaaacccctggtctgattctgaatcagatatgagcagtaatgaaagtaatgttgatgtccctccacgagaaaaggayccacgaagagctgcaacaaaagccaaattcacgatggatttggattcagatgaagatttctcaggttctgatggaaaggatgaagatgaagattttttcccactagatactactccacctaagaccaaaattccccaaaaaaatactaagaaagcactgaagccacaaaagagtgccatgtctggtgaccctgaaagtgatgaaaaggacagtgtgccagcttctccaggccctcctgctgctgacttgccagctgacactgaacagttaaagccatcttccaaacagactgtggcagtgaagaagacagccaccaaaagccagtcttcaacatccactgctggtaccaaaaagagagctgtaccaaaaggaagcaaatcrgattcagccttgaatgctcatggccctgaaaagcctgtgcctgccaaagccaagaacagccgcaaaaggaagcagtcctcttctgatgattctgactctgattttgagaaagtagtctccaaagttgctgcaagcaagaaatccaagggagagaatcaggatttccgagtggacttagatgaaactatggttcctcgagcaaaatctggacgagcaaagaagcctatcaaatacctggaggagtcagatgatgatgatcttttctgatggggagagtattgttttagcaatggttttactgagccnnnnnnnnnnnnnnnttatttgaagttcttctctaaccccctctgaatttagtttggggaagactttttataagactatcctacgtggccatcagctttatattctatatactgtctcagtgatgaccatctcatgacactgttttcttctctgatttatctatgttttgtgtctatttctttggtctctaaaatcttgcttgagattcttttggattccacataattatatgctcacttcagcatgacagaatttattgatcatatgttaagctaaaatgggtatgttttcaaaagtgtgcctctacctcctttcctatgtctactaagatatgaatgaggtagcaaggtgtttctttttcagttgtgtaggtagaattccatcacatagaactagacagttctagtatggatacagtgttcaacctatgatttgatgcatttttgtttcagccataaaaaattactcataactcttttaaacatcatcagagkctgaagaaagtcatcttgactgtgtctccatgatctcagtacatgtagcaagaaaaagagagttagtctggttatgtggcatcctgactttccttatgatacctttcgaacatgagatcacagggaggagacagaagtgctcagacttacatctgcatgctcaattgtgaaatgtggtccattgcaagtgtacagagttccaacttgtgaacgccaaaagcattctgtgctrttgtatgttacttaagtgctgttatcaatgtgttttgtaaatatttaatatgtctttctatrtagcttattccaacaattttgtacattaataaatatttaaacattgt

SEQ ID NO: 313 - DNA, 568 bp, Cricetulus griseus
  misc_feature: Qualifier WAN008DMP_at
atacgactcactatagggcgaattgggtaccgggcccccccntcgggtcgacccacgcgtgcgcccacgcgtccgatcgtcaccatggacgccgagatggagttcaactgcgagatgaacaagagaactgggcaacccatgatccacatctatctggataaggagacaggaaagcctaaaggtgatgcaacagtgtcctatgaagatgcacgaactgccaaggctgctgtggaatggtttgatggaaaagattttcaaggaagcaaacttaaagtttctcttgcccgaaagaagcctccaatgaacagcatgcggggtggcatgccacctcgagagggcagagggatgccaccaccacttcgtggaggtcctggtggcccatgtggtcctggaggacccatgggacgcatgggaggccgtggatgagacagaggaggcttccccccacggggaccccgaggctcccgagggaacccctctggaggaggaaatgtccagcaccgatctggagactggcagtgtcccaatccgggctgcggaaaccagatctccgcctggagaacagaat

SEQ ID NO: 314 - DNA, 496 bp, Cricetulus griseus
  misc_feature: Qualifier WAN008EL3_at
gtcgacccacgcgtccgtggcggacgacgtagatcagcaacagactaccaataccgtagaggagcccctggatctcatcaggctcagtttggatgagcgaatttatgtgaagatgagaaacgaccgagagcttcgaggcagattacatgcttatgatcaacatttaaatatgatcctgggagacgtagaagaaactgtgacgacgatagagattgatgaagagacatatgaagagatatataaatcaacaaaacggaacatccccatgctcttcgtccggggagatggtgttgttctagttgcccctccgttgagagtcggctgagacagaaaatggtcctgtgtaggaaaatggagactttgcctcttgagatgtacagaacactcacagagagaagctcatgtgtattctgatagtcagaaatgacccgaggatccctccaccctcaaagaagttcatttgcgagtaacttacaacctctccagctaaaatggc

SEQ ID NO: 315 - DNA, 1000 bp, Mesocricetus auratus
  misc_feature: Qualifier U10249_at
cccgcggggatgtcgtacaagccgaacttgaccgcgcacatgcccgccgccgccctcaacgccggaagtgtccactcaccatctactagcatggcgacatcctcccaatatcgccagctgctgagtgactacggaccaccgtcactaggctacacccagggaactggaaatagccaagtgcctcagagtaaatatgcagaactgctggccatcattgaagagttggggaaagagatcagacccacttatgcgggaagcaagagcgccatggaaagactaaaacgaggcatcattcatgcccgaagcctggttcgggagtgcttggctgaaacggaacgtaatgccaggtcctagcccctggccagtctgaaggcccatcttgctaccccttggagatgagaggctttgttcaaaatggcagttttcctgccatggtcattaagctctgaacccacattcaaaagactgagaagacattttgcagttactgatgatgtgcattttaagtagttaggaacaatccaagcattgattttaaagatgtttgtgaagccacttcacagcaagctattgttttcccccaaataccagtgtccccttaatctccctttggatacatttgccatttgcatcaccccagttgacttcctttccaggaggtcacctgcctctgaggacctgagtgcaaaccacagcacgtttgtttagtagctggcccgcctgtgtacaccctgcttcacggagcttctctgcttaagtgtttgcatgactgagtgctttgaagtcaatcttaaaaatgcacaagttacagatacagaagaagagcgatctccaacctaccaagcgccctgcaaatgtccatcctgagactgtagttctcggttccatgtttactgtgagatgatcacaacatctggaagaaaatgactgaaactgttgcatctttgtatttattacttgatgtaataaagcttattttcattaacagtttgtattaaga

SEQ ID NO: 316 - DNA, 600 bp, Cricetulus griseus
  misc_feature: Qualifier WAN008E5I_at
gtcgacccacgcgtccggttcatagttgaagaaaaatggcaagagatcatgtcctcagaccaggtggaattacttaaaatttaaagcctgaaaattccaattttggcggggggtggatggaaaaggaacccaattttcttatggacagatatttttaacataatggcacaaagtcttagaatattattatgtgccccatgttccctgttcttcgtttctgcattttcttcacttgcagacagacttggctctcgatgaacttttatcacaaattgaaatatatttttttcaactgccagtcaaggctgagaggctcaaccacctcaacattggatacatcacttgccaatgtacataccttgttatatgcagacatgtatttcttacgtacactgtacttctctgtgcaattgtaaacagaaattgcaatatggatgtttctttgtattataaatttttcccgctcttaatgaaaaattactgtttaattgacatactcaggataacagagaatggtggtgttcagtggtccatgattctgtaatgcttcacacaggcagttttgaaatgagaatcattttcccttttcttatgatggag

SEQ ID NO: 317 - DNA, 2855 bp, Cricetulus griseus
  misc_feature: Qualifier WAN013I73_at
ccgcgggcgcccggccgcgcgggcagcctcgggacaatggggccgcagcgcttgctgctcgtcgccgctggcctcagcctgtgcggtcccttgctgtcttcccgtgtccccgtgcgccagccagaatctgagatgacagatgccactgtgaacccccgctcattttttcttaggaatcccggtgaaaatacatttgaactgattcccctgggggatgaggaggagaaaaatgaaagcaccttgccagagggcagggcaatctacttaaataaaagccactctcctgcaccacttgctcccttcatctctgaagatgcctcagggtatctgaccagcccctggctgaggctcttcatcccctcggtttacacctttgtgtttgtcgtcagccttcctctgaacatcctggccatcgcagtgtttkkcttgaagatgaaggtcaaaaagccggctgtggtatacatgctgcacctggccatggctgacgtgctgttcgtgtccgtgctycccctgaaaatcagctactacttttccggcagtgattggcagtttgggtctgggatgtgtcgcttcgccactgcagcgttttattgtaacatgtacgcctccatcatgctcatgactgtcataagcattgaccggttcctggcagtcgtgtaccccatccagtccctgtcctggcgcactctgggcagagctaacttcacttgcctggtcatttgggtgatggccatcatgggggtggtgcctcttctcctcaaggagcagaccacccgggttccagggctcaacatcaccacctgccaygatgtcctcaatgagaccctgctgcagggcttttactcctactacttctcggccttctccgctgtcttctttcttgtgccattgatcatttccacgatatgctacatgtccatcattcggtgtctcagctcctcctcagttgccaaccgcagcaagaagtcccgggctttgttcctgtccgctgctgtgttctgcgtcttcatcgtctgctttggacccaccaacgtcctcctgatcatgcactacctgctcctgtctgacagccctgccacagagaaggcctacttcgcttacctcctctgcgtctgtgtgagcagcgtgagctgctgcatcgatcccttgatttactactacgcctcctccgagtgccagaggmmcctctacggcatcttgtgctgcaaagaaagctccgaccccaacagttacaacagcaccggccagctgatgccgagtaaaatggatacctgctctagtcacctgaataacagcatatacaaaaagctattagcttaggggaggattgctagaaggtcacatagaaaaggttggaaaagtggacagcccgggatcctggcaagaactatatttacttcacaagccatgactgacttgcatgtctacttcttatwagtgctatcaagcataattatcagaaaggtaatgggaaccagagagagatttccagtgctgcggtagcaaccgtcacttcttgtatatctaggtgacttaattatataggtggtgtgcgcacacatatttgcaatgcagtatgaaatcaatgctttgacactttcttgtttattccctagcaattactatggaaataattggattctctgagttataataaacaaagtctgttaacaytgaacagctgaaggtatctaatgatagggaaggagtccatagtttakacttcacacagcttttgcccatatgtatttttttcagttatttgatggtaatgtttaaagwatagaagggtgaaacagtattatctgtataggggaagatctagtgcttttcatcttgaattaccaaagccgttggaaagcctattttgatatggggtatcatttttgcaatttacatactgaatacatggaccaagactgagcataaaactcaccaggatcataagaaaccttacaaagcagccaagggggcctctgacagcagatgcatacttaaccagctgtgcctcccasagagtggtgatggggaccaccaggcccactccttcctccaaggcwtcccattagctatgagttgactgtatctggattgaagctggcaagataggatatgacatccasggagggaatagccacaaaagtcgtctgcacatctgaggagcagagaaggtgtgtttatatccagtagctgtcctgcaaggctggctcttgcacagacacacccatgtgccctgggtcactctggtggatactgggcctgcagactgaccactgcaggagattaactgagtcttgcctttactctagcaaagcagatagctgacatggccaatgtgatgtatacactgcaaatacaccattgtacatagaagtgtgtcagccacacatcgccaagcatatttcacataagcaaggcctaacagctaaacagctttggagatctgagtttctgcatcagtagctgtaggttaggataaaaacacagtttaagatgtatatttttaagaaacaaacccttaagtctacaataattagacactatttatttacaaatgttttattaaaaattgcccaaatcagacaggcattgaggcacatgcctttaagnnnnnnnnnnnnnnnnnnnnnntaggcagatctctgtgcattcaggtcagcctggtctacatgatgacttctaggtcatctagggctatatagtgagaccttggcttaaaacaataatgacatnnnnnnnnnnnnnnnnnnnnnnnnnnnnnnnnaaaacccacatttttagaaagcaagaaaacacaaaaagttacctgaatccagttttaagaaatacataggtcagtctggttaaaaaaaaaa

SEQ ID NO: 318 - DNA, 1137 bp, Cricetulus griseus
  misc_feature: Qualifier AF061256_at
gggcggcgacggtttcctggtggccgcgcgctgctctgtgagcggcgggtggcagacggacctgggccctcaccccagacgcaccgcggatctggcatggctcacctgatgacaatgcagttgctgctcctgctgatatgggtatctgagtgtgcccaatcaagagctactcgggccagaactgaactgctcaatgtttgcatggatgcaaagcaccacaaagaaaagccaggccctgaggacaatttacacaaccagtgcagtccctggaagaagaattcctgctgttccaccaacacaagccaggaagcccacgaggacatttcctacctgtaccgattcaactgggaccactgtggaaagatgacattggaatgcaagcgacactttatccaggatacctgtctctatgagtgttctcctaacttgggaccctggattcagcaggtggaccagagctggcgaaaagagcgaatccttgatgttcctctgtgcaaagaggactgtcagcgatggtgggaggactgccgcacctctttcacctgcaagagcaactggcacaaggggtggaactggacctcggggtataaccagtgccctgtgggagcctcctgtcgccacttcgacttctatttccctacacctgctgctctgtgtgaggaaatctggagtcactcctacaaactcagtaactacagccgagggagtggccgctgtatccagatgtggttcgacccagcccaaggcaaccccaacgaggaagtggcaaggttctatgctgaggccatgagtggagctgggcttcacggggcctggccactaatgtgcagcctgtctttagtgctgctctgggtgttcagccgagttcctttaaccttctgatccccaggaactccctgccgggcttagactcccagctcccaacctcctttgtggtggggcctctgacaggcattcaatatctctcttatgaattatttgggtgtgaatgggaatataattattttgcatcctacttaccactgattgaagttgtttaaacttggttagttccctgctctaacacttactgtgggcaagttaaataaacttaattttcctgtgctgttccacnnnnnnnnnnnnnnnnnnnnnnnn

SEQ ID NO: 319 - DNA, 542 bp, Cricetulus griseus
  misc_feature: Qualifier WAN008EMJ_at
gtcgacccacgcgtccggtgatgcgtacgagccaggcagagggttgaaatgaagaccagccaaggaaatcacaccgttagtcaccctgcatggacatggtcaaattactatccacttgcgcttcctggtctttgtcatcatccagcatatggattttagactaacttgacgttaagatgttttgttttgtttttcctgctccctaagccttctcattgagtcaccccttctcagtactgcagaatcctcctcccaaattaattttctcctttcagcatcgtgatagggatgtgtttgtgacacattgtcattcatagcctgtgctcctttgtgatgacgtccctaaattctgtgatcacatcacgacttgtgttatgagtagagaggaatgggaccccaaaattggacactagaaattggtgggtggtgctcataactgaaaacacttagcttattgaagtgcctctatttacatgttctttagttataatatgtatttttctaacagaaatacacgtctgtaattgatgtttattatactt

SEQ ID NO: 320 - DNA, 408 bp, Cricetulus griseus
  misc_feature: Qualifier M96676_at
atggcctgtggtctggtcgcaagcaacctgaatctcaaacctggggagtgtctcaaagttcggggcgaggtggcccctgacgccaagagctttgtgctgaacctggggaaagacagcaacaacctgtgcctgcacttcaacccccgtttcaacgcccatggggacgccaacactatcgtgtgcaacagcaaggataacggagcctgggggactgagcaccgggagcctgccttccccttccagcctggaagcactgtggaggtatgcatcacctttgaccaggctgacctgaccatcaagctgccagatgggcatgagttcaagttccctaaccgtctcaacatggaggccatcaactacatggcggcagacggtgacttcaagattaagtgcgtggcctttgagtga

SEQ ID NO: 321 - DNA, 524 bp, Cricetulus griseus
  misc_feature: Qualifier WAN008EKF_at
gtcgacccacgcgtccgtgcagaccttgtatgaccacttctggtgggaccgactatggctgcctgtgaacttaacctgggctgatctagaagacagagatggacgtgtctatgccaaagcctcagacctctacatcacacttcccctggccttggtctttcttgtcattcgatacttctttgagctttatgtggctacacccctggctgccctcctgaatgttaaggagaaaacccgactacgagcacctcccaatgtcaccttagaacatttctacctgaccaacggcaagcagcccaagcaggtggaggttgagcttttgtctcggcagagtgggctctctggccgccagatagaacgctggttccgccgccgccgcaaccaggacaggcccagccttctcaagaagttccgagaagccagttggagattcacattttacctgattgcctttgttgcgggcatggctgtcattgtggataaaccctggttctatgacttgaggaaagtttgggagggctatc

SEQ ID NO: 322 - DNA, 2449 bp, Mesocricetus auratus
  misc_feature: Qualifier U21937_at
tgaatctgcggctttcatgaagtctccctacaaggtccttcactttgctcctaaacgtggatttttttttcttccccctgccattctagtctccccgcctcttccttgcagccggactccctcctccggtccctgcccacacgcgcactcctctgccccgcgttggccccagcgcccagccctccagccagagggagccagtcaccagacggcagcacctggctggagaggttgggcgggccgcgagggtggggatccgcgggaaccggcgagccgggagctggagcagaagctggaccaagcgcgagcaggagaatggagtctcccgacagcctgccgaggctgatgtgaaattggaccatctgcttccagttggttctgtttcctcttcttcttgtattttcttccctcgccatccacagtggagtgaattatcagatcttgctccgctcccagaaagacgaccaccatggagtgagcccaccctgtccgccacaaggaaaagcacagagaagacatgacgatggccaagttaactgaatccatgacgaatgtcctggaaggcgattccatggaccaggacgtggaaagcccagtggccattcaccagccaaagttgcctaagcaggccagggacgaccttccgagacacatcagccgagacaggacaaagaggaaaatccagaggtatgtgaggaaggatgggaagtgcaacgtccaccatggcaatgtgagggagacctaccggtacctgacagacatcctcaccaccctcgtggacctaaagtggagattcaacctgctcatcttcgtcatggtctacacggtgacgtggctcttctttgggatgatctggtggctaattgcatacatccggggagatatggaccacgtagaggacccctcatggactccctgcgtcaccaacctcaatgggtttgtctctgcttttttattctcaatagagacagagaccaccatcggttatggctaccgggtcatcacggacaaatgccccgagggaattattctcctcttaatccagtccgtgttggggtccattgtcaatgcattcatggtaggatgtatgtttgtgaaaatatcccaacccaagaagagggcagagaccctggtcttttccacccacgcagtgatctccatgcgggatgggaaactgtgcctgatgttccgggttggggacctgaggaattcccacatcgtggaggcttccatcagagccaagttgatcaaatccaaacagacttcagagggggagtttatccccctcaaccagacagatatcaacgtagggtactacaccggagatgaccgtctctttttagtgtcaccattgatcattagccatgaaattaaccaacagagtcccttctgggagatctccaaagcccagctacccaaagaggaactggagattgtggtcatcctggagggaatggtggaagccacaggaatgacgtgccaagcccggagctcctacatcaccagtgagatcctgtggggttaccgcttcactccggtcctgacactggaagacgggttctacgaagtcgactacaacagcttccacgagacctatgagaccagcaccccgtcccttagtgccaaagagctagctgagctggctaacagggcagagctgcccctgagttggtctgtgtccagcaaactgaaccagcacgcggaactggagacagaggaggaagagaagaacccggaagagcagacggagaggaatggtgacgtggcaaacctagagaatgaatccaaagtgtaggccctgctgggtcggcctctccctccctctcagacgtgacccttccttgtctctctcattccccgcccccttccgtcgctctctttggctctgatcccggtttgtttacgtttaattttggcatttccagaaagcaaatcctcgaggtgtaaaatgcctacctgccctctctcggatgttcaggtggacaagcagacagggatttggttgcggtacaagtacctttcacttgtggcccaagcctggcctctctcctctcccttcaccccccccaggagagacttagacgcttacccccacccctgcactgtccaatgcaagtcactcaaaagagctgtcaaagggtttttacccagtgtcacaaaggtcacctgttcaccatggccataggtcacacagaacatgtcccttcagctttattgagcctggtccatctcacctagcacagggcaggcgcttgcccgcacagacgctgagggcgcaggacccagctgagctaaggttagaccactgtacatagatctgccccgtggaattactctcctttggcactcagtaatagaacatgacatgcagtggggcggagctcctggcaatgtacatagatgcatcattcctgtagctttagacgtgcaactttagagacaagaagca

SEQ ID NO: 323 - DNA, 4278 bp, Cricetulus griseus
  misc_feature: Qualifier WAN013I93_at
gaattcccccggaccggcgcgagcgcagcgcgagcgggagctgcggcggactgcggggcggccccggtcccggccccctccgtccccggcctgccctgccctccggcacccctgctcgccgcctcccgggccatggggaacactgcgatcgccaagaaaggcagcgaggtggagagcgtgaaagaatttctagccaaagccaaagaagactttctgaggaaatgggagaaccctcccccgagtaatgctgggcttgaagattttgaaaggaaaaaaacccttggaacgggttctttcgggagagtcatgctggtgaagcataaagccactgagcagtactatgccatgaagatcttagacaagcagaaggtggttaagctgaagcaaatagaacatactttgaatgagaaaagaatactacaggcggtggagtttccgtttcttgtccgactggagtattcttttaaggataattctaatttatacatggttatggaatatgttcctgggggtgaaatgttttctcatctgagaagaattggaaggttcagtgagccacatgctcgattctacgcggctcagattgtgctcacgtttgagtacctccactccctggacctcatctacagagatctcaagcctgagaacctcttaattgaccaccagggttacatccaggtcacagacttcgggtttgccaagagagtgaagggcaggacctggacactgtgtggcaccccagagtacctggcccctgagatcatcctcagcaagggctacaataaggcagtggactggtgggcactgggagtgctcatctatgagatggccgctggctaccccccattctttgcagaccagcctattcagatttatgagaagatcgtttctggaaaggtccggttcccctcccacttcagctctgacctcaaggacctcnnnnnnnnnnnnnnnnnnnnnnnnnnnnnnnnnnnnttcggaaacctgaagaatggcgtgagtgacataaagacccacaagtggtttgccacgactgactggattgctatttaccagagaaaggttgaggctccattcataccaaagttcagaggctctggggataccagcaacttcgatgactatgaagaagaagaaatccgtgtctctataacagaaaaatgtggaaaagaattttgtgaattttagggagggaaaagatggcctcgagctcacactagtctttgcacgctgtggaggtggaaggtggagccgagccctgctcattgaagcagttaccaagttccttcattccagcaactgagtgaggtccgtatcgcatcatcagtgtgcactctgcaaccacctgtgtaccagggcaccgtccgcacgcattgtccgtgccatgacacagtgtgcaccactttcctactgttgagttgtttttctccaccctcctacattcttttcattatcttctctgaccagtactccactttattttctccgtgtttcaggtggcagtgttatggctgtgtgatatttaaaaggaaagctacgtgttgcttcttgtagttttgagcgatatttttgctgaccagtggcttgaagataaactctctaatgattatttttattttgagtagctcggactcagttttgccaaaactcttataatttttgaagatggaatgtcttatcaccaagggaatatgcataagtgaagataataacttttcaggagtcactgatgtggcaataaattcagagtggctagcctagcagtatggactcctggaccagaggcttcatttccctttgccttttaatattcttctctctgtgagaaattaagtgaccagacnnnnnnnnnnnnnnnnnnnagaagtgaggggcgtggctccttgcccggccgtctccctggtaccaacatctctgtgggttagaagggcattccgaggtttctaagccttcactcttccatgagggttttagccagtatttaggagacatgactctaaagctatgcaacgtgaaatgtctcagacagtcctcatcggcagtgtcttaagggaaacatgcgaactaaaggaacacagatttggatagatgctaagtcgagggaagccttgatccctgcttacagcgtagagctactgtcctttcagggaagtggtctgtttgctttggttcataacacgtaaggacaagaagttgctgttgcttactgttgtgacgtattaagatgagggcaccgtggcaacttcagattttatcccctgctttggcagagggggggggaggcaaagcttaagaaactgccacatttgtagtttaattttgaggtgtgatattatctttagatacatcggaatttctaacctctttttttctttgtagacagagtatatgatcatgcagatacagttttaatatttgtgtttccttaatttttttatactttttgccaattgacttcacaatgttgccatcatgaggaaatttcaagcactcttgcacatttagttcagtgttctttgtgaataaatggcttaacttttttaactattttttccattgcttttattttcctgtctcattttctctctgtttcagtgacctaccttaaatgatcaactcacccaaatggtttaaaatgttccattttgtgagcaatctacaagcatataaggttgttttaaatcaaacaagccctaatgtgtatatatatatgtgtgtgtgnnnnnnnnnnnnnnnnnnnnnnnnnnnnnnnnnnnnnnnnnnaaaccttgatctgcttccttactggtgcaggtaggtcattgacgccacccctgttcatatgcacccacccctcactaaacccttgaagacactgagaagtcctacaagagaccttctctccacctatctgcatgtcttttggttgtcaagtgtttctgcatggtagtgtccacaaagacaaatgatgatttcagttggtccacctgtatttaaaatgtgcaagaaaaattgaaatactctgctgtagcaagttttatgtaacaaagnnnnnnnnnnnnnnnnnnnnnnnnnnnnnnnnnnnnnntgtacaaaatactttagttttttgtatttcatttaaacccaagaacatgctaatcattgtgttttatgtatgctacaaattctctggtagaattgttgtatattattgtaaaattatttcaataaatcatggggatgacaatttgattattatactttagttttcaaaattgaacagatttctatgaatcctaaaaatatttttttctatgaaatcattagtacccagctagagtccgcccaggtatggcccctagacatannnnnnnnnnnnnnnnnnnnnnnnnnnnnnnatttcactgtctgtttgaagtacatgagcgaactgtcagatgtgtcaaaggggtctatttctgactaatcaagtgcactaaatcagaatattcttagcatactgagagttcccatttttagccacagggtagccaaaaggattttctttaaaacttaaaataataagtcagtggcaaaaaccagtgtttaactcaaattatattacacaaaacagaagaaattatgtttttgcccagaaaataaagaaacagcgtggtgcaggaaggagcctggatgacagtgaaacttactggagtgttgtcagttgcagacagcagaagagctggacaggcactttctgggtcctttccaattccccatgttaggtcaaggtcccctctttatctgatacccttaagggacaacatactctgaagtaagtttctgtacagtgatacatttctgtgcatccttcctcttacctcttacactatcctgaaaatcctttgactatcataagacccttgtctgtcccgttccatactcccccactgatgctaatatacacttgatatcaaactgtcagcctaccaaaaagatattgtggcttatgggtattgctgtcttgttcttggtatgttcctatactgattgcccctcagtctgaaaaaaaatgctcattgtaagcctaaaactagatgattttgctttcccactcactgttttttctttccttctgcttgttgtaagaatccaatgaaataatgtatgtaaaagcaccttgtaaactgtaagctttcggtgtaagatgtaaggtgtgttgttttttcattaattctttgtnnnnnnnnnnnnnnnnnnnnnnnnnnnnnnnnnnnnnnnnnnnnnnnnnnngttttttaattcatcaataaccgcaaaattaaagtatctttaaag

SEQ ID NO: 324 - DNA, 829 bp, Cricetulus griseus
  misc_feature: Qualifier WAN013I1G_at
cgggagcgcccgccgccgccaggcagactgacagagaccagccatggcagacgagccgaagcccatcagtccgcttaagaacctcctggccggcggctttggcggcatgtgcctggtgtttgtggggcaccccctggacacggtcaaggtgcggctgcagacgcaaccgccaagtttgccaggacagccacctatgtactctgggaccatcgactgtttccggaagactcttattagagagggcatcacagggctgtatcggggcatggctgcacccatcattggagtcacccctatgttcgctgtgtgcttctttgggtttggtttggggaagaaactacagcagaaatctccagaggatacacttaactacccacagctgtttgcagctggaatgtkrkskggtgtgttcaccacgggaatcatgacccctggagaacggattaaatgcttgctgcagattcaggcttctacgggggaaaccaaatacactggtaccttggactgtgcaaagaagctctatcaggagtttgggatccgtggcttctacaaagggactgtgctgaccctcatgcgagatgttccagccagtgggatgtatttcatgacatatgaatggcttaaaaatctcttcactccagagggaaagagtgtcagtgacctcagtgtaccccggatcctcgtggctggaggctttgccgggatcttcaactgggctgtcgcaatccccccagatgtgnnnnnnnnnnnnnnnnnnnnnnnccctcctgggaaatatccccatgggttcagaaacgtgctgagagagctgatccgggatgaaggagtg

SEQ ID NO: 325 - DNA, 270 bp, Mesocricetus auratus
  misc_feature: Qualifier L00180_at
atccatcacacattttgtttgttttacttcttactagggcactgagaaagcacttctgaagcttcaggagttctttcctgaaatgcagattctggcagttagtggtaactactgcactgacaagaaacctgccgccataaactggatcgagggaagaggaaagacagttgtgtgtgaagctgttattccagccaaggtggtgagagaagtaagtggtggatgaatgtctatcctactttaaaggcttagtatctctaattgggctgggac

SEQ ID NO: 326 - DNA, 189 bp, Mesocricetus auratus
  misc_feature: Qualifier L00330_x_at
atactttttcagggatgttaagttagaagatacttactttgacagagatgtggaaaaggcatttatgaaggctagttctgagctctttaaccagaaaacaaaggcatctttgcttgtatcaaatcagaatggaaatatgtacacgtcttctgtatacggttcccttgcttctgttctggcacagtaagt

SEQ ID NO: 327 - DNA, 600 bp, Cricetulus griseus
  misc_feature: Qualifier WAN0088T2_at
gtcgacccacgcgtccgcccacgcgtccgcttcgatttaagcacattcctcgattccagcaaagccctacaacatgaccgagatgagcttcctgaacagcgaagtgttggctggggacttgatgtcccccttcgaccagtcgggtttgggggctgaagaaagcctaaggctcttatatgactacctggaggtggccaagcacttcaaacctcatgggttctccagcgacaaggctaaagcgggctcctccgaatggctggctgtggatgggttggtcagtgcctcatacaccggcaaggaggatgcattctccgggacagactggatgttggagaaaatggatcttaaagagtttgactttgatgctctgtttcgaatggatgacctggataccatgccagatgagcttttggccacgttggatgacacatgtgatctttttgcccctctagtccaagagactaacaaggagcccatgacagtgaacccaattggccatctcccacatagtgcaacaaaaatagaccaagttgcccnctttgcattcttgcaacctttttcctgttccccaagggggcctgtctttcacttcagaatatt

SEQ ID NO: 328 - DNA, 2214 bp, Cricetulus griseus
  misc_feature: Qualifier M27838_s_at
ctaaaacatcccaagtagggagacttctcattggtcagctaggtaacctggggtagccagtgaggccctgggccgacctctgaggtcatcccgcatacagggtcacagcacctaaaagaacagaagcaagccgaattatctcctaactctcttggccccaagctgcctcttcgcgatggcctcagccccgcctggcgtcgcacgtcagcactgcgggccacacccctgcgcacgggttgggtccggcgatatgatgaaacttcccgcacgcgttacagtagcccggctgctctaaggtaagaagcagtcctctgctccttcgtgtccctgtgctgcctcctctcccgcctcctctggccctagccaccagcgagcagagtgcctgcagttctcctgcaggatgtgtggcatttgggccctctttggcagcgatgactgcctttccgtgcagtgcctgagtgcaatgaagattgcacacagagggccagatgcatttcgttttgagaatgtcaatggatataccaactgctgctttggatttcaccggttggccgtggttgaccccctgtttggaatgcagccaataagagtgaagaaatacccttatctgtggctctgttacaatggtgaaatttacaaccacaaggcgctacaacaacgctttgaattcgagtatcagaccaatgtggatggtgagattatcctgcatctctatgacaaaggaggtatcgagcaaaccatttgtatgttggatggggtgtttgcattcatcttgctggacactgccaacaagaaagtttttctgggcagagatacctatggagtcaggcccttgtttaaagccatgacagaagatggatttctggctgtgtgttcagaagctaaagggctcgtttccttaaagcactccacaactcccttcttaaaagtggagcccttccttcctggacactacgaagttttggatttaaaaccaaatggcaaagttgcatctgtggagatggtgaaataccatcactgcagagatgaaccactgcacgccctctacgacagcgtggagaaactcttccaaggctttgagttagaaacggtgaagagcaatcttcgtatcctgtttgacagtgctgtcaggaagcgcttgatgacggacaggaggatcggctgccttttatcagggggcctggactcgagcttggttgccgcctccctgctgaagcaactcaaggaggctcaagtacagtatccactccagacatttgcaattggcatggaagacagccctgatctactagccgctagaaaggtggcaaattatattggaagcgagcatcacgaagtcttatttaattctgaagaaggcattcaggccctggatgaggtcatattttccttggaaacttacgacattacaacagttcgagcatctgtaggtatgtatctaatttccaagtatattcggaagaacacagacagcgtggtgatcttctctggagaagggtcagatgaacttacacagggctacatatatttccacaaggctccttctccagagaaagcagaggaggagagtgagagactgctgaaggaactctacctgtttgatgttctccgagccgaccgaactactgctgcccacggtcttgaactgagagtcccgtttctggatcatcggttttcctcctattacttgtcactgccaccagaaatgagaattccaaaaaatgggatagaaaaacatctccttcgagagacgtttgaggactccaacctgctacccaaagagattctctggagaccaaaagaagccttcagtgatgggatcacctcagttaagaactcctggtttaagattctacaggattatgttgaacatcaggttgatgatgaaatgatggcaacagcagcccagaagtttcccttcaatactcccaaaactaaagaaggctattactaccgccagatctttgaacgccattacccaggccgggctgattggctgacccattattggatgcccaagtggatcaatgccaccgatccttctgcccgaactctcacccactataagtcagctgccaaagcttagatattctctaaactctagtgtaaaagtgttcttctcactctgaaggcagagaccgtgagacaaacacagtaatgcaaatcaaccatcaactgctcaggcttacataggcatggaaagaaataaaaaca

SEQ ID NO: 329 - DNA, 600 bp, Cricetulus griseus
  misc_feature: Qualifier WAN0088J7_at
tcgacccacgcgtccgggagacagaggattatagacagttcaggaaatcggtcctggcagatcaggggaaaagctttgctacttcatctcaccggaatactgaggaggaaggacccaaatataagtccaaagtttcactaaaaggcaatagagaaagtgatggatttagagaagaaaaaaattataaactgaaagagactggatacatagtggaaaggcctatcactgcaaaagataagcacaaggaagaagacaaaggttctgaaagaataacagtaaagaaagaaacacagtcacctgagcaggtaaagtctgaaaagctcaaagacctctttgattacagaccccctctacacaagaatctagatgcacgagaaaagtctatcttcagagaggagagcccgcttaggatcaaaatgatagccagtgattctcatcgtcctgaagtcaaactcaaaatggcacctgttcctcttgatgattctaacaggccagcttccttgactaaagacaggctacttgctattacgcttgtccattctgtcaagaaagagcaagaattccgatccatctttgaccacattaagttgcctcatgcca

SEQ ID NO: 330 - DNA, 855 bp, Cricetulus griseus
  misc_feature: Qualifier WAN013I3P_at
gcctcccagcgccgagcggagctgcggaggagaaagctgctcatgaactccgagcagcgcatcaaccgcatcatgggctttcacaggcccgggagcggcgcggaagaagaaaatcaaacaaaatcaaagccccaggacagtgacaaactgaactcccttggtgttccttcagtttcaaagagggtagtgctcggtgattcagtcagcggtggaacatctgaccagcccagtggtgtggcagaagttaagggtacccagctgggagacaaattggactcattcattcagccaccagagtgcagtagtaacgatggtgtggagctccggcagcggagcagaggtgacccgacagcaggctcggcccagagggcttctcaccatggcctagaacagtacctgtccagatttgaggaagcgatgaaactgcgcaagcagctgatcagtgagaagcccagccaggaagatggaagtacaacagaggaattcgactcttttcgaatattccgattggtggggtgtgctcttcttgctcttggcgtcagagctttcgtctgcaaatatttgtctatattcgctccatttcttactttgcaacttgcgtacatgggactatacaaatattttcccaagggtgagaagaagataaagaccacagtgctgacagctgcgctgttgctatctggaattcctgctgaagtgataaatcggtccatggagacctatagcaaaatgggtgaagtcttcactgatctctgtgtctactttttcacttttattttttgtcatgaactgcttgattattggggttcagaagtaccctgaaacctgaagaactgagaacaagtttacaaagtg

SEQ ID NO: 331 - DNA, 894 bp, Cricetulus griseus
  misc_feature: Qualifier WAN013HZ9_at
aaacagacaactgctaaatgaaagaattaaactagaaggtattattactcgagtagagacttacttgaatgagaatctgaggaaacgcttggaccaagtagaacaggaacttaatgaactgagagagacagaaggtggtactgttcttactgccacaacatcagaacttgaagctataaataaaagagtaaaagatactatggccagatcagaagatttggataattccattgacaaaacagaagctggaattaaagagctccagaagagtatggagcgctggaaaaatatggagaaagagcacatggatgccataaatcatgacaccaaagagctggagaagatgaccaaccgacagggcatgctgctgaagaagaaggaagagtgcatgaagaaaatccgggagttggggtcccttcctcaggaagcctttgaaaagtaccagacactgagcctgaagcagttgtttcgaaaacttgagcagtgcaacacagaattgaagaagtacagtcacgttaacaaaaaagctttagatcagtttgtgaatttctctgagcagaaagaaaagttgataaagcgacaagaggaactggataggggctacaaatcaatcatggaattaatgaacgtgcttgaacttcgaaaatatgaagctattcagttaactttcaaacaggtatctaagaacttcagtgaagtattccagaagttagtacctggtggcaaaacaactttggtgatgaagaaaggagatgtggagggcagccagtctcaagatgaaggagaagggagtggcgaaagtgagaggggctctgggtcacagagcagtgtcccatcagttgaccagttcacaggagttggaatcagggtgtcatctacaggaaagcaaggtga

SEQ ID NO: 332 - DNA, 600 bp, Cricetulus griseus
  misc_feature: Qualifier WAN008DDZ_at
gtcgacccacgcgtccggtttcgaaatgtgtccctaaagtgcctcactgagattgctggtgtgagtgtaagccaatacgaggaacaatttgaaactctgtttacacttacaatgatgcagctaaagcagatgcttcctttaaatactaatattcgagttgcatactcaaatggaaaagatgatgaacagaactttattcaaaatctcagtctatttctctgcacctttctaaaggaacatggtcaacttttagaaaaaagactgaatctcaaggaagcactcatggaggctcttcattatatgttgttggtgtctgaggtcgaagaaactgaaatttttaaaatctgtcttgagtactggaatcatttggcagccgagctctatagagagagtccattttctacatctgcttctccattgttatctggaagtcagcattttgatattcctccaaggagacagctgtacctaactgtgttatcaaaggtccgtttattgatggttagtcgcatggctaaaccagaggaggtactggttgtagaaaatgatcagggagaangtgtaagagagttcatgaaggataccgattccataaatntg

SEQ ID NO: 333 - DNA, 363 bp, Cricetulus griseus
  misc_feature: Qualifier WAN008EQP_at
cggggctatctacacatgagctcacctctctgctagagaaagagctggagcagagccagaaagaggcctcggatcttctagagcagaaccgacttttgcaggaccagttaagggtggccctgggccgatagcatagtgcccgggagggctatgtgctacaggctacatgtgaacgaggctttgctgccatggaagagacacaccataagaagattgaagacctacagaggcaacaccagcgggaactggagaagctgcgagaggaaaaggaccgcctcctggctgaggagacagctgccaccatctcatccattgaagccatgaacaatgctcatcgggaggagatggaacgggagttagaga

SEQ ID NO: 334 - DNA, 566 bp, Cricetulus griseus
  misc_feature: Qualifier WAN008CQI_at
gtcgacccacgcgtgcgggcacgcgtccgggcggaggagccgctgggcatagaggtcgaccagttcctggaagacgtgcggctgcaggagcgcgccaccggtggcttgttggccgaggcccccaatgaaaagctcttcttcgtggacactggatccaagaaaaaagaaccaaacaagaggacctgggtccagaagaagtctcagcgtctccagaaaccccttcgggttgaccttgcccttgagaatccttctaaggtccctgctcccaaagacatcctcgcacaccaggtccccaatgccaagaagctcaggcgaaaggagcagctatgggagaaactggcgaagcagggtgagctgcccagggaggtgcgcaaggcacaggcccggctcctcagccctcctgcaccaaaggccaaacgcgggctacaggacatcattgagcggcccttctatgacctctggaacccaaacaaccctctggacaagccgttggccggtcaggatgcattttttctggagcataccaagaagaaaggagtgaggcggncaccacgcctccatgtcaa

SEQ ID NO: 335 - DNA, 600 bp, Cricetulus griseus
  misc_feature: Qualifier WAN008CSC_at
gtcgacccacgcgtccgagagggttctggaagcaaaggagctggcactacagcccaaagatgacatcgtagatagagcaaaaatggaagatactttgaagaggaggtttttctacgatcaagcttttgctatttatggaagtgtcagtggtctctacgacttcgggccagtaagatgtgctttgaagaacaatatcatccagacctggaggcagcactttatccaagaggagcagatccatggagatcgactgcaccatgctcacccctgagccagttctaaagacctctggccatgtagacaaattcgctgacttcatggtaaaagatgtgaagaacggggagtgtttccgcgctgaccacctgctgaaagctcatttacagaaattgatgtcagataagaagtgttctgctgagaagaaatcagatatggaaagtgtcttggcccagctcgataactatggacaacaagaacttggggatctttttgtgaactataatgtaaagtctcccactactggcaatgacctgtctcctccggtaccttttaatttaatgtgcaagaccttcattgagccacgaggaaatatgcctggatatc

SEQ ID NO: 336 - DNA, 324 bp, Cricetulus griseus
  misc_feature: Qualifier WAN013I4U_at
atgannnnnnnnnnnnnnnnnnnnagccagcctctggcctccaagcaggagaaggacgggactgagaagcgaggccggggtaggccacgcaagcagcctccggtgagtcctgggacggcgctggtagggagtcagaaggagcccagtgaagtgccaacaccgaagagrcctcggggccgaccaaagggaagcaaaaacaagggcgctgccaagacacggaaagctaccacagctccagggaggaaaccaaggggcagacccaagaaactggagaaggaggaagaggagggcatctcccaggagtcctcggaggaggagcagtga

SEQ ID NO: 337 - DNA, 2061 bp, Cricetulus griseus
  misc_feature: Qualifier Y00365_at
gtcaacttctcagagttctcgaagaagtgctcagaaaggtggaagaccatgtctgctaaagaaaagggaaaatttgaggacatggcaaaggctgacaaagctcgttatgaaagagaaatgaaaacctacatcccccccaaaggggagaccaaaaagaagttcaaggaccccaatgcacccaagaggcctccttcggccttcttcttgttctgttctgagtatcgcccaaaaatcaaaggagaacatccagggctgtccattggtgatgttgcaaagaaactgggagagatgtggaacnnnnnnnnnnnnnnnnnnaagcagccctatgaaaagaaggctgctaaactgaaggagaagtatgaaaaggatattgctgcttacagagctaaaggaaaacccgatgcagcgaaaaagggggtggttaaggcagaaaagagcaagaaaaagaaggaagaggaagatgatgaggaggatgaagagnnnnnnnnnnnnnnnnnnnnnnnnngaagacgaagatgaagaagaagatgatgatgatgaataagttggttctagcgcagtnnnnnnnnnnnnnnnnnnnnnnnnnnnnnnnnnnctcattaaagcatttaacccccctgtacacaactcactccttttaaagaaaaaaaaattgaatgtaaggctgtgtaagatttgtttttaaactgtacagtgtcttttttttgtgtgttaacacactaccgatgtgtcttggatggtcctgtcctggtggtattttcaatgaccactaacctgcctggtacagtctgggggttgtaaattggcatggaaatttaaaggcaggttcttgtttacacagcacaaattagttatatatggggacggtannnnnnnnnnnnnnnnncnnnnnnnnnnnnnnnnnnggttttttttttccatcttcagttgtctttgatgcagcttatacgaaaataattgttctgttaactgaataccactctgtaattgcaaaaaaaaaattgcagctgttttgttgacattctgaatgcttctaagtaaatacaatttttttttattagtattgttgtccttttcataggtctgaaatttttcttctcaaggggaagctagtcctttgcttttgcccattttgggtcacatgaattattgcagtgtttcttttcgtatagttagctggagaaaagcttttatctacacaccctgcatattgtgatgggtaacattttcatccatagttgaaagcccaaaattgtgattagttggataagagatagtacataaaccatgtgcaaaactgagagtgatcaattctatcacaccatggaataagaatcgaacaatctgaaagtctgccttgaaggactagtagaaaattgtgttctaatccttacgtgaggactcttcttaacgtcattactgtgtaatggcagttacattctgcagttccctattaaagacctgagaatgtatccctcaaaagcgtgagctaaaatacaagactgccatattaaactttgtgacattggtcctagcgaaggctatgataagaacgctggctgtaatgcctttgcccatctatctaaatacaggattgctcaggaaacttgactgtttaaaaaaaaagtatttttaattagttgagccagcttttcttaaaattatgccacatataaaattaagggtatattttcctacattgtgatttgtcccttataaatcagatacaaggaaagatgataaactttgcatattagtaccagttgtccaatccatttggtttttctttataaaacccaaactcattagtcatgtttaatctgcttagcagttagggaacaatttggcaattttgtgttttttttttgagattatcattctcttaaagtgccagtgtttttaaaatagcgttctgtaattttacacgctttgtgatggagtgctgtttgttatataattttgacatgggttctttacattgcgtttgtttatgtaatttgaggaggaatactgaacatgagtcctggatgatactaataaactaataattgcagaggtttt

SEQ ID NO: 338 - DNA, 8305 bp, Mesocricetus auratus
  misc_feature: Qualifier D45419_at
aggagagagaggcgtctcaagatggcggctcccagagcctcctgcccgagcttttaagcgggagcatccgggcaaatagttagagcggcagcgctcagtggcctccagattctcgggagtaggtgctctgcagttttgagaagctttaaaggacacgagccgattgagaagggggggaaagagccaaagggaaggaggatagttaagatggcggcctccatggagtcgtccaccgctgtctaaggagccgcttctccgtgaaagctgccttagacgaaagggggtgtgtgaagaattaagggtctccttcccgctcagtgacttattcccactgcatccttaagcggaactatggcttcggctgtgtcccccgccaactnnnnnnnnnnnnnnctgcagccccgctggaaacgagtggtgggttggtcgggtccggtgccccgaccccgccacggccaccgtgccgtggctatcaaggagctcatagtggtgtttggcggcggtaacgaaggaatagtggacgaactacacgtgtacaacactgcaaccaaccagtggttcatcccagctgtgagaggggatattcctccagggtgtgcagcctatggctttgtgtgtgatggtactcgcctgctggtatttggtgggatggtggagtatggaaaatacagcaacgacctctatgaactccaggccagtcgctgggaatggaagagactgaaagcaaagacacccaaaaatgggcctcctccatgtcctcggcttggacacagcttctcccttgtgggcaacaaatgctacctatttgggggtctagccaatgatagtgaggaccccaagaacaacattccgaggtacttgaatgacttatatatcctcgaactgcggccaggctctggagtggtagcttgggacatccccatcacttacggagtcctgcctccacctcgggagtcgcatactgctgtggtgtacactgagaaggataataagaaatcaaagctggtgatctatggagggatgagtggctgcaggctaggggatctttggaccctggacatcgagacactgacatggaataagcccagccttagtggggtggcaccccttcctcgaagcctccattctgcaactaccataggaaacaaaatgtatgtatttggtggctgggtgcctcttgtcatggacgatgtcaaagtggccacacacgagaaggagtggaagtgtaccaacacactggcttgtctcaaccttgataccatggcttgggaaaccatcctgatggatacactggaagacaacattcctcgagctcgagctggtcactgtgctgttgccatcaatactcgtttgtatatttggagtggccgtgatggctaccgcaaggcctggaacaaccaggtctgctgcaaggacctgtggtatctggagacagaaaagccaccacccccggcccgagtacaactcgtacgagctaacactaattcactggaggttagctggggtgcagtggcaacagccgacagttaccttctgcagctccagaaatatgacattcctgccacagctgctacggccacttcccctactcccaatccagtcccatctgtgcctgccaaccctcccaagagccctgcgcctgcagcagctgcacctgctgtacagccactgacccaagtaggcatcacacttgtgccccaggctgccgctgcacccccaagcactaccaccatccaggtcttgccaacagtaccaggcagctccatttctgtgcccactgcagccagggctcaaggtgttcctgctgttctcaaagtgactggtcctcaggctacaacaggaaccccactggttactatgagacctgctggtcaggctggaaaagcccctgtcactgtgacctccctgcctgctagtgtgcgaatggttgtacccacacagagtgcccaggggacggtgattggcagcaacccacagatgagtgggatggctgcattggctgctgccgctgctgccacacagaagattcctccttcctcagcacccaccgtgctgagtgtcccagccggcaccaccattgtcaagacagtggctgtgacacctggcacaaccactcttccagccactgtgaaggtggcctcctcccctgttatggtgagcaacccagccactcgcatgctaaagactgcagctgcccaagtggggacatctgtgtcctctgctgccaacacatctacccgccctatcatcacggtacacaaatcaggcaccgtgacagtggcccagcaagcccaggtagtgaccacagtggtcggcggagtcaccaagaccatcaccttagtgaagagccccatctctgtcccaggaggcagtgccctgatttccaatctcggaaaagtgatgtcagtggtccagaccaaaccagttcagacttcagcagtcacaggccaagcctctacaggccctgtgactcagatcatccagaccaaagggcctctgccagcagggactatcctgaagctggtgacatcagcagatggcaaacccacaaccatcatcaccaccacacaggctagtggggctgggtccaagcctaccatcctgggcatcagtagtgtctctcccagcaccaccaaacctggtacgactaccatcatcaagaccattcccatgtcagccattatcacccaggcaggcgccacaggtgttaccagcactcctggcattaagtcccccatcacaattatcaccaccaaggtgatgannnnnnnnnnnnnnnnnnnnnnnnnnnnnatcactgctgtccccaagattgctactggccatgggcagcaaggagtgacccaggtggtgctgaagggagcccctgggcagccaggcgccatcctccgcactgtgcccatgagtggcgtccgcctggtcacccctgtcactgtctctgctgtcaagccagctgtcaccacattggttgtgaagggcaccacaggtgtcacaaccctaggcacagtgacaggcactgtctctactagccttgcaggagctggggcccatagcaccagtgcctctctggccacacctatcactaccttgggcaccattgccactctctcaagccaggtgatcaatcctactgccatcacagtatcagctgcacagaccacactgacagctgctggtgggcttacgacacccacaatcacaatgcagcctgtctcccagcctacccaagtgactctgatcacagcacccagtggggttgaggcccagcctgtacatgaccttcctgtatccattttggcctcacctactacagagcagcccacagcaacagtcaccatcgctgactcaggccagggtgacgtacagcctggcactgtgacactggtgtgctccaacccaccctgtgaaacccatgaaacaggcaccaccaatacagctactaccactgttgtggctaaccttggaggacatcctcagcctacccaggtgcagtttgtttgtgacagacaagaggcagctgcttctcttgtgacctcagctgtgggacaacagaatggtaatgtggtccgtgtctgttcaaaccccccttgtgagacccatgagacaggcactaccaacactgccacaacagccacttcgaacatggctgggcannnnnnnnnnnnnnnccccccttgcgagactcatgagacaggcaccaccagcactgccactacagcaatgtccagcatgggcactgggcaacagcgagacactcgtcatacctctagcaatcccactgtagtgcggatcactgtggctcctggggcactggagagaacccagggtactgtgaagcctcagtgccaaacacagcaggccaatatgaccaacaccaccatgactgtgcaggctaccagatctccgtgcccagctggcccactgctcaggccaagtgtggcactggaggctgggaaccatagccctgcctttgtacagctagctcttccaagtgttagagttgggctgagtggccccagcaacaaggacatgcccacagggcaccagctggagacctatcatacttatacaaccaataccccaaccacagccctctctattatgggtgctggggagcttggtacagctcgattgatccccacatctacttatgaaagcctccaggcaagctctcccagcagcaccatgactatgacagctctagaggcactgctgtgcccttcggctaccgtgacccaagtctgctccaatccaccgtgtgagacccatgagacgggcaccaccaacactgccactacctccaatgcgggcagtgcccagcgggtatgttccaacccgccttgtgagactcatgagacgggaaccacacacacagctaccactgccacatcaaatggaggtgcaggccagcctgagggtggacaacagcctgccggtggccgtccctgtgagacacaccagaccacttccactggcaccactatgtcagtcagtgtgggtgccctgcttcctgatgccactccctctcatggaaccttggagtctggcttagaggtggtagcagtgtccaccgtcacctcccaggctggtgccacattgctggcttctttcccaacacagagggtatgctccaaccctccttgcgagacccacgagacaggcaccacgcacacagccaccactgtcacctctaacatgagctcaaaccaagaccctccaccagctgccagtgaccagggagaggtggtgagcacccaaggtgacagcgcaaacatcaccagctccagtggtatcacaacaactgtgtcatccacattgccacgagcagtgaccactgtgacacagtctacaccagtgccaggtccctctgttccgaacatctcatcactgactgagaccccaggggctctgacttccgaagtccccatcccagccacgataacagtgaccatagccaacacagaaacttctgacatgcccttctctgctgttgacatcctgcagcccccagaggaactccaggtctcaccagggcctcgtcagcagctgccaccacggcaactcctgcagtctgcctctacccccctgatgggggagtcctccgaggtcctgtcagcctcccagacccctgagctccaggccgccgtggatctgagcagcactggggacccatcttcaggccaggagcctaccagctctgctgtcgtggccactgtagtggtccagccacccccacccacacagtctgaagtagatcaattatcacttccccaagagctgatggctgaggcccaggcgggtaccaccacccttatggtaacagggctcaccccagaggagctggctgtgactgctgctgctgaagcagctgcccaagctgcagccactgaagaagctcaagccttagccatccaggctgtgctccagnnnnnnnnnnnnnnnnnnatggcaggcactggggagcccatggatacatctgaagcagcagcagcagtgacacaagcagaactgggtcacctttcagctgagggccaagagggtcaggccaccaccatacccattgtgctgacacagcaggagcttgcagcccttgtgcagcagcagcagcagctccaagaagtccaagctcaagcccagcagcagcaccacctccctactgaggctctggccccagccgacagtctcaatgacccatccatcgagagcaactgcctcaacgagctagctagtgctgtacctagcactgtggccttgttaccctcaacagccactgagagcctggctccatctaacacatttgtggctccccagcctgttgttgtagccagcccagcaaagatgcaggctgcagctaccctaactgaagtggacaatggcatcgagtccctgggtgtgaaaccggacttgccacccccacccagcaaagcccctgtgaaaaaggagaaccagtggtttgatgtgggggtcattaagggtaccagtgtaatggtaacacactattttctgccaccagatgatgctgttcagtcagatgatgactcaggcacaatcccagactataaccagctgaagaagcaggagctacagccaggcactgcgtataaatttcgtgttgctggaatcaatgcttgtggccgggggccctttagtgagatctcagccttcaagacgtgtctacctggtttcccaggggctccttgtgccattaaaatcagcaagagcccagatggtgctcacctcacctgggagccaccgtctgtgacctccggcaagatcatcgagtactctgtgtacctggccatccagagctcacaggctggtggtgagcccaagagctctaccccagcccagctagccttcatgcgagtatactgtgggcctagcccttcctgcctcgtgcagtcctccagcctctccaatgcccacattgactacaccaccaagcctgccnnnnnnnnnnnnnnnnnnncccgcaatgaaaagggctacggccctgctacacaagtgaggtggttgcaagaaaccagtaaagacagctcgggcaccaagccagccagcaagcggcccatgtcgtctccagaaatgaaatctgctccaaagaagtctaaggctgatggtcagtgaagaagctggccagcatctggatcctccagacccctctgcttcaggaacgccctatagggcctggcccaccctcccaccctgcccacccagcctttgcacttcaccctcaccagccactggctaccattcaatctccccccctttccctctctttttacaataaagaaagcacattttatcattgctgttgggaggaagcagaaggcagattggaaagagcgatgagtaagcaaatgagtgcaagttgtgctccttccttttccccacctgctgccctccctgagctgagtatgactagacagttcggagactttgggaagagaaattaaagctgaaaacaaagaagatagtacccacacttaagcctgcacttgtttgcagctcatcgcccttggcctgagaggcagccaaaggctggaggggggaggagtatcccccaagttccctcaggctttccccagcaaagtctgtctggttctattgtgttgtgttctatcactgtgatttccaggcaccttccatcccttctacaggcttagacagatctgtcttgcccagtaaccccatcggaagtataacacatctcagagacctttctgctaccacttaacacctctctcccttcctctttacaaaaagatgaggggaaagaaagaaggagccttgattagtctgagcagtggctgtgaaatcctcccaatgggcacagagtgccccccagtgatcctggctttggcccaccttgcttgagcctgggggccacatcaacaactgctgcatttgttgtatttttaagttgaaattaatgaagttaacattttttattgtaattttagctttagtgtgtgggggcttatccctttttgttagctgtgtacagaatgtgtatcgtattttctttcttccattttccccccttggctaatcattggcagggatcttttgcggaaaagctgagagagcagctagggcaggagagaaacagagtgtcctgggtaggacctggaagaccctgcctagttccattttcctgttactagacccctgcctacttttggaacagtgtgagacattgacctttattcccctgtgggttagcacttgacatgtagaggcttgcccttgaatgtggagtccaccttcatcagcatcactctcagagactgaccttgaacaatacagcaggggaaggaaacaagtggaacctggccaccttgaaagcagcccaggaagggggtgggtatgccatgtatcccacagaggctgggggtaggtacatgacacaaactcagtaattaatttcatagcattgcccccaagcactggattttttttctctccaggacccaagttttaggcggtgagtaatgcttgcaagcaagttactgaagaacttagaaatggacagccaatgtgttcagccactcaggcccataccccctgaggcctctagtgtattgtatcatatttctctgtggaaaatgtcatgtttagtcaagttggagccctctcgcctggcccctttgttttccctatcctttctcccaagcacccattgatttctcttctgagaacagtactaccccattcatctattgtagagtaatccctgtgactcaatattaccatagtgcgatgttgttttgtgctattttgaacaattaaaagacttttttgaaat

SEQ ID NO: 339 - DNA, 367 bp, Cricetulus griseus
  misc_feature: Qualifier WAN008E3O_at
gtcgacccacgcgtccgggccttcacagacatctctagacccaaggacttgtgccccaggaggagccgaggctaatgggtgcccgcctcagccctgaggccaacgctgaggtgccccgtgaggcccgcgaggcccttagtttccacggggatgccactggcacacaggtgcatctggatgaccagaggagcacagcgcataggctatccacgtttcatgatggcattgtgttcagccagcgtccagtgtggcccggtgagcgtgtagcgctgcgcgtgctgcgacaagaagacggctggtgcggtggcctccgcgtgggcttcacgcgcctggaccctgcaaatgtggccgcatcctgcctgccgcc

SEQ ID NO: 340 - DNA, 286 bp, Cricetulus griseus
  misc_feature: Qualifier WAN008EV8_at
ggcaccggcgcgggccatggcttcatcctctctgttatccgcgttggctgtgcggttgctgcgccccgcgcccggctgccacccgcgcctgcagcccttccacctggcggcagtgcgaaatgaagctgtcgtcatttctggaaggggggggggtcatcagatcaagcaagaagtgcggcaggaggtggaagattgggtggcctcatgcaacaagcggccacacctgagtgtcattctggttggtgacaatcctgccagtcactcctatgtgctcaacaaaaccagg

SEQ ID NO: 341 - DNA, 567 bp, Cricetulus griseus
  misc_feature: Qualifier WAN008D66_at
tcgacccacgcgtccgctctttaatcgcttggtaccttcaaaaggaaatggagtaagaaaattctgtgatatccagatccgaaggttacggaggttaggcattgaaaagactgaccccaccacactgacggatgacgagataatcagatttgtaagactggaaattgatccagaaaccataacgtggcagagagtgctggacaccaatgatagattcctgaggaagatcactattggacagtctccaacagagaaaggccacacacgcacggctcagtttgatatctctgtggccagtgaaattatggctgttctggccctcactacttctctgaaagatatgatagagagagtgggcaaaatggtggtggcatccagtaagaaaggcgaacccatcactgttgatgatctgggcgtgagtggagcantggcggcgctgttgaangatgcaatcaaacccaatcttatgcagaccttatagggcacaccggtgtttgtncacgctgggccctttgncancattgcacatggganttcctccattattgcanaccggattgcactcaa

SEQ ID NO: 342 - DNA, 1675 bp, Cricetulus griseus
  misc_feature: Qualifier WAN013I09_at
aagatgaaaaaggacggtcatactacgtagaccacaattctagaacaaccacatgggccaagcccaccatgcaggaagacccaagatcgaagatccctgctcatctgagagcaaagacaccagtggacaatgacctgggacctttacctcctggatgggaagaaagaacccacacagatggaagagtcttctatataaatcacaatacaaaaaagacacagtgggaagatcctcgcactcagaacgtggcaacaactggaccagcagtgccctactccagggattacaaaaggaagtacgagttcttccgaagaaagctgaagaaacagcagaacgacattccaaacaaatttgaaatgaagcttcgccgtgcaaatattctggaggactcttaccggagaattatgggtgtcaagaaagccgacttcctcaaggctcgactctggattgagttcgatggtgaaaagggacttgaytatggaggagtygctcgagaatggttcttcctcatttcaaaggaaatgtttaacccttattacgggctcwttgaatattctgctwcggataatyacaccctycagataaatccaaactcgggcctgtgtaatgaagatcacctctcttacttcaagttcattggtcgtgtggctggaatggctgtttatcatggcaagctgctggacggctttttcatccgccccttttacaagatgatgcttcagaagctgataaccctgcatgacatggagtctgtggatagtgaatattacagttcactgcgatggattcttgaaaatgacccgacagagctggacctgagatttatcatagatgaagaactttttggacagacacatcaacatgaactgaaaactggtggatcagagatcgttgtcaccaaywagaacaagaaggagtatatttaccttgtgatacaatggcgttttgtgaaccgaatccagaagcaaatggctgctttcaaagagggattctttgaactgataccacaggacctcatcaagatattcgatgaaaatgaactggagcttctcatgtgtggtctgggagatgtggatgttagtgactggaaggagcatacaaagtataaaaatggctacagcgtaaaccaccaggtcatccattggttctggaaggctgttttaatgatggatgcagaaaaaagaatacgcttgcttcaatttgttaccggcacatcccgtgtgcctatgaatggatttgctgaactctatggctcgaatggaccacagtccttcacagttgaacagtggggcacccctgataagctgccaagagcacatacctgctttaatcgcctggacctgccaccctacgaatcgtttgacgaactctgggataaacttcagatggcgattgaaaacactcagggctttgatggggtcgattagattacaagtaacaagctgtggtgtcttaaacaccatagtttttaagcaaaagcaaaattgcatcttaacattttccagagtacttctataagatagtcactggctcttccccgagagaccagagtacacagttcatccaaggtttcagccaccaccatactgaagtgttcatttgtccagttactttgtcctttcattggggttcacacgacagggcatttaagtcctgtgtgcctggctgtatctca

SEQ ID NO: 343 - DNA, 599 bp, Cricetulus griseus
  misc_feature: Qualifier WAN008CWD_at
gtcgacccacgcgtgcggcacaacatcacgtaatagcctggctggggaggcaaaactggaacctaccataaagaagatccactaaaaaggctttgagtggcgtgcaattaaactcagatatcaacgtgctctacgagtacctattaatatttgttcatctacatgtggaaaacgggacaaaggaaatgacaggcagaagttgtgaccaaggtcatcgggggaaatgagctaaaaggtgcctactcacagacagcattatgaaattaaaggaattccaacagaagaaagtagctactgcacataaccttcccagtaccaaagccatctatttcagaagcttggaggagaaattgacccaaaataaactcatcttgaaggaagagttgaaaacattgcttcacttgtgtcagtctcgggaagatgtggaactggcaaaaagtgtcatttacaggtgccactcagagaacagaaacttcactgtgggggaatataagtttggaccagttttcatgaggctatgctacgagttgcatcttgaggactctgcagtggagctcatcaaagacaagcacttacgaggtgtcttcctagactcaaca

SEQ ID NO: 344 - DNA, 637 bp, Cricetulus griseus
  misc_feature: Qualifier WAN013I3J_at
ctagcactgcccgcagcttcctggcctaagtgcgtctgcccacccagaacaactnnnnnnnnnnnnnnntctagcaatggccttcgcaaatctgcgcaaagtgctcatcagtgacagcctggacccctgctgccggaagatcctgcaagatggagggctgcaggtagtggagaagcagaacttgagcaaggaggagctgatagccgagctgcaggactgcgaaggccttattgtccggtctgccactaaggtcaccgctgatgtcatcaatgcagcagakaagctccaggtggkgggcagggctggcacaggcgtggacaacgtggatctggaggctgccacgaggaagggcatcctaktcatgaacacccctaatggaaacagcctcagcgccgcagaactcacctgtgggatgatcatgtgcctggccagacagattccccasgcaacagcttcgatgaaagatggcaaatgggaccggaaaaagttcatggggacagagctgaacggaaagacactgsgaattcytggcctgggcagaatyggaagagasgtggccacccgaatgcagtcctttgggatgaagactgtaggctatgaccccatcatttctccagaggtggcgggctcctttgga

SEQ ID NO: 345 - DNA, 600 bp, Cricetulus griseus
  misc_feature: Qualifier WAN008EBY_at
gtcgacccacgcgtccggcaaaatgaagctgcgctaccccatcaacgaggaagcactagagaagattgggacagaacccgattatggggcactctatgagggccgcaatcctggcttctatgtggaggcaaaccctatgccgactttcaagtgtgtagtaaaagccctgtttgactacaaggcccagagagaggatgagctgacctttaccaagagtgccatcatacagaatgtggaaaagcaagatggtggctggtggcgaggggactatggtgggaagaagcagctgtggttcccctcaaactatgtggaagagatgatcaacccagtcatcctatagccagagagggagcacctggatgagaacagcccactgggggacttgctacggggggtcttagatgtgccagcctgtcagattgccattcgtcctgagggcaagaacaaccggctcttcgtcttctccatcagcttggcatcggtggctcactggtccctagatgttgccgccgactcacaagaggaactgcatgactgggtgaaaaagatcccggaagtggcccagactgcagatgccaagcttactgaggggaagatgat

SEQ ID NO: 346 - DNA, 1094 bp, Cricetulus griseus
  misc_feature: Qualifier WAN013I0O_at
acgatcccagcaccagaccccgccagattttagcggtcgtcgtcgggtggcaaggacggactaggaataactgctctgtctrgaggcgtgtcttgcgttgcgccgccctttcctgcaccgctaagcytccctgatcgctcttgttttccggagcgctgcttctctttcccgcgccgtctacacctccttcccaccagrgataatggcgacagctgaggtactgaacattgggagaaaactctatgagggtaagacaaaagaagtctatgaattgttagataatccaggaaaagtcctcctgcagtccaaggaccagattacagcaggaaatgcagctagaaagaaccatatggarggcaaagctgcaatctccaataagattaccagctgtatttttcagttgttacaggatgccggtatcaaaactgctttcaccaagaaatgtggggagactgctttcattgcaccccaatgcgaaatgattccaattgaatgggtgtgccgaagaatagcaactggatcttttctcaaaaggaaccctggtgtaaaggaagggtataaattttacccaccaaaagtagagatgttcttcaaggatgatgccaataatgacccgcagtggtctgaggagcagctcattgctgcaaagttctgctttgctggacttgttataggccagactgaagttgacatcatgagtcatgctacccaagctatatttgaaatcctggagaagtcctggcttccccagaactgtacactggttgatatgaagattgaatttggtgttgatgtaaccaccaaagagattgttctggctgatgttattgataatgattcctggagactctggccatcaggggatcggagccagcagaaagacaaacagtcttaccgtgacctcaaggaagtaactccggaaggactgcagatggtaaagaagaactttgagtgggttgcagatcgagtggagttactcctgaagtcagatagtcagtgcagggttgtagtgctgatgggttccacttctgaccttggtcactgtgagaaaattaaaaaggcttgtggaaacttcgggattccatgtgaacttc

SEQ ID NO: 347 - DNA, 535 bp, Cricetulus griseus
  misc_feature: Qualifier WAN008E9C_at
gtcgacccacgcgtccgcccacgcgtccgcagcggccgggcggttgggccgtgaagggagagtcttttcggcgctgaggactggcgctgaggaggcggcggtggctcctggggcgtttgagcgggctcactcgagcccgcgggccgacgcggactcaggcccagccggcggaaccgccccggactccccgcgggcttttctagccgccatggaggacggtgtctacgagcccccagacttgactccggaggagcgcatggagctagagaacatccgacggcgtaagcaggagctgctggtggagatccagcgcctgcgagaggagctgagtgaagctatgagtgaggtggagggtctggaggccaatgagggcagtaagaccttgcagcggaaccggaagatggcaatgggcaggaagaagttcaacatggaccccaagaaggggatccaattcttggtagaacacgaacttctgcagaacacacctgaggaaattgcccgcttcctgtacaagggtgaggggctgaacaagaca

SEQ ID NO: 348 - DNA, 600 bp, Cricetulus griseus
  misc_feature: Qualifier WAN0088WG_at
gtcgacccacgcgtccgcgcgctttcctcgcggatcgaagagactctagctacagcttgtggctggaagggagacggaggccgcagctcagggaaagtgaagctgcagtagtggtggtaggaagatgtcgggcgaagacgagcagcaggagcaaactatcgccgaggacctggtcgtgaccaagtataagatggggggcgacatcgccaaccgggtactccgatctttggtggaagcctccagctcaagtgtatcagtgctgagcctgtgtgagaaaggcgatgccatgataatggaagagacagggaaaatcttcaagaaggaaaaggaaatgaagaaaggtgttgccttccctaccagcatttccgtaaataactgtgtgtgtcacttctcccctttgaagagtgaccaggactatatactcaaggaaggtgacttggtaaaaattgaccttggggtccatgtggatggcttcattgctaatgtggctcacacttttgtaattggtgtggctcatgggacccacgtaacaggccggatagcagatgtcattaaggcagctcatctttgtgccgaagctgccttacgactggtcaaacc

SEQ ID NO: 349 - DNA, 600 bp, Cricetulus griseus
  misc_feature: Qualifier WAN008EFI_at
tcgacccacgcgtccggagaatgtgacccatgaagacttccaagttacagaggaagtgaaggccctgaccgcggagattgtgaagaccattcgggatatcattgccctgaaccctctgtacagagagtcggtgttgcagatgatgcaggcaggccagcgggtagtggacaaccccatctacctgagtgacatgggcgctgcactcacgggggctgagtcccatgagctgcaggatgtgctggaagagaccaatatacttaagcggctctacaaggccctatcactcttgaagaaggagtttgagttgagcaagcttcagcaacgcctgggccgagaggtagaagagaagatcaagcagacgcacaggaagtacctgctgcaggaacagcttaagatcatcaagaaggagctgggactggagaaggatgacaaagatgccatcgaggagaaattccgggagcgcctcatggagcttgtggtccctaagcatgtaatggatgtggtggatgaggagctaagcaagcttgcactgctagacaaccactcctctgagttcaatgtcacccgcaactacctggactggctgacatccatcccatg

SEQ ID NO: 350 - DNA, 578 bp, Cricetulus griseus
  misc_feature: Qualifier WAN013HYE_at
gtgtctttgtattttgatactaagaggtaccaggaagcgttacatttgggttctcagctgctgcgggagttaaaaaagatggatgataaagcccttttggtggragtacagcttttagaaagcaaaacataccatgctttgagtratckgccgaaagcccgagctgccttaacctytgctcgaaccacagccaatgccatctactgcccccytaaattgcaggctaccttggacatgcagtcaggtattattcatgcagcagaagagaaggactggaaaactgcttactcatacttctacgaggcatttgaaggctatgactctattgatagccccaaggctatcacatctctgaagtacatgttgttgtgcaaaatcatgctcaacaccccataagatgtccaggctttggtgagtggaaagcttgcacttcgatatgcagggagacagacagaagcattgaaatgtgtggctcaagctagcaaaaacagatcactggcagattttgaaaaggccctgacagactaccgggcagagctccgggatgacccaatcatcaacacacacttggctaagtt

SEQ ID NO: 351 - DNA, 564 bp, Cricetulus griseus
  misc_feature: Qualifier WAN013HWW_at
cggagagkckcggagmcgctctttcysgmaggatcgccgcgatggccgcccagggagagccgcaggtccaattcaagctcgtcctggtgggcgacggtggcaccgggaagacgacgtttgtgaagcgccacttgacgggcgagttcgagaagaagtatgtagccaccctgggcgtggaggtgcacccgctcgtcttccataccaacagaggacccattaaattcaacgtgtgggacacagccggccaggagaagtttgggggcctgcgcgatggctactacatccaagcccaatgtgccattataatgtttgatgtaacatcaagagtcacctacaagaatgtacctaactggcatagagatctggtacgggtgtgtgaaaacatccccattgtattgtgtggcaataaagtggatattaaggacagaaaagtgaaggcaaaatctattgtcttccaccgaaagaagaatcttcagtactatgacatttctgccaaaagtaactacaactttgaaaagcctttcctctggcttgccagaaagctcattggagaccctaacttgg

SEQ ID NO: 352 - DNA, 505 bp, Cricetulus griseus
  misc_feature: Qualifier WAN008DUB_at
gtcgacccacgcgtccggccaatttggagcatcaactccactgtgctacagcagtgctgagatggttgcgatcctgctgctcctctgtcttcccttcatcttctgcctggccacaccaagaatcaggaaaatgctgtcttgtgggatgtgtacatctaatgtccagcttcctgggaaggtagccatagtcaccggtgctaacacaggcattgggaaggagactgctaaagacctggcccaaagaggagcccgtgtgtatttagcttgccgggatgtgcagaaaggggaacaggtggctagcgagatccaagccaccacagggaataatcaggtcttggtaaggaaactggacctagctgataccaagtctattcgagcctttgccaaagacttcttagctgaggaaaagcacctccacatcctaatcaacaatgcaggagtgatgatgtgcccctactccaagactgcagatggctttgagatgcacattggagtcaaccacttg

SEQ ID NO: 353 - DNA, 600 bp, Cricetulus griseus
  misc_feature: Qualifier WAN008E6V_at
tcgacccacgcgtccgcgcgccggtgaggcttactcgtggcttcacacgcttgcttcccggtcttatggccggtctgactctgtttgtgggccgcctgccgccctcggctcgcagtgagcagctggaagagctgttcagtcaggtcggtccggtgaagcagtgcttcgtggtgaccgagaaagggagcaaagcatgccgaggctttggctacgtcactttctccatgctggaagatgttcagagggcgctcaaggagatcactacttttgaaggttgcaagatcaacgtgactgttgccaagaagaaactaaagaacaagtccaaggaaaagaggaaaaatgaaaattcagagtccccaaagaaggagccaaagcctaaaaaagccaaggtagcagataagaaagccagattaattatccggaacctcagctttaagtgttcagaagatgacttgaaaacagcatttactccctatggaactgttctggaagtgaatatcccaaggaagccagatggaaagatgcgtggttttgcttttgttcagctcaaaaaccttcttgaagcaggaaaagctctcaaaggcatgaacatgaaagaga

SEQ ID NO: 354 - DNA, 600 bp, Cricetulus griseus
  misc_feature: Qualifier WAN008DNJ_at
gtcgacccacgcgtgcggcgggagcggcggcggcagacgcgtgtggcgaccggcgcgggaaagcggcttcctagttggggaagcgttagccccggggcagctgtcaccggtgtgctactggaaggccccttcagcgagcttctaggagcttgagacggtcacggtgccctccaactctcagctggaaggaaagatggttgaagcagatcgcccagggaagctcttcattggcgggctcaatacggaaacaaatgagaaagccctggaagcagtatttggcaagtatggacgaatagtggaaatacttttgatgaaagaccgtgaaaccaacaaatcaagaggatttgcttttgtcaccttcgaaagcccagcagatgctaaagatgcagccagagatatgaatgggaagtccttggatgggaaagccatcaaggtggagcaagctaccaaaccatcatttgagagtggcaggcgtggaccacctccacctccaagaagcagaggccctcccagaggtcttcgaggaggaagtggaggaactaggggacccccttcacgtggaggatacatggatgatgggggctattccatgaactttaa

SEQ ID NO: 355 - DNA, 479 bp, Cricetulus griseus
  misc_feature: Qualifier WAN008EPC_at
gtcgacccacgcgtccgcccacgcgtccgggcccatatcagtggccttgtggctgccaaggtgatcccgtcacctttcaagtatgcggacattgtcaccaccaccactcacaagactcttcgaggagccaggtcaggactcatcttttatcggaagggagtgcggaccatagaccccaagactggccaagagatcccttacacatttgaggaccgaatcaactttgctgtgttcccatccttacaggggggcccccacaaccatgccattgctgcagtagctgtggccctcaagcaggcctgcacccccatgttccgggagtatgccctacaggtgctgaagaatgctcaggccatggctgatgccctgctaaagagaggctactcactggtgtctggtggcactgacacccacctggtgctggtggacctgcgacccaagggcctggatggagcccgagctgagcgggtgttggagct

SEQ ID NO: 356 - DNA, 584 bp, Cricetulus griseus
  misc_feature: Qualifier WAN013I0Q_at
gggaagagctgaagcaggcgcttttggctcggcgcggtccgctgcaatccgtggaggaacgcgccgccgagccaccatcatgcccgggcacctacaggaagggktcggctgcgtggtcaccaaccgattcgaccagctatttgacgacgaatcggaccctttckakgtactgaaggcrgcagagaacaagaaaaaakargccggcgggggcggcgttggtggccctggggccaadakcgccgctcadgccgcggcccadaccaactccaacgcggcgggcaaacagttgcgtaaagagtcccakaaagatcgcaagawccckctgccccccagcgtcggcgtggctgacaaaaaggaggagacgcagccgccggtggcgcttaagaaagaaggaataaggcgagttggaagaagacctgatcaacaacttcagggtgaagggaaaataattgataggagaccagaaaggcgaccacctcgggaaagaagatttgaaaagccacttgaagaanagggtgaaggaggtgaattttcagttgatagaccaattattgaacggcctatccgaggccgaggtggtcttg

SEQ ID NO: 357 - DNA, 240 bp, Cricetulus griseus
  misc_feature: Qualifier WAN008ETE_x_at
tcgacccacgcgtccgagaagagctgacaaagcaaaaagaaataataacagcccaagacaatataattaaagaaaaacgtgcagaagcggctaaacacaatttgcaaaacaatgattctgaacttaaaattaaggagttagaccatagcattagcaaacataagcgggaagctgacgatgctgctgcgaaggtatccaaaatgttgaatgattatgactggattaatgctgagaaacacc

SEQ ID NO: 358 - DNA, 600 bp, Cricetulus griseus
  misc_feature: Qualifier WAN008D4B_at
gtcgacccacgcgtccgggggatgattatgaagttattcctaacagtaacttctatgtatctagaacagcctacagagataacacttctgtctattatataagtgggaagaagaggacatttaaagatgttggaaatcttcttagaagccatggaattgacttggaccataatcgatttttaatattgcagggtgaagtcgaacagattgctatgatgaaaccaaaaggccagactgaacatgatgagggtatgcttgagtatttagaagatataattggctgtggacggctaaatgaacctattaaagtcttatgtcggagagttgaactattaaatgaaaacagaggagaaaagttaaacagagttaaaatggtggaaaaggaaaaggatgccctanaaggagagaaaaacatagccattgagttccttaccctggaaaatgaaatatttaaaaaaaagaatcatgtctgtcaatattacatttatgatctacaaaaaagaattgctgaaatgaaaactcaaaaggaaaaaattcatgaagatactaaagaaatcactgagaagagcaatatgctttcaaatgaaatgaaagctaana

SEQ ID NO: 359 - DNA, 468 bp, Cricetulus griseus
  misc_feature: Qualifier WAN0088NR_at
gtcgacccacgcgtccggccaagactcatactgtgtgtctcagtttgcttgtcaccatgtttctgtacaacttgaccttacaacgggccactggcatcagctttgccatccatggcaacttctctgttcctggagggtcagatggtccaagtggagtgctgatctgtgctgaaaattatattacctataagaactttggtgaccagccagatatccgatgtccaattcccaggagacggaatgacctggatgaccctgaaagaggaatgatttttgtttgctctgccacccataagaccaagtccatgttcttctttttggctcaaacggagcagggagatatctttaagatcactttagagacagatgaagatatggtaactgaaatccgcctgaagtactttgatactgtacctgttgctgctgcaatgagtgtgcttaaaacaggcttcctttttgtggcatcag

SEQ ID NO: 360 - DNA, 418 bp, Cricetulus griseus
  misc_feature: Qualifier WAN008F2D_at
caacatactggcacagtgttagtcgcgcgtcatnacgcactncactataggggcgaattgggctaccggggccccccctcnagtcgacccacgcgtgcggggggatgtcacaaaggatgaattcaaaagactgtttgctaaataccgagagcccggagaagtttttatcaacaaaggcaaagggttcgggttcattaaacttgaatctanggccttggctgaaatagcgaaagccnaccttnatgacgcccgcatgagagtatacagcttcgggttcgctttgccacacatgctgcaaccctttctgttcggaatctgtcaccctatgtttctaacgaactgttggaagatgcctttagccaatttggtcctattgaaagggctgttgtaattgtgggatgatcgtggcaaatctaca

SEQ ID NO: 361 - DNA, 1747 bp, Cricetulus griseus
  misc_feature: Qualifier U22819_s_at
taacacgttggcggcggctggcacgggccggtgaggcggtaccaggtgggggccgtcgggtgtcatgggcggtggcggcggcaccgcctccgcgtctccctgagccggacctcacgggggactcagcgccaagctgggcgatggacgagagcagcgagctgggcggtctggagaccatggacaccctcacggagctgggcgacgagctgactctgggggacatcgatgaaatgctacagttcgtcagcaatcaagtgggagagttccctgatttgttttcagagcagctatgcagctccttccctggtggtggtagcagtggcagtagtnnnnnnnnnnnnnnnnnnnnnnnnnntgnnnnnnnnnnnnnaggcaatggtggtgcagccacagatcctggagtgcagcggtcgttcagccaggtcccattacctaccttctccccctcaactgcctcccctcaggctctagccttgcaggtcaaagtctctcctaccccacccagggcaacccctgttctccagcctagaccccagccccagccgcagccccagccttcagctcagcttcagcaacagacggtaatgatcaccccgacattcagcactgctccacagacaaggatcatccagcagcctttgatataccagaacgcagcaaccagcttccaagtcctgcaggctcaagtccaaagcctggtgacatcctcgcaggtacagccagtcacgatccagcagcaggtgcaaactgtacaggctcagcgggtgttgacacaaacagccaatggcacgctgcagacccttgccccagcaacagtgcagacagttgctgcaccccaggtgcagcaggtcccggtcctggttcagcctcagatcattaagacagattcccttgttttgaccacactgaaaacagatggcagccctgttatggctgcagtccagaacccagccctcactgccctcaccacccccctccagacgggcgcccttcaagtaccaactctggtgggcagcaatgggaccattctgaccacaatgcctgtgatgatgggacaagagaaagttcctattaagcaagtacctggcggtgtaaagcagctagagccccccaaagaaggagagaggaggacaacacacaacatcattgaaaagcgatatcgatcctctatcaatgacaaaatcatagagttgaaggacttggtcatgggaacagatgccaagatgcacaaatctggcgttctgaggaaggccattgattacatcaaatatttgcagcaggtcaatcacaagcttcgtcaggagaacatggtactgaagctggcaaatcagaaaaacaagctcctgaagggcattgatttaggcagtctggtggacagtgatgtggacttgaagattgaagactttaatcagaatgtccttctgatgtctcccccggcctctgactcggggtcccaagcaggcttctctccctattctattgactctgagccaggaagtcctctgctggatgatgcaaaggtatgagcttttgacatttctcaccctcagaatctcttgttttccctaaggcattagtggccaggtgactggatcactgcttaaaacaatgtggggtggagaggaccaggaattctgttatgttagctgcatttaaagtggtgaaaactggtggtgcatgcctgaaatcccagtgggagggaggatgaggcaggaagatcttgagatggtaccaagaccatctccaa

SEQ ID NO: 362 - DNA, 215 bp, Cricetulus griseus
  misc_feature: Qualifier WAN0088ZO_x_at
gtcgacccacgcgtccgttcaacaatttaaggatagtgatctctctcatgtgcagaacaaaagtgcctttttatgtggagtcatgaagacttacaggcagagagagaaacaagggaccaaagtagcggactccagtaaaggaccagatgagtcaaagattaaggtatgtgtttgagaactttctgtagctttaagtgtgtgtgttttgcttgtgg

SEQ ID NO: 363 - DNA, 600 bp, Cricetulus griseus
  misc_feature: Qualifier WAN0088JV_at
gtcgacccacgcgtccgcggctgcctacccatcagcacgtggcccatcccgcagacatcctgttgggctctcagttcctctacaccttcttctcgaagacccatggggacttgcacagcctggtgcgcagccgccgcggtattcccgagcccgaggctattgtgcttttccggcagatggctgctatggtggcacactgccaccaacatgggcttgtcttgcgtgacctcaagctgtgtcgctttgtcttcagcaaccgtgagaggacgaggctggtgctggagaacctggaggatgcttgtgtgataactggaccagacgactctctgtgggacaagcatgcatgccctgcctacgtgggaccagagatactcagctcccgaccatcctactctggcaaatcagctgatatctggagcctgggcgtggcactgttcactatgctggctggtcactatccgttccaagactctgagccggctttgctctttggcaagatccgtagagggacctttgccctgcctgaaggcctatcagccccagcccgatgcctgatccgatgtctccttcgaaaggaaccttcacagcgactagtggccc

SEQ ID NO: 364 - DNA, 129 bp, Cricetulus griseus
  misc_feature: Qualifier WAN008DKJ_x_at
gtcgacccacgcgtccgcacagttctttgccagaagctaaatcatggcaatgaccaccagtctcccagtagcagtaactataatggcctggtagagagctttgagctgggttccggggggcacactgat

SEQ ID NO: 365 - DNA, 597 bp, Cricetulus griseus
  misc_feature: Qualifier WAN008EJY_at
tcgacccacgcgtccgtttctttgtatagcttttggagcctgttctggtacttgctctgtagataaggctggctttctcatagacttgagacaatgctgaaaactaaatgtgagcattaagcctgtatactctccctctttgatatctttaaacattgtctccttataacaggtcttctggactggaacatacagtataactcaggatgaatgcaaacctaaaatgatgcccctgtgtttgtcacctgactgctgagataacaggcatgtaccctcctcttgtattatctgcaggtgattagagaagttgaagaattgaaagcatttgtatacagcgaactcaataatttcttcactgagtcgatttttctgtgaaaaaaagtagggaacaaaattcagtcatatttttattatgttcaatcaatcatacacttttctatgccttaaaaataaagacttttttttctgtatcaaactctaaggggttgatttaaaatggtggagaaataaggaaagatgacaatttttagacacataatgtcatggtaaaattttagacctaaattctctttaaatgattatgttgaaataaaaatg

SEQ ID NO: 366 - DNA, 7887 bp, Mesocricetus auratus
  misc_feature: Qualifier AJ225170_f_at
gggtggaaggatttcatggcaggctctatgggcctcagagatccaggtctagggacctgaatacacagaatgtctcagtactgcatatttcattccatactccagagtatttctttttcatttttcaaaaaagtggattgtcaggaatgtttccagtaacatgctttatttgaaaattgattgttgacaatttcccacatatgtaatatactctgaaaaaccttctctctcatgtctaaactctttaacataatcttgtaaaggcctgctgtgcacaacaagtccttttcaagtagaagggagttggctgcctaatgtttttccaaccgcaaactatcagtggcccttggtaagaaacaaatgtactattacaggagtccagttctcttccataacttctgccagacagctaatggctagattatgccaggtataaacaaatannnnnnnnnnnnnnnnnnnnnnncaatacatgactagtttaacacagagtatcagtaaagagacagtccagtaaaaatggggctgggacaggtaaataataattttaagagataacacaaaacacacatacacacagagaaggagagacacacatacacacacagagagagggtggcagagacagagagatgagcccaaagacaacatttgtttggattgtaaaaaatgtatatcaaatctggaaaacagtgtggagcattctgagtcttctaaaaataaatatgtactgtgtgactcagctgtacttcaccttgtaatatgcctaaatgacttgaaattctatttcacagatactttctcatccatgttgattgctactgtattcactacagctaggaaaaggaaacaaattaaatgttctatagctatctaatggacaatgaaatagtggtaatacacacgttgtaataacattccatgataatgaaaaaaggaaatcatgacatttgtagataaactgcaggcatgaaaaagtatcctactgagtgaggtaccccagacctaagaatacaaaagtggcatgttatgtttcttctgttattcctagctctaaatcttccagagaaaactaagacttcatttgagaccttgaaactctcatacatgtaacagcatttactgaaataagtgttcactaaattaaagacattacagggtgaacatattgtaagtattagagggaggaggaaagggaagtgattaatgatgaaaatattttataatctcaaaaataaaatataaagataataatgtgatgtaatttaaagaaagagtcacaaagaccaactaatgccttataatggtgtagtgggaaaacaggttgtcaatccaaaataccagtgggtgaatggtcggttattccatgtgttatagaggaaatcgatggaggataaacagtactgtagaatcctggatttcttcttggttgtcactaaaaaaggatcacaagacactatggctcagcataacacaggtattggttttaatgtgtaaacactgcaactgggcagggagagcctttgtttagcaaacataaaggaatattttcagcctgaatgtagagccccttatcacaattagttttgttagtgtcattatttttatagactgtttcatttgatttggttcttttaattaatgatatttttatttatttcgaggaatgggagcaattttcctactgtatgtactgttaaacttcagcaactcatgcagcactgtacagttgttgaatggttgttgaattcaatgtacaagaagaataaagtagtcaatgtatggaaagtattttaagaagcacagtgacctttacccaacagtatagttctggaagggaaattattatttgtatgctcaaacaagatagttctttgtttctaacaggaatgctcaattttcagacaaacataacctgaccttactgctaatcacattagttttgagatatgaggtcatacatcatttggcagggccaaagaggaaacactagacagatgtagcttagtgtagatcaaactgattggttccgtgaaactccctctaactatctgtataaaaagctttgtcagttgagttcttttcaaccactcacctcttcgagcttctgtggaaaagcaaagtcaggcaccaccatggtaaagattctggtgctggctttggtctttagtctggctcatgctcaggattttgcagaggtaaattcatttgctctattcatatatcttagcttcatcaacagcatatttctgtggatgtgacacatcactcagttagtccatgcaagactatatttaagaaataagttttctgactgtcacagaggtacagtttacctattagattcctctcactgaaacaattggaataaggaaacaatgggtgatatttgcagagcataagagggcaaaacatgagcacttctgcaaagtctcagctcaaaatgttctatgttgattaaatcatcatattaccctgagttattctacatccctagaactaatcttgaactaatgaacaaaatattattcattgacatttatagtgagtttacataattccaagaactgggaccagagatgtgaatttataccactggtatatagtttttctttgcttcatttgcctttgttgcttttttcagcttcaaggaaaatggtataccattgtcattgctgctgacaatcttgaaaagatagaagaaggaggaccactgagattctattttcgtcatattgattgttataaaaactgcagtgaaatggaaatcacattttatgtcatgtaagcaaattgtcaagcttgtaaattgagcagactatttatgcattctgcaacctttatgttaagcttacagggcatgttctgtttctggttgtgaaaatgaagctcatctttagcattgtgttaacattattcccagttgatcattgtctccaatttcgtgagtgtttcccttaaaaagcctagttgttggaagaatgttgttgaatgttatagtcaaatattggatgcaggggaatctaagcttccaatttctagtaagtcatcaggaatatgtgagccttttggtaaaagctgtgcatgttatgttattcaatctatgatgtcatgaaatagtcctccacaaatagtcgagaaaaatctaatttgcgattccctttctctgaagctctagaaccaaacattatctctctgacatttggataactgtattcattgaaagactgcatgacaccataagaactaaaaatgcttagaactatttctattttttaggttagcaataaaagtacttaacacatgcataataaattgtgtacatattatatcattggacaagcacaagagaatatttgaaatcacattcatttaataataaagtttggaagagagacaaattagagccttcatagcattaacaaaatgagaacaaatgaaggaatttatctgtatgattaataacaagatgatactatctagacaagttttaggaaaagtgaggtaagcatacatattttggtcctagctctgtacactcttaacagatgctagtgcactctgaagttcgttataacacttgaaggtctttaagatgaattcaaattttacacagaaaaaattatggtttctctgtttggtttttagtacaaacaaccagtgctccaagaccacagtcattgggtacttgaaaggaaatggaacctaccaaacccagtgtaagtacaggatctgtgtgtttcctttatgaacaaagcaatgctgatgtacatattcatgttgatacttcagacacccataggtgcatggatttaggaaaattttggctacttttaaagttatcaatccttccaatgaactgagatatgcttcatagtcagataacattgtacgtcatctgaagtttacgtggagaagaacactgttacagaaaggtgtgagtaatcataggaaaatatctccaaatagtttgaatgcatgaaaatttatttccagttagtattcagaagcttttctttcatatggtgtctcagattgttactggatgtgtctgccagtcactaataggactcagtgacctgaatatctttgcaaacttcatgcctcatgtcctcaagtacttacattgtgggtgttatctgtcattcttgatttctagaacatttaaatgaacagtgcgatttcaaagattatttctctcatagtttgtagagatcataacatttagataatcagatgataaatttcaattttaaaaggcagttagttgaagatctggttaagattttcatgctgagtacttcaaactttaatatttacttaaacaatggcaacactattcaactgaatcaatcacaatttgatgtttagtttgatttaaatgaacagagggcacattcttgtggcaggtctcatgctctactaaatttacatgtatattaaaaagcagtcctgcaatataaaatgaacacaccctataaatccttttattttcatgtggaactacttctcatatttaataaatgttagagagtggaaataatccatgcatgcttgcattccaatgagcacgcatnnnnnnnnnnnnnnnnnnaggcatacagaaacagatagatagacagataggcaattgcacacaatagcaatgtctgtcaaacacaaaaagtccatcctactttcaagatgacaacttttgtacacatgacaaattactatgttctatttttccacatggaaacatttgcccactgagtatttccatggtatggcaccagatatttacacattgaggaaatcaacaagtggcctttgggatctggttaaaagcagacagaatatccaacagattgttcccaaagttttaacacaagaaaatcagagggagattggagtatccgtacttaggattcatatcagatttgaagagcagaaatgtttgctcaaaatactttgatgaaatatcccaaatgaaggactacaggaatagctttgtacagatgtgtcatatgataatagataagagatgattgctagagtctgggcctccattaatcttcagcagtgatactcagttttgaaatatttcctttattacagttgaaggtaacaatatatttcaacctttgtatataacatcagacaagattttctttaccaacaagaacatggatagagctggccaggaaacgaacatgattgttgttgctggtaagaatggtagtggtttctctgaaataatagcagggtcactgaagactaggggcaacagcagaggctcttgggagagtttatgttcatgtttcctcagtggaaagagtaaggcaaataatattttcccaaagtttagaccattttgagtattatatgatcaagtgtgtactaaatttaaacatgatccttatacttttggttttgaactcagcctaaccaacagtggagaggctacctcaaatgcccttccccgatattgtgattgatgaattccttgtatgccatcctagaaccttcatgcagtggctgaaggaagcagagtcagacacacacagctaaacactgaagtcaactcctggaacccagttgcagaatgggaggagtaatgagcaaaggggttaagacttggcagtcatggaaacagcagaccagatcaagggggagctcatggacaccagactgattgttgagaggccaacatgggactgatccagataccctgaatgtagctgccagtgaggaggcatctacagtctttggggcctcaggcaacagatagcatttatccctggcatccagatggactttggacaaccattctacaggaagggatgttgtcacagcctggacacatgggggaaggcctaggccctgccatggatgatatgacagtcttgggagatccctcaagaatggcctcaccctccttggagagtggagtctggattgagtggaaagttgtagcagacttgaaggggacaggggaggaggggagggagagtgagctgggattgatatgtgatgcaagattgattctaatttaaatttaaaaaacaaacacacaaaaaccatgatccttagtgaatagactcggtgagttgtgaaggttccactggcatgcatttttgtctctttgctgacatatttttgaacatttagaggactaagatgagtgaattctctagaaaggtagttattcttttttggtgcatcatgtaacaatggaacatgatgtcataccacaagttagaggtctttcaacgtgtccatggaacactgggaaaattacaactgtaattgactgtattgacccatccaggacaggtgaatttggtgaagttaggatttttccattgccaataacagtgggacactaaggaaacaatgtaaattttgctgaattcaactgaccatgtcattgacattgtccttcatgcaatgtaggaaaaggtaatgctttgacacctgaagaaaatgaaatacttgtgcaatttgctcatgaaaagaaaattccagtggaaaacattctcaatattcttgctacaggtaacatcaatgaatatcttttagctataatatatatatttataaatttacatatagatatatatattgagttgtctatctgtctgcctgcctgtctgtctgtctgtctagtcagatgcctgattcatattgtatctagtcgtattgtctttcaggtgtcctttccagcaataatcattggggaaactgttcctatattcttataacacttctgtcactccttaccccactcacgttctttttcagatacttgtcctgaataaaggccttctgtaggtgagtaaaagttgaaggcatgatagccatactgatatttgaatcgatttttcctcctaacaagaaaattagaaataagtcttgacaaaggatgaaaggaactcaaaagtaagcaatataaagagagaatatcattcttcttttaaagctgaaaatgttacaatgttaaaagtgaaaggtagaataatttatgtataagacaatattaagttcttttattcagaattgataatatttttatatttagtaaatttacatcaaaaatgttacttgttatttgaaaaatcgtactttacttatgattgtgaaatataatttgtcacagatacaccacaagcatgtttatattttcaatatattctgtgaaacctcaatctacattttcatgtgggggacacagaatgacattattcactataaactgtgctcatnnnnnnnnnnnnnnnnnnnnnnnnnnnnnttccaggaaatgagctgtttgtgtacaatcacttctgtatactctataatgagatatctaatgttacattcatgcactcttaaaacgactatgttcaaccatgttttaatttttgctttcatgctgccaaggtgagaacaggatggcaattatgcatcatattctcttgatcatggaatcagcatcacaaataaatcaccatttcattctacatgaattgtcccatctcctgtagagccaggataccacatatcaaattcctggcttagctttcctttcccatcacatgatgtattacctgttcttattttttgactgattaaataaattgtttcaagaaacaataaatgcctcgcaaaatgtatctcattcaaaaagcccataaatagtcactaagataattacaataatcactccggtgtaacaaatgatatccgcatcattacaaactattgtagtaattgaaatttcatcattcctggatagccatgggcaagaacagtgcagaagtgaagtttatgttgtgttccatatttactttcagttttaacttgagatttgcagttgaatggttctaacacactgaaataatttgtataacaaactctgtggtt

SEQ ID NO: 367 - DNA, 600 bp, Cricetulus griseus
  misc_feature: Qualifier WAN008D0D_x_at
gtcgacccacgcgtgcggagtaggatcaaattatatgaactctttccaggcttctcttccaaacttgagtattaaggagccttcatatagttcaagggtgagtctggagccagtgtgcggagtgctagcatgaaccggtttcagagatgtaataaaagtgtgcatgggatttcatgtgttgtaaaagctgaacaaattatgtagacatgtattttattaagaatgtgtgcaacttcttatgattataaaccttaaaaaatgctcagctggtgggtagtgatggcgcacggctttattcccaacactcgggaggcagaggcaggcagatctctgtgagtttgagaccagcctggtctacaagagctggttccaggacagcctccaaagccacagggaaaccctgtctctaaaataatgtgtctgttactaggtattactactattaatattttgggatacttccttttgctctcactcttatgtgcattagtaaatgtgtctatttatatattggactttttttttcattttttgtttctttgaagatccacttttaaaataaaagtagatagtatgtacctatagagagaaaaacttaca

SEQ ID NO: 368 - DNA, 2999 bp, Cricetulus griseus
  misc_feature: Qualifier AF281019_at
ggttcggtgcatcggcgcgacggacgggcgggcgggcaggcgctgtctctctcccccctgccggcggctgcgaccgtgggagcccccctgactttcgtcaaacttgctgctttgcccccgtggacatgactccttagctggcattgtgcatgccagttgtgtgtggtggaagcctccctgggtgctcagatggaggagccaatggcgttttctcccctgcttggcggtgaccggagtcccgctgatgactccttaaaaaaatatgagcagagtgttaaactttcaggcattaaaaaagacattgagaagctttgtgaagctgtacctcagcttgtcaatgtgtttaaaattaaggacaaaattggagaaggcacattcagctctgtttatttggccacggcacagctgcaagtaggacatgaagagaagattgcactgaaacacttaattccaacaagccaccctgtaagaatcgcagcagagctccagtgtctgacagttgcgggggggcaagacaatgtcatgggagttaagtactgcttccggaagaacgaccatgtggttattgccatgccgtatctggaacatgggtcatttttggacattttgaattctctttccttccaagaagtacgggaatatatgtttaatctcttcatagctctgaaacggattcatcagtttggtattgttcaccgtgatgtgaagcccagcaattttttatataatagacgtctgaaaaagtatgccttggtggactttggtttggcccaagggactcatgacacaaaaatagagcttctcaagtttgtccaatctgaggctcagcaggaagattgctcacaaagcaagtaccacggagtcactggacaaaagggctcactgagtcgcccagcacctaaaaatctggatcagcagtgtgctgcaaaaacttctgtcaaaagatcctatgccagcgcacaaactcacattaagcaagggaaagacgcaaaggagggatctgtaggcctttctgtccagcgctctgtttttggagaaagaaatttcaatatacacagctccatttcacatgagagccctgcagagaaactcataaagcaatcaaagactgtggacataatctcaagaaagccagcaacaaaaaagacagccatgtctacaaaagctgtggctagtgtgaccatgtctacaaaagctgtggctagtgcgatgaggaaaaccaccagtccttgcccggctgtcctgacctgcgactgttatggaacagataaagtctgcagcatttgcctgtcgaggcggcagcaggttgcccctcgggcaggcacaccaggattcagagcaccagaggtcctgacaaagtgtcctaaccaaaccacagccattgacatgtggtctgcaggtgtcatactcctctccctgctcagtgggcggtacccattttacaaggccagcgatgatttaactgctctggctcaaatcatgacaattcgaggatccagggaaactatccaggctgctaaagcttttggcaaatcagttttgtgtagcaaagaagtcccagcacaagacctgaggaaactgtgtgagagactaaggggtctagaccccaccactcccaagttagcagttgatccaccagggtgtgcctctctagagaacactgaccataaagcttcttccctcggacagacgcctccagcacagcactcagagggttccttgtgtaaaggggacagtgatgactgtgggagtcgtcccagggagtgcactaccaacttggaaggctgggactcggtacctgatgaagcctatgacctgcttgacaaacttctggatctaaacccagcttcaaggataacagcagaggcagccttattgcatccattctttaaagatatgagctcctgaggtggttttccatttacnnnnnnnnnnnnnnnnnntgaggtggggtagataatcacaggttcttgtttgcatccaagtccagggaaggatgactaacttatttttaaagttacaatattttgctctgatgtcagattgtaagcagatggggaattattcaagatccagggggaggcctctatgtgatcttagagttaaaccaccaagtatagtcagatctttagttccctggttttctctgcttctggagcagccttagcctgatgcacgggtcaaggaacgggtcataaaggtcacagatgtgacagggagtaaatgagtgaaaaagactgtttccagatgtctgtggcctacgctgtgtatgcctggggaactggctcttggtggtgtacaggagagacagtgatctttcctataggtgtgcatataaacacagttaccgcagggacataggggaaatgacaccactcaatatagttgtttcctgtaagttacacagtgcttactgcagagtttctggttcttcattaggtgtatttatttatattgggaacctaataatgtgcagattgttgacgaggcagaagtctttttctcgtacagctattttgtcacactatcttagtttaattaggtaattgccacaaggaaaggtgagggtacaactgggaacattactgggttttgtcttccaggagtacccagacttctgcatttctgacctaagttgaccttgctcagagagtaatggtcattgcagtctcaggtggattgggaggaaatacaatgacgtagacaaagcagcaaagggcttgcacggccatctatgcattttttacattctgaactgccctgtgtttgtgatagaatgccagggttcccaggggtcaggaggataaacttgctgaagagttcccttcgtataatcacaggtcatcagtatattcactttcctgttttgaatcctgatagatcaatgtgtgtacacactttgtcttatgctgtatttttttaagcttgaatgtttttggaatatgtatttaataaatgtttcctttcagattcnnnnnnnnnnnnnnnnnnnnnnnnnn

SEQ ID NO: 369 - DNA, 600 bp, Cricetulus griseus
  misc_feature: Qualifier WAN008E4T_at
gtcgacccacgcgtccgcccacgcgtccgcccacgcgtccgcgccgcggtcgctgcacacttccaccgttgccttgcgagcttttgaccccgaggtcggagtctctgcggacagaatgaggcatttaaagagaacttactgtcctgactgggatgaaagagattgggattatggaacgtggaggaacagcagcagtcataaaagaaagaagagatcacatagcagtattcgagaacataagcgatgcaaatataatcactccaaaacgactgatagctattatctgggaagcagatccatacatgagagagagtatcatagtcgacgctacattgacgaatacaggaatgatgacatgggatatgagccaggctattattactatggagaacatgaaagcagatatcagaaccatagtagcaagtcctctggtagaagtggaagaagtagctacaaaagtaaccacaggactcaccaccgcacttcacatcatcgttcacttgggaagagtcaccgaaggaaaagaaccaggagtgtagaggatgatgaggagggtcacctgatctgtcagagtggagacgtactaagtgcaagatatga

SEQ ID NO: 370 - DNA, 556 bp, Cricetulus griseus
  misc_feature: Qualifier WAN008DJD_at
gtcgacccacgcgtgcgggtcgcttctccaggacgtggtgtgtccaggcgtgcggcggcctgcaggaggcccacccgggtcatttcttgagcctgtctctggtgctttctgaagtgaatcctgcgtgacgcttgtcgtcgtcgtctgtgggaacctgcgggcctcgggcgggagagatggatgcaagttcacacgacaagcagccctctgaagccagggagtgtgtcgttaataattctggaaatgcctcttttatcttgggaaccggaaagtctgtgacgcctcataagcgtgtagaagacgcgactcctaatctttgcacccccgttacggttaagtcacctttgaacttctccacgataaccgtagagcagttgggaatcacccctgaaagctttgttaagaccccttcaggaaagtcagcttcccttcagaagaccaggagacgttctacagttggtgtccggggctctccggaaacaaattgcctgatccgtttcattgctcaacagcgaagcctaaagaatgctgcaagatctctgttgactcagaattc

SEQ ID NO: 371 - DNA, 581 bp, Cricetulus griseus
  misc_feature: Qualifier WAN013I2T_at
cgcagaangcggcggtagcgggtggcttgtggtgcagctttaccacagaaggaaccgcgcaggccttantgtgagtggtcactgtagatcccgggaacctggtggccttagtccttcaggtggatcagcgtgcgacatgggaaagaaaaccaagnnnnnnnnnnnnnnntcttcttccgaggatgaggaggagtatgtggtggaaaaggtgttagacagacgcatggttaaggggcaagtggagtatctgttgaagtggaaaggcttttctgaggagcacaatacttgggaacctgagaagaacttggattgtcctgaactaatttctgagtttatgaaaaagtataagaagatgaaggagggtgaaaacaacaagcccagggagaagtcagaaggaaacaagaggaaatccagtttctctaatagtgctgatgatataaaatctaaaaaaaagagagagcagancaatgatntcgctcgaggctttgagagaggactggaaccaganaagatcatttggggcaacagattcctgtggtgacttaatgtttcttaatgaaatggaaagacacanatgaanc

SEQ ID NO: 372 - DNA, 1628 bp, Cricetulus griseus
  misc_feature: Qualifier WAN013I6G_at
acctcattgcgttacttacctcgactcttagctgtcgggaacggtaaccgtgacccgctgtctgcttttgtcaccttcgccttctaacccgtmgmyaccaygcgtgrgkgcmtctccatccacgttggccaggctggtgtccagatcggcaatgcctgctgggagctctactgcctggaacatggsatccagcctgacggccagatgccaagtgacaagaccatcgggggaggagatgactccttcaacaccttcttsrgkgrgacargcgctggcaagcatgtgccccgggccgtgttcgtagacctggaacccacagtcatcgatgaagttcgcactggtacctaccgccagctcttccaccctgagcagctcatmmcaggcaaggaagatgctgccaataactatgcccgtggccactacaccatcggcaaggagatcattgatcttgtcttggacagaattcgyaagctggctgaccagtgcacaggtcttcakggcttcttggttttccacagctttggtgggggaactggctctgggttcacctccctgctgatggagcggctctctgtcgattatggaannnnnnnnnnnnnnnagttctccatctacccagccccccasgktttccactgctgtggktgagccctacaactycatyctyaccacccacaccaccctggagcactctgattgtgccttcatggtagacaacgaggccannnnnnnnnnnnnncnnnnnnnnnnnnncattgagcgccccacctacactaaccttaaccgcctcattagccagattgtgtcttccatcactgcttccctcagatttgatggagccctgaatgttgacctgacagaattccagaccaacctggtaccctaccctckcatccatttccctctggccacatatgcccctgtcatctctgctgagaaagcctaccatgagcagctttctgtagcagagatcaccaatgcctgctttgagccagccaatcagatggtgaaatgtgaccctcgccatggtaaatacatggcttgctgcctgctgtaccgtggggatgtggttcccaaagatgtcaatgctgccattgccaccatcaagaccaagcgcagcatccagtttgtggactggtgtcccactggcttcaaggttggcattaattaccagcctcccactgtggtacctggtggagacctggctaaggtccagagagctgtgtgcatgctgagcaacaccacagccatcgctgaggcctgggctcgcctagatcacaagtttgatctgatgtatgccaagcgtgcctttgtgcactggtacgtgggtgagggcatggaggagggggagttctctgaggcccgtgaggacatggctgccctagagaaggattatgaggaggtgggtgtggattctgttgaaggagagggtgaggaagaaggagaagaatactaatctattctttctgcccctggatcatgtcaaactcctagaacttcagcttcaacattagctgataggcattgattcctctaattgtgtcatcttcattctgtgatcatgtatttgtctgtggccctgtatggtctttccatgatgtctcaaagtaaaaagctttaagacactt

SEQ ID NO: 373 - DNA, 1053 bp, Cricetulus griseus
  misc_feature: Qualifier WAN013HZA_at
aagttgttggcagttagtaagaatcccagcaaacctcattttaatcactatatgtttgaagcaatatgcttatctataagaataacctgcaaagctaaccctgctgctgttgtgaactttgaggaggctctgttcctagtgttcactgaaatcttacagaatgatgtgcaagaatttatcccctacgtttttcaagtgatgtctttgcttctggaaacacacaaaaatgatatcccgtcttcatatatggccttgtttcctcacctccttcagccagtgctctgggaaagaacaggaaacatccctgctctagtgaggctccttcaggcattcttagaaagaggctcaagtacaatagcaagtgctgcagctgacaaaattcctggattactgggtgtcnnnnnnnnnnnnnnnnnntccaaagcaaacgatcaccaaggcttctatcttctcaacagcataatagagcacatgcctcctgagtcagttgaccaatacaggaagcagatcttcattctactgttccaaagactgcagaactccaagaccaccaagtttatcaagagtttcttagtgtttattaatttatattgcattaagtatggggcattagcattacaagaaatatttgatggcatacaaccaaagatgtttggaatggttttggaaaaaatcattattccagagattcaaaaggtatctggaaatgtggagaaaaaaatttgtgcagttggcataaccaaattactcaccgaatgtccaccaatgatggacacagagtacaccaagctatggactcctttgttacaatctctcatcggcctttttgagttgccggaggacgacaccattcctgatgaagagcattttattgacatagaagatacaccagggtaccagactgccttctcacagctagcatttgctggcaaaaaggaacatgatcctgtagggcagatggtgaataacccgaaaatccatctggcacagtcacttcacaaactgtctactgcctgtccggggagggttccctcaatggtgagcaccagcctc

SEQ ID NO: 374 - DNA, 650 bp, Cricetulus griseus
  misc_feature: Qualifier WAN008EZN_at
gtcgacccacgcgtccgcccacgcgtccgcgcagcgcgtcctcggggacacccgcactgctatggctaccggtggcggcgtggcctccagagaggggctgcgctacgccgaatacctgcccccctccacccaaaggccggacgccgacatcgaccacgccgcgggaagaaggctgatagctgtaggactaggtgttgcagctgttgcatttgcaggtcgctatgcatttcaaatgtggaaacctctagaacaagtaatcacagaaacagcaaggaagatttcatcaccgagcttttcgtcctactataaaggaggatttgagcaaaaaatgagtaggcgagaagctagtcttattttaggtgtaagcccatctgctggcaaggccaagattagaacagcacacaagagaattatgattttgaaccatccagacaaaggtggatctccttacttagcatccaaaataaatgaagcaaaggacttgctagaatcgtccaccaaacttaaatgatgctgaaagatcctaccaaaggaaaaggtgcaaggctctagaagtcttcctacagaagactatggaacatggacttgcttcatattaaccaactctgttcttctgtcattaagtgtgcagcaataaaagatatgcacct

SEQ ID NO: 375 - DNA, 580 bp, Cricetulus griseus
  misc_feature: Qualifier WAN013HWL_at
agccggtgctaagaagtcgctctgagatcgcgccgcttargtttttctgttcctttttttcttcctgtttatctccaagcagccagcggcccatagaaacatgaccaccaatatgggccccttacatccatactggcccaggcacttgaggctggacaactttgtgcctaatgacctccctacctggtatatcctggttggcctgttctccgtctctggggtcctaattgtgaccacttggctgctnnnnnnnnnnnnnnnngttgccccacttggaaaatgtcgtcgactggccctgtgctggtttgctgtgtgtgcattcattcaccttgtgatcgarggttggttctctttctaccatggcatccttcttgaagaccaagctgtcttatcccaactctggaaagagtattccaagggagacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttctccaccagaaatccttccgctttatcctacagctcgtggtctctctgggccagatgtac

SEQ ID NO: 376 - DNA, 600 bp, Cricetulus griseus
  misc_feature: Qualifier WAN008CLU_at
gtcgacccacgcgtccgcccacgcgtccggccactgcccctgtttggtggcctttctggcaaaagaatagagagatggtttataaagtcaaattggtggaatggcttagccaagaactagaagtttctgtcctggaggacccgggagataggctgacatcagcagaaattctgtcatacatcaccgagatgtctctcatggaggcctcctgctggactctgctggactctcactcacagccaaggcagcttttctggagtttctctggattcttcagagccaaagatgaaaatgccccacaagctgtaaggtcaaagggcacacccaccacagtgctccagcaagggcgtgttttttaggactccccccagtgcacacaacgtttcttgtaaatgtaataggtgagggatgcatgcggccccttaccatatgactcagtcaagcttcctccgaattagagacttttggggaagttctctctattgatgatcagcactagggaacagaacagaacatcgacgttcagggatcactgcagctttgaccctcgctggagtggtcccctctcaacaaatttccccacagtgttttccaggtgaa

SEQ ID NO: 377 - DNA, 604 bp, Cricetulus griseus
  misc_feature: Qualifier WAN013HW1_at
ctggtttgacaagttcaaatatgatgacgcagaaaggaaattctacgagcagatgaatgggcctgtgactgctggctcccgccaggagaatggtgccagcgtgatcctccgtgacattgcaagagccagagagaacatccagaaatccctggccggaagctcaggccctggggcctccagtggacctggtggagatcatagtgagctcattgtaaggattgccagtctggaagtggagaaccagaatcttcgaggcgtggtacatgatctgcagcagtccatttccaagttggaggtcaggctgaacactctggagaagagttcacctactcacagagccacagcccctcagacgcaacatgtgtctcctatgcgtcaagtggagcscccaaccaagaargcggccacaccggcagaggatgatgaggacgatgacattgacctgttcggcagtgatgaggaggaagaagataaggaggcagcccgactacsaraggagaggcttcgtcagtatgcagagaagawagccaagaaaccctcactggtggccaaatcctccatcctcttggatgtcaaaccttgggatgatgagacaaatatggcc

SEQ ID NO: 378 - DNA, 690 bp, Cricetulus griseus
  misc_feature: Qualifier WAN013HVQ_x_at
gttggnnnnnnnnnnnnnnnnnnnnnnnnngattgcggctgttgttcgagcgctaacagcggcgcggcggtcccagcaagggcctcagacttcggctttgggtgaaaaaaaatggcccgaaccaagcagaccgctaggaagtccacgggtgggaaagccccccgcaaacagctggccaccaaggcagccaggaaaagcgcgccctctaccggcggggtgaagaaacctcaccgctacaggcccgggaccgtggctstragagagatccgtcgctaccagaagtcgactgagctgctcatccggaagctgcctttccagaggttggtgagggagatcgcccaggatttcaaaacggacttgaggtttcagagtgcagccattggtgcccttcawgaggccagtgaagcgtacctggtggggttgtttgaagacaccaatctgtgtgccatccatgccaagagagtcaccatcatgcccaaagacatccakttggctcgccggatacggggggagagagcttaagtcaaggcggtttttatggcattttgtagtaaattctgtaaaatacttwrgtttaatttgtgactttttttgtaagaaattgtttataatatgttgcatttgtacttaagtcattccatctttcactcangatgaatgggaagagtgactgtgcagacctcagtgacg

SEQ ID NO: 379 - DNA, 204 bp, Cricetulus griseus
  misc_feature: Qualifier WAN0088TK_x_at
gtcgacccacgcgtccgcggacgcgtgggccgagttggagcctgaagcccctgcgggtgatggtgataagaaaggcaatgcagagggtagcagtgatgaggaagggaaactggtcatcgacgagccagccaaggagaagaatgagaaggggatgctgaagaggnagaatgcaggggacattgctggagggactcccctaacgtc

SEQ ID NO: 380 - DNA, 529 bp, Cricetulus griseus
  misc_feature: Qualifier WAN013I1P_at
acctggaggtggcaattttggaggtagccctggttatnnnnnnnnnnnnnnnnnntatggtggtggaggacctggatatggcaaccagggtgggggctacggaggtggttatgacaactatggaggaggaaattatggaagtggaaatkrcartgattttggaaattataaccagcaaccttctaactatggcccaatgaagagtggaaactttggtggtagcaggaacatgggaggaccatatggtggaggaaactatggtcctggaggaagtggaggaagtgggggctatggtggaaggagccgatattgagcttcttcctatttaccatgggcttcactgtataaataggagaggatgagagcccagaggtaacagaacagcttcaggttatcgaaataacaatgttaaggaaactcttatctcagtcatgcataaatatgcagtgatatggcagaagacaccagagcagatgcagagagccattttgtgaatggattggattatttaataacattaccttactgt

SEQ ID NO: 381 - DNA, 3249 bp, Cricetulus griseus
  misc_feature: Qualifier X83575_at
ggcacgagcatgaaaccagcgaaagctaagatgcccaggaaacctgtattaaaaaaaggatctcagacaaaccttaaagatccagttggggtgtattgtagggtgcgcccactgagttgtcctgatcaagaatgctgtgtagaagtggtcagtaatacaacggtgcagctccatactcctgagggttatcgacttaacagaaatggagactataaggagactcagtattcatttaagcaggtatttggcactcacaccactcagagggaactttttgaagttgtggctagtccattggtagatgacctcattcatggcaagaatggtctgttgttcacatatggtgttacaggaagtggaaaaactcacacaatgacaggttctccagggtccggaggtctgcttcctcgttgtttgaacatgatctttaacagcataggatcatttcaagcaaaacgttatgtttttaaatctaatgataggaacagcatggaaatacagtgtgaagttgatgccttattagaacggcagaaaagagaagctgtatccattccaaagaccccctctagcaagagacaagcagatccagaattttccgatatgataaatgtacaagaattttgcaaagcagaagaagttgatgaagacagtgtctatggtgtatttgtctcttacattgaaatctataataattacatatatgatctattggaagaggtgcagtttgatcccataaagcccaaactcccacaatctaaaatactgcgggaggataagaaccacaatatgtatgttgcaggatgtacagaagtagaagtgaaatctacagaagaagcttttgaagttttttggagaggtcagaaaaagagacgtattgctaacacccatttgaatagagaatccagccgttcacatagcgtgttcagcattaaactcgtccaggctcccctggatgctgatggagacaatgtcttacaggaaaaagaacaaattaccataagccagttgtctctggtagatcttgctggaagtgaaagaaccaaccggactaaagcagaagggaacagattacgtgaggctggtaacattaatcagtcactgatgacgcttagaacatgcatggaagtccttcgagagaaccagacatatggaactaacaagatggttccgtatcgagattcaaagctaactcatctgttcaagaactactttgatggggaagggaaggtgcggatgatcgtgtgtgtgaatccaaaggctgaagattatgaagaaagcttgcaagttatgagatttgctgaagtaacccaagaagttgaagtagcgagaccagtagacaaggtgatatgtggcttgacaccagggagacgatatagaaacctgcctcggggcggcccagttggagatgaacctttggtgactgaagtgagtctacagagcttcccaccactgcctccatgcaaacttttggatatcaatgatgaggagacccttccaaagctgatcgaaactttggagaaacgacatcgcctacgccaagtaatgactgaggagcttaacagacaatgtatgactttcaaagccttattaaaagaatttgacagttctctttcaaataaagaaaactacattcaggaaaaactaaatgaaaaagaaaaattgatctcaggacagaaatcagaaatagaacgactggaaaagaaaaataaaacattggagtataagatcgagattttggagaaaacaaccacgatctatgaagaagataaacgcaatctgcagcaggaacttgagagccagaatcagaagcttcagcggcagttttctgacaagcgcagattagaagccaggttgcaaggcatggtgacagaaacatcgatgaaatggcagaaggaatgtgaacggtcggtggcagccacccagctggagatgcagaataaactgtgggtcaaagatgaaaagctcaaacagttgagggccattgtgactgaaccaaaacctgagaagccagagagaccctcccgagaaagggaccaggaaaaaatcattcagagatcggtctctccttcgcctgtgcctctttctagtaacaatattgctcagatttccaacggccagcaactcatgagccaaccacagctacacaggcgctctaactcttgcagcagcatttctgtagcttcctgtatttcggaatgggagcagaaactatctccgttcagcacacctgtcaatgtcacctctcttnnnnnnnnnnnnnnnnaggagccaggacaaagtaaaacatgtatcgtgtcagacagaaggcgagggatgtactggactgaaggcagggagatggtccctacattcagcagtgagataggcgtagaagaggaccattgccacaggaacnnnnnnnnnnnnnnnnnncacagaaggtcccgctctgcagggagcagatgggtagatcataagcctgcctctaatgtgcaaactgagacagtcatgcagccacatgtccctcatgccatcacagtatctgttgcaaatgaaaaggcgctagctaagtgtgagaagtacatgctgacccaccaggaactagcctccgatggggagattcagactaaagtaattaagggtgatgtttataagaccagaggtggtggacaatctgttcagtttactgatattgagactttaaaacaagaatcgccgactggtagtcgaaaacgaagatcatccacagtagcacctgcccaaccagatggtacagagtctgaatggaccgatgtagaaacaaggtgttcagttgctgttgagatgagagcaggatctcaactgggacccggatatgagcatcatgcacaacctaagcgaaagaagccttgacctgatagccctggtggccgaagaatgtttttgtttgtgttggtgatttctccaaagctgtgccagacgcagtatctgaatcatcctacagaagaatgtatctggatcacattgtattttttattgtgacttttccaactgttctgaatattttctaaggttttataaaaaaaaaaaatgctgctattgattagccgcaaaaaatgcactttagacatatttaagaatttaaagatggtgagtgggcaatggtaaaaccattttaggaagatgtttagaattctgtatgtatatattcactttctgcttttatatatgccaaaagaattgaaataaaaagcactaaaaannnnnnnnnnnnnnnnnnnnnnnnnnnnn

SEQ ID NO: 382 - DNA, 2703 bp, Cricetulus griseus
  misc_feature: Qualifier U11790_at
ctttcgagcttcttggatttgttttttacctgctgttgttcccgagcccatggagtcgcttcccgctcgattgtttcctggtctctccatcaagattcaacgcagtaatggcttaattcacagtgccaacataagcacagtgaacgtggagaaatcctgtgtttcagtggaatggatagaaggaggtaacacgaaaggcaaagagattgattttgatgatgtggctgcaataaacccagaacttctacagcttcttcccttacacccaaaggacaatctgccactgcaggaaaatgtaacagttccgaagcaaaaacgcagatcagtcaactccaaaattccagctccaaaagaaggtcttcgaagccgttccactcgcatgtccactgtcccagaggttcgaattgccactcaggagaatgaaatggaggtggagctgcctgtggctacaaactctcgaaagcagttttcggttgccactggcctccctaggccctcctgtcctgcaatgacagaattaccgttgtcgatggtcagcgaggaggcagaagagcaagtccatcccacccgaagcacatcttctgcaaaccctgctcggaggaaatcatgtattgtgaaggaaatggaaaaaatgaagaacaagcgggaagagaagagggcccagaactcggaaataagaataaagcgagctcaggaatatgacagcagctttccaaactgggaatttgcccggatgattaaagaattccgggttactatagaatgtcacccacttactctgactgatcctacggaagagcacaggatctgcgtgtgtgtgaggaagcgcccactgaataagcaagaactggccaagaaagaaatcgatgtgatttctgttcccagcaagtgtttgctctttgtgcatgaacccaaattgaaagtggacttgacaaagtatctggagaatcaggcattctgcttcgattttgcctttgatgaaacagcttcaaatgaagtcgtctacaggttcacagcaaggccactggtacagacgatttttgaagggggaaaagcaacctgctttgcctatgggcagacgggcagtgggaagactcacacaatgggtggagacctgtctggtaaatctcagaatacatctaaagggatctatgcaatggcttcccgggatgtcttccttctgaagagtcagcctcgctaccggaacttaaacttggaagtttatgtgacattctttgagatctacaatgggaaggtgtttgatctgctcaacaagaaggccaagctacgtgtactagaagacagcaagcagcaggtgcaggttgtgggacttcaggagtacctggttaactgtgctgatgatgtcatcaagatgctcaacatgggcagtgcctgcaggacttctggacagacttttgccaactccaattcttcccgctcccatgcctgcttccagattcttcttcgagccaaagggagattgcatggcaaattctccttggtggatctggcagggaatgaacgaggggctgacacttctagtgctgaccgccagactcgcatggagggtgcagagattaacaagtctctcctagcactgaaggagtgcatcagggcgctgggacagaacaaggctcacaccccattccgtgagagcaagctgactcaggtgctaagggattcattcattggggagaactcgaggacttgcatgattgccatgatctcaccaggcataagctcctgtgaatatactttaaacacgctgagatatgcagacagagtcaaggagctgagcccccacagcgggctcagtggagagcagccaattcaaatggaaacggaagagatggaagccagctctaatgggacctcgctggcagtcaatttcaaggaagaagaggagctgtcttcccagatgtccagctttaatgaagccatgagtcaaatcagggagctggaagagagggccatggaggagctcagggagatcatacagcaaggaccaggctggcttgagctctctgagatgactgatcagccagactatgacttagagacctttgtgaacaaagcagaatctgccctgactcagcagactaagcacttctcagccctgcgagaagtcatcaaggccctgcgtgtggccatgcaactggaagaacaggctagcaaacagatgaacagcaagaaacggcaccaatgatgacttcaataaagatctgtttatgtcacaccagcctcttcccctatccagcaaactttgggcaccttctggctttggttagggactgagttggatgggttttggtaaatgccaggtaaggggcatcaggcccaggaagactcggagagagaattgctgttctcttgcttagtttggagcaggaggaaggagcttttcatgactgcttccccttttcctccctatcagagggggtgctgtttgggtagtctccctgcggcagcaccctgcccctctgcaacagctgctgatagacaacctgcaaggtccaggctctgggagagaaattggtcctcgtccagcttctactgcctttggtctgtggtatcacccttgctctgagcactcttgtgtcagaaaaaaaaaagtttttgagacctctgttttagttttagaaatccaagatttccatttcctgctgtatgtatttttatgttgtacctaacaataaagaaaga

SEQ ID NO: 383 - DNA, 600 bp, Cricetulus griseus
  misc_feature: Qualifier WAN008D31_at
gtcgacccacgcgtccgcctggtttggcctggaagcttttgcttgcatgggacatacctaccaagatgggtctgctcgtgcagaagtgtcccaggcctgcaacttcctcctgtcccagcagcatggcagatggaggctggggggaggactttgagtcttgtgagcagcggcgttatgtgcagagtgccaagtcccaggtccataatacgtgctgggccctgatgggcttgatggctgtcaggcatcccgacatcactgctcaagagagaggaatcagatgtctgttggagaagcagctctccaatggagactggcctcaggagaacatctctggggtcttcaacaagtcctgcgccatcagctacacaagttacaggaacatcttccccatctgggccctgggtcgcttctccaacctgtaccctgacaattccctcgctggccacctttgaatgcacatgcgtcagcctggatcatacagatccaggcaaggctgggggttcttggctagccctgccctatgccttgttctctatttctgtaaacatgaagctggggatagggtcanggttggtgtctttatgcacttggcagtgtaag

SEQ ID NO: 384 - DNA, 526 bp, Cricetulus griseus
  misc_feature: Qualifier WAN013HZX_at
catcaggatggtgcaggmcaagagctggaccctgaagaagcactttgaaggcttccctactgacagtaacttcgagttgaagactactgaactcccacccttaaataatggggaggtcttgctggaagccctgttcatctctgtgratccttamrtgagaattgcatcgaaaagactgaaggagggtgataggatgatgggtgagcaagtggccagagttgtggaaagcaaaaatccagccttccccgcaggaaccatcgtagtggctttatcaggctggacatcacattccatttctgacgggaatggactgaggaaactgccagcagagtggccagacaagttaccactgtctttggctttggggacagttggcatgccaggcctcactgcctactttggcctgcttgacatctgtggtgtgaagggtggagaaacagtgatggtcaacgcatcagcaggagcagtggggtctgtagtggggcagatagctaagctcaagggctgcaaagttgttggtgcagca

SEQ ID NO: 385 - DNA, 526 bp, Cricetulus griseus
  misc_feature: Qualifier WAN0088X5_at
gtcgacccacgcgtgcgcccacgcgtccggctgaaggagctgagccgggagggcgcggtgtgcggtgaggctgacagcggtctccgttagatcgagtgatggcgcagcagctcgcccgagagcaaggcatcaccctgcgcgggagcgcggagatcgtggccgagtgggtttcgtttggcgtcaacagcattttgtatcagcgtggcatatacccgtcggaaacctttactcgagtgcagaaatacggactcaccttgcttgtaactactgaccccgagctcataaagtatctcaataatgtggtcgaacagctgaaagagtggctctacaagtgctctgttcagaagctggtggtggtcatctcaaatattgaaagtggtgaggtccttgaaagatggcagtttgatattgagtgtgacaagactgcaaaagaagatggtgtttgtggagaaaagtcacacaaagccattcaagatgaaatccgttcagtgatcaggcaaatcacagctaccgtgacctttctgcc

SEQ ID NO: 386 - DNA, 600 bp, Cricetulus griseus
  misc_feature: Qualifier WAN008D06_at
gtcgacccacgcgtgcggcaagtagaggaggagtttctggacatggcagtgctgaaggactacatagcatatgcacatagtaccatcacgccccgactgagtgaggaggccagccaggctctcattgaggcctatgtgaacatgaggaagattggtagtagccggggaatggtttctgcttacccccgacagttagagtcattaattcgattagcagaagcccatgctaaagtaagattttcagacaaagttgaagcaattgatgtggaagaggcaaaacgccttcaccgggaggctctaaagcagtctgctactgaccctcgcactggcattgtggatatatctattcttactacagggatgagtgccacttctcgtaaaagaaaagaagaattagcagaagcattgaggaaacttattttatctaaaggtaaaacaccagctttaaagtaccaacaactatttgaggatattcggggacagtctgacacagcaattaccaaagatatgtttgaagaagccctgcgtgcattggcagatgatgacttcttaacggtgactgggaaaactgtacgcttgctctgagccatcagttgctgc

SEQ ID NO: 387 - DNA, 304 bp, Cricetulus griseus
  misc_feature: Qualifier J00061_at
cagatctggaaatggaccccaactgctcctgctccaccggcagcacctgcacctgctccagttcctgtggctgcaaagactgcaagtgcacctcctgcaagaagagctgctgctcctgctgcccagtgggctgctccaagtgtgcccagggctgcgtctgcaaaggggcatcggacaagtgcacgtgctgtgcctaatgggaggacgatgccgcctcccacgtgtaaatagtgcccggagctctaccctgtttactaagtccccttttctacgaaatatgtgaataaaaaaccaatgtgattct

SEQ ID NO: 388 - DNA, 600 bp, Cricetulus griseus
  misc_feature: Qualifier WAN008CSN_at
gtcgacccacgcgtccgattttgtcggggctgggctttaatggctttgaagaaaatgggaaggcaaaatgggacgcgtgtgccaacatgaaagtgtggctctttgaaacaactcctcgattcaacggcaccatcgcctccttcaacatcaacaccaatgcctgggtagcccgttacatcttcaaacgcctcaagtttcttggaaataaagaactctcacaaggcctctccttactgttcttggccctctggcacggcctacactcaagatacctgatttgcttccagatggaattcctcattgttattgtggaaaagcaggtcaccagcctaatccaggagagccccaccctgagcagcctggcctccgtcactgccctgcagcccttctactacttggtgcagcagaccatccactggcttttcatgggttactccatgactgccttctgtctcttcacatgggacaaatggcttaaggtatacatgtctgtctacttccttggacatgtcttcttcttgagcctactattcatattgccttacattcacaaagcaatggtgccaagaaaagagaagttaaaaaaaagggaataataga

SEQ ID NO: 389 - DNA, 2378 bp, Cricetulus griseus
  misc_feature: Qualifier WAN013I62_at
cgcagcggccgccgccgtccgcccgccctcagccagctgctcggtgccactcgggcagcgtctgcggcggctcgacgaggcgctgacggggcggcggcggacggccggagctcctcggggttcttcggcggcgactagttctcatggggctggccgagtcattgcgtccgtgcgctgtgaggacgttcacattcaaggagttcggaagttcctggatagtgtgcttgggaggaactgccataactggattccatctctagagtttttgtagcacaccgagagccatgaacagcttcaataaggacgagtttgactgccatatcctcgatgaaggctttacggccaaggacattctggaccaaaaaattaatgaagtatcctctgatgacaaggatgctttttatgtggcggaccttggagacgttctgaagaagcacctaagatggctaaaagctctccccgtcactcccttttatgcagtcaaatgtaatgacagcagagcgttagtgaacaccctagctgccattacagttgactgtgcaagcaagactgagatacagttggtacagggccttggagtgcctcccgagagagtcatctatgcaaatccatgtaagcaagtgtctcagatcaagtatgccgccagcaatggagtccagatgatgacttttgacagtgaaattgagttaatgaaggtcgccagagcacatccaaaagttaccaagttggttttgcggatcgccactgacgattctaaagcagtgtgtcgmctcagtgtaaagtttggtgccacactcagaaccagcaggcttctcttggaacgggcaaaagagctaaatattgatgtcattggtgtcagcttccacgtggggagtggatgtactgaccctgagaccttcgtccaggccttgtcggatgcccgctgtgtctttgacatgggaacagaagttggtttcagcatgtatctgcttgatattggtggtggctttcctggatctgaggatacsaagcttaaatttgaagagatcaccagtgttatcaacccagctctggacaagtacttcccgccagactctggagtgagagttatagccgagccaggcagatactacgttgcctcagctttcacactggcagtcaatatcatagccaagaaaatcgtatcgaagggctctgacgatgaagatgagtccagtgagcaaaccttcatgtattatgtgaatgatggagtgtatgggtcatttaactgcattctttacgatcatgcacatgtgaagcccctgctgccgaagagacccaagccagatgagaagtattactcatccagcatctggggaccaacatgcgatggccttgaccggattgtggagcgctgtaatctgcctgaaatgcatgtgggtgattggatgctctttgagaacatgggtgcatacactgttgctgctgcatctactttcaacgggttccagaggccttctatctactatgtgatgtcgaggccaatgtggcagcttatgaagcagatccagaaccatggcttcccaccagaagtggaagagcaggatgttggcactctgcccatctcttgtgcccaggagagcgggatggaccgtcatccagcagcctgtgcttctgctagtatcaatgtgtagatgccattcttgtagctcttaccttcaagtttagcttgaattaagggatttggggggaccatttaacttaattactgttagttttgaaatgactttgtaactacagagtagggcttggcacagatgaagtatggaaggctaggagatgggtcacacttacctgtgttcctatggaaactttgaatatttgttttatatggatttttatcacttttcagacatactaacgtgtgcccctcagccactgagcaagcatttgtagcttgtacatttggcagaatgggccaaaagctaatgttgnnnnnnnnnnnnnnnnnnnnnnntcttgaaataattgggcatcagggaatgtttgcaatgtcccttaaagaagggacacacttcctgcacaggctgctgtagtcatacgagtgagtccaaacatagcagagaaggaaaaggtggatgggacaaggctataccctctacacagcatcagtttgcctagccagtccttaaagtgtgtacttggctgtgaagttggtgaggaacgtcatctgcctagcgacctgatatcctggccacaccccagggaaggcatctaaggctttttgaaccttgcctgcagaagccttgaatgttcctcaacccttgacttcgcctgcatcaccacctagtaacagttggaagtatcatacagcacttggcaatcaacttcctgtaataaatctcaacagcagcaactac

SEQ ID NO: 390 - DNA, 2798 bp, Cricetulus longicaudatus
  misc_feature: Qualifier AB041733_at
cccttcacaggtggcgtcacgggtcctctgcgccgcgcccgaggtcagcaagcaaggacaggcgcctcccagcctgcagccggagccgggcctcgcagcagctcggggactgcagctcaagccacacgttccttggcccacacctgcctccgagtcacgctctgccctgtgaggtgcagcatttgacaagtcattccccagcccccacaagccacgttcccagcttcggagctaacaatccttagctttgagactccctcccttctgcctccatacataagnnnnnnnnnnnnnnnnnnnnncttaccatagttgattttctcttgtgtaaaattaaatttggagttccgggtgccgtgcagaaagattaagactcagcacccgtttgattcaaaacaagttcatgcttattagtctgttggaaaagtgaatcccagcacacacaggatactatggctgaacacggggctcatatcacaactgcttcggtggtggacgaccagccatctatctttgaggtggtagcacaggacagtttaatgacagcagtgagacccgctcttcagcatgtggtcaaggttcttgcagaatcaaatcctgcacactatggcttcttctggaggtggtttgatgaaatcttcactctgctagactttctgctccagcagcattatctttctagaaccagtgcctcattttctgaacatttttatggcttaaagcggattgtagcagggagctcacagcagcctcagagaccagccagtgctggtcttcccaaggagcacctctggaaatccaccatgttccttgttcttctaccctatctgaaagtaaagctggagaagctggcttccagcttgagagaagaggatgaatactctattcaccccccttcctctcactggaaacgattctacagagccttcctggcggcctacccatttgttaacatggcctgggaaggttggtttcttacacaacaacttcgatacatcctaggaaaagctgagcatcactctcccctgctgaaactggctggagttcggctaggtcgactgacagctcaggacatacaagctatagagcatagactgtctgaagccagtgtgatgcaggaccctgtcagaagcgttggtgagaagataaagttagctctgaagaaagctgtgggaggcatcgccttatccctctccaccggcctttctgtgggtgtcttcttcctgcagttccttgattggtggtactcttctgagaaccaggaaaccatcaaatcactgactgctctgcctaccccaccgccacctgtacacctagactacaactctgattctcccctgctacccaaaatgaagacagtgtgccctctgtgtcgtaaaactcgggtgaatgatacagttcttgccacctctggctatgtgttttgttaccgctgtgtgtttaattatgtgaggagtcatcaagcctgtcccatcacaggctatccgacagaagtccagcatctgattaaactctactctccagagaactgaaagaaattagcctattatcacacagctcaagttctgtactgtaaggccacaagactgctgtggtatggcaaacattgatacttccctaattcacaactacagtaattttcagtaactatggattcctttaaaagtatatagccactcggagcgttggtggcacccgtctttaatcccagcaccctggaagcagaagcaggctnnnnnnnnnnnnnnnnnnaccagtctggtctacagagcgagtgccaggataggctccaaannnnnnnnnnnnnnnnnnnnnngggggagggggagaaggatatagccacttgatcctaatgttttgatttgctctctagcaagctggttaagcaggagaattgggattgcaggtggtgtgagggccagtgtggaggtggattaaagaagaccactaagtgttgcttgtcctcaagagtggggttttcagctgaatgtggtcgtgtcatatctgcactcaggaagcaggattttgagttcaaggacagcttaggccaccatagggagcctgtcaaaaaagtaaaaagataaaagtaaaaagaaagtgagagactatgattaagagggataaaaagctcacaggaaaacccaggtcagaccttatacaacaggtggtttgagaaataggccttctctttaaaaaatatttctttacctgtgaagtgtagaggctttgtcagatggcatacactatgaaatgacttcctctttctagaaatatttagggacattttagagaaacaaccccttccatataccagacttactaacatttgattatcaaattaacaaatgctaattgtcagcatttggagattccttcatgagccatacctgtctctcttgggaaacaggttaaagaaaaatagggtttgttaatggggatgagtctggaaatttcaatgtgaatgaaagctagcaagcaatgatgagatgtacgaaaatacttatctaatgtttaccaaatgcttatctaatgaacatttaatgaggagcaaaagctttctacttgctcttatgagttttctcaattgacagtttgctcaattgacatgaatcacccagggtattatgaaagtttctatgaaacatttaagtaaattttaacaatacatgattctgggaaagaactgttcatgctggcacgattatgtggaaactcaaaagattactccaggggtttaaaataaattttcagatcacctnnnnnnnnnnnnnnnnnnnnnnnnnnnnnnnnnnnnnnnn

SEQ ID NO: 391 - DNA, 260 bp, Cricetulus griseus
  misc_feature: Qualifier WAN008DUC_at
agagggaagtgggagtgatgaagatgagaatgatgagggcaatgaagaagaccatagcagccctcctagtgaagtgggctgcaagaagaagaagggtaaagttcttagcagaaacagtgctgatgatgaggaactgaccaatgatgggctgaccctatctcaaagcaagagtaatgaggactcgctgattcttgagaagagtcaaaactggagttcccaaaaaatggaccatattctgatttgctgtgtttgtcttggag

SEQ ID NO: 392 - DNA, 500 bp, Cricetulus griseus
  misc_feature: Qualifier WAN008ET0_at
gtcgacccacgcgtccgactttgtgggttgggctttggacattctgtctccaaacctgatctctaccagcatgcttgggcgcgtcaagtacaacctcaactccctgtcccatgatacagctgcggggctgatacagtatgcgttggatcagggtgtgaatgtcacccaggtatttgtagacactgtaggaatgccagagacataccaggctcgattgcaacagcgctttccagggatggaggtgacagtcaaggccaaagcagactccctgttccctgtggtcagtgctgcaagcatctttgccaaggtggctcgagaccaagctgtgaagaactggcagtttgtagagagtttacaggatctggattctgattatggctctggctatcccaatgatcccaagacaaaagcctggttgaggaaacacgtggaccccgtgtttggcttcccccagtttgtccggttcagctggagcacagcccaagccatcctggaaaagg

SEQ ID NO: 393 - DNA, 534 bp, Cricetulus griseus
  misc_feature: Qualifier WAN008EPM_at
gtcgacccacgcgtccgctggtggataaagagagggaggggaggggctgaactgtgaagattgtggccccagaaccatgtctactcgggagtcctttaacccggaaagttatgaattggacaagagcttccgcctaaccaggtttactgaactgaaaggcacaggctgcaaagtgccccaagatgtcctacagaaattgctggagtctttacaagagaaccacttccaagaagatgagcagtttctgggagctgttatgccacggcttggcattgggatggatacttgcgtcattcctttgaggcatggtggtctttccttggttcaaaccacagattacatttatcctatcgtcgatgacccttacatgatgggcaggatagcatgtgctaatgttctcagtgacctctatgcaatgggcgtcacagaatgtgacaatatgctgatgctccttggagtcagtaataaaatgactgacagggaaagggataaagtgataccgctaattatacagggttttaaagatgcggcaga

SEQ ID NO: 394 - DNA, 1434 bp, Mesocricetus auratus
  misc_feature: Qualifier M74776_at
cgggaacccactgcaaatggcctggtcaaccagaacaatgatgtcacttgctctgtacacctgtttcctctggctcctcaccagtggcctcaagactgtccaatccttgcatgattctcaccgaggcctagctcctactaatgttgactttgccttcaatatgtatcgtcacctatcgaccttggatcctcacaagaatatcttaatttcccctgtgagcgtctccatggccttggccatgatgtcccttgtcgccgtaggctccaaaaagaaccagtttttccaggatgtgggcttcaacctcacagagatcagtaaagaagagatctatcagagcttcgagaagctcagtcaccttctcagcaaggcagacagcagcttggagatgcgcatgggcaataccatgttccttgaccagagcctgaatatgagggactcattcttaacagacatcgaacactatcatgaatcagaggccttgactaccgatttcaaggacggggctgatgcccgtgagcatatcaacaggcatgtggagactaagacacagggagaaattacacatgtgttctcagaccaggatagtccagctcccctcaccctggtcaactacaatgtcctcaaaggcatgtgggagctccccataagcccagagaataccagggatgaggacttccatgtaagcgagaatagcacggtcagggtgcccatgatgttccagtcaggtgtcatcggttatcttcatgactcagagatcccctgccagttggtacagatgcagtatttgaaaaatgggactaccttcttcatccttccagacgagggccagatggacgctgtcaccgctgcacttcatagggacacagttgaaaggtgggacaagctgctgaccaaaaggctggtgaacctgtacatccccagagtctacatgtctggtacctacaacctcgaagatgtgctggagggcatgggcattacaggcttattcaccaaccaaacagatttcttagacatctcccaggatcctccacagaaagcatcaaagatagtccacaaggtcatgctgcaactggatgagaaagatgaaccgcctgttaccaccacagaggctcccccacagacaacatctgaaccgctcaccctcaccttcaacaaaccnnnnnnnnnnnnnnnnnnnnnnnnnnncacttggagcagcctgttgttgggcaaaatcatgaatccagcttagtgcatgctggagaagctatggtaccgcctgactttggggggtagaagcctttccccagtcttctccaggttcttccgaacccaactaggtatccctacttttagatatcctgtaatgtataaccattgttgctactcccaagacaggtcaactgataaagtagcactgagatagagggaactgttattaaagggtgtttttgc

SEQ ID NO: 395 - DNA, 1959 bp, Cricetulus griseus
  misc_feature: Qualifier Y12074_at
gccgcagccagctgtctcctcctagcgctgctggggcggcgggctatctaggccggcgcgtctctatggctgcaggggcggtagttccgcaggctatctctgcaccatggctcaggcgagagaaaatgtcagtttattcttcaagctatactgcttggcagtgatgactctggtggctgcagcttataccgtagctttaagatacacaaggacaacagctaaagaactctacttttcaaccacagccgtgtgtgtcacagaagttataaagttactgataagtgttggacttttagctaaagaaactggaagtttgggtagatttaaggcatctttaagtgaaaacgtnnnnnnnnnnnnnnnggaactgatgaagttaagtgtgccatcactggtgtatgctgttcagaataacatggctttcctagctcttagtaatttggatgcagcagtataccaggttacctatcaactgaagattccctgtactgctttatgtaccgttttaatgttaaatcgaacactcagcaaattacagtgggtttcagtttttatgctgtgtggtggggtcatacttgtacaatggaaaccagcacaagctacaaaagttgtggtagagcagagtccattattaggctttggtgcaatagctattgctgtattgtgctctggatttgcaggagtttattttgaaaaagttttaaagagttcagatacttccctttgggtgagaaacattcaaatgtatctgtcagggatcgtggtgacgttagttggtacctacttatcagatggagctgaaataaaagaaaaaggatttttctatggttacacgtattatgtctggtttgtcatcttccttgctagtgttggaggcctctacacttctgttgtggtcaagtacacagacaacatcatgaaaggcttctcagcagctgcagccattgttctttctaccattgcttcagtgatgctgtttggattacagataacactttcctttgcaatgggagctcttcttgtatgcatttccatatatctctatgggttacccaggcaagacactacctgcattcaacaagaagcaacttcaaaagaaagagtcattggtgtgtgattggaatcttaagatactcctataacgactaaactgtcaataataaattagagccttaagtcaaccccagatggtagcttaaacaatatcaacaaaattactgtatggcattaaggtacagagaactgtatgagtgaatgcttaacagatttatttagagtgtttggtatcaagctgtattatttcagaatgaaggactttactacagagagagagtgtgtgtgcatgtgtgtatgtatactgtgtacacagagcatggagtttacatggtatcaagaggcatgcacagaggagacaacacagggttacaacatcaggtgaaactacaatattcaatcaaggtttaagatttggtatctgaaggttcaagtgccaaagccatttctgtcacactgttctctcgttcaggtaccaggaggggaagcttagccttccttgttgtccagttcatggatagtactttgtccctgtagtagcaaaaaatctagctgcttttatcaaagtgaagaatgaataagataagacagttcagaacaaaaccagtgtgtaacttctaaaacagaacctatttcatggctcagacactgacttctgtcgtttgaatnnnnnnnnnnnnnnnnnnnnnnnnnnnnnnnnnnnnnnnnnnnnnnnnnnnnnnnnnnnnnnagctgatatacttttcagaaaattttctaagtcgcttttatactttcttttttataaagtatggttacctgttgggctctcaatttgtgaactttaagtgatttaaaatatttctataatgttaacggggaaatccagcaataaatttatttatannnnnnnnnnnnnnnnnnnnn

SEQ ID NO: 396 - DNA, 967 bp, Mesocricetus auratus
  misc_feature: Qualifier AJ245700_at
gtaggttcctggccatgctggctctggtctttgtcgtcgtcatggtgtggtactccatctccggagaagatacgtactttgatctcttttatcttcccaactcagagaagaaagagccatgcttccagggtgaggcagagaggcaggcctctaagattttcggcaaccattctagggaacagcccatcttcctgcagcttaaggattatttctgggaaaagataccatctacctatgagttgccctttgggaccaaaggcagtgaagaccttcttctccgggtgttagccatcactagctattctatacctgagagcatacagagcctccggtgtcgtcgttgtgttgtggtggggaatgggcaccggttgcggaacagttcactgggaagtatcatcaacaagtacgatgtggtcatcagattgaacaatgctcctgtggctggttacgaggcagatgtgggctctaaaaccaccatacgtctcttctaccctgagtcggcccacttcgaccccaaaatagaaaacaacccagacacgctcttggtcctggtagctttcaaggcaatggacttccactggattgaaaccatcttgagtgataagaagcgggtacggaaaggtttctggaaacagcctcccctcatctgggatgtcaaccctaaacagattcggattctcaaccccttctttatggagnnnnnnnnnnnnnnnctcctgagcctgcccatacagcagtcacgaaagatcaagcagaaaccaatgacaggtctgctagccatcaccttggccctccacctctgcgacctggtgcacatcgctggcttcggctacccagacgcctacaacaagaagcagaccatccactactatgaacagatcacgcttaagtctatggcggggtcagggcacaatgtctcccaagaggccctagccatcaagcggatgctagagatgggcgctgtgaa

SEQ ID NO: 397 - DNA, 565 bp, Cricetulus griseus
  misc_feature: Qualifier WAN008EN4_at
gtcgacccacgcgtccgccctgccaaaaaagggtcagaactgaatgtaaatgtggggctgagacttgccgaggttacctcaactgaaacttaaagaaatggagcctcatgattgttttgttttgttctaagaactgaaaaagtatttgggatgcctttctatcatcaagattatatgtatatgttaatgtacaacttgtgtctcaaccaaagtacattactattgcttctgaagaggactgcagggccacataaatagaatgtatttggtggttacatactaaatatggaaagtgaactgtttccaaatcaccatacacttagttaagaaatctgaatagacaagtgtcccctttagtgtttgaaaagcaatgaggcaatggctgttactatgacaagcagataattcaatttaggtaaatgtacagattttatttttttttactctattgttcctacttaatactcactgatcttgtatttgagacaaataggcggcactgaatatttttactatattttctacttttgcattaaaacattggcatcttaatgatatatttcag

SEQ ID NO: 398 - DNA, 1097 bp, Cricetulus griseus
  misc_feature: Qualifier X98066_at
gagacgcgcttcttcggccgtgccccggcgctggctgattgttgggcggccggcgccatgtctgtgagcgagatcttcgtggagctgcagggctttttggctgcggagcaggacatccgagaggaaatccgaaaagttgtgcagagtttagaacaaactgctcgagagattttgaccttgcttcaaggggtccatcagggcactgggtttcaggacattccaaagcggtgcttgaaagctcgagaacattttggtacagtgaagacacacctcacatctctgaagaccaagttccctgcggaacagtattacaggtttcatgagcactggcggtttgtgctccagcgtcttgtcttcctggcggcctttgtagtatatttggaaacagagaccctggtgactcgagaggcggttacagagattcttggcattgaaccggatcgggaaaaaggatttcatctggatgtggaagattatctctcaggagttttaattcttgctagtgagctgtcgaggctgtctgtcaacagtgtgactgctggagactactctcggccccttcacatctctactttcatcaatgagctggattcaggtttccgccttctcaatctgaaaaacgactccctgaggaagcgctacgacggcttgaagtacgatgtgaagaaagtagaggaggtggtctatgatctttccatccgaggcttcaataaggagacagcagcagcttgtggtgaaaaataggagcctctccctgtggctggccttgccgactaggcatttgccagggagggctagcacagtgcctctcctctagttagcacgccagttgctaaacactgcgctttattttcgtaaccagctgtgtggcgtgaatatcagaattgagatactttttggatctaaaaaaaaaaaatcctttccactgataggattttctttgttgacgtttcagtgaacttgctgtgtaatttgttgtagtatcattttgtcaaaagtgtaaatgttggtctcttggtaaaagtccttttcttgcttnnnnnnnnnnnnnnnnnnnnnnnnnnnnnnnnnnnnnnnnnnnnnnnnnnnnnnnnnnn

SEQ ID NO: 399 - DNA, 293 bp, Mesocricetus auratus
  misc_feature: Qualifier D86467_at
tcatgagatacctctaagctacagtcatgtgcctgaagtttctcttctgaccacaggtaccttctggatcccactacgtggtccaagtgccatgagcctaagaatattgtggagtggaatgtgacgctattttctaccctcttggcgtttggtggacttgaattcatcttgtgtcttattcaagtcataaatggagtgcttgaaggcatgtgtagctattgctgctcccaccaacaggtgagaatttgtgtgacaacgttgaggtctagccccgtagtgttcagttgccacaa

SEQ ID NO: 400 - DNA, 1777 bp, Cricetulus griseus
  misc_feature: Qualifier WAN013I9O_at
gagccagcgcccgagcgcagacgcgcccgccatgagggagattgtgcacattcaggcgggccagtgcggnnnnnnnnnnnnnnnnaagttttgggaagtgatcagcgatgagcacggcatcgaccaggccggaggctacgtgggcgactcagcgctgcatctggagcggatcagcgtctactacaatgagtcatcttctcagaagtatgtgcctagggctgcgctggtggacttggaacccggcactatggacagtgtgaggtctgggccttttgggcagctcttccggcctgacaacttsatcttcggacagacgggcgccgggaacaactgggccaagggycactacactgagggcgccgagctggtggactcagtgctggatgtgattcgcaaggagtgtgaacactgcgactgcctgcagggcttccagctcacccactcgttgggcggckgcacaggatcakgcatgggcaccctgctcatcagcaagatccgggaggagtacccggaccgcatcatgaacaccttcagcgtcrtgccgtcgcccawrgtctyakacaccgtggyggaaccttacaatgccacattgtcggtacaccagctggtagagaacactgacgagacctactgcatcgacaacgaggccctctacgacatctgcttccgcactctcaagctgaccacacccacgtacggggacctcaaccatctggtgtctgccaccatgagcggtgtcaccacatccctgcgttttcccggccagctcaacgctgacctgcgcaagttggctgtgaacatggtgcccttcccacgcctgcacttcttcatgcccggcttcgccccactcacagcccggggcagtcagcagtaccgtgccctgacagtgcccgagctcacacagcagatgttcgatgccaagaacatgatggctgcctgcgacccacgccacggccgctacctgaccgttgccaccgtctttaggggccccatgtccatgaaggaggtggacgagcagatgctggccatccagaacaagaatagcagctactttgtggagtggatccccaacaacgttaaagtggccgtgtgtgacatcccaccccggggcctgaagatggcttccaccttcattggcaatagcactgccatccaggagctattcaagcgcatctccgagcagttctcggccatgtttcgccgcaacgcttttctgcactggttcaccggcgagggcatggacgagatggagttcaccgaggctgagagtaacatgaacgacttggtgtctgagtaccagcagtatcaggacgccacggtcaacgacggggaagaagcttttgaagatgaggatgaagaagagatcaatgaatagggggcctttaagatgctatggtgcacacctgctcttagccctgatggtgtgagcacggtgccctggtctacacatcacaggcccctctcaaccaaatacacaccactgtnnnnnnnnnnnnnnnnnnnnnnnnnnnnnctttcctttaccagacaattgacagtcctaccagaccagggtgatttcatacaggaacactgaaagcaaagttagggggctgtaaaaggctgaagacagagaccaccttccgttaagtgcccagccatcagagttagcagggaagttagaataaagatttgtgtttcatccttggtgataaaatctaaagcaccgcattgttgccttaattgaatatacaatatggaactttgtgacaatccattcataataaaatgctaaacc

SEQ ID NO: 401 - DNA, 600 bp, Cricetulus griseus
  misc_feature: Qualifier WAN008CS2_at
gtcgacccacgcgtccggcgccgccgggatcctgctctccatctacgcctaccacgtggagcgggagaaggagagggacccggagcaccgggccctctgcgacctggggccctgggtgaagtgctccgccgccctggcctccagatggggtcgaggatttggtcttttgggttccatttttggaaaagatggtgtattaaaccagccaaacagtgtctttggacttatattttatatactacagttattacttggcatgacggccagtgcagttgcagctcttgtcctcatgacctcctccatcgtgtctgttttggggtctttgtacctggcctacattctgtactttgtgctgaaggagttttgcatcatctgcgtcaccacatatgtgctgaacttcattctcctcatcatcaattacaaacgactagtttacttgaatgaggcctggaagcgacagctgcagcctaaggaagactgacggctggcggattcccacccaactgtctcaaacacttctttcattaagtttattgtgcaagtttttttttttttttaaaatacacaacagacacttttccctgagaatctataactgat

SEQ ID NO: 402 - DNA, 179 bp, Cricetulus griseus
  misc_feature: Qualifier AF022945-rc_f_at
tgcgaagccctcaagcacagatgcaaatgtactctctgggactcactggctggcactgatattcgcagtcagatccgtagcacggatccaggagctccacgcactctccatccaccaactcaaagccatcatagcagagacattcaaagccgcccttggtgttgacacagcgctgccgg

SEQ ID NO: 403 - DNA, 392 bp, Cricetulus griseus
  misc_feature: Qualifier WAN008EB0_at
gtcgacccacgcgtccgcatgtactctgcaggctgcgtcgtcaccatctctggacgcatgaccttcacgagcaacaagtccatggagattgaggtcctggtggatgctgaccctgtggtggacaactcacagaagcgataccgggccgccagtgccttcttcacctatgtatccctgaaccaggagggcaagccgctgcctgtacctcagcttgtggtgagtgtcctaaaccatgggctgcccttaggtctaatgaagtcttgagaagaagcaccctgtaagctgtaagctataggctggacagatgggtcagacctctgagctcgtcaccatggctggcacactgtctggagctcctggcaatgtgctcatgtgctggagtgatgtcttag

SEQ ID NO: 404 - DNA, 526 bp, Cricetulus griseus
  misc_feature: Qualifier WAN008CI5_at
gtcgacccacgcgtccgggcgttcggcagactcacgcaccaactgtgaggagccgggggtttgtgcctgggaaggccgcgtaccgggaggtgcccgactggtgggagctgtggccggcggagcccataagcccggcgcagtcccgaactcggtcgcctcttgcccctgtaggcactgtcatggcgcagttcgtgttcgagagcgacttgcactctctccttaaactggacgcgcccatccccaatgcacccgttgcgcgctggcagcgcaaagcgaaggaagccaccggcccagctccctcgcccatgcgggccgccaacagatctcacagcgccggccggaccccgggccgaactcctggcaaatctaattccaaggttcagagcacccctagcaaacctggaggtgaccgctatatcccccatcgcagtgcttctcaaatggaggtggccagcttcctcttgagcaaggagaaccagccagaagacggggagacgcccaccaagaaggagcatcagaaagcctg

SEQ ID NO: 405 - DNA, 350 bp, Cricetulus griseus
  misc_feature: Qualifier WAN008DZY_at
tgtcactcgtgtctctgaagtcaaacctcggatggtggtggccacatatacttgtgatcagtgtggggcagagacttaccagccgatccagtctcccaccttcatgcctttgataatgtgtcccagccaggagtgccaaaccaaccgctcaggagggcggctttacctgcagactcgtggttccaaatttattaaattccaagaaatgaagattcaagaacatagtgatcaagttcctgtgggaaacatccctcgtagcatcacagtggttttacaaggagagaatacaaggactgccctgcctggtgaccatgtcagtgtcactggtatcttcttgccagtcctgcgta

SEQ ID NO: 406 - DNA, 600 bp, Cricetulus griseus
  misc_feature: Qualifier WAN008D2Z_at
gtcgacccacgcgtccgtctctgtgatttcatcactgtccacactcccctcctgccctccaccacaggcttgctgaatgacagcacttttgcccagtgcaagaagggcgtgcgagtagtgaactgtgctcgagggggaattgtggatgaaggtgccctgctccgtgccctgcagtctggacagtgtgctggtgctgcactggatgtgtttacagaagagccaccacgggaccgggccttagtggaccatgagaatgtcatcagctgtccccatctgggtgccagcaccaaggaggcccagagccgctgtggggaggagattgcagtccagtttgtggacatggtgaagggaaagtctctaacaggggtcgtaaatgcgcaggctcttaccagtgccttctctccacacaccaagccttggattggtctggcagaagcgttgggcactctgatgcatgcctgggctggctcgcctaaagggaccatccaggtggtgacacaaggaacatctctgaaaaacgctgggacctgcctgagccctgcagtcattgttggacttttgagggaagcgtcaaagcaagcggatgtgaacttggtgaac

SEQ ID NO: 407 - DNA, 1689 bp, Cricetulus griseus
  misc_feature: Qualifier WAN013I9V_at
ctcctcttcctcacatctgagcctcttgatcctaccgtgttgccaaaatgtcgctttctaacaagctgactttggacaagctggacgtgaaggggaagcgggtcgtgatgagggtggacttcaatgttcctatgaagaacaatcagataacaaataaccaaaggatcaaggctgctgtcccaagcatcaaattctgcttggacaatggagccaagtcagttgtccttatgagccacttgggccggcctgatggtgttcccatgcctgacaagtactccttagagccagttgctgcagaactcaaatctctgctgggcaaggatgttctgttcttgaaggactgtgtgggcccagaagtagagaatgcctgtgccaacccagcagctggcactgtcatcctgctggagaacctccgctttcatgtagaggargaagggaagggaaaagatgcttctggcaacaagattaaagctgagccagccaaaatcgatgctttcagagcctcactatccaaacttggggatgtctatgtcaatgatgcttttggaactgcacaccgagcccacagctccatggtgggtgtgaatctgccacagaaggctggtggatttttgatgaagaargagctgaactactttgccaaagctttggagagtcctgaacgccccttcctggctatcttgggaggagctaaagttgcagacaagatccagctgatcaataatatgctggacaaagtcaatgagatgatcattggtggtggaatggcnnnnnnnnnnnnnnnnnnnnnnnnnnnnnnggagattggaacttctctgtatgatgaagaaggggccaagattgtcaaagatctcatggccaaagccgagaaaaatggtgtgaagattaccttgcctgttgactttgtcactgctgacaaatttgatgagaatgccaagactggccaagctactgtggcctctggtatacctgctggctggatgggcttggactgtggtactgagagcagcaagaaatatgctgaggctgtggctcgagctaagcagattgtttggaatggacctgttggggtttttgaatgggaagcttttgcaaggggaaccaagtccctcatggatgaggtggtaaaagccacttctaggggttgcatcactatcataggtggtggagacactgccacttgctgtgccaaatggaatacagaggataaagtcagccatgtcagcactgggggtggtgccagtctagagctcctggaaggtaaagtccttcctggggtggatgctctcagcaatgtttagtattttcctgccttttggttcctgtgcacagccagcctctaagtcaacttagtgtgtttccacatctccatttggtgtcagctagtggcggagatgcagcaccaggaacccttaaacagttgcacagcatcttaggctcgtctttactgcatcgggatttgtctacattcttcaagatcccattttaatcccttagtggctaagtgcattctagagtgcatctatttatattctgcctgaaaagaaagtgagctataaaagcttagttctctttgagtannnnnnnnnnnnnnnnnnnnnnnnnnnnnnnnnnnnnnnnnnnnnnnnnnnnnnncagctgtaggtttggagaagttgatgctctattaataaagatgtccactgacaa

SEQ ID NO: 408 - DNA, 141 bp, Cricetulus griseus
  misc_feature: Qualifier L00365_at
tgctgactcttgcagcaccgagctcatgaggagagtccggcgcttccagatcgcccagaacaaatgcctggtcatcaagtatgccaaagacacgcgctatagcagcagcttctccacacatgaccggtcagtcccaccacc

SEQ ID NO: 409 - DNA, 222 bp, Cricetulus griseus
  misc_feature: Qualifier AF022941_x_at
aacaattccacagcctcccggactgctgcacgtccccacgggccaaggatcctttgcccgtggggacgtgcagcagtccggaggcttgtggaagtgtttatttatattgtccttttttaccgaagatatgcatactccatcgatgttgtattcacagtggctgaggaattcttgtacccagtttctttggcttcaagtccgattaaaagaccgtgtgaaacg

SEQ ID NO: 410 - DNA, 521 bp, Cricetulus griseus
  misc_feature: Qualifier AF081141_at
aagcttttaccgccaagaggatcccagagaagaggctggagagctacaagagaatcaccagcagcaagtgtcccaaagaagctgtgattttcatcaccaagctcaagagagagatctgtgctgaccccaagcaggactgggtccagacatacacaaaaaaactggaccagagccaagctaaatcagaagctgcaactgtgtataaaacagcacctttgaatgctaacttgacccatgagtctgcagttaatgcatccactactgcctttcccaccacagacttgagaayttctgtaagagtgaccagtatgacagtgaactagtgtgacctggaatgtgatgccttaagtaatattaactttatttaacttattaatgtttcaatatccccctccctgtatacttggatttcttagatgcatgacgtggtttcattgttttccctctgtgaaccctggttctccaccctaaatgttatgagagatgacttttgtaaagatgagnnnnnnnnnnnnnnnnnn

SEQ ID NO: 411 - DNA, 2894 bp, Cricetulus griseus
  misc_feature: Qualifier AF254572_at
ggcacgaggtgggtgcgcagcgcagctggctgtcccgggaggaccccagctcggacgggcgagccttgtgtagctagctccccgagtgcctggactggaggcggtggaaagcgccaggcagcggccagacctggcaagcttcggcgtgagacatgccatcgtatctcacaaggcagaagacccgaaaaattttctcatgggttggcaggccattgccagatcgaaaacacaaccaccaaatctacaaagaaatgcgaatgaaagtcaatgattgttccactgagattcacatcaaggttggacagtttgtattgattaaaggaaataataatgcaaagccctatgttgctaaactgattgaattatttgaagatggatctaaatttcattccaagaaatgtgctcgagtacagtggtgtgtccgattctctgagattcctatctctaaaaggcatttgttaggccggaagcctgctgcacaggagatattctggtatgacgatcccgactgtggtaacaacatttgtgtggagaccatcattggtcctgttcaggtagtggctttagccccagatgaagtgatccctatggatcagaaaagtgaggacacactgtttgtgaagctatcctggaatacaaaaaactttgaaccactgtcaccagaagtacttgcagcattgaagaaactagaagatagccctaagtgccagaaacctttagaaggcaagagtaagaatgtaaaaaacccttcttggaacgcaacagaacaaatggtcaaaaggattgaatcaagtcactctacctccaaatcttgccagacatctgctcatcctgtctcccccaatgcaaggaagccgctggaactcagtgactcagacattcccaggaagcctaacatgaggctatcacagaagatcttgtgtgactccttggattctcaaaaaaattctaaacgaaaaacagccttctctgagacagcctcaccacctaaaagatgtcagcctggtgaaatcaagaacttgtcacctttgaaaactctggaaaagaatggacaggctcattccttttgtgacaaaggtagcatgattctaagagcgcaagacccatctttcataactacaaagattagtatggagagggcactcgaccctgtcaggagcagattgaggccttcagtgatgctcacttcaattctgacaccaaagcggactgggagggaggcgggaaaacaagaagctcacacagaacccacccgtacttcccatcgtgttcatagaaggagctctctcttgactttgaatcggattaggcagcagctttggcttctcgatgatgataagagtgaccaagaagaggaagagtccatatcctcagcagaggtgtcagactccactagtgaggaggaagatgattctattccttcccttctaaagagaaattcccagccgcagtccaggaaccggagaatggcctcaaagccatcattgcagaccccctccaagacaccaaagaaaactactcatcatccaactcctcagattcggggtagaaatatcgctgtccaggagccaacaagtgtgcttgaagaggcccggctgatgctgcatgtttctgctgtgcctgactctcttccctgtcgagagcaggaattccaagacatctatagctttgtggaaagcaaacttcttgatgggactggagggtgtatgtacatttctggggttcctgggacagggaagacggccactgtaaacgaggtcatacgctgtctgcagcaggcagcccaaacaaatgatgttcctccctttgaatatgttgatgtcaatggcatgaagctgacagagccccaccaagtctatgtgcagattttacagaagctgacgggccagaaggcaacagctaatcatgcagcacaactactggcaaagcgattctgcagccaagggtcccaacaggagaccactgtgctgcttgtggatgagcttgaccttctttggacttccaaacaagatgtcatgtacaacctctttgattggcctactcacaaaggagcccggctgatagtcctggccattgccaacaccatggacctgccagagaggatcatgatgaaccgtgtatctagtcggctgggtctaaccaggatgtccttccagccctattctcacaaccagctgaaagagatcctagtgtcccgactcaagcatttaaaggcctttgaagatgatgccatccaattggtagccagaaaggtagcagccctctctggagatgcacggcgctgcctggacatctgcaggcgtgccaccgagatttgcgagctttcccataatcatggcaactctctgggcccagtgactgtgtcccacttaatggaggctatagatgaaatgttctcctcatcctacatcactgccatcaaaaactgttccctcctggaacagggcttcctgagagccattattgcagaattccgtcgatctggactagaggaagcaacatttcaacagatatacagtcaacatgtggctctgtgcaggatggagggactgccttaccctaccatgtcggagaccatggctgtgtgctcaaatctgggttcttgtcgcctcctcctggtggagcccagcaggaatgacctactacttagagtaaggctcaatgtgagccaggatgatgtgctctatgctctgaaagaagagtgaagagcttcacagacttctgctgtttctcagcctatttatttttcttcagaacatgaataatagcaaaatatacagaatggaaatgattgttgtagattttggcatttatgttatttagtttgctttttccagagtgtttgagaaatttgtggtaatgtactatgtatataaataaataaaaataaatat

SEQ ID NO: 412 - DNA, 124 bp, Cricetulus griseus
  misc_feature: Qualifier L00366_x_at
ctttcttcttcccaggaacaccatggacgccctgccagcctgcctgctccgggatgtggcccaggaggccctgggtgcggctgtcattggcatnnnnnnnnnnnnnnntgtaagttggcttgaa

SEQ ID NO: 413 - DNA, 1578 bp, Cricetulus griseus
  misc_feature: Qualifier M12329_at
agtctatataagacctctcctggaggcggactcacactgcttctccccgggactccttggtagtctgctagtgggaggtcctcgtcaccctctcttaactcagacctcgcggaccgcttcctagacaacatgcgtgagtgcatctccatccacgttggccaggctggtgtccagatcggcaatgcctgctgggagctctactgcctggaacatggcatccagcctgatggccagatgccaagtgacaagaccatcgggggaggagatgactccttcaacaccttcttcagtgagacaggcgctggcaagcatgtgccccgggctgtgttcgtagacctggaacccacagtcatcgatgaagttcgcactggtacctaccgccagctcttccaccctgagcagctcatcacaggcaaggaagatgctgccaataactatgcccgtggccactacaccatcggcaaggagatcattgatcttgtcttggacagaattcgtaagctggctgaccagtgcacaggtcttcagggcttcttggttttccacagctttggtgggggaactggctctgggttcacctccctgctgatggagcggctctctgtcgattatggaannnnnnnnnnnnnnnagttctccatctacccagccccccaggtttccactgctgtggttgagccctacaactccatcctcaccacccacaccaccctggagcactctgattgtgccttcatggtagacaacgaggccannnnnnnnnnnnnncnnnnnnnnnnnnncattgagcgccccacctacactaaccttaaccgcctcattagccagattgtgtcttccatcactgcttccctcagatttgatggagccctgaatgttgacctgacagaattccagaccaacctggtaccctaccctcgcatccatttccctctggccacatatgcccctgtcatctctgctgagaaagcctaccatgagcagcttacagtagcagagatcaccaatgcttgctttgagccagccaaccagatggtgaaatgtgaccctcgccatggtaaatacatggcttgctgcctgctgtaccgtggggatgtggttcccaaagatgtcaatgctgccattgctaccatcaagaccaagcgtaccatccagtttgtggactggtgtcccactggcttcaaggttggcattaattaccagcctcccactgtggtacctggtggagacctggctaaggtccagagagctgtgtgcatgctgagcaacaccacagccatcgctgaggcctgggctcgcctagatcacaagtttgatctgatgtatgccaagcgtgcctttgtgcactggtacgtgggtgagggcatggaggagggggagttctctgaggcccgtgaggacatggctgccctagagaaggattatgaggaagttggggcagacagtgctgaaggagatgatgagggtgaagaatattagcccatgtgctgcaattttacacttcattgtcttgtgtttgtctttgtatgtatatgactgtcttacactatcaataaaggtgtttccatattaagtggg

SEQ ID NO: 414 - DNA, 300 bp, Cricetulus griseus
  misc_feature: Qualifier WAN0088J9_x_at
aggtcgacccacgcgtccgtgataatgtcacttagaaaggctaacttggtttattgttcagtgggggtagaaaaagcttcctaaaactgaaatatttcaaggaaactgatcttcaaattctgacattggttattctaagcaactgaaccaattgctcacttggtcataaaataagcaaaaactagattcttcagtgtagcagagactccccagactcattcagttctacttttacaaataagattttacatgtattaggaagattttgtgcaacagaaaagttgtacagaaagtacattc

SEQ ID NO: 415 - DNA, 600 bp, Cricetulus griseus
  misc_feature: Qualifier WAN0088K2_at
gtcgacccacgcgtccgcagggaaggccattgttctttaagtgtagctaggggctgactcatatctcagggttttccatgtttgtgaagtggacagttctttgactaggatgtgacttactgtttcccatagtgatgcgaaaaccagcatggaatgacatggtttgaagctggtggaggattaggagcctcatgggcacactgtttctgtacggataagctgtgtctgggcataagatggaggcagaacagacactgcacaggagcaggttgccactctggggaggttctgttcagcaatctgtggttttgatatatatcaaaatcttgtgtaatcaaaagttttcaaagcttcacagtaaatttggacatacacactaaatttctaagaatccaccctcttgtcacactggtaagacataatttgaaaatgaaagcgacagctccatcattaagtgtgttccatgaggatcaggggatttggcttcagtgtctgcctttgccatatgtcatgtagggttctctgggccatggtcccttctctgtagctgatagtcctactctacagctgtcagtctttatcacaaatgccacagtatca

SEQ ID NO: 416 - DNA, 562 bp, Cricetulus griseus
  misc_feature: Qualifier WAN0088ON_at
gtcgacccacgcgtccgcgcctatgcaataattaaagaagagcttgatgaagactttgagcagctttgtgaagagatccaggagtctaggaagaaacgaggttgtagttcctccaaatatgcaccatcttattaccatgtcatgccaaagcagaattctcctcctgctggtgataaaaggtcagctcaagagcccagtgaaaagctgaaggcaccgtgtcctcctgtggcttgcagtacccctgctcagttgaagaggaaagttcataataagtcaaagtggcatgtaggtaacaaaataaagcgaaggaaactttcacaagcaaaagacaatggccaaactgctgtgaataatcaagtcaggagtagtgatacggaggaaagtcagcccacaagcacagagcacaatgagctgggaaacacgggagagtcttccgtagaagagaatgagaagcagaatgtctccgaaaacaacctagacctgaaaaatacctcgagtacctccaatattgagaatgagcttgaagaatctaatgaaattacagagtgtgcagacttgagaa

SEQ ID NO: 417 - DNA, 600 bp, Cricetulus griseus
  misc_feature: Qualifier WAN0088Q6_at
gtcgacccacgcgtgcgtaggagaagtccaattccaacccacactgagcttccggtgtttagagattctaagatgcctgagccagccaagtccgctcccgccccgaagaagggctccaagaaggccgtgaccaaggcccagaagaaggacggcaagaagcgcaagcgcagccgcaaggagagctactcggtgtacgtgtacaaggtgctgaagcaggttcacccggacaccggcatctcctccaaggccatgggcatcatgaactcgttcgtcaacgacatcttcgagcgcatcgcgggcgaggcttctcgcctggcgcattacaacaagcgctcgaccatcacgtcccgggagatccagacggctgtgcgcttgctgctgcccggggagctggccaagcatgcggtgtccgatggcaccaaagccgtcaccaagtacaccagctccaagtgaaaaggacgtcccacgaggagtagacctgatgatgtcttttctaccactcatcagtgctggacgttgtttcatttggcaattgagacaatgganataggtgatactagaagactagtggctgtattttcaaaacttgaggaacaactc

SEQ ID NO: 418 - DNA, 495 bp, Cricetulus griseus
  misc_feature: Qualifier WAN0088S8_at
gtcgacccacgcgtccggctgaaccttgaggggccctctgaccaggagaccaagttggatcttatcagtaaaggagaggaaccaagaggaggaagagaggaatccggggtaccagctcgcagctctccacccagcagcaaaaaccactctatcaaggctatacttaagaatatctcagtcctggctttgtccgtctgcttcattttcactgtcaccattgggttgttccctgctgtgactactgaggttgaatccagcattgccggcaccagtgcctggaagagctactacttcattcctgtggcttgtttcttaaatttcaatgtctttgactggctaggccggagtctcacagctatttgcatgtggcctggtaaggatagcctctggttgccagttttggtgggctccaagatcgtgtttattcccctgcttttgctatgcaaagtgaaacatcgctactacttgcgttccatctttacccacgacgcctgg

SEQ ID NO: 419 - DNA, 600 bp, Cricetulus griseus
  misc_feature: Qualifier WAN008906_at
gtcgacccacgcgtgcgggttatcatcatggccaccaactgcgagaattgtgggcatcggactaatgaggtgaagtccggaggagcggtagagcccttgggtaccaagattaccctccacatcacagatccctcagacatgaccagagacctcctcaagtctgagacgtggggtgtggaaatcccagagctagagtttgaactgggaatggctgtccttgggggcaaattcaccactctggaagggcttctgaaagacatcagagaactgaacccattcacactgggcgatagctcaaatcctgaccagtcggagaaactgcaggagtttagccagaagttggatcagatcatcgagggcaagatgaaggcccactttactatggacgatccagcaggaaacagttacctgcagaatgtgtatgcccctgaagatgatccggagatgaaggtagagcggtaccagcgtacctttgaccaaaatgaggagctcgggctcaatgacatgaagacagagggctatgaggcatgcctggccccacagcggtagcagtgcacagctcacaggccagcttcagtgccaccagcaccacaggcgtat

SEQ ID NO: 420 - DNA, 600 bp, Cricetulus griseus
  misc_feature: Qualifier WAN00893Z_at
gtcgacccacgcgntccgaatagatttccaaggcagaaggggttgaaggcaagcccatgactgctatcacgggccatgggcatggcagatgggcccagccattgctgtgcagagtgtagcaatagagagcccctcactgtcgcatggccctccccagagcatgcccaacccaggagtctgtctcattgtttatctaaccaccaagagagcccctcccttaaaacagtatgccctcacaaacctaccatgtgaatgaactgggaaaggggcatgacccccaggtgtgtatagctgtgagtttcaataatctagcactatgttggacccactccccttccacccagccagctttgctgtcagatacaagtcctgttccctgtttgtcctcttccagggactgtagcctggaggcttccacacagcccacacatctcttaggctccattataatcactttcctagagacgctggcctgtctgtgaaggctgagcttgtgtgtggtgttgggtttatgcctcctgctccctcccaccctgtatctgggcatagttcacatcaagtgtgtatccgccaggctccaggacctttctggatttgcca

SEQ ID NO: 421 - DNA, 117 bp, Cricetulus griseus
  misc_feature: Qualifier WAN008BNE_x_at
gtcgacccacgcgtccgcttggaagatccctgggctgagatgggattgtccagtgggtggggcagagggtatcttctcacgaaatgaactctgtatggaaattgtcccttttctgac

SEQ ID NO: 422 - DNA, 600 bp, Cricetulus griseus
  misc_feature: Qualifier WAN008BNO_at
gtcgacccacgcgtccgcccgcccgaccacctcggtgccggatcctagagctgccgctggaagcagaggcttgtgaggggaggcggagagaaaaggggtgcgggaaatttaaaaagatgggaaaggcgcttcccattttccttagcccctttcctgttgatttctggagcaacgctcctttcttctcggacgaagagaatgaagaccctgctttatttaacagtcatcttaggggaagtgccatcgttcttccagaagggagatctggccccagatcttggattctgagaccagtgcgagatccccgctttgcgccctgtccgccctggagctggatttgtcatcagtttagaatagcttggctacgatgtgtggtgtggatggatggacgggcctcgcaagtgcaaacaggacaatccaggttacctggcaaagcaaccaccagtgcaacagctctccaagatgaagcctaggtcactcactgacatggctaaaagcatctangaagtctggatcattgtggtgcctctctccagaaaatggtgacaaaaatggaaatcaaccagcacatcaggatccttgttccttctgtggcaggac

SEQ ID NO: 423 - DNA, 600 bp, Cricetulus griseus
  misc_feature: Qualifier WAN008BRX_at
tcgacccacgcgtccgatctacaccaccttttccttgcatgctctgctggttgaccactacatacaaggggcattttatccacgaggaggttccagtgagattgccttccatatcatccctttgattcagcgggccgggggcgctgtcctcactagggccactgtacagagtgtgctactggactccactgggagagcctgtggtgtcagtgtgaagaagggacaagagctggtgaatatctattgccccgtggtcatctccaatgcaggaatgttcaatacctaccagcacttagtgccggagagtgcccgctgtctgccagatgtgaagaagcagctggcaatggtacggcctggcatgagcatgctctcaattttcatttgcctgaaaggcaccaaggaggacctgaagcttcagtccaccaactactatgtttattttgacacagacatggacaaagcgatgcaacactatgtctctatgcccaaggaaaaagctccagaacacattcccctttcttttcattgcctctccatcaaccaaggacccaacctggggaggaccgattcccanaccgatccacaatgactgtgctggtg

SEQ ID NO: 424 - DNA, 465 bp, Cricetulus griseus
  misc_feature: Qualifier WAN008BSS_at
tcgacccacgcgtccgggcgattctctctcagcccacaccctcgcttgttgtggaccataggcgcttaaaaaatcttttggagactgttgttaaaaaaagtcaggaatataatatattccaactggaaaacttgtatgcagttatcagccagtgcatttatgagcatcgcagggactatgacaaaacagctcttgttcagaaaatggagcaagcagtagaaaacttcaattgttccagatcatgatgtcattgcaccaagtatttttatattcaattcctatttgattgtcttcanatatgtccacataactgatgcaagataaaatgaaatcttgcattttgaagaaaaattaaaatagngaaagtattttaattccctggtacttatgtacatgtggtaagataactgataagtcttggatgtttgagcagaacactgtnacaatagtnattgntcaagtttg

SEQ ID NO: 425 - DNA, 600 bp, Cricetulus griseus
  misc_feature: Qualifier WAN008CRT_at
gtcgacccacgcgtccgcttcaatcggcgtttccactttaccagcgttcttgcaaactttgaccgccggcgccggaaatactacgcatgcgccatccctttgcatggtgagcctcctgattctagctgccgcagcggcaactgtggtgatcttgctgctagtcgtgagactatgggcggtcattcggccccttcacgtcgtttcccgagagtctctcacacccttgatagtagctggatccggtgaacataccactgagattctgaggctggttggaagcttgtccggggcttactcacctaggcactatgtcatcgctgactctgatgaaatgagtgccaagaaaattgaatcttttgaacatgctcgaaccaagagagactctcctcctgagcactgccttcaccgaattccaagaagccgggaagtgcggcagtcctggctctcctcggtgttcaccaccttgcactccatgtggttttcctttcccctggtttaccatctgaagccagatttggtgctgtgtaatggaccaggaacatgtgttcccatctgtgtgtctgccctgctccttgggatacttggaataaagaaagtgat

SEQ ID NO: 426 - DNA, 600 bp, Cricetulus griseus
  misc_feature: Qualifier WAN008CSG_at
gtcgacccacgcgtccggccaacattgcacatgggaattcctccatcattgcagaccggattgcactcaagcttgttggccctgagggcttcgtagtgactgaagcgggattcggagcagacataggaatggaaaagttcttcaacatcaagtgccgatattctgggctccagccacacgtggtggttctcgttgccactgtcagagctcttaaaatgcatggcggcggccccacggtcactgctggactgccccttcccgaggcttacaccaaagagaacctggacctggttgaaaagggcttcagtaacttgaggaaacaaatagaaaatgctaggatgtttggagtgcccgtggtcgtggccatgaatgcattcaagacagatacagacgctgagctggacctcatcgcccgcctctccagagaacacggggcttttgatgctgtcaaatgcacccactgggcagaaagggggcggagagccttatccctggctcatgccgtccagagagcatcacatgcccccagcaacttccggctcctctatgacctcaagctctccattgaggataaaatcaggatcatcgcacagaagatct

SEQ ID NO: 427 - DNA, 600 bp, Cricetulus griseus
  misc_feature: Qualifier WAN008CT2_at
gtcgacccacgcgtccgaaagaatttcatctgaatgagagtggtgacccgtcatcaaagtccacagagatcaaatggaaatctggaaaggatttgacaaaacgctcaagtcaaacacagaataaggccagcaggaagaggcagcacgaagagccagagagcttctttacctggtttactgaccattctgatgcaggtgcagatgagttaggagaggtcatcaaagacgatatttggccaaaccccttgcagtactatttggttcctgatatggatgatgaagaaggagaggcagaagatgatgatgatgatgatgaagaggaggaaggcttggaagatattgacgaagaaggggatgaagatgaaggtgaagaagatgaagatgatgacgagggggaggaaggagaggaggatgaaggcgaggatgactagcacagaagactgatggattccaaccctcttttttattttcttttattttcaattttctccagtccctgggagcaagttgcagtcatttccctcccctccccaatccctcttgtgctcagtcgccctgtttttgaggtctcttctcaacaccatggctctcaacttattt

SEQ ID NO: 428 - DNA, 600 bp, Cricetulus griseus
  misc_feature: Qualifier WAN008CTA_at
gtcgacccacgcgtccggacgaaggcagtgggagggagaaccccttccaaaccggcctctgtgaagaaagcagccgccgcccagagcagcagctcctccgaaagttccagtgacgaggatgagaagcccaagggcaagggcactgctaaaccacaggccagcaaggccaatggcactccagcttctcagaatggaaaggcaggcaaggaaagtgaggaggacgaggaggaagaagagacgaaaatggcagtttccaagccaggaaagaaacagaagcggaatgagacagcagaggaagcagggacgcctcaagctaagaaagttaagctccagactcccaacacatttccaaaaaggaagaaagcagaaaaaagggcatcttccccattccgaagggtcagggaggaggagattgaggtggatgcacgcgtagcagacaattcctttgatgccaagcgaggtgcagctggagactggggtgagagagccaatcaggttctgaagttcaccaaaggcaagtccttccgccatgaaaaaacgaagaagaagcgaggcagctaccggggaggctccatctctgtccaggtcaattctgtcaag

SEQ ID NO: 429 - DNA, 600 bp, Cricetulus griseus
  misc_feature: Qualifier WAN008CXZ_at
gtcgacccacgcgtccgcccacgcgtccgtctgaggccagagagctgctacagctagcagatgccttggggcccagcatctgcatgctcaaaactcatgtcgatattctgaatgatttcactctggatgtaatggaggagttggcagctctggcgaaacgccatgaggggttaatatttgaagacaggaaattcgctgatataggaaacacagtgaaaaagcagtatgaaggtggcatctttaaaatagcttcctgggcagatctagtaaatgcccatgtggtaccaggctcaagagttgtgaaaggtttgcaggaagtgggcttgcctttacatcgagcatgcctgctcattgcagaaatgagctctgctggttccttggccactggaaactataccaaagcagcagttgggatggctgaggagcactgtggatttgtggttggctttatttccggctcccgagtaagcatgaaacccgagtttcttcatttgaccccaggagttcagttagaaacaggaggggatcaccttggccaacagtacaatagtcctcaagaagtgattggcaagcggggttctgatgtcatcattgtaggcc

SEQ ID NO: 430 - DNA, 599 bp, Cricetulus griseus
  misc_feature: Qualifier WAN008CZP_at
atcgtgtggaaatgttatccgctcaatacgactcactatagggcgaattgggtaccgggccccccctcgaggtcgacccacgcgtgcgctcagggtgaggccatgggctccgtgcccgagttttactccaagaggaggcactacgatgcacagccctccaactcgctggaccaatatggattacgattgggcatgtggtactggaaagatgaaaccaaaactctggaatttagaagtgggacgcctgcagtagagctcaaggataaaggcaagaaagggaaggcggttcacttcgctgaaatggacggtccggcttctgaaaggttgacagataaaaggtttccctcaagggacgaaaagtcactgaaagtcttggagaaacgatatcagctgggcaatatcacgctggatgatgtcaagtttgttgctttgcttttcctccaagacactgaaatgcagcgggtctgttctttcacaacatttatgagagagaaaatatcagatactcaaaaagactggaaattctttgagtccttttatactttctgtacatgcatcgcttggattgtcttccgacgtcagtacctgacagagatcga

SEQ ID NO: 431 - DNA, 600 bp, Cricetulus griseus
  misc_feature: Qualifier WAN008D7X_at
gtcgacccacgcgtccgtggtgggcagggccaaggtcagtcctcagatgagagctcttctgctccatcctgtgtgcctcaagcacttgacttttctccaaacaagagtgaatctcctccttcccatggctccaccacagaggcagctcaaggtgaaacagagcctcatgaagacaacacaagccccactgagggtcttctcctggacatcaagctggaccaactcaccatcactcctgaagcaggaggcagagacctagctgaccgccctctcatagacttcagcaacacccctgagtccaacatggctttgggacccagcagctggcccctgattgaccttgtaatgaatactccagacatgggtagaaatgatgtggggaaacctccaaaagctgagcttgggcagctgattgacctgagttccccactgatccatctgagccctgaggctgacaaagaaaatgtggattcaccacttctcaagttctgagcaaagcacaactctgccctttactctgtttatttctgttgtggctttaagcccattagaagcaaatttgaaaggaactgtatttgattcggaggaggccttgtgtct

SEQ ID NO: 432 - DNA, 600 bp, Cricetulus griseus
  misc_feature: Qualifier WAN008DBR_at
gtcgacccacgcgtccgtgaccgagtctgcaagagtcaccttctggattgctgcccccatgacatccttgctgggacgcgcatggatttaggagaatgtaccaaaatccacgacttggccctccgagcagattatgaaattgcaagtaaagaaagagacctgttttttgaattagatgcaatggatcacttagagtccttcattgcagagtgtgaccggagaactgaacttgccaagaagcggctggcagaaacacaggaggagatcagtgcagaagtttctgcaaaggctgaaaaggtacatgagttgaatgaagaaataggaaaacttcttgctaaagcagagcaactaggagctgaaggaaatgtggatgagtcccagaagatccttatggaagtggaaaaagttcgtgcaaagaaaaaagaagcagaggaagaatatagaaataccatgcctgcatccagttttcaacagcaaaagctccgagtctgcgaggtttgttcagcttaccttggtctccatgacaatgaccgtcgtcttgcagatcacttcgggggcaagttacacttggggttcattcagatccgagagaagcttgat

SEQ ID NO: 433 - DNA, 600 bp, Cricetulus griseus
  misc_feature: Qualifier WAN008DGK_at
gtcgacccacgcgtccgcagagacagagaacagcagagggaggagaaggagaagctgagggaagagacgcggcaagccaaggaggaggctcgaagaaggaaggaggaagagaaggagctgaaggagaaggagcggagggaaaagcgggagaaggatgagaaggagaaggcggaaaagcagcggctcaaggaggaacgacgcaaggagcgacaggaggctctggaggctaaactggaggaaaaaaggaagaaagaggaagagaagcggttacgggaagaagagaagcgcattaaggcagagaaggcagagatcaccaggttcttccagaagccaaagactccacaggcccccaagaccctggccggctcctgtggtaagtttgccccctttgaaatcaaagaacacatggtgctggctccacggtgccgggctaccttggaccaagacctttgtgatcggctggaccaacttctacagcagcagagtggggccagctccttcctcagtgacctgaaaggccggctgcccctgaggtctggacccacccatgtgtgcagccgcaacaccaacatcatggacagggtcattgtggagagcaag

SEQ ID NO: 434 - DNA, 511 bp, Cricetulus griseus
  misc_feature: Qualifier WAN008DK1_at
gtcgacccacgcgtccgagctcctcgatggtgctggctgccgctggaggggtggaacaccagcggctcctagaacttgcccagaagcacttcagcagcgtctctcgagagtatgaagaggatgctgtacctggtgtaactccatgtcgcttcactggcagtgagatccgtcatcgtgatgatgccctgcctttagcccacgtggccattgctgtagaaggacctggctggtgtaacccggacaatgtggccctccaagtggccaatgccatcattggccactatgactgcacttatggtggaggagtgaacatgtccagccctctggcttccgttgctgtgtctaacaagctttgccagagtttccaaaccttcaacaattcctactctgagactgggctgctaggtgcacactttgtatgtgatggcatgagcatcgatgacatggtcttcttcctgcaaggccagtggatgcgcctgtgcaccagtgccacagagagtgaagtgacccg

SEQ ID NO: 435 - DNA, 515 bp, Cricetulus griseus
  misc_feature: Qualifier WAN008DO3_at
agggataggtacaatcaccaccaccctggaaccaagggtctctcacaccattgctcagtgctgaactataactgccacccatccccacataccaaatagagcagtggcaacctctccctcccccgacctaactcttggaacttagtcctcctcctttcttcccattaagtctgagaggtaagagctaggttatggaaggaagggtttggccagaagtgggaatagccaagtgccccggacctctcatcttccctccgtcctgctttccacctcccttgggtacacacacagccttttgggaacagccggggccaggactgggtcacctatgagctgaatcagcctctcctcctgagtcccagggcccctgcagttcccgagtctgttttgtcctgcagcctcttgccctctggttccactttatatccaccttttccttttgttcaattcttatctttatttcttttattatcaaatgatgtggtctatggaaaaaataaaaatctgacttagtt

SEQ ID NO: 436 - DNA, 556 bp, Cricetulus griseus
  misc_feature: Qualifier WAN008DRM_at
tcgacccacgcgtccgtatggcttcaactccagctacctgaagaagtggtgtcctactggaggaacgagtttgactggaggaagcaggtggagaccctcaaccagtaccctcacttcaagaccaagattgaagggctggacatccacttcatccacgtgaagcctccccagctgccctcaggccgcaccccaaagcccttgctgatggtgcacggctggcctggctccttctatgagttttacaagatcatcccgctgctgactgaccccaaggcccatggcctgagtcacgagcatgtgtttgaagtcatctgtccctccattcctggctatggcttctcagaggcatccagcaagaaaggcttcaatacggtggccactgctaggatcttctacaagctgatgtcacggctgggcttccataaattctacattcaaggcggggactggggctccctcatctgcaccaacatggcccagctggttcccaaccacgtgaaaggcctgcacttgaacatggcttttatctcaagaagtctctacaccctgacccctc

SEQ ID NO: 437 - DNA, 470 bp, Cricetulus griseus
  misc_feature: Qualifier WAN008DXL_at
tcgacccacgcgtccgggctagggccgtgcacgtgttatctgtgcagcctctctctttggagaacttatctctgaggggctgctgtcaagatgcctggttcctttctggcaagcagcaggtagctaaagaaaaccagcaggtagcaaaggatgtcacactacatcaggccttgcttcgtctgccccagtaccagactgatctcttgctcaccttcaatcagcccccgtaaggaaggaaagggtgggaaactcgcgatctcccccgagccccctcgcccccctgacaattaggctgggtaggacaagcaggaagaaggaagattggctggtggattccttcaaggccgaggtcctctagggaataggaactgtaatcctgcaaacctaatgccggtccattgtctttctccctctgttccctcaccctaaactctaacaggaggtctcttggccctgaaaatctatcatgt

SEQ ID NO: 438 - DNA, 448 bp, Cricetulus griseus
  misc_feature: Qualifier WAN008E4X_at
gtcgacccacgcgtccgctgaccactcctcaggctctgccacgcttgctgtgtggggagtggaaagacctgagaaagtccttgtgctgggatctgatcagggaaagaatgcccttcaacatgaccttgggacccgggcagccagggggagattttaccacaaaaccaccaggcactaagtaagtgcttagcttagtgcagggtggctctggtcctgatagcacctgatgctctttcccttagaattggggtgacccacgaggcagacagggcacatcaaaccaccattgccttgaacacacctaaagtaaggcccatgtactttgccatatcctccaagtcttctactctaaggacagttaaattatgtgggcctgagaaactgacaagcttactattgggaacctgttcagaaaaccttgcccattctttaagtcaaataaagacta

SEQ ID NO: 439 - DNA, 600 bp, Cricetulus griseus
  misc_feature: Qualifier WAN008E4Z_at
gtcgacccacgcgtccgctttgtatttggcactggacattcagcaccatctgccagtccagcatttggtgctaacccgaccccaacatttggacaaagtcaaggtgccagccaacctaaccctccaagctttggctctatatcatcttcaacatcgttgttttctgctggttcccagcctgcaccaccacctacttttggggcagtgtcaagcagcagccaacctcctgtgtttgggcagcagcctagtcagtctgcatttggctctgggacagctcctaattccagttctgttttccaatttggcagcagcaccacaaatttcaacttcacaaacaataatccgtcaggagtgttcacatttggtgcaaatcccagcacgccagcagcctcagcccaaccctcaggttcagggggctttcaattcagccagtctccagcatcatttacagtggggtcaaatgggaaaaatatgttctcttcttccggaacttcagtttctggtcgcaagataaagactgctgtcagacgcaggaaataaaggtcacgaaggtgttatacccaacagttttatcaacggctggtgcctgctntcagatac

SEQ ID NO: 440 - DNA, 600 bp, Cricetulus griseus
  misc_feature: Qualifier WAN008E5L_at
gtcgacccacgcgtccgcccacgcgtccgcggcgctgttccagtgtgtggcggccgtgttcattgctcagctgaatggggtgtccctggacttcgtgaagatcattaccatcctggtcacagctaccgcatccagcgtgggcgccgcaggcatccctgccggcggggtcctcacgcttgccatcatcttggaagccgtcagcctccctgtcaaagacatctccttgatcttggctgtggactggctagtggacaggtcctgtaccatcatcaatgtggaaggtgacgccttcggggccggactgcttcaaagttatgtggatcgaaccaagacgccgagctcggagcctgaactgatccaggtgaagaacgaaacgtctctgaatccactgcccctggccccagaggaggggaacccccttctcaagcagtaccgaggacccgccggggacgctgccgcgtgtgaaaaggagtctgtcatgtgaatagcaggaggggctttctgtgccctggtggcgtctcattggaagtggatatcatganggctggagaaactccaggggaggagtttacacagaccgggaaactaggggtccctgtc

SEQ ID NO: 441 - DNA, 600 bp, Cricetulus griseus
  misc_feature: Qualifier WAN008E65_at
tcgacccacgcgtccgcgcccaccaagtgacgtgagcgtccactccgagctcacggtccgcaggtttccatcttccgcaatctgctgtgatggctgcctcggtccctgccgccgtttccctctccccactgctgtctattctcctgggcctgctgctcctctctgctccgcctggcggcagcggcctgcacacgaagggcgcccttcccttggacacaatcactttctataaggtcatccccaaaagcaagttcgtcttggtgaagttcgacacccagtatccctatggtgagaagcaggacgagttcaagcggctggctgaaaattcggcctccagcgatgatctcttggtggcagaggtggggatctcagactacggtgacaagctgaacatggagctaagtgaaaaatataagctggacaaagagaactacccagtattctacctcttccgggatggggactttgagaaccctatcccatacagtggggcgattaaggtcggagccatccagcgctggctaaaggggcagggggtctacctgggcatgcctggttgcctgcctgcgtatgatgccctggcaggtgagttcatcaaag

SEQ ID NO: 442 - DNA, 418 bp, Cricetulus griseus
  misc_feature: Qualifier WAN008E6I_at
ctctgcactgcacactgctgctcaagacgctgggtggctactcttggaccctgggtcctgctcaaaccccactggacccaccccagtcctcagacactttgatctctgtggaccctgcagaggatgaagcccagcagactgcagcccaagtcgcaggggtcctgggtggcctgcttacacccctcttcctccgtggcgtgctggcctacctcatttggtggacggctgcctgccatctgctctccagcctctttggcctctatttccaccagcacctggcagcttcctaatgtgatgccactaggtgctggcatctgattcctgtccaccagggctgggactgtcccctgtgtatgtacttcaaaccctagatgacctagggataagggtggggactccccaccctctctcacctcag

SEQ ID NO: 443 - DNA, 545 bp, Cricetulus griseus
  misc_feature: Qualifier WAN008EED_at
gtcgacccacgcgtccggtagtgaaagcggtgtaccgaactgctttggccctgcaggtaggttccctgtgctggcagctggacatccgcgagtgggaggacctgcgagcgatcgcgagggatccttgtctgccaggaggtggaagaacatcctccgcacaggttcaacatttggactcatctgatgacagctcctgactccaagtgactgacatacacctgttgtttcttgggtaacataccaggggctgcgatggacctggttctcaatgttgccgatcactacttctttactccatatgtgtatccagccacgtggccagaggacaacatcgtccgacaaactattagtctcctgattgtcacgaacctgggtgcttatatcctctacttcttctgtgcaaccctgagctattattttgtctatgatcattcattaatgaagcatcctcagtttttatagaatcaagtctctcgagagatcatgttcacggttaagtctttgccctggataacgatccctaccatttcattgttcctgctgga

SEQ ID NO: 444 - DNA, 572 bp, Cricetulus griseus
  misc_feature: Qualifier WAN008EK5-rc_f_at
gctgccatgtgggtgctgggaattgaacccaggtcctctggaaaagcagtgactgctcttaaccaccgagccatctctccagctgccttgtttatgtattttgagtctgataaagtctgtacaaaatttagcatgtctgggggatttcagtggtgacttagtgctgacttttattttttttgtgtttagattcagaaggtttgtaacaaaaaattatgggaaagatacacgcaccggaggaaagaagtttctgaagaaaaccacaaccatgcaaatgaaaggatgttatttcatggtaagttctcaccctgtcctccctgtgagattggagcttttgttgtcattgtcatcattatcttcttgctgtgtagccctggctatcctagaactcactatatagaccaggcaggcctcaaactccctgagatctgcctgccaaattcatgcatcatcactgcccagcaacttatttatgtcttcttaataaagataatatttctgtaacttttatatttgtgtacatattttcagtttcttttgtgataatgtaccttacaatgaacatttttttt

SEQ ID NO: 445 - DNA, 600 bp, Cricetulus griseus
  misc_feature: Qualifier WAN008EMN_at
gtcgacccacgcgtccgcacttacatatttaagggtcattggaatgtcagatgttaatgattggacactgaacatggtagtgacttgtgcctgtaatttcaatacttgggaggtgtaggcagatgattgctttgtgttaagtccaacctggtctgcagaataataatctgttgtaaacccataaaactggggaaaagtagggtcttaaaatatataaactaatgttatgttttgtaaatattaattggagggttttgtttggctcaaggcaaaaactaataaaatgctggttgatagtactgccccttactgcagtgccttccttccagctttgactaagaaggctcaggtttcatttcattattgtggttgatttcagagcctgttgttagcaaagtcagcagaatgtagaacggtgattgtcattgggttgacacttggctgggggtagtggcctttgttcattaccgtaactagttcaagctggactggaactcactttgcagaccagttagtctcaaatccacaacaacacttgcttcagcctcccaaatgttttgattaaaggcatgttaattgattttgtggtttctgatgact

SEQ ID NO: 446 - DNA, 422 bp, Cricetulus griseus
  misc_feature: Qualifier WAN008EP0_at
gtcgacccacgcgtccgggtgcttagggtcctacccagcaccagtgagagaaactctggagccatcatatgctctttaacgtgagatcactgcgttggtactataatgagctctttttaacgctagctttcatttagactggttttgtcgacagccacttaacctcctttagtaccatgaagctggactcttaaatggaggagttctgggtcactctacttcacgcattggtgtaaaaagtcacagcttctacttgcttcatctggacatcagactgctgcgtctaagaaaacagtagtaccagttccccacagctagtgggctggaggctcataatggatggctcattggttaaaagcactgtggctcttccagaggatcagcattcgattccccagcactcacatggtggcttacaacca

SEQ ID NO: 447 - DNA, 502 bp, Cricetulus griseus
  misc_feature: Qualifier WAN008ET3_at
tgaactctatgaaaaacgagatctcatcaaaaacctaaagacgatgaaagaagatcttatggaaagtatccaaaatccacaaggggaaatatgcagcacaaatacaggaagacatttcagagctaatgaataaaactatgacagtaaaagattcaatcgctgagaaaacatattttattgaagaagagaaaaagaaacatgaaaaactaagaaaagaaatagaggtacagcataagagatatgatgcaattcttaagcgtttgcattgtcagatgagcaagattcagttaaataggaggaaatggcaatggaacatccaacaactggaaaaaactgcagctgacttaaaaaagcgccttggagtgaaagaagcccatgaactacagaacagaccagcagcattgtgggaaagaataccgtgaagatcctttggtcacttgtgaaccagcacccagcggcaaaggcctgtctcaatggccgatattttcttttgtcccccttc

SEQ ID NO: 448 - DNA, 600 bp, Cricetulus griseus
  misc_feature: Qualifier WAN008ETA_at
gtcgacccacgcgtccggctgtcagggtctcagtttctcatcagctctttcttacatgccttcatgtagggagaactaaggtagagagagacaggngaaaaagtaactggcctgggctctagctgtcccctgccgactaaaggcttcttacgtcccatcccggtatatccatgctggcttctctatggagctgacactcagcaggtgttcatgttagtcctttgtcttccaaacacccatttgccagagtcagtgtcattttaggtggccatttctgttgaagcacttgtatcataagacgttggatttggactagtgacctttagaccatgtccttgtagctacacaccaagggctcccaggagaccaaaatagttaaacattacttgactacaactgagtcctctgggtaatggccactgtttcttgtactgtaactgacatagttgcttagtttgccctttcaccccagcatctggcaacagtgctattagtacaagctgagacactatgctttcaggaagctagaggctccctgaacctgaacctgaacctgggggagccagaagatgtgctcaagtgctggctgcagcccaagcc

SEQ ID NO: 449 - DNA, 650 bp, Cricetulus griseus
  misc_feature: Qualifier WAN008F1A_at
gtcgacccacgcgtccggccctaatgatgaagggaagatgttcatgaggccagggaaactgtccgactactttccaaaaccataccccaaccctgaggctgccagagctgctaacaatggagccttacccccggacctcagctacattgtgcgagctaggcatggtggtgaggactatgtattctccttgctcactggttactgtgagccccccactggggtgtcattgcgagaaggcctctacttcaacccttactttcctggccaggccattggtatggctcctcccatctacaatgaagtcttggagtatgatgatggtaccccagctaccatgtcacaaatagccaaggatgttgccaccttccttcgatgggcatcagagccagagcatgaccatcggaaacgaatggggctcaagatgttgttgataatgggcttgctgctgcccctgacctatgccatgaagcggcataaatggtcagttctgaagagtcgaaagctggcatatcggccacccaagtgaccctgtccagtgtctgcttgtcctcttgcctgaagaagccctcaacagcccaggaagtcctaggccttttcaggcctgctccttttcatccaatagaaggaaatggggtaggagaccaggctct

SEQ ID NO: 450 - DNA, 608 bp, Cricetulus griseus
  misc_feature: Qualifier WAN013HV4_at
cgaggtgcctttcctggatccctcctcactggacccgagtgtggtggcagctcgggctggggcccctggtttccagagggtctgggctgtttcggctgctgtaactagatttagttcagtggtgctctggaggagaccgggagaagggttgaagtgtgaggctgggtggatgggaaaattagttttggtcttcacatctctggtttcatttttgctgtcgtagcgcttgttgctgtctctatgttaaaatgccaaaaatgatctagttgtcgctttcctcctgctccagcactccacagcagcttctggctttgtgaggtgtttgtctcagccagtgccatgacctggcaggaacagatctgggttcccactgtctcccatcatgtgtccgactatagtccgtcgggacaagcttctgcaaaactaacctgctttgtccggtttcccccagacaccacctctcgcttccacttgccgcctgagccttcctggtctctctctggctagtgctgcacagwgtctgcttctgctgtcctggaggtctcctgaagttgtgtggkcaccmararggccaaggacgcamtagccctgagctgagctgggctcat

SEQ ID NO: 451 - DNA, 873 bp, Cricetulus griseus
  misc_feature: Qualifier WAN013HVE_at
acaatggagtagtcttgtccaccgagagcagtgtggcagtatatggaacactaaacctaactccaaaaggcaaacaggctccgggaggccatgagctgagctgtgacttctgggagctggtggggctggccccagctgggggggctgataacctgatcaatgaggagtctgacgtagacgtccagctcaacaaccggcacatgatgatccgaggagagaacatgtccaaaatcttgaaagcacgctccatgatcaccaggtgcttcagggatcacttcttcgacagaggctactgtgaagttactactccaacactggtgcagacgcaggtggaaggtggggccacactcttcaagcttgactattttggagaagaagcgtttttaacccagtcctcgcagctgtacctggagacctgccttccggccctgggggatgttttctgtattgcccaktcatacagggctgaacagtccagaacacgaaggcacctggctgaattcactcatgtggaagctgagtgtcccttcctgacctttgaggacctcctgaaccgtttagaggacctagtgtgtgatgtggtggacagagtcttgaagtcaccagtggcaagcatagtgtatgacctcaacccgaactttaaaccccctaaacggcctttcaggcggatgaactattcanaagctatcgagtggctgagggagcacgatgtaaagaaagaagacgggacactctatgaatttggagatgatattcccgaagctnnnnnnnnnnnnnnnacagacaccattaatgaaccaatcctgctgtgtcgggttcctgtggagatcaagtccttctatatgcagcgatgtgcctgagatcctcgactta

SEQ ID NO: 452 - DNA, 499 bp, Cricetulus griseus
  misc_feature: Qualifier WAN013HW5_at
ctccgcagccatgagcagcaaagtgtcccgcgacaccctgtacgaggcagtgcgggaggtcctgcacggcaaccagcgcaagcgccgyaagtttctggaaacggtggagttgcakatcakcctgaagaactatgrcccccakaaggacaaacgtttctcgggcwccgtcaggctcaagtccacccctcgccccaagytytcggtgtgcgtcctgggggaccagcagcactgcgacgaatccaaggccgtagacatcccccacatggacatcgaggcgctgaagaagctcaacaagaacaagaagctggtcaagaagctggccaagaaatatgatgcctttttggcctccgagtccctgatcaagcagatcccacgtatcctgggcccaggcctaaacaaggcaggcaagttcccttccctgctgacacacaatgaaaacatggtggccaaagttgatgaggtgaagtctactatcaagttccagatgaagaaagtgctg

SEQ ID NO: 453 - DNA, 554 bp, Cricetulus griseus
  misc_feature: Qualifier WAN013HX8_x_at
agtgggctttcggatcatgtctggtggctccgcggattacaacagagaacatggcggcccagagggaatggaccccgatggtgtcatcgagagcaactggaatgaaattgttgataactttgatgatatgaatttaaaggagtctcttsttcgaggcatctatgcttacggttttgagaagccttcakctattcagcagagagctattattccttgtattaaagggtatgatgtgattgctcaagctcagtcaggtactggcaagacagscacatttgctatttccatsctgcaacagktggagattgagttcaaggagacccaagcactagtattggyccccaccakataactggctcaacagatccaaaaggtaattktggctcttggagattatatgggagcaactkgycrtgcttgcrttggaggaacaaatgtycsgaatgagatgcwgaagttgcasgctgaarctsctyatattgktgkgggyacaccwtgaasagtcgtntgatatgctaaacagaagatacctttctccaaatggatcaaaatgt

SEQ ID NO: 454 - DNA, 671 bp, Cricetulus griseus
  misc_feature: Qualifier WAN013HXG_at
acgtgtgaaaannnnnnnnnnnnnnnnnnnnnnnnnnnncnnnnnnnnnnnnnnnnnnnnnnnnnnnnnnnnnnnnnnnnnnnnggcgcacccgtacctaactcargagaagctgattgagtactgccaatccaaaggcatcgtggtgaccgcgtacagtccccttggctctcctgacaggccctgggccaagcctgaagacccttctctcttggaggatcccakgattaaggcratkgcrtccaagtacaataaaacaacagctcargtgctgatccggttccccatacaaaggaacttggtggtgatccccaagtccgtgacaccagcaagaattgctgagaacctgaaggtctttgacttcgagctgagcaaggaggatgtgaccactctactcagctacaacaggaactggagggtgtgtgccttgatgagctgtgctaaacacaaggattaccccttccacgcakaagyctgaagcttcggatgcctgctctctcccacgagacttgcacctgctcttcctgtwtcatctgtccttgtgggtgtastgtagactgtgtccctctrcactgtsyggaacctggaagatcagacawcgagggtctgttagtttgatgtagtctccacagagcattatcagtagctgagtgattttcttcagcctttct

SEQ ID NO: 455 - DNA, 586 bp, Cricetulus griseus
  misc_feature: Qualifier WAN013I03_at
gccggcagactcgccgccatgggccgtgtgatccgagggcagaggaaaggcgccggatctgtgttccgcgctcacgtgaaacaccgtaagggcgccgcgcgcctgcgtgctgtggacttcgctgagcggcacggctacattaagggtatcgtaaaggatatcattcatgaccccggccgcggcgcccccctcgcgaaagtggtctttcgggatccctaccgatttaagaagcgcacggagctgttcatcgccgcagagggaatccacacagggcagtttgtgtactgcggcaagaaggcccagctaaatatcggcaatgttttgcccgtgggcaccatgcctgagggtactatcgtgtgctgtctggaggagaagcctggagacaggggcaagctggcccgagcatccgggaactatgccacagtcatctcccacaacccggagaccaagaagactcgagtgaagctgccttcggggtccaagaaggttatttcctccgccaacagakctgttgttggtgttgtggccggtgggggtagaattgacaaacctatcttaaaggctggtcgtgcctaccacaagtaca

SEQ ID NO: 456 - DNA, 555 bp, Cricetulus griseus
  misc_feature: Qualifier WAN013I06_at
atggcgggtgaaaaagccgagaagcctgacacaaaagagaaaaagccagcagccaagaaggctggtggtgatgccgatggtgcagccaaaaagggtgaccctaaggttaaaaaacccaagaaggggaagccccattgcagccggaaccctgtcctggtgagaggaattggcaggtactcccgatctgctatgtattccagaaaggccctatacaagaraaaatactcagctgcaaaaaccaaggktgaaaagaagaagaaaaaggraaaggkccttgctactgtcactaaaccagttggcggtgacaagaatggtggtactcgggtggktaarcttcgaaaaatgcccaggtattaccctactgaagatgttcctcggaagctgctgagccatggcaagaagcccttcaktcakcatgtaaggaagctgcgtgccagcatcactccagggactgtcctgatcatcctcaccgggcgccacaggggcaagagggtggtcttcctgaagcarctgggcagcggcttgctacttgtgacaggacctcttgccctcaac

SEQ ID NO: 457 - DNA, 557 bp, Cricetulus griseus
  misc_feature: Qualifier WAN013I0L_at
atatgtcttcastggctctcgcctcaagctctattkgcctaaggagacttgcctcatcaccttcctgctggcaggcattgagtgtcccaraggagcackaaacctaccaggcttggtgcaggaaggagagccattcagtgaggaakccacacttttcaccaaggaactagtgctgcagcgagaggtggaggtagakgtykagagcatggacaasgctggcaacttcwtckgctggctgcacatggatggggctaacctgtccgtcttgctggtggagcaagcgctttctaaggtccacttcactgccgagcgcagcgcctactaatagcccctcctatctgctgaggaagccgccaagcagaggaaggagaaggtctgggcccactatgaggagcatcctgtagaggaggcgttgcctgtgctggaggagaaggagcgttctgccagctacaagcccgtgtttgtgactgagatcacagacgacctgcacttctatgtgcaagacgtggagactggcacccagctggaaaagctgatggagaacatgcgcaatgacg

SEQ ID NO: 458 - DNA, 459 bp, Cricetulus griseus
  misc_feature: Qualifier WAN013I2L_at
ggtgattcctcttcccttggccatacgtgaaagagagctctgcagccataatcctaatagcagctggctgccatagctagaagcccatgggaannnnnnnnnnnnnnntgtcaactacagggccatgatctcttgtgcactcaggcaggctctgcatcattcaaggggctgtgctctggttcttgcaggcacagtctccccgctaccccagctctcttagaaatggaccaggaaggaggtcacagtgaccagtaccatggatctcccacccacacatctctgttcacctccatagttcctgagactgtcatcaccatcccctctttatcccatgtagccattgtccagaacaagcacccctccctattctctgccacctgtgtggcaagaccactccatcttccnnnnnnnnnnnnnnnnnggctgcatgctgctatccctcctatgtggaccatcctc

SEQ ID NO: 459 - DNA, 579 bp, Cricetulus griseus
  misc_feature: Qualifier WAN013I3N_at
ggcagcttgtgaggttttgtgtggtctcgtcgccagcacagccgggcctacaggcaagcaaccatgtctaarggacctgcagttggtattgatcttggcaccacctactcctgtgtgggtgtcttccagcatggaaaggtggagataattgccaatgaccagggtaaccgaaccacgccaagctatgttgcttttactgacacagaacgattaattgggtggagaagactttgataacagaatggtcaaccatttcattgctgagtttaagcgaaagcacaagaaggacatcagcgagaacaagatagctgtcaagcgtctgcgtactgcctgtgaacgggcaaagcgcaccctctcctccagcacccaggccagtattgagattgattctctctatgagggaattgatttctatacctccattacccgtgctcgattcgaagaattgaatgctgacctgttccgaggcactctggaccctgtagagaaagcccttcgagatgctaaactagacaagtcacagatccatgacattgtcttggtggggggttccaccagaatccccaagattcaaaagct

SEQ ID NO: 460 - DNA, 3443 bp, Cricetulus griseus
  misc_feature: Qualifier WAN013I81_at
ggcccagctggcgcgccgcagctctgggcctgcgcgcaaaggggttaggggtcacggcggcgtagtttgtggcgggaaaagcagtttgatgcgggatggattttaagcggcgacaaggaccaggccctggggtgcccccaaaacgggctcgtgggggtctctgggatgaggacgagccatcgcaatttgaggagaacttggcgttgctggaggagatagaggctgagaatcggctgcaggaggcagaggaggagctgcagctgcccccacagggccctgcaggtgggcagttttccactgcagacattgacccacgctggaagcggcccgccctgtgtgccctggatcccaacacggagcccctcatcttccagcagctagagattgaccactatgttggctcagctgtgccactgccaggaggacccccaacgtcctgtaattcagtgcccatactacgggcctttggggtgactgatgagggcttctccgtctgctgccatatccatggctttgcaccctacttctacacccctgcacctcccggttttggggccgagcacctgagtgacctgcagcgagagctgagcacagccatcagccgggaccagcgtggtgggaaggagctgtcggggccagcagtgctggcaatagagctgtgctcccgtgagagcatgtttgggtaccatggtcacggcccctctccatttctccgcatcaccctggcgctaccccgccttgtggcaccagcccgccgcctcctggaacagggtatccgtgtgccaggcctgggtaccccaagcttcgcaccctatgaggccaacgtggactttgagatccggttcatggtggatgctgacatcgtgggctgcaactggttggagctcccagctgggaagtatgtttggaggacagagaagaaggctacacagtgccagctggaggtggatgtgtnnnnnnnnnnnnnnnnnnnnnnnnnnnnnnnnnnnnnnnnnnnnngtattgcgccccttcgtgtacttagctttgacatcgagtgtgctggccgcaaaggcatcttccctgagccggagcgtgaccctgtgatccagatctgttctctggggctgcggtggggagaaccagagccgttcttgcgcytggcactcacactgaggccctgtgcacccatccttggtgccaaagtgcagagctatgagcgggaagaagacctgctccaggcctggcccaacttcatccttgccatggatyctgatgtgataactggctataatattcagaactttgacctcccatacctcatctcgcgcgcgcaaaccctgaaggtggaccgcttccctttcctgggccgwgtgactggtctccgctccaacatccgagaytcctccttccagtcgaggcaggtgggccggcgggacagtaaggtggtcagcatggtgggtcgagttcagatggacatgctgcaggtgctgctgcgggagcacaagctgcgctcctacaccctgaatgctgtcagcttccacttcctgggtgagcagaaggaggacgtgcagcacagcatcatcaccgacctgcagaacgggaatgagcagacacgccgccgcctggccgtgtactgcctgagggacgccttcctgccacttcggctgcttgagcgccttatggtgctggtgaacaatgtagaaatggcgcgtgtcacgggtgtgccccttggatacctgctcagccggggccaacaggtcaaggtcgtgtctcagctgctgcgccaggccatgcgccaggggctcctgatgcctgtggtgaagactgagggcggtgaggactacacgggagccactgtcattgagccccttaaagggnnnnnnnnnnnnnnnnnnnnnnnnnnnnnnnnnnnnnnnnnnnnnnnnnnnnnnnnnnnnnnncataatctgtgctacaccacactgctacggcccggggctgcccagaagttgggccttaaaccagatgagttcatcaagacacccactggagatgagtttgtgaagtcatctgtgcggaagggcctcctgccccagatcctggagaatctgctgagtgcccggaagagggccaaggctgagctggctcaggagacagaccccttgcggcggcaggtcttggatggacggcagctagcactgaaagtgagtgccaactctgtgtatggcttcactggtgccgaggtgggcaagctgccatgtttggagatctctcagagtgtcactgggttcgggcggcagatgattgagaaaaccaagcaactagtggagtccaagtacaccctggaaaatggctacaatgccaatgccaaggtggtctatggtgacactgactctgtgatgtgccgatttggtgtctcctctgtggctgaggcaatgtctctgggtcgagaggctgcaaaytgggtatctagtcacttcccatcacccatccgactggagtttgagaaggtttacttcccatacctgctcatcagcaagaagcgctatgctggcctgctcttctcctcccagcctgatacccatgacagaatggactgcaagggcctggaggctgtgcgcagggacaactgtcccctggtggccaaccttgttacatcctctctgcgacgaatccttgtggaccgggatcccgatggggcagtggcccatgcaaaggatgtgatctcagacctgctgtgtaaccgcatagacatctcccagctggtcatcaccaaggagctgacccgagcagccgcagactatgctggcaagcaggctcatgtggaactggctgagaggatgaggaagcgtgaccccggcagtgcgcccagcttgggtgaccgcgtcccctatgtgatcatcggtgctgccaaaggcgtggctgcctacatgaagtcggaggaccccctgtttgtgctggaacacagcctgcccatcgacactcaatactacctggggcagcagctggccaagccactgctgcgaatttttgagcccattctgggtgagggccgtgcagagtctgtgctgctccgaggagaccacacacgctgcaaaaccgtgctcaccagcaaggttggtggcctcctagccttcaccaagcgccgcaactgttgcattggctgccgctcagtgatcaaccatcagggagctgtgtgtgaattctgtcagccccgggagtcrgagctctatcagaaggaggtgtcacacctgaatgccttggaggaacgcttctcgcgcctctggacacagtgccagcgttgccagggcagcttgcatgaggacgtcatctgcaccagccgcgactgtcccatcttctacatgcgcaaaaaagtgcgcaaggacctggaggaccaggagcggctgctgcagcgttttggacctccgggcccmgaggcctggtgactcgacgagggacacggaataaagttcagacttttt

SEQ ID NO: 461 - DNA, 3384 bp, Cricetulus griseus
  misc_feature: Qualifier WAN013I8D_at
atggcggaggcctcggagaggctttaccgggtggagtacgctaagagcgggcgcgccnnnnnnnnnnnnnnnnnnnagagtatccccaaggactcgctccgcatggccatcatggtgcagtcacccatgtttgacggaaaggtcccacactggtaccacttctcctgcttctggaaggtgggccactccatccggcagcctgacgttgaggtggacggattctctgaacttcggtgggatgaccagcagaaggtcaagaagacagcagaggctgggggcgtggcaggcaaaggccaggacggaagtggcggcaagtcagagaagacgctgggtgactttgcagcggagtatgccaagtccaacaggagcacatgcaagggctgtatggagaagatagaaaagggccaggtgcgcctctccaagaaaatgctggacccggagaagccacaactgggtatgattgaccgctggtaccatccaacttgctttgtcaagaaccgggaggagctgggcttccggcctgagtnnnnnnnnnnnnnnnnnnnnnnnnnnagcctcctctctgcggaggacaaagaagtcctgaagaagcagctcccaggagtcaagagtgaaggaaaaagaaaaggtgatgaggtggatggagcagatgaggtggccaaaaagaaatctaagaagggaaaggacaaggacagtaagctggaaaaggccctcaaggcccagaatgacctgatctggaannnnnnnnnnnnnnnnnnnnnngcatgttccaccagtgacctaaaggagttgcttatcttcaaccagcagcaagtaccatctggagagtctgcgatcttggaccgagttgctgatggcatggcgtttggggccctcctgccctgcaaggagtgttcaggccagctggtctttaagagtgatgcttactactgcactggggatgtcactgcctggaccaagtgcatggtcaagacacagactcccagccgaaaggaatgggtgacaccaaaggaattccgagaaatatcctacctcaagaaattaaaggtcaaaaaacaagaccggatattccctccagagaccagtgccccagcgccgccgcacctgccaccctctgtcacctccgcacccacggctgtgaactcctcttgcccagcagataagcccctgtctaacatgaagatcctgactctcggtaaactctctcagagcaaagatgaagcaaaggccacgattgagaaactcgggggcaagttgacgggatcagccaacaacgcttccttgtgtatcagcactaaaaaggaggtggaaaagatgggtaagaagatggaggaagtgcaagcagccaacgtgagggttgtgtgtgaggacttcctccaggacgtggctgcctccactaagagtctccaagagctgctctccgcccacagcttatcctcttggggggctgaggtgaaggtcgagcctgttgaagtggcggcccccaaggggaagtcagctgcaccctccaagaagagcaagggcctgtacaaggaggaaggtgtcaacaagtctgaaaagaggatgaaactaactctaaagggaggagcggctgttgatcctgactctggtctggaacactctgcacacgtcctggagaaaggcgggaaggtgttcagtgccacacttggcctggtggacattgtgaaagggaccaactcctattacaagctgcagcttctggaggatgacaaggagagcagatactggatcttccggtcctggggccgagtgggcactgtcattggcagtaacaaacttgagcagatgccatccaaagaggatgctgttgagcacttcatgaaactatatgaagagaagactggcaatgcctggcactcaaagaacttcacaaagtatcccaaaaagttctaccctctggagattgactatggccaggatgaagaggctgtaaagaagctgacggtaaaacctggcactaagtcaaagctcccgaaggnnnnnnnnnnnnnnnncgggatgatctttgacgtggagagcatgaagaaggccttggtggaatacgagattgaccttcagaagatgcccttggggaagctgagcaaaaggcagatccaggccgcctactctatccttagtgaggtccagcaggcagtgtcccaaggcagcagtgactcccagatcctggatctttccaatcgattctacaccctaatcccccatgacttcggaatgaagaagcccccactcctgaacaatgcagacagcgtgcaggccaaggtggagatgctggacaacctcctggacatcgaggtggcctatagtcttctcaggggggggtctgatgacagcagtaaggaccccattgatgtcaactatgagaagctcaaaactgacattaaggtggttgaccgagactctgaggaagctgaggtcatcaggaagtatgttaagaacactcatgccaccacacacaatgcctatgacctggaagtgatggatatctttaagatagagcgtgagggagagagccagcgctacaaacccttcaagcagctgcacaatcggaggttactgtggcacgggtccaggaccaccaactttgctggcatcctgtcacagggtctgcggatagccccacctgaagcacctgtgacaggctacatgtttggaaaagggatctattttgctgacatggtgtccaaaagtgccaactactgccatacatctcagggagacccgattggcttgatactgttgggagaagtcgcccttggaaacatgtacgaactcaagcatgcgtcacatatcagcnnnnnnnnnnnnnnnnnncacagtgtcaaaggcttgggcaaaacgacccctgacccttcggccagcatcaccctggagggtgtasaggttccactggggacagggatcccatcgggtgttaatgacacctgcctactatataacgagtacatcgtctatgacattgctcargtgaacctgaaatacctgctgaaactgaagtttaattttaagacatccctgtggtgaagtgygaggggtgtgctgcacccakcacccaagcctakgggtaaacttccccctggtgcctccacaccacagacaccacaaaacctcagccacaccgctgcctaatggtggcaccctcacccgatctccttagggttctatatagacaggttaaagggctctgggttcaagtcccttgtccgtgttgcactcggtgggagggttgttaggggttttatcctaccangcatagccctgatagacacggctgacagtagarggaaagttggggagargtttttgttttgtttgcctagactagtcttgagggggaaaagccacgtcacagccacaatgtctgcc

SEQ ID NO: 462 - DNA, 1623 bp, Cricetulus griseus
  misc_feature: Qualifier WAN013I8N_at
cctcggaggcacgcgtccgtgctcctcgttggccatggcggactacctgattagcggaggcacatcttacgtgcccgacgacgggctcacagcgcagcagctcttcaactgcggggatggcctcacctacaacgattttctcattcttcctgggtatatcgacttcactgccgaccaagtggatttgacctctgctctaactaagaagatcaccctgaagaccccactggtttcctcacctatggacactgtcacagaggctggaatggccattgcaatggcgcttacaggaggnnnnnnnnnnnnnnnnnnnnactgtacacctgaattccaggccaatgaagttcggaaagtaaagaaatatgaacagggattcataactgatcctgtagtccttagccccaaggatcgtgtgagggatgtttytgaagccaaagccaggcatggcttctgtggtatccccatcacagatacaggccggatggggagtcgactggtgggcatcatttcttcaagggatattgattttctcaaggaggaagagcatgaccgtttcttggaggagatcatgacaaagagggaagatttggtggtggcccctgcaggcatcactctgaaggaggcaaatgaaattctgcagcgcagtaaaaagggaaagttgcccattgtgaatgaaaatgatgagctggtagccatcattgctcggacagacctgaagaagaatcgtgattacccattggcttccaaagatgccaaaaagcagctactatgtggggcagccattggtactcatgaggatgacaagtataggctggacttactggctcttgctggtgtggatgtagtggttttggactcttcccagggaaactccattttccaaatcaatatgatcaaatacatgaaagagaaataccccaatctccaagtcattggaggcaatgtagtcactgctgctcaagccaagaacctcatagacgcaggtgtggatgctctgcgagttggcatggggtgtggttccatctgcattactcaggaagtgttggcctgtggtcggccccaagcaacagcagtgtacaaggtttctgagtatgctcggcgctttggtgttcctgttattgctgatggaggaatccaaaatgtgggtcatattgccaaagctttggctcttggagcttctacagtcatgatgggctccctcttggctgccaccaccgaagcccctggtgagtacttcttctcagatgggatccggctaaaaaagtaccgtggtatgggttctcttgatgccatggacaagcatctcagcagccagaaccgatatttcagtgaagctgacaaaatcaaagtggcccaaggagtttcaggagctgtgcaggacaaagggtctatccacaagttcgtcccttatttgattgctggcatccagcattcctgtcaggacattggtgccaagagtttaacccaagtcagagccatgatgtactctggggaactcaagtttgagaagagaacatcctcagctcaggtggaaggtggtgtccacagccttcattcgtatgagaagcggcttttctgaaaagagatccagtatatgccttgaatttttcaataaaagttttg

SEQ ID NO: 463 - DNA, 613 bp, Cricetulus griseus
  misc_feature: Qualifier WAN013I8R_at
agtggatccccgtcaccaagctgggccgtctggttaaggacatgaagatcaagtccctggaggagatctacctgttctccctgcccattaaggaatccgagattattgatttcttcctgggcgcatccctaaaggatgaggttctaaagatcatgccagtgcagaagcagactcgggctggccagcggaccaggttcaaggcttttgtcgctattggggactacaacggtcatgttggtcttggtgttaagtgctccaaggaggtagccactgccatccgaggggccatcatcctggccaagctttccattgtccctgtgcgaagaggctactgggggaacaagattggcaagccccacactgttccatgtaaagtgacaggccgctgtggctctgtgttggtgcgtctcatccctgcccccagaggcactggcatcgtctctgctcctgtgcctaagaagctactgatgatggctggtattgatgactgctacacgtcagccakgggctgcactgccaccctgggcaactttgccaaggccacctttgatgccatctccaagacctacagctacctaacccccgacctctggaaagagactgtgttcaccaa

SEQ ID NO: 464 - DNA, 262 bp, Cricetulus griseus
  misc_feature: Qualifier WAN013IAQ-rc_x_at
agggcaggcagagtatatacaggatacgcttaaattaaaacacaaataataattaagacacacagagtgaaggaagggctaaggcagaaggtggggagggggcctcctggcccactgcagaagagggtgaggcctcctgtgggcacattaggnnnnnnnnnnnnnnnnnnnnnnnnnnnnnnnnnnnnnnnnnnnnnnnnnnnnnnntcgtctgcctccgtttgcgaccctgagaggttccgggtccacsggkktstcctca

SEQ ID NO: 465 - DNA, 559 bp, Cricetulus griseus
  misc_feature: Qualifier WAN008BSN_at
tcgacccacgcgtccgcccacgcgtncgcgggcccctantacgcctgcgcgcaccggcaggcgtctcgaggcgggtggtggacgatggcggcggcccangccgnggagganatgcggacgcgcgtggttctgggagagttcggggttcgcaacgtccacaccaccgacttccccggtnactactngggttacgacgacgcctgggaccaggaccgnttcganaagaatttccgtgtggatgttgttcatatggatgaaaatacactggagttcgacatggngggaattgatgctgccattgctaatgctttccggagaattctgttagctgaggtgccaacaatggcagtggaaaaggttctggtttataataacacatccattgtccaggatgagatccttgctcaccgcctggggctcatccccattcttgctgatccacgtctgtttgaatataggaacccaggggaggaagaangaacggagatagacacactgcagtttcggctacaggtcangtgtaccaagaaccccaatgctgctaaggattcctctgatc

SEQ ID NO: 466 - DNA, 600 bp, Cricetulus griseus
  misc_feature: Qualifier WAN008CTZ_at
gtcgacccacgcgtccgtggngaaaaattaaagatgcatttgagcgaaacccagaacttcagaacctgctgctagatgacttctttaagtcagcagttgacaactgccaggactcctggcggcgggtggtcagcactggggtgcaagcaggcattcccatgccctgcttcactactgccctctccttctatgatgggtacagacacgaggtgctaccagccaacctcatccaggctcagcgggattactttggggctcatacctatgaactcttagccaaaccaggagagtttatccataccaactggacaggccacggtggcagcgtgtcatcctcttcatacaacgcctagtaggactgtgtcgtcaccctccctcccaccaacaggacagtccctgtggtatgcagcactcctctttgtcctagttcctgctcagatcttttagccaagtgtcggctttgattgccgaagaagtcgctgaagtcttgtcccatagttctgaaccaccatgtgccccgcctcaaccttttgcactgcccaggaacttatctcgtgcagcactgcgtgactgctccctgactgagctgtgccatgtggg

SEQ ID NO: 467 - DNA, 246 bp, Cricetulus griseus
  misc_feature: Qualifier WAN008CVL_x_at
gtcgacccacgcgtccgcgcctccctcattcccacccctcggctccacttcctcatgactggctatacccctctcaccacggaccagtcagtggccagtgtgaggaagacaaccgtcttggatgtcatgaggcgactgctacagcccaagaacgtgatggtgtccacaggccgggaccgtcagaccaaccactgctacatcgccatcctcaacatcatccagggggaggtggaccccacccaggtc

SEQ ID NO: 468 - DNA, 600 bp, Cricetulus griseus
  misc_feature: Qualifier WAN008DQC_at
gtcgacccacgcgtccgcagaagttgcagcacatcaatcctttgttacccacttgccttaacaaggaggagagcagaacttgtaagtttgtgtccagtttcatgtctgagttgtctccagtcagagcagagctgcttggattcctcacacacgcgctcttgggagatagtttggctgctgaataccttatattgcatctcatctcaacagtatatacaagaagagatgttcttccactaggaaaatttacagttaacctgagtggctgcccacaaaatagtaccttcacagaacacctatatcggatcattcaacaccttgttccagcctcttttcgtctacagatgacaatagaaaacatgaaccaattgaaattcattccccacaaagactatacagccaatcgattggtcagcggtctcctccagctgcccaacaacacgtccctggtcattgatgagactcttctagagcaagggcagttggacacctcaagtgttcataatgtgactgcgctgagcaacctgataacctggcagaaggtggattatgacttcagttaccaccagatggagttcccgtgcagtatcaatgtgctcg

SEQ ID NO: 469 - DNA, 547 bp, Cricetulus griseus
  misc_feature: Qualifier WAN008DTT_at
gtcgacccacgcgtccggtcccaactgcaaaaattatctccagagaagtgtcggatggcattgttgccccaggatatgaagaagaagccttgaagatactttctaaaaagaaaaatggaaactactgtgttcttcagatggaccagtcctacaaaccggatgaaaatgaagttcgaacactctttggtcttcgtttaagccagaagagaaataatggtgtcattgacaagtcgctatttagcaatattgtcaccaagaataaagatctgccggagtctgccctcagagacctcattgtagccactgttgctgtcaagtacactcagtccaactctgtgtgctacgccaaggacggacaggttattggcatcggagcaggacagcagtctcggatacactgcacgcgccttgcaggtgataaagcaaactcgtggtggctgcgacaccatccacgagtgctttccatgaagtttaaaaccggggtgaagagagcggaaatctccaatgccattgatcagtatgtgacggacaccattggtgagggaga

SEQ ID NO: 470 - DNA, 523 bp, Cricetulus griseus
  misc_feature: Qualifier WAN008DUQ_at
agctctgcagatctctgatgtggtcatggcctccctgttgcggatgttccaaagcacagctgggtctgggggagtgcaggaagatgccctgatggcagttagcacgctggtggaagtattgggtggtgaattcctcaagtacatggaggcctttaaaccattcctgggcattggattgaaaaattatgctgagtaccaggtgtgtttggcagctgttggcttatttggagacttgtgccgagccctgcagtctaacattttacctttctgtgatgaagtcatgcagctgctcctggagaacttggggaatgagaatgtccacaggtctgtgaagccacagattctgtctgtgtttggtgatattgctcttgccattggtggagagtttaaaaaatacctagaggtcgtattgaatactctgcagcaggcctcccaagcccaggtggacaagtcagactttgacatggtggattatctgaacgagttaagagaaagctgcttggaagcttatacaggaatcgtc

SEQ ID NO: 471 - DNA, 535 bp, Cricetulus griseus
  misc_feature: Qualifier WAN008DXJ_at
gtcgacccacgcgtccgcccacgcgtccggtgagttgctaggacataacaagaacattcgcacaatagtgacaggcattgagatgttccacaagagccttgagagggctgaggcaggggataacctgggtgctctggtccgaggcctaaagcgggaagatttgaggcgaggcttggtcatggtcaagccaggctccatccagccccaccagaaggtggaggcccaggtttacatcctcaccaaggaggagggtggccgccacaaaccatttgtatctcattttatgcctgtcatgttctccctgacttgggacatggcctgtcgtgtcatcttgcctccaaggaaggagcttgccatgcctggagaggatttaaagctcagcctagttttgcggcagcccatgatcttagagaaaggccagcgtttcactttgagggatggcaacaagaccattggcactggtgttgtcattgacataccagccatgactgaagaggacaagaacatcaagtggagctgaatctggacttctcag

SEQ ID NO: 472 - DNA, 535 bp, Cricetulus griseus
  misc_feature: Qualifier WAN008DYV_at
acaggcgccatggttgtgctgcacgtgctgttcgagcatgcggtcggctacgcgctgttggccctgaaggaagtggaggaaatcagcctgctgctaccgcaggtggaggagtgtgtacttaaccttggcaaattccacaatgtcgttcgtctggtcgccttctgtccgttttcttcatcccaggttgccttggaaaatgccaatgccgtgtctgaaggtgttgttcatgaggacctccgtctgctcctggagacatacctgccgtctaaaaaaaagaaagtattgctgggggttggagaccccaagattggtgctgcaatacaagaggagttggggtacaactgccagactggaggtgtgatagctgagatccttcgaggagttcgtctgcacttccataatctggtgaagggtctaacagacctgtctgcgtgtaaagcccagctagggttgggacacagctattctcgggccaaagtgaaatttaatgtgaaccgggtggacaacatgatcatccagtccataagccttctgg

SEQ ID NO: 473 - DNA, 600 bp, Cricetulus griseus
  misc_feature: Qualifier WAN008DZD_at
ggtcgacccacgcgtccggccactttgcagccgtttttcacgttgcagttggatattcagtctgacaagatacgcacagtccaggatgcattggaaagcttggtggcaagagagtctgtccaaggttacaccaccaaatccatacttgagttgaagtcagtcgcagagtgactctggagaagctccctcctgtcctcgtgttgcacctgaagcgcttcgtctatgagaagacgggtgggtgtcagaagctagtcaagaacattgattatcctgtggacttggagatcagcagagaacttctttctccaggagtcaaaaataagaattttaaatgccacagaacctataggctgtttgcagtggtgtaccatcatggcaacagtgccacgggcggccactatactacggatgtcttccagattgggcttaatggctggctgcgaattgatgaccaaacggtcaaagttattaaccagtaccaggtggtgaagccaactgctgaccgcacagcctacctcctgtattaccgccgtgtggacctgctgtagccgtgtccgtgtgcctgtgtgtgtgcctgacgctgcttcctacaattcccaa

SEQ ID NO: 474 - DNA, 578 bp, Cricetulus griseus
  misc_feature: Qualifier WAN008EHW_at
gtcgacccacgcgtccgcccacgcgtccgtgaggataagctgaggccctagctctcccctgtgctggtggtgcccggctgcccgccgtgatgcagtggtccttgggccggcggtgggcatggatcgccctatttctggctgttgccgcggtgctgacccaggcgggggggctgtggctgggtactcagaacttcgtcttccagcaagaagagatagcgcagcttgctcgacagtacgctggactggaccatgagctggccttctctcggctgatcgtggagctgcggaggctgcacccaggccacgtgctgccggatgaggagctgcagtgggtgtttgtgaacgccggcggctggatgggcgccatgtgtcttctgcacgcctcgctgtctgagtacgtgctgctcttcggcaccgccctgggctcccgtggccattcggagcgatattgggctgagatttctgacaccatcatctctggcactttccaccaggggagagagggcaccacgaaaagtgaagtcttctacccatgagagacagttgtacacggacctggaaaggcaacggccttggga

SEQ ID NO: 475 - DNA, 2947 bp, Cricetulus griseus
  misc_feature: Qualifier WAN013I6N_at
tccgccgcagccgcagccatcgtcggcgcccctcgctcttctcctgtgcccgtgagaatccgtcgccatccgccactatggtgaacttcacggtagaccagatccgtgccattatggacaagaaagccaacatccggaacatgtcagtcatcgctcacgtggaccacggcaagtccacactgacggactccctggtgtgcaaggcgggtatcatcgcctctgcaagagccggtgagacacgcttcacagacacccgcaaggacgaacaggagcgctgcatcactatcaagtccacggccatctccctcttctatgagctctctgagaatgacctgaacttcatcaagcagagcaaggatggatctggctttctcatcaacctcatcgactctccaggccatgtggatttctcctcagaggtgacagctgcacttcgtgtcaccgatggagctcttgtggtggtggactgtgtgtctggtgtgtgcgtgcagactgagaccgtgctgcggcaggccattgccgagcgcatyaagcctgtcctgatgatgaataagatggaccgtgccctgcttgagctgcagctggagcctgaggaactataccagaccttccagcgcrttgtggagaatgtcaacgtcatcatttccacctatggcgagggcgagagtggacccatgggaaatattatgattgaccccgtcctgggcactgtgggctttggctctggcctgcatggctgggccttcactctgaagcagtttgcggagatgtatgtggctaagtttgcagcaaagggtgagggccagctggggcctgctgagcgggccaagaaagtggaggacatgatgaagaagttgtggggagatcggtattttgatcccgccaatggcaaattcagcaagtccgctaacagccctgatgggaagaaactgccacgcaccttttgccagctcatcctggaccccatcttcaaggtgtttgacgccatcatgaacttcakaaaggaggagacggccaagctgattgagaagctggatatcaagctggacagcgaggacaaagacaaggagggcaagcccctgctgaaggctgtgatgcgnnnnnnnnnnnnnnnnnnnnnnnnnnnnnnnnnnnnnnnnnncacacgwcckcccttcccccgtcaccgcccagaaataccgctgcgagctgctctacgaggggccacctgatgatgaggcgrccatgggcattaaaagnnnnnnnnnnnnnnnnnnnntnnnnnnnnnnnnnnnnnnnatggtgccaacctscgacaaaggcnnnnnnnnnnnnnnnnnnnnnnnnnnnnccggggtagnnnnnnnnnnnnnnnnnnnnccgcatcatgggccccaactacacgcctgggaataannnnnnannnnnnnnnnnnnnnnnncagagtnnnnnnnnnnnnnnnnnnccggtatgtggagccaattgaggmcttgccctgtannnnnnnnnnnnannnnnwtggtgtagannnnnnnnnnnnnnnnncagnnnnnnnnnnnnnnttnnnnnnnnnnnnnnnnnnnnnnnnnnnnnntgttcagcgtcagccctgtsgtsaragtggctgtggakgccaagaacccagctgacctgcccaaactggtggagggcctgaagcrgctggcaaaatctgaccctatggtgcagtgcatcattgaggagtctggggagcacatcattgctggagcaggtgagctgcacctggaaatctgcctcaaggacctggaggaggaccacgcctgcatccccatcaagaaatctgaccctgttgtctcataccgggagacagtgagcgaggagtcaaatgtgctgtgcctgtccaagtcgcccaacaagcacaaccgattgtatatgaaggcccggcccttccctgatggcctggccgaggacatcgacaagggtgaagtgtctgctcgccaggagctcaaggcacgcgcacgctatctagctgaaaagtacgaatgggatgttgctgaagcccgcaagatctggtgctttgggcctgatggcactggccccaacattcttaccgatatcaccaagggtgtgcagtacctgaatgaaatcaaggacagtgtggtggctggcttccagtgggccactaaggagggcgctctctgtgargagaacatgcgtggtgtgcgcttcgatgtccatgatgtgaccctacatgctgatgccatccaccgaggaggtggtcagatcattcccacagctcgtcgttrtctgtatgccagtgtgttgactgcacagccccgcctcatggagcctatctacctggtggagattcagtgtcctgaacaggtcgtgggtggcatctacggtgtcctgaacaggaagmktggccatgtgtttgaagagtcccakgtggctggcacccccatgtttgtagtcaaggcctatcttccagtcaacgagtcctttggttttacagctgacctgcgctccaacactggtggccaagnnnncccccagtgtgtgttcgaccactggcagattctgcccggggacccgttcgacaacagcagccgtcccagccaagtggtggctgagacccgcaagcgcaaaggtctaaaggagggcatcccagcactggacaacttcctggacaaactgtaggcaggcttgcacagccacacactgcacagtgcccacccatcagaagacaccttgagactgtccccataatgctgctctggaggtggctggggccaccctgccattgagcactaacacttgatgccgactctatttatttcggaatttccgaggcagtggagtcgccctctgcaggctggactggctggcgggccatggggtgggcaggacacagctctttatcattttcagagggaaaaatgctcagatatcaaacatctaaataaatgcattcagaggaaaaaaaaaa

SEQ ID NO: 476 - DNA, 1248 bp, Cricetulus griseus
  misc_feature: Qualifier WAN013I9E_at
gcacacggacatttgctctctcaggaaggttttggcagcaaccatgtccactttcgtggagctcagtaccaaagccaagatgcccatcgtgggcctgggcacctggcagtctcccccaggccaagtcaaggaagctgtgaaggtggccattgatgctggataccgccacattgactgcgcctatgcgtattacaatgagcacgaagtgggagaggccattcaagagaagatcaaagagaaggctgtgagacgggaagacctcttcatcgtcagcaagttgtggcccacctgctttgagaggaaactgctaaaggaggcctttcagaagaccctcacggacctgaaactggactatctggacctgtatcttattcactggccacagggacttcagcccggaaaggagttatttcccaaagacgatcaaggcaacgtcctcaccagcaaaataacgttcttggatgcctgggaggtcatggaagaactggtggatgaagggttagtgaaagccctgggtgtctccaacttcaaccacttccagattgaaaggatcctgaacaaacctggattgaaacataagccagtgaccaaccaggtcgagtgtcacccatacctcactcaggaaaaactgatcgagtactgtcactccaagggcatcactgtcacagcctacagccctctgggttccccaaataggccttgggccaagccagaagacccttcactcctagaggaccccaagattaaagagattgctgcaaagcacaagawaacctcggcccaggttctgattcgattccacatccagaggaatgtggtggtgatccccaagtctgtgacaccagcacgtatacatgagaactttcaggtctttgacttccagttgagtgatcaggagatggccaccatccttggcttcaacagaaactggagggcctgcctgctgcctgagacagtaaacatggaagaatatccctacgatgcggagtattgaagctaactcgcctgatgaggcttccctctttgctgatgcacacctcaatcatttctggtgtgccatttgagccaagcctgcctgatgtcatgctctaggcaacgctgtacttaaaatcctgtgttcaaggaagtgtgactctgagaagtgaatgtgcatttctttcactgannnnnnnnnnnnnnntgtttgccaaaaatcaggaactctggtaactttggaggacataagaatagaacaataaaagccaagcc

SEQ ID NO: 477 - DNA, 1106 bp, Mesocricetus auratus
  misc_feature: Qualifier AF004814_at
gccgccgcctctaccgccgcgacggtccgggccgcggtcgcccagggactttgaatatgtcggggattgccctcagccgacttgcccaggaaaggaaagcttggaggaaggaccacccttttggctttgtggctgtcccaacaaagaaccctgatggcacgatgaacctgatgaattgggagtgcgctattcctggcaagaaggggactccatgggaaggaggcttgttcaaactacggatgcttttcaaagatgactatccgtcctcaccaccaaaatgtaaatttgagccaccactgtttcacccaaatgtgtatccttctggcacagtgtgcctgtccatcctggaagaagacaaggactggaggccagccatcacgatcaaccagctctttataggaatacaggaacttcttaatgaaccaaatatccaagaaccagctcaagcagaggcctacacaatttactgccaaaacagagtggaatatgagaaaaggttccgagcacaagcgaagaagttttgcccctcctaagcagcggcacttgttgctcccatgacggaggaagggattggcttggcaagaacttgtttacaaacttttgcatatctaagtcgctgcgtacaagttactagtcacctgggagggttgagcgggcgccattttccatttctgccactggcatttacagtctcaattcgctgaattgccccagttttcatacagggtctcttccttcagtcttttgtatttttgattgttatgtaaaacttgcttttattttaatattgatgtcagtatttcaactgctgtaaaatgataaacttttgtacttggtaagcccctaggagctagtttcttcgcgctcggatgcaggcatgcttcccactgttctctggcctccagctggctgtatgacagatccacactgtccctctctccccatcctcgtcctcctcagaaacctgggctgttgcttatgagcctcagatccaaagttggccagcgtctccattctgcacctcttcctttgtgtttatatggcgttttgtctgtgttgctgtttagagtgaataaactgnnnnnnnnnnnnnnnnnnnnnnnnnn

SEQ ID NO: 478 - DNA, 260 bp, Cricetulus griseus
  misc_feature: Qualifier AF022942_at
tggcgcgctgtcttcccgcccgcgtcagggacctgcccgactcagcggctgccatggcatcagatgaaggcaagcttttcgtgggagggctgagcttcgacaccaatgagcaggcgctggagcaggtcttctccaagtacgggcagatctctgaagtggtggtggtaaaggacagagagacccagcgatccagaggctttgggtttgttacctttgagaacatcgatgatgctaaagacgccatgatggctatgaatggg

SEQ ID NO: 479 - DNA, 1747 bp, Cricetulus griseus
  misc_feature: Qualifier AF093673_at
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagtccagcaatgcagccgggaccagcgttgcaggccgtgttgctggcggtgctgctgtcagaaccacggagttcgaagggtcggctgctgagcgggcagctggtctgccggggagggactcggaggccttgctataaagtcatttacttccatgatgcttttcaaagactgaactttgaggaagccaaagaagcctgcaggagggatgggggacagctcgtcagtattgaaacagaagatgagcagagactgatagaaaaattcattgaaaacctcttggcatctgatggtgatttctggattggcctcaggaggctggaggtgaagcaggtcaacaacacagcctgccaggacctttatgcttggacagatgggagcacatcacaatttaggaactggtatgtggatgagccttcttgtggcagtgaggtctgcgtggtgatgtaccatcagccatcggcaccacctggcatcgggggctcatacatgttccagtggaatgacgaccggtgcaacatgaagaacaatttcatttnnnnnnnnnnnnnnnagaagccaagtacaacaccttctataaggcctggaggtgaagcaactgagccaccaacaccagtacttccagaagaaacacagaaagaagacaccaaagaaacattcaaagaaagcagagaggctgctttgaatcttgcctacatcctaatccccagcattcccctgttcctcttactagtggtcacttcagctgcatgttgggtttggatctgtagaagaagaaaacaagagcagccagatcctaccacaaaggagcaacacaccatttggcccactcctcaccaagaaaacagccccaacctagatgtttacaatgtcatcagaaaacaaagcgaagctgatttaactgagccccgcccagacctgaagaacatctcatttcgggtgtgttctagtgaagcccctcctgatgatatatcttgtgactatgacaacatggctgtgaacccttcagaaagtgggtttgtgactctggcaagtatggagagtggatttgtgaccaatgacatttatgagttctcccctgacagaatggggaggagcaaggagagtggatgggtggaaaatgaaatatattactaagacatatgaaaaactggcaaaaatgagaaaacaaaagcaaaagcctcctatttctcacaaggaaaatgttcagaaagtttatgaagatgctcagaccagttcttaaggtgagcacagaatctccacaagttcctactggggtccccaagttctggtatgtatcccttatcccagagcctcacccagctcagccttgtgagaaggcaccttgcctaggggctgccatacagcagaccctcaataactgtcagccagtgtgctgcaactgatgttcaagggctgatgcttccaatggtcaggataaaggtgtcaccaaggagtttggaaagcccgcctctaggctccttgctctctacagcagcacgtgcaatcatagaaagacataaactctggaatcttctcaaaggctatgtatggaagccccaggctggcctgcccagcaacaattcccatatcttcccctagcccaaataaaatccaat

SEQ ID NO: 480 - DNA, 292 bp, Cricetulus griseus
  misc_feature: Qualifier S74024_at
tcatatcttatgaaccactttgatttgccaacatgtgatagctgcagagatgctgatgacaaacacaagctgataaccaaaacagaagccaagcaggaataccttctgaaagactgcgatttagagaagagagagcctgcgcttaggtttatcgtgaagaagaaccctcgtcactcacagtggggcgatatgaagctctacttaaagttacaggttgtgaagcgggctctggaagtttggggtagccaggacgcgctggaagacgcgaaggaagtgaggcaggaaaaccgag

SEQ ID NO: 481 - DNA, 323 bp, Cricetulus griseus
  misc_feature: Qualifier WAN0088IR_at
tcgacccacgcgtccgggctgcagccctacgcttttctccgtaattctgcagggtcgtcctggcaaccagttgctcagccaatcagcacttggtactacgatccgccctcaacatctggagcaatggcggtaagaagagccctgattgtattggcccattcagagaggacatcgttcaactacgccatgaaggaggctgctgtagagattctgaagaagaaaggatgggaggtggatgaatctgacctctatgctatgaactttaaccccatcatttccagaaaggatatcacaggtaagctgaaggacgcaaagaactttca

SEQ ID NO: 482 - DNA, 600 bp, Cricetulus griseus
  misc_feature: Qualifier WAN0088T3_at
gtcgacccacgcgtccgcggacgcgtgggcggacgcgtgggtttcagaactacagaaagtacagtgaacatgacatgaacaaagttctggatctggaaatgaagggtgacatcgagaagtgcctcactgcccttgtgaaatgttccaccagcaccccagctttctttgctgagaaactttatgaagccatgaagggagctggaactcgccataaggcattgatcaggattatggtctcccgttctgaaatcgacatgaatgaaatcaaagcattttacctgaagaagtatggaatctctctttgccaagccatcctggatgaaaccaaaggagactatgaaaagatcttggtggctctgtgtgatggcaactagatgtccccgctgctcagaagcactgtgatgagagcatctcttagctgttgctttcctctatcacacaagcttaagtaagagggttgctttagcagataacaagctaattacccactttgaataatataacatatgaataagttttacatcatacctctgtacaatagagaaataattgttttattatgttttccccagattataaatccctataagcaagttactc

SEQ ID NO: 483 - DNA, 251 bp, Cricetulus griseus
  misc_feature: Qualifier WAN0088YL_f_at
gtcgacccacgcgggcgtgtgattccgtctttgcggtgggagtcatggccactcgtcgagctctgcacttcgtcttcaaagtggggaaccgcttccagacggtgcatttctttctagatgtcctggggatgcaggttctacgccatgaggaatttgaagaaggctgggggggctgcatgtaatgggccttatgatgggaaatggagtaaaacaatggtgggatttgcgcctgaggatgatcattttgttgc

SEQ ID NO: 484 - DNA, 600 bp, Cricetulus griseus
  misc_feature: Qualifier WAN008D29_s_at
gtcgacccacgcgtccgcccacgcgtccggaccaaggaaaatggcagacggtttttcgcttaatgatgccttagctggttctggaaacccaaaccctcaaggatggcctggtgcataccaacctggggcagggggctacccaggggcttcctatcctggggcctaccctgggcaggctcctccaggggcttatcctgggcaggctcctcctagtgcctacccaggcccaactgcccctggagcttaccctggctcaactgcccctggagcttaccctggcccaactgcacccggagcttaccctggatctgcacctggagccttcccagggcaacctggagcacctggggcctaccccagtgctcctggggcctatcctgctgctggcccctatggcgcccccactggagcattgacagtgccctataagctgcccttggctggaggagtcatgcctcgaatgctgattacaatcatgggcacagtgaagcccaatgcaaacaggattattctaaatttcatgagagggaatgacattgccttccactntaaccctcgcttcaatgaaaacaacaggaaagtcattgtgtgcaacaccaa

SEQ ID NO: 485 - DNA, 509 bp, Cricetulus griseus
  misc_feature: Qualifier WAN008D85_at
gttcgaccccactgcgctccggggcagggaagggccttaggaaactaacatgctttcactactgagggggacggacggacggacggacgcactcgggtctcgctggcgctgttctgaaaccatctggctggctttctgaggtcaggctgggcgggggctgtcattggccgcgctgccgatcacactggccctcatccctccagcgatctcgcgctgttctgtaaattcctggtgctaacatcccatgccgctcccccgtggcccccccagccgcctgcccagtccagggctggtccaagaatgacgtgggggatggcctctgtgtaatgcgctggtgattttttccttcccctttatgccccttaaaattttaatttttgccccaaacatgccgggccaactaaagggtggtgagagggatttgattcccaccatgcccacaagaaccaacctgcagtaaacacttctgtgggccgccgcgcatagcacgccgcgggcacctactgccc

SEQ ID NO: 486 - DNA, 438 bp, Cricetulus griseus
  misc_feature: Qualifier WAN008DKX_at
gtcgacccacgcgtccgctgaaacaagaacccaagaagggagagccagagaagcagtgagccctggagaagctccagcgggtcacagctgcctcctgccaccctgcaccacatcaacgctacgagaaacgcacactgttcaccattgccattgtggttctaatgcaggcaaatacactgtttgattatgcctagttctacgttcctttcagctcactcgtatctttagtgcaattaacgtctgttctgatttatgttgtgtcctgagagctgaagtcttaagctttggtggcctttgaagagtgacagcaagttttacctaaccctgtgggggggaggttgccagcagaatgccttgtagttatttgatagaatttattactgtatatttaacagatctgcgtatatatatatattataataaaagaatgtgagatga

SEQ ID NO: 487 - DNA, 600 bp, Cricetulus griseus
  misc_feature: Qualifier WAN008DQD_at
tcgacccacgcgtccggtgccatgacaatgcagttcatcagccaccgattcccagaagaccatgaccccaccattgaagacgcttataagatccggattcgcatcgatgatgagcctgccaatctggacattctggatacagctggacaggccgagttcacagccatgcgggatcagtacatgagagcaggagaagggtttatcatctgttactccatcacggatcgtcgaagtttccacgaagttcgggagtttaaacaacttatttaccgcgtccgacgcacggatgacacaccagtggttcttgtgggaaacaagtctgacttgaagcagctgagacaggtctccaaggaagagggattgtctctggctcgagaattcagctgtcccttttttgagacctccgctgcataccgttactacattgacgatgttttccatgctcttgtacgggaaatacgtaagaaagaaaaggaactagttttggccatggagaaaaaatctaaacccaaaagcagtgtatggaagatgctgaagtcaccattcaggaagaagaaggactctgtaacttgaggggagtgtaaaggatctgctgtgaac

SEQ ID NO: 488 - DNA, 506 bp, Cricetulus griseus
  misc_feature: Qualifier WAN008E7S_at
gtcgacccacgcgtccgcggacgcgtgggcgtgcccgtgcccaccccgaccccacgtgtcatgcccaacagggcctttgttgctttgagcccgaggtcatgccttgacctcaagcaagagctgtagccagcccttgcagtgcagaccagaccacgggcaacggcaggggccgtccccacgagggcagcccctccgcacagttctagatgcgtatgcacagtatttattccttcctgcgctcctagtggtcgtgactttcgcagcagcagctcccctggcttcccacgggtgagacctccctgtggacccgagaaccacccgctgttcccgcagctgtcctccgcaatgccaaacgcaaggttccagagagcacttgctctccaagtttcctgagccgttcttttgaccccgggcctgggcactggggagcaggtgtgcctacccaggcccgactgcgtgcctcccaagggatcatggcaccttgagcacatcctagtttcctgaga

SEQ ID NO: 489 - DNA, 226 bp, Cricetulus griseus
  misc_feature: Qualifier WAN008EUZ_x_at
tcgacccacgcgtccgccaccatggcgctgactgtgggagggaccatctactgcctgattgccctctactggcttcccagcccaaaataagtgaattagcctttgggagactcttcgtggcacaaaccctttgatttttcaaattcctgggtttttgttttggggggggtttgttttttttccacttgggatgggctactaacatttttgcaaacaaaggaaaaga

SEQ ID NO: 490 - DNA, 539 bp, Cricetulus griseus
  misc_feature: Qualifier WAN013HUG_at
tactctagattaaccatcccagtccttctgtcagcctccgatgccatcatgcagcctgattaggagcaaaggaaagggggaaaggagaagcaactaatcgtcttttcccgatcgtcaggaccctaaagaatggccgagccttgggggaacgagttggcgtccgcagctgccaggggggacctagagcaacttactagtttgttgcaaaataatgtaaacgtcaacgctcaaaatggatttgggagaactgcgctgcaggttatgaaacttggaaatccagagattgccaggagacttctactnnnnnnnnnnnnnnnnnatttgaaagaccgaactggtttcgctgtcattcatgatgcagccagagcaggtttcctggacacggtacargctttgctggaktttcaagctgatgttaacatcgaagataatgaagggaacctgcccttgcacttggctgccaaagaargccacctccctgtggwggagttccttgtgaagcacacaaccagcaatgttggggcatcggaatcataagg

SEQ ID NO: 491 - DNA, 579 bp, Cricetulus griseus
  misc_feature: Qualifier WAN013HUW_at
gaccgaattggcatttccaagtcagagttagttgcaatgttggaggaagaagagctgagaaaggccattttagtggtgtttgcaaataagcaggacatggagcaggccatgacaccctcagaaatggcaaatgcacttgggttacctgctctgaaggaccgaaagtggcaaatatttaaaacatcagcaaccaaaggcactggtcttgatgaagccatggaatggttagttgaaacgctgaaaagcagacagtaagcccattcacatcaggtcctgtgaagcaaatgctacgccacacactcactgcaaccagccagatgtgcttctgtgactgttagaactgtgtgattgctggcatgtacagaactgactccaatgtttgtaataaatataaaaaacaagtatttggtgagagggttagcttgattgaattacccaaatggtttatgtaatgtaaaatattcttccttgctttcttgtgttaaggcatatgttctatttgtatggaattcttactcaaatacaattctattaaagaatgtacacttttggggggaaccctcctatttttaaattagt

SEQ ID NO: 492 - DNA, 582 bp, Cricetulus griseus
  misc_feature: Qualifier WAN013HX5_at
aagtccccgaggtgtgccagctgggtcccacagcttgcggtggtgcttctccagggactcaggtgtgtgcacagcaggggtactcggcggtccggtgctgctcgcaggcaagaagagatcatttaaagattgagaccaagactgaaagaatggctgacctccatcagctcaaggacaatgaggtattgatggcctttgcctcctatgcaactattgttcttaccaagatgatgttcatgagctcggcgactgcattccagaggctaaccaacaagatttttgccaacccakaagactgcgctggatttggcaagggagagaatgccaagaaatttcttcggactgatgagaaggtggaacgggtgcgaagagcccacctaaacgatattgaaaacatcgttcccttccttggtatcggcctcctctactccttgagtggtccakacctctcaacggccctcatgcacttcaggatctttgttggtgctcggatctaccacacaatcgcctacttgactccccttcctcarccaaacagggcctttgcattttttgctggctatggagttactttgtcaatgg

SEQ ID NO: 493 - DNA, 612 bp, Cricetulus griseus
  misc_feature: Qualifier WAN013HXQ_at
gttgcggacgttttgctcgcgaaccacagcytgcggacatggcgaaccaggtgatcagatgcaaggctgcagtcgcctgggaggccggaaagcctctctccatagaggaagtakaagtggcccctccaaaggctcatgaagttcgaattaagrtyattgccactgccgtctgccacaccgatgcctataccctgagtggagctgatcctgaggggtgtttcccggtgatcttgggacatgaaggtgctggaattgtggaaagtgttggtgaaggggtgactagggtgaaggcaggtgatactgtcatcccgctttacgtcccacaatgtggagaatgcaaattttgtctgaatcctaaaacaaacctttgccagaagataagagtgactcakgggaaagggttaatgcccgatggcacgaccagattcacctgcaaaggaaagcctcttttccatttcatgggaaccascacatytctctgagtacacagttgtggctgacatctctgttgctaaaattgacccttcggcacctttggataaagtatgccttctcggttgtggcgtttcaaccggctatgggtgctgctgtgaacactggcaaggtggagcc

SEQ ID NO: 494 - DNA, 561 bp, Cricetulus griseus
  misc_feature: Qualifier WAN013HZO_at
tcagcgctaggcgcccggcccagcttctttttcaaaatgtctactgtccacgaaatcctgtgcaagctcagcttggagggcgatcactccacacccccaagtgcctatgggtcagtcaaaccctacaccaactttgatgctgagagggacgctctgaacatcgagacggctgtcaagaccaaaggagtggatgaggtcaccattgtcaacattctgactaaccgcagcaatgcacagagacaggacattgccttcgcctatcagagaaggaccaaaaaggannnnnnnnnnnnnnnnnngtcagccttatctggccacctggagtcggtgattttgggcctattgaagacacctgcccaatacgacgcttctgaactgaaggcctccatgaagggcctggggactgatgaagactccctcatnnnnnnnnnnnnnnnnnnnnnnnnnnnnnnnnnnnnnnnnnnnnnnngagtgtacaaggaaatgtacaagaccgatctggagaaggatatcatttctgacacatctggtgacttccgaaagctgatggtcgcccttgca

SEQ ID NO: 495 - DNA, 604 bp, Cricetulus griseus
  misc_feature: Qualifier WAN013I1X_at
tccacttccagactgaagccgtctgtacagagcctcaattcagttcctgggaagacaagaaaataaaaagayacttctttaaaaatggcgatggtatcakaattcctcaagcaggcctggtttattgacaaccaagaacaggaatrtgtkcaagctgtaaaatcatccaaaggtggtcctgggtctgccgtgagtccctacccatccttcaatccatcctcagatgttgctgccttacacaaagctatcatggttaaaggtgtggatgaagcaaccattattgacattcttaccaagagaaccaatgctcagcgccagcagatcaaggcagcatacttgcaggaaactggaaagcccctggatraaatgctgagaaaagcccttacaggccatctggaagaggttgttttggctctgctaaaaactccagcccagtttgatgcasatgaactccgtgcggccatgaaggggcttggaacagatgaagacactctcattgagattttggtatcaagaaataacagagawatcagagaaatcaacagartctacagagaagagctaaagagagatctggccaaagacatcacttcggatacttctg

SEQ ID NO: 496 - DNA, 593 bp, Cricetulus griseus
  misc_feature: Qualifier WAN013I2Q_at
tccggttgccgccatcttcctcgggcggaagccggcttctaggtgggagttcagtggcgggtgccgtgggtagcgcggacggtcggccggtgccatgccgtcatcaccgctgcgggtggcggtcgtgtgctcgagtaaccagaaccggagcatggaggcrcacaacatcctcagcaagcggggtttcagtgtccggtcctttggaacaggaactcatgtgaagctcccaggaccagcacctgacaagcccaatgtttatgacttcaaaaccacatatgaccagatgtacaatgatcttctcaggaaagacaaagaactctatacgcagaacggaattttacatatgttggacagaaataaaaggatcaagccccggcccgaaaggttccagaactgcaaggacctgtttgacctgattctcacttgtgaagaaagagtctatgaccasgytgtasannnnnnnnnnnnnnnataacaggagacctgccagtcagtgcatgttggcaatgyggacatccaggacaaccatgaasaggccacccykggggcatyccttmtctgcgayctctgccagtgtmtccagcacactgagg

SEQ ID NO: 497 - DNA, 579 bp, Cricetulus griseus
  misc_feature: Qualifier WAN013I33_at
tctcagatctctcggccttgccagggcccaccagagaagactgacgctctggtccggccatacctatgggggaccaaccttgtgcctccgggagatccacgctcccaccaggaaatacgcgggaacccaagcctccaaaaaagcgctgtctcctggctccacggtgggattatccagaagggactcccagtggaggtagtaccactctcccctcggcgcctcctcctgcatctactggactgaagtctcaccctcctccgccggagaagtagtgaaatagcttttttcgcagcctaaatcagtctttgaaagattgcagtatgactccatttccttggtgcatttgtaaaatagttcacccattgaagaccatgaagactctttccaatgctaccctgcttttgctggtgtcccgcacctggaagttgtgatgctcaaggctcatctcatattttctaccccattgaccatttctctatttggctgtgctactgggagatcatatttacaaactgagtattatgatgtgatgactgtgatttcacctccagaatttggatgaatttatgacgtgggcaa

SEQ ID NO: 498 - DNA, 483 bp, Cricetulus griseus
  misc_feature: Qualifier WAN013I3V_at
atctgagttgcagctggtggcgcagcgcatccgcagtttccccgacttccccatccccggcgtgctgtttagggatatctcgcccctcctgaaggaccccgcctccttccgagcttccatccgcctcctggggaggggaccttaagtccacgcatggcggcgagatcgactacatcgcaggcctagactccaggggattcttgtttggcccctccctagctcaggagctgggcctgggctgtgtgctcatccggaagcgagggaagctgccaggccccacagtgtcagcctcctatgctctcgagtatggcaaggctraactagaaatccagaaagacgccttanaacctggccagaaagtggttgttgtagatgatctcctggccactggaggaaccatgtgcgctgcctgtgagctgctgggccatctacaggctgaggtggtggagtgtgtgaccctggtggagctgacctcacttaagg

SEQ ID NO: 499 - DNA, 1419 bp, Cricetulus griseus
  misc_feature: Qualifier WAN013I4Q_at
ccgagccgagaatgggagtagagccgactgcttgattcccacaccaatctcctcgccgctctcacttcgcctcgttctcgtggctcgtggccctgtccaccccgtccatcatcccgccggccaccgctcagagcaccttccaccatggccacctcagcaagttcccacttgaacaaarrcatcaagcaaatgtacwtgtscctgccccagggtgagaaagtccaagccatgtatatctgggttgatggtacyggagaaggactgcgctgcaaaacccgcaccctggactgtgagcccaagtgtgtagaagagttacctgagtggaattttgatggctctagtacctttcagtctgagrgctccaacagtgacatgtatctcagccctgttgccatgtttcgggaccccttccgcaaagagcccaacaagctggtgttctgtgaagtyttcaagtacaaccrgaagcctgcagagaccaatttaagrcacwcgtgtaaacggataatggacatggtgagcaaccagcacccctggtttggaatggaacaggagtatactctswtgggaacagatgggcacccttttggttggccttccratggcttycctgggccccaaggtcygtattactgtggtgtgggcgcagacaaagcctatsgcagggatatcrtggaggctcactaccgygcctgcttgtatgctggggtcaagattacaggaacawatgctgaggtcawgcmtgcccagtgggaattccaaataggaccctgtgaaggaatccgcatgggagatcatctctgggtggcccgtttcatcttgcatcgagtatgtraagactttggrgtaatagcaacctttgacyccaagcccattcctgggaactggaatggtgcaggctgccataccaactttagyaccaagrccatgcgggaggagaatggtctgaagcacatcraggaggccatygagaaactaagcaagcggcaccggtaccayattcgagcctacgatcccaaggggggsctggacaatgcccgtsgtctgactgggttccacraaacgtccaacatcaacgacttttcwgctggygtcgccratcgcagtgccagcatccgcattccccggactgtcggccaggagaagaaaggttactttgaagmccgcygcccctctgccaattgtgacccctttgcagtgacagaagccatcgtccgcacatgccttctcaatgagactggcgacsagcccttccaatacaaaaactaattagactttgagtgatcttgagcctttcctagttcatcccaccccgccccagctgtctcattgtaactcaaaggatggaatatcaaggtctttttattcctcgtgcccagttaatcttgcttttattggtcagaatagaggagtcaagttctt

SEQ ID NO: 500 - DNA, 1021 bp, Cricetulus griseus
  misc_feature: Qualifier WAN013I51_at
tttctgtctaacactgaggttcgccgggtcatggtgccagcctgactgagaagaggacgctcccgggagacgaatgaggaaccacctcctcctgctgttcaagtacaggggcytggtgcgcaaaggggaaraaaagcaaaagacgaaaatggctraattcaagatccgtccagccactgcctctgactgcagtgacatcctgcgactgatcaaggaactggctataatatgaatacatggaagatcaagtaatgttaactgagannnnnnnnnnnnnnnatggttttggagaacaccccttctaccactgcctgattgcrgaagtgcctaaagagcaytggacccctgaaggacacagcattgttggtttcgccatgtactattttacnnnnnnnnnnnnnnnnggcaarttactktatcttgaaracttctttgtgatgagtgattacagagggyktggyatasgatcagaaattttgaagaatctaagccaggttgccatgaagtgtcgytgcagcagtatgcacttcttggtagcagaatggaatgaaccatctatcaacttctacaaacgaagagstgcttctgacctgtccagtgaagagggatggaggctcttcaagattgacaaagagtacytgctaaaaatggcagcagaggagtraggagtgctggtgtagatggtgacaacctccattatactttagaatacattctcaacttatcttgctctctatcttgtttgtagtgaaatagtcgaatgagcacccattcnnnnnnnnnnnnnnnnnnnnnnnnnnnncatgtttgaaatgcagtctgtttaaaatggcactcatgtatgtggtttcgaggcagaattctcgaacatctttcagtgaacaacaaggtgatatgaccttactatatatgaaaaacaaaacttcattcttgtgagtcatttaaatgtgtacaatgtacacactggtacttagagtttctgttttgatccttttttaataaattactctttgatttaatt

SEQ ID NO: 501 - DNA, 1964 bp, Cricetulus griseus
  misc_feature: Qualifier WAN013I66_f_at
agccccctcttctcctccccctcccgccttcctcacttctctctgggtgcccattggttggcgcgccccgcggctaggatggcagtgggaggggaccctctttcctaacagtgttataaaagctgcgcccttggcggggtccggtcctctgccactcttgctcsgggacyccagagacarcagcrctcccrcggttcgcmtscaccgygccctygctctcctwtcggagccagtccttgyagctgcagcagcccagyccaccttcgcyctcsaaaccatrtcyaccaggtccgtgtcctcgtcctcttaccgcargatgttcggtggymccggcacctcgarccggcmgagctccaaycggagctatgtgaccacgtccacccgcacctacagcctgggcmrcgcrctgcgccccagcaccagtcgcagcctctattcctcatcycccggtggcgcctatgtgacccgatcctctgcggtgcgcctgcggagcagcatgccsggcrygmggctgctgcargaytcggtggacttctcgctggcsgacgccattaacaccgagttcaagaacacccgcaccaacgaraaggtagaactgcaggagctgaaygaccgcttcgccractacatcgacaaggtgcgcttyctrgagcagcagaacaaaatcctgctagccgagctcgagcarctcaagggycagggcaagtcgcgcctgggcgacctctatgaggaggagatgcgggagctgcgccggcaggtggatcagctcacyaacgacaaggcacgcgtcgaggtggagcgtgacaayctrgcwgaggacatcatrcggctgcgagaaaaawkgcmkgassagmkrstsswgrswgwggragcsgagagsaycctgsagycctkcmgacaggatgttgacaatgcctctctggcacgyctygaccttgaacgtaaagtggaatccttgcaagaagagattgcctttttgaagaaactgcatgatgaagagatccaggagctacaggcccagattcaggagcarcatgtccagattgaygtggatgtttctaagccygacctcactgctgccctgcgcgatgtccgccagcagtatgaaagtgtggctgccaagaacctccaggaggcrgaggaatggtacaagtccaagtttgcygacctctctgargctgccaaccggaacaatgatgcyctgcgccaggcaaagcaggagtcaaatgagtaccggagacaggtgcagtcactyacctgcgaagtggaygcacttaaaggaactaatgagtcyctggaacgccagatgcgtgagatggaagagaattttgcccttgaagctgctaactaccargacactattggccgcctgcaggatgagattcagaacatgaaggaagagatggctcgtcaccttcgtgaataccaagacctgctcaatgtyaagatggctcttgacattgagatwgccacctacaggaagctrctggaaggcgaggagagcaggatttctctgcctcttcccaacttttcttcmctgaacctgagagaractaatctggagtcactccctctggttgacacccactcaaaaagaacactcctgattaagacrgtggaaacyagrgatggacaggtgatcaatgaaacctctcagcatcaygatgaccttgaataaaaattgcacayactcygtgcaacaacgcagtaccagcnnnnnnnnnnnnnnnnnnttygtatcttaaggaaacagctttcaagtgcctttactgcagtttttcaggagcgcaagwwasatttgggatagaaataagctctagtttctaacaactgacaccctaaaagatttagaaaaggtttacaacacaatctagtttacgaagaaatcttgtgctagaatacttttcaaagtattttgaataccattaaacgctttcccagtatacgaccaactgacgcttata

SEQ ID NO: 502 - DNA, 1437 bp, Cricetulus griseus
  misc_feature: Qualifier WAN013I96_at
gtctactgatggtgctgaaggcacctcacagattccagcttcggaacaagagactctggttagaccaaaaccattgtttctgaagttgttaaagtctgtcggggcgcagaaagatacctatactatgaaagagattatattatcttggcagtatataatgactaaacgattatatgatgagaagcagcaacacattgtgtattgttcaaacgatcttctaggagatttgtttggagtgccgagcttctctgtgaaagaccacaggaaaatacatataatgatctacagaaacttggtggtcgtaagccagcaagagactcttcagtccggcacatcagtgagtgagagcagatgtcagcctgaaggaggcagtgaacagaaggatcctgtgcaggagccacaagaagagaagtcgtcttcagattcagtttctagaccatctacctcatctagaaggagaacaattagtgagacagaagaaaatgcagatgagctacctggtgatcgacagaggaagcgccacaggtccctttcctttgatgaaagcctggctttgtgtgtgttaagagagatatgctgtgagagaagcagcagcagcgagtccacagacaccccctcaaatcaggatcttgatgatggtgtaagtgaacactcgggcgattggctggatcaggattcagtttctgatcaattcagtgtagaatttgaagttgagtctcttgactcagaagattacagcctgagtgaaggagggcaggagctctcagatgaggatgacgaggtctatcgagtcacagtgtatcagtcaggagaaagtgatgtagattcctttgaaggagatccggaaatctccttagctgactattggaaatgtacctcctgcaatgaaatgaatcctccccttccacctcwctgcaacagatgttggacyctkcgtgagaaytggcttccrgaagataargrgaargataraggggacrysyctgaagargccaarctggaggcagaaggcttagatgtgtcctgatggggaaaaaagctacakcgartgattstaaggagtcctgcactgaggarartgatgataargaaatatatacctcccagtcrcaagagagtgaggactattcccagccstcaacktccagcagcattgtttatagcagccargaagaygtcaaagagtgggagaaggaagagacascagacaaagargagartgtggaatctggcttctctcttaatgccattgaaccatgtgtgatttgccaagggcggcctaaaaatggttgcattgttcatggcaaaactggacatctcatgtcatgtttcccatgtgcaaagaagctaaaaaaaaggaataagccctgcccagtgtgcagacaaccaatwcaaatgattgtgctaacttattttagctagctgacctgcccataa

SEQ ID NO: 503 - DNA, 908 bp, Cricetulus griseus
  misc_feature: Qualifier WAN013I9K_at
aaagtcacagcttgtccaaagacccgggnnnnnnnnnnnnnnnnnagtctgagaagtccagcaccagcaccagcaccagcaccagcaccagcaccagcaccagcaccagcaccagcaccagcaccagcaccagcaccatgcctatgatactgggatactggaatgtccgcggtctgacaaaccccatccgcctgctcctggaatacacagactcaagctatgaggagaagaaatacaccatgggggacgctcctgactytgacagaagccagtggctgaatgagaagttcaagctgggcctggactttcccaatctgccctacttaattgatgggtcccacaagatcacccagagcaacgccatcctgcgctacattgcccgcaagcacaacctgtgtggagagacagaggaggagaggattcgtgtggacattgtggagaaccaggctatggacacccgcatgcagctcatcatgctctgttacaaccctgactttgagaagcagaagccagagttcttgaagaccatccctgagaagatgaagatgtactctgagttcctgggcaagcggccatggtttgcaggggacaaggtcaccttatgtggatttctcgcttatgatgtccttgatcagtatcaaatgtttgagcccaagtgcctggatcccttcccaaacctgaaggacttcttggcccgctttgagggcctgaagaagatctctgcctacatgaagaccagccgcttcctccggagacctatattttcaaagatggcacagtggagtaacaagtaggcccctgctacacgggctctcatatggaggacctgtgcacactggatcctcctggccctggggacagctggtcttctgcactgctgcctcaaggttctcacttctttctccttt